(19) United States
(12) Patent Application Publication
(10) Pub. No.: US 2025/0206757 A1
(43) Pub. Date: Jun. 26, 2025

(54) 5,8-DIHYDRO-1,7-NAPHTHYRIDINE DERIVATIVES AS GLP-1 AGONISTS FOR THE TREATMENT OF DIABETES

(71) Applicant: GASHERBRUM BIO, INC., South San Francisco, CA (US)

(72) Inventors: Hui LEI, Shanghai (CN); Xichen LIN, Shanghai (CN); Qinghua MENG, Shanghai (CN); Haizhen ZHANG, Shanghai (CN)

(21) Appl. No.: 18/848,072

(22) PCT Filed: Mar. 20, 2023
(86) PCT No.: PCT/CN2023/082518

§ 371 Date: Sep. 17, 2024

(30) Foreign Application Priority Data
Mar. 21, 2022 (WO) PCT/CN2022/082067
Apr. 7, 2022 (WO) PCT/CN2022/085519
Mar. 9, 2023 (WO) PCT/CN2023/080471

(51) Int. Cl. C07D 519/00; A61K 31/4375; A61K 31/444; A61K 31/4709; A61K 31/501; A61K 31/5025; C07D 471/04

(52) U.S. Cl. CPC C07D 519/00; A61K 31/4375; A61K 31/444; A61K 31/4709; A61K 31/501; A61K 31/5025; C07D 471/04

(57) ABSTRACT

The present disclosure relates generally to GLP-1 agonists and pharmaceutical compositions comprising the same, as well as methods for treating a GLP-1 associated disease, disorder, or condition.

The application claims the benefit of International Patent Application Number PCT/CN2022/082067, filed on Mar. 21, 2022, International Patent Application Number PCT/CN2022/085519, filed on Apr. 7, 2022, and International Patent Application Number PCT/CN2023/080471, filed on Mar. 9, 2023, each of which is incorporated herein by reference in its entirety.

### FIELD

This disclosure relates to GLP-1 agonists, pharmaceutical compositions, and methods of use thereof.

### BACKGROUND

Incretin metabolic hormones, including glucagon-like peptide-1 (GLP-1) and glucose-dependent insulinotropic polypeptide (GIP), are important in the regulation of glucose homeostasis. Medicaments targeting this family of intestinal peptides, such as GLP-1 agonists, have been shown to suppress glucagon production, decrease gastric motility, and increase satiety.

Diabetes mellitus refers to a group of metabolic disorders characterized by persistent hyperglycemia. The most common form, type 2 diabetes mellitus (T2DM) is an acquired condition that accounts for more than 90% of diabetes cases. Typical onset occurs in obese or otherwise sedentary adults and begins with insulin resistance. Though lifestyle changes can be useful in management of this disorder, patients with T2DM may be required to take antidiabetic medications, including dipeptidyl peptidase-4 inhibitors, SGLT2 inhibitors, and sulfonylureas, among others.

In healthy individuals, the incretin hormones glucose-dependent insulinotropic polypeptide (GIP) and glucagon-like peptide 1 (GLP-1) provide tandem modulation of insulin secretory response to glucose ingestion. While this incretin effect is significantly diminished (if at all present) in cases of T2DM, GLP-1 retains insulinotropic properties, even as endocrine pancreatic response to GIP is effectively halted. As such, incretin mimetics and other GLP-1-based therapies can help stimulate insulin production in T2DM patients.

### SUMMARY

The present application describes heterocyclic GLP-1 agonists, as well as pharmaceutical compositions comprising the compounds disclosed herein. Also provided are methods for treating GLP-1-associated diseases, disorders, and conditions.

In one aspect, provided are compounds of Formula I:

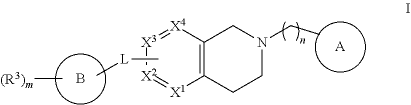

or a pharmaceutically acceptable salt or solvate thereof, wherein:
ring A is

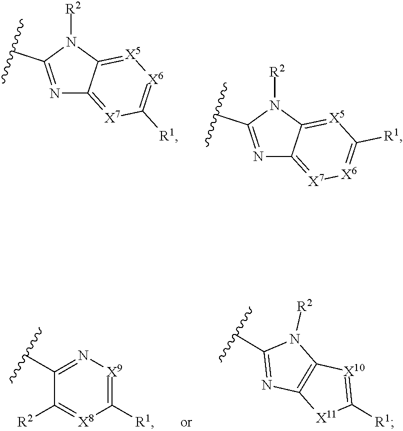

ring B is C₃₋₁₀ cycloalkyl, heterocyclyl, aryl, or heteroaryl;
one of X¹, X², X³, and X⁴ is C covalently bonded to ring B via L; and the remaining of X¹, X², X³, and X⁴ are each independently N or CR⁴; provided that no more than two of X¹, X², X³, and X⁴ are N;
X⁵, X⁶ and X⁷ are each independently N or CR⁵;
X⁸ and X⁹ are each independently N or CR⁶;
X¹⁰ is N or CR⁶ and X¹¹ is S, O, or NR⁹;
n is 1, 2, or 3;
m is 0, 1, 2, 3, 4, or 5;
R¹ is —C(O)OR⁹, —C(O)N(R⁹)₂, —C(O)N(R⁹)S(O)₂R⁹, —NR⁹C(O)R⁹, 5- to 10-membered heterocyclyl or 5- to 10-membered heteroaryl, wherein the 5- to 10-membered heteroaryl or 5- to 10-membered heterocyclyl is optionally substituted with 1-4 R¹¹;
R² is C₁₋₉ alkyl optionally substituted with —O—(C₁₋₉ alkyl), —S—(C₁₋₉ alkyl), —S(O)₂—(C₁₋₉ alkyl), C₃₋₆ cycloalkyl, 3- to 6-membered heterocyclyl, phenyl, or 5- to 6-membered heteroaryl; wherein each C₁₋₉ alkyl, —O—(C₁₋₉ alkyl), —S—(C₁₋₉alkyl), —S(O)₂—(C₁₋₉ alkyl), C₃₋₆ cycloalkyl, 3- to 6-membered heterocyclyl, phenyl, or 5- to 6-membered heteroaryl of R² is further optionally substituted with one to five Z¹;
L is a bond, C₉ alkylene, C₂₋₉ alkenylene, C₂₋₉ alkynylene, —O—C₁₋₉ alkylene, —NR¹⁰—C₁₋₉ alkylene, —C(O)NR¹⁰—C₁₋₉alkylene, —NR¹⁰C(O)—C₁₋₉ alkylene, 3- to 6-membered heterocyclylene, —O—, —S—, —S(O)—, —S(O)₂—, —NR¹⁰—, —C(O)NR¹⁰—, —NR¹⁰C(O)—, —C(O)—, —OC(O)—, —C(O)O—, —NR¹⁰S(O)—, —S(O)NR¹⁰—, —NR¹⁰S(O)NR¹⁰—, —NR¹⁰S(O)₂NR¹⁰—, —NR¹⁰C(O)NR¹⁰—, —OC(O)NR¹⁰—, or —NR¹⁰C(O)O—; wherein each C₁₋₉ alkylene, C₂₋₉ alkenylene, C₂₋₉ alkynylene, —O—C₁₋₉ alkylene, —NR¹⁰—C₁₋₉ alkylene, —C(O)NR¹⁰—C₁₋₉ alkylene, —NR¹⁰C(O)—C₁₋₉ alkylene, or 3- to 6-membered heterocyclylene of L is independently optionally substituted with one to five Z¹;
each R³ is independently halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹⁰C(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R³ is independently optionally substituted with one to five Z¹;
each R⁴ is independently hydrogen, halo, cyano, nitro, oxo, —OR¹⁰, —SR¹⁴, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹⁰C(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R⁴ is independently optionally substituted with one to five Z¹;
each R⁵ is independently hydrogen, halo, cyano, nitro, oxo, —OH, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl of R⁵ is independently optionally substituted with one to five substituents independently selected from halo, hydroxy, and cyano;
each R⁶ is independently hydrogen, halo, cyano, nitro, oxo, —OH, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl of R⁶ is independently optionally substituted with one to five substituents independently selected from halo, hydroxy, and cyano;
each R⁹ is independently hydrogen, C₁₋₉alkyl, C₂₋₉ alkenyl, C₂₋₉ alkynyl, C₃₋₁₀ cycloalkyl, heterocyclyl, aryl, heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R⁹ is independently optionally substituted with one to five R¹¹;
each R¹⁰ is independently hydrogen, C₁₋₉ alkyl, C₂₋₉ alkenyl, C₂₋₉ alkynyl, C₃₋₁₀ cycloalkyl, heterocyclyl, aryl, heteroaryl, —C(O)R²⁰, —C(O)OR²⁰, —C(O)N(R²⁰)₂, —S(O)R²⁰, —S(O)₂R²⁰, —S(O)N(R²⁰)₂, or —S(O)₂N(R²⁰)₂; wherein each C₁₋₉ alkyl, C₂₋₉ alkenyl, C₂₋₉ alkynyl, C₃₋₁₀ cycloalkyl, heterocyclyl, aryl, or heteroaryl of R¹⁰ is independently optionally substituted with one to five Z^{1a};
each R¹¹ is independently oxo, cyano, halo, hydroxy, C₁₋₉ alkyl, C₁₋₉ alkoxy, C₁₋₉ haloalkyl, C₁₋₉ haloalkoxy, C₃₋₉ cycloalkyl, —C₁₋₉ alkyl-C(O)OR¹², —C(O)OR¹², —C(O)N(R¹²)₂, —SR¹², or —S(O)₂R¹²;
each R¹² is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R¹² is independently optionally substituted with one to five Z^{1a};
each Z¹ is independently halo, cyano, nitro, oxo, C₁₋₉ alkyl, C₂₋₉ alkenyl, C₂₋₉ alkynyl, C₃₋₁₀ cycloalkyl, heterocyclyl, aryl, heteroaryl, -L¹-C₁₋₉alkyl, -L¹-C₂₋₉ alkenyl, -L¹-C₂₋₉ alkynyl, -L¹-C₃₋₁₀ cycloalkyl, -L¹-heterocyclyl, -L¹-aryl, or -L¹-heteroaryl; wherein each C₁₋₉alkyl, C₂₋₉ alkenyl, C₂₋₉ alkynyl, C₃₋₁₀ cycloalkyl, heterocyclyl, aryl, or heteroaryl of Z¹ is independently optionally substituted with one to five Z^{1a};
each L¹ is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²⁰—, —S(O)—, —S(O)₂—, —S(O)NR²⁰—, —S(O)₂NR²⁰—, —NR²⁰S(O)—, —NR²⁰S(O)₂—, —NR²⁰S(O)NR²⁰—, or —NR²⁰S(O)₂NR²⁰—;
each R²⁰ is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R²⁰ is independently optionally substituted with one to five Z^{1a};
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₃₋₆ cycloalkyl, heterocyclyl, aryl, or heteroaryl; wherein each C₁₋₆ alkyl, C₂₋₆ alkenyl, C₂₋₆ alkynyl, C₃₋₆ cycloalkyl, heterocyclyl, aryl, or heteroaryl of Z^{1a} is independently optionally substituted with one to five substituents selected from C₁₋₉ alkyl, oxo, halo, hydroxy, and cyano;
provided that when ring A is

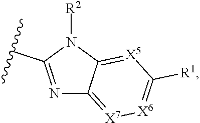

then R¹ is other than —C(O)OH.

Also provided herein are pharmaceutical compositions comprising a compound of Formula I, or a pharmaceutically acceptable salt or solvate thereof, and a pharmaceutically acceptable excipient.

Also provided herein are methods for treating type 2 diabetes mellitus in a patient in need thereof, the methods comprising administering to the patient a therapeutically effective amount of a compound of Formula I, or a pharmaceutically acceptable salt or solvate thereof, or a pharmaceutical composition thereof.

Also provided herein are methods for treating type 2 diabetes mellitus in a patient, the methods comprising administering to a patient identified or diagnosed as having type 2 diabetes mellitus a therapeutically effective amount of a compound of Formula I, or a pharmaceutically acceptable salt or solvate thereof, or a pharmaceutical composition thereof.

Also provided herein are methods for treating diabetes mellitus in a patient, the methods comprising determining that the patient has type 2 diabetes mellitus; and administering to the patient a therapeutically effective amount of a compound of Formula I, or a pharmaceutically acceptable salt or solvate thereof, or a pharmaceutical composition thereof. In some embodiments, the step of determining that the patient has type 2 diabetes mellitus includes performing an assay to determine the level of an analyte in a sample from the patient, wherein the analyte is selected from the group consisting of hemoglobin A1c (HbA1c), fasting plasma glucose, non-fasting plasma glucose, or any combination thereof. In some embodiments, the level of HbA1c is greater than or about 6.5%. In some embodiments, the level of fasting plasma glucose is greater than or about 126 mg/dL. In some embodiments, the level of non-fasting plasma glucose is greater than or about 200 mg/dL.

In some embodiments, the methods further comprise obtaining a sample from the patient. In some embodiments, the sample is a body fluid sample. In some embodiments, the patient is about 40 to about 70 years old and is overweight or obese. In some embodiments, the patient has a body mass index (BMI) greater than or about 22 kg/m². In some embodiments, the patient has a BMI greater than or about 30 kg/m².

In some embodiments, the methods for the treatment of type 2 diabetes mellitus comprise a reduction in fasting plasma glucose levels. In some embodiments, the fasting plasma glucose levels are reduced to about or below 100 mg/dL.

In some embodiments, the methods for the treatment of type 2 diabetes mellitus comprise a reduction in HbA1c levels. In some embodiments, the HbA1c levels are reduced to about or below 5.7%.

In some embodiments, the methods for the treatment of type 2 diabetes mellitus comprise a reduction in glucagon levels.

In some embodiments, the methods for the treatment of type 2 diabetes mellitus comprise an increase in insulin levels.

In some embodiments, the methods for the treatment of type 2 diabetes mellitus comprise a decrease in BMI. In some embodiments, the BMI is decreased to about or below 25 kg/m².

In some embodiments, the compound of Formula I, or a pharmaceutically acceptable salt or solvate thereof, or a pharmaceutical composition thereof, is administered orally.

In some embodiments, the methods of treatment for type 2 diabetes mellitus further comprise administering an additional therapy or therapeutic agent to the patient. In some embodiments, the additional therapy or therapeutic agent is selected from the group consisting of an antidiabetic agent, an anti-obesity agent, a GLP-1 receptor agonist, an agent to treat non-alcoholic steatohepatitis (NASH), anti-emetic agent, gastric electrical stimulation, dietary monitoring, physical activity, or any combinations thereof. In some embodiments, the antidiabetic agent is selected from the group consisting of a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor, or any combinations thereof. In some embodiments, the biguanide is metformin. In some embodiments, the anti-obesity agent is selected from the group consisting of neuropeptide Y receptor type 2 (NPYR2) agonist, a NPYR1 or NPYR5 antagonist, a human proislet peptide (HIP), a cannabinoid receptor type 1 (CB1R) antagonist, a lipase inhibitor, a melanocortin receptor 4 agonist, a farnesoid X receptor (FXR) agonist, phentermine, zonisamide, a norepinephrine/dopamine reuptake inhibitor, a GDF-15 analog, an opioid receptor antagonist, a cholecystokinin agonist, a serotonergic agent, a methionine aminopeptidase 2 (MetAP2) inhibitor, diethylpropion, phendimetrazine, benzphetamine, a fibroblast growth factor receptor (FGFR) modulator, an AMP-activated protein kinase (AMPK) activator, or any combinations thereof. In some embodiments, the GLP-1 receptor agonist is selected from the group consisting of liraglutide, exenatide, dulaglutide, albiglutide, taspoglutide, lixisenatide, semaglutide, or any combinations thereof. In some embodiments, the agent to treat NASH is selected from the group consisting of an FXR agonist, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21) agonist, a niacin analogue, a leukotriene D4 (LTD4) receptor antagonist, an acetyl-CoA carboxylase (ACC) inhibitor, a ketohexokinase (KHK) inhibitor, an ileal bile acid transporter (IBAT) inhibitor, an apoptosis signal-regulating kinase 1 (ASK1) inhibitor, or any combinations thereof. In some embodiments, the compound of Formula I, or a pharmaceutically acceptable salt or solvate thereof, or a pharmaceutical composition thereof, and the additional therapeutic agent are administered as separate dosages sequentially in any order.

Also provided herein are methods for modulating insulin levels in a patient in need of such modulating, the method comprising administering to the patient an effective amount of a compound of Formula I, or a pharmaceutically acceptable salt or solvate thereof, or a pharmaceutical composition thereof. In some embodiments, the modulation results in an increase of insulin levels.

Also provided herein are methods for modulating glucose levels in a patient in need of such modulating, the method comprising administering to the patient an effective amount of a compound of Formula I, or a pharmaceutically acceptable salt or solvate thereof, or a pharmaceutical composition thereof. In some embodiments, the modulation results in a decrease of glucose levels.

Also provided herein are methods for treating a GLP-1 associated disease, disorder, or condition, the method comprising administering to a patient in need thereof an effective amount of a compound of Formula I, or a pharmaceutically acceptable salt or solvate thereof, or a pharmaceutical composition thereof. In some embodiments, the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof. In some embodiments, the disease, disorder, or condition is selected from the group consisting of type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof. In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

Before the present compounds and methods are described, it is to be understood that the disclosure is not limited to the methodologies, protocols, cell lines, assays, and reagents described, as these may vary. It is also to be understood that the terminology used herein is intended to describe embodiments of the present disclosure, and is in no way intended to limit the scope of the present disclosure as set forth in the appended claims.

### Definitions

It must be noted that as used herein, and in the appended claims, the singular forms “a,” “an,” and “the” include plural references unless the context clearly dictates otherwise.

Unless defined otherwise, all technical, and scientific terms used herein have the same meanings as commonly understood by one of ordinary skill in the art to which this disclosure pertains. Although any methods and materials similar or equivalent to those described herein can be used in the practice or testing of the present disclosure, exemplary methods, devices, and materials are now described. All publications cited herein are incorporated herein by reference in their entirety for the purpose of describing and disclosing the methodologies, reagents, and tools reported in the publications that might be used in connection with the disclosure.

Provided herein are heterocyclic GLP-1 agonists for use in the management of T2DM and other conditions where activation of GLP-1 activity is useful.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, the term “halo” or “halogen” means —F (sometimes referred to herein as “fluoro” or “fluoros”), —Cl (sometimes referred to herein as “chloro” or “chloros”), —Br (sometimes referred to herein as “bromo” or “bromos”), and —I (sometimes referred to herein as “iodo” or “iodos”).

As used herein, the term “alkyl” refers to saturated linear or branched-chain monovalent hydrocarbon radicals, containing the indicated number of carbon atoms. For example, “(C₁₋₆)alkyl” refers to saturated linear or branched-chain monovalent hydrocarbon radicals of one to six carbon atoms. Non-limiting examples of alkyl include methyl, ethyl, 1-propyl, isopropyl, 1-butyl, isobutyl, sec-butyl, tert-butyl, 2-methyl-2-propyl, pentyl, neopentyl, and hexyl.

As used herein, the term “alkylene” refers to a divalent alkyl containing the indicated number of carbon atoms. For example, “(C₁₋₃)alkylene” refers to a divalent alkyl having one to three carbon atoms (e.g., —CH₂—, —CH(CH₃)—, —CH₂CH₂—, or —CH₂CH₂CH₂—). Similarly, the terms “cycloalkylene,” “heterocyclylene,” “arylene,” and “heteroarylene” mean divalent cycloalkyl, heterocyclyl, aryl, and heteroaryl, respectively.

As used herein, the term “alkenyl” refers to a linear or branched mono-unsaturated hydrocarbon chain, containing the indicated number of carbon atoms. For example, “(C₂₋₆)alkenyl” refers a linear or branched mono unsaturated hydrocarbon chain of two to six carbon atoms. Non-limiting examples of alkenyl include ethenyl, propenyl, butenyl, or pentenyl.

As used herein, the term “alkynyl” refers to a linear or branched di-unsaturated hydrocarbon chain, containing the indicated number of carbon atoms. For example, “(C₂₋₆)alkynyl” refers to a linear or branched di-unsaturated hydrocarbon chain having two to six carbon atoms. Non-limiting examples of alkynyl include ethynyl, propynyl, butynyl, or pentynyl.

As used herein, the term “cycloalkyl” refers to a saturated or partially unsaturated cyclic hydrocarbon, containing the indicated number of carbon atoms. For example, “(C₃₋₆)cycloalkyl” refers to a saturated or partially unsaturated cyclic hydrocarbon having three to six ring carbon atoms. Non-limiting examples of cycloalkyl include cyclopropyl, cyclobutyl, cyclopentyl, and cyclohexyl. Cycloalkyl may be partially unsaturated. Non-limiting examples of partially unsaturated cycloalkyl include cyclohexenyl, cyclopentenyl, cycloheptenyl, cyclooctenyl, and the like. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

As used herein, the term “heterocyclyl” refers to a mon-, bi-, tri-, or polycyclic nonaromatic ring system containing indicated number of ring atoms (e.g., 3-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, the heteroatoms selected from O, N, S, or S(O)₁₋₂ (e.g., carbon atoms and 1-3, 1-6, or 1-9 heteroatoms of N, O, S, or S(O)₁₋₂ if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl groups may be partially unsaturated. Non-limiting examples of partially unsaturated heterocyclyl include dihydropyrrolyl, dihydropyridinyl, tetrahydropyridinyl, dihydrofuranyl, dihydropyranyl, and the like. Heterocyclyl may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyl include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, the term “aryl” refers to a mono-, bi-, tri- or polycyclic hydrocarbon group containing the indicated numbers of carbon atoms, wherein at least one ring in the system is aromatic (e.g., C₆ monocyclic, C₁₀ bicyclic, or C₁₄ tricyclic aromatic ring system). Examples of aryl groups include phenyl, naphthyl, tetrahydronaphthyl, and the like.

As used herein, the term “heteroaryl” refers to a mono-, bi-, tri- or polycyclic group having indicated numbers of ring atoms (e.g., 5-6 ring atoms; e.g., 5, 6, 9, 10, or 14 ring atoms); wherein at least one ring in the system is aromatic (but does not have to be a ring which contains a heteroatom, e.g. tetrahydroisoquinolinyl, e.g., tetrahydroquinolinyl), and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, tetrazolyl, chromanyl, 2,3-dihydrobenzo[b][1,4]dioxinyl, benzo[d][1,3]dioxolyl, 2,3-dihydrobenzofuranyl, tetrahydroquinolinyl, 2,3-dihydrobenzo[b][1,4]oxathiinyl, isoindolinyl, and others.

As used herein, the term “haloalkyl” refers to an alkyl radical as defined herein, wherein one or more hydrogen atoms is replaced with one or more halogen atoms. Non-limiting examples include fluoromethyl, difluoromethyl, trifluoromethyl, 2-fluoroethyl, 2,2-difluoroethyl, 2,2,2-trifluoroethyl, chloromethyl, dichloromethyl, chloroethyl, trichloroethyl, bromomethyl, and iodomethyl.

As used herein, the term “alkoxy” refers to an —O-alkyl radical, wherein the radical is on the oxygen atom. For example, “C₁₋₆ alkoxy” refers to an —O—(C₁₋₆ alkyl) radical, wherein the radical is on the oxygen atom. Examples of alkoxy include methoxy, ethoxy, propoxy, isopropoxy, butoxy and tert-butoxy. Accordingly, as used herein, the term “haloalkoxy” refers to an —O-haloalkyl radical, wherein the radical is on the oxygen atom.

As used herein, the term “compound,” is meant to include all stereoisomers, geometric isomers, tautomers, and isotopes of the structures depicted. Compounds herein identified by name or structure as one particular tautomeric form are intended to include other tautomeric forms unless otherwise specified.

As used herein, when a ring is described as being “aromatic,” it means the ring has a continuous, delocalized π-electron system. Typically, the number of out of plane π-electrons corresponds to the Hickel rule (4n+2). Examples of such rings include: benzene, pyridine, pyrimidine, pyrazine, pyridazine, pyridone, pyrrole, pyrazole, oxazole, thiazole, isoxazole, isothiazole, and the like. When a ring system comprising at least two rings is described as “aromatic,” it means the ring system comprises one or more aromatic ring(s). Accordingly, when a ring system comprising at least two rings is described as “nonaromatic,” none of the constituent rings of the ring system is aromatic.

As used herein, when a ring is described as being “partially unsaturated,” it means the ring has one or more additional degrees of unsaturation (in addition to the degree of unsaturation attributed to the ring itself; e.g., one or more double bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like. When a ring system comprising at least two rings is described as “partially unsaturated,” it means the ring system comprises one or more partially unsaturated ring(s), provided that none of the constituent rings of the ring system is aromatic.

The term “tautomer” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer.

The term “GLP-1R” or “GLP-1 receptor” as used herein is meant to include, without limitation, nucleic acids, polynucleotides, oligonucleotides, sense and antisense polynucleotide strands, complementary sequences, peptides, polypeptides, proteins, homologous, and/or orthologous GLP-1R molecules, isoforms, precursors, mutants, variants, derivatives, splice variants, alleles, different species, and active fragments thereof.

The term “GLP-1 associated disease” as used herein is meant to include, without limitation, all those diseases, disorders, or conditions in which modulating glucagon-like peptide-1 (GLP-1) receptor signaling can alter the pathology and/or symptoms and/or progression of the disease, disorder, or condition.

The term “GLP-1 agonist” or “GLP-1 RA” as used herein refers to an agonist of the glucagon-like peptide-1 (GLP-1) receptor. GLP-1 RAs enhance glucose-dependent insulin secretion; suppress inappropriately elevated glucagon levels, both in fasting and postprandial states; and slow gastric emptying. Karla et al., Glucagon-like peptide-1 receptor agonists in the treatment of type 2 diabetes: Past, present, and future, Indian J Endocrinol Metab. 2016 March-April; 20(2): 254-267. GLP-1 RAs have been shown to treat type 2 diabetes. Examples of GLP-1 RAs include, but are not limited to, albiglutide (TANZEUM®), dulaglutide (LY2189265, TRULICITY®), efpeglenatide, exenatide (BYETTA®, BYDUREON®, Exendin-4), liraglutide (VICTOZA®, NN2211), lixisenatide (LYXUMIA®), semaglutide (OZEMPIC®), tirzepatide, ZP2929, NNC0113-0987, BPI-3016, and TT401. See, also, for example, additional GLP-1 receptor agonists described in U.S. Pat. Nos. 10,370,426; 10,308,700; 10,259,823; 10,208,019; 9,920,106; 9,839,664; 8,129,343; 8,536,122; 7,919,598; 6,414,126; 6,628,343; and RE45313.

The term “pharmaceutically acceptable” as used herein indicates that the compound, or salt or composition thereof is compatible chemically and/or toxicologically with the other ingredients comprising a formulation and/or the patient being treated therewith.

The term “administration” or “administering” refers to a method of giving a dosage of a compound or pharmaceutical composition to a vertebrate or invertebrate, including a mammal, a bird, a fish, or an amphibian. The method of administration can vary depending on various factors, e.g., the components of the pharmaceutical composition, the site of the disease, and the severity of the disease.

The terms “effective amount” or “effective dosage” or “pharmaceutically effective amount” or “therapeutically effective amount,” as used herein, refer to a sufficient amount of a chemical entity (e.g., a compound of Formula I, or a pharmaceutically acceptable salt or solvate thereof) being administered which will relieve to some extent one or more of the symptoms of the disease or condition being treated, and can include curing the disease. “Curing” means that the symptoms of active disease are eliminated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. In some embodiments, a “therapeutically effective amount” of a compound as provided herein refers to an amount of the compound that is effective as a monotherapy or combination therapy.

The term “excipient” or “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21^{st} ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutical composition” refers to a mixture of a compound of Formula I, or a pharmaceutically acceptable salt or solvate thereof as described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to, rectal, oral, intravenous, aerosol, parenteral, ophthalmic, pulmonary, and topical administration.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “preventing,” as used herein, is the prevention of the onset, recurrence or spread, in whole or in part, of the disease or condition as described herein, or a symptom thereof.

The terms “subject,” “patient” or “individual,” as used herein, are used interchangeably and refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the patient is a human. In some embodiments, the subject has experienced and/or exhibited at least one symptom of the disease, disorder, or condition to be treated and/or prevented.

The terms “treatment regimen” and “dosing regimen” are used interchangeably to refer to the dose and timing of administration of each therapeutic agent in a combination of the disclosure.

The term “combination therapy” as used herein refers to a dosing regimen of two different therapeutically active agents (i.e., the components or combination partners of the combination), wherein the therapeutically active agents are administered together or separately in a manner prescribed by a medical care taker or according to a regulatory agency as defined herein.

The term “modulation,” as used herein, refers to a regulation or an adjustment (e.g., increase or decrease) and can include, for example agonism, partial agonism or antagonism.

It is understood that the substituents as defined herein are not intended to include impermissible substitution patterns (e.g., methyl substituted with 5 fluoro groups or a hydroxy group attached to an ethenylic or acetylenic carbon atom). Such impermissible substitution patterns are well known to the skilled artisan.

### Compounds

Provided herein is a compound of Formula I:

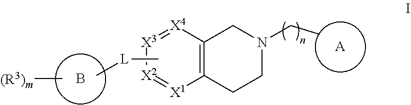

or a pharmaceutically acceptable salt or solvate thereof, wherein:
ring A is

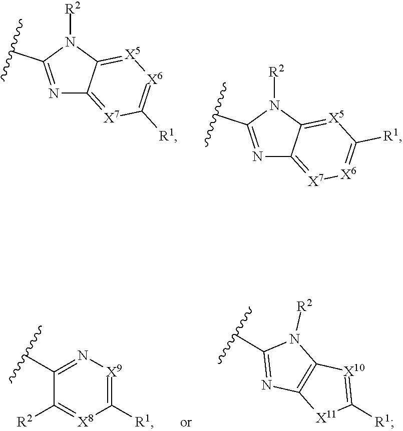

ring B is C₃₋₁₀ cycloalkyl, heterocyclyl, aryl, or heteroaryl;
one of X¹, X², X³, and X⁴ is C covalently bonded to ring B via L; and the remaining of X¹, X², X³, and X⁴ are each independently N or CR⁴; provided that no more than two of X¹, X², X³, and X⁴ are N;
X⁵, X⁶ and X⁷ are each independently N or CR⁵;
X⁸ and X⁹ are each independently N or CR⁶;
X¹⁰ is N or CR⁶ and X¹¹ is S, O, or NR⁹;
n is 1, 2, or 3;
m is 0, 1, 2, 3, 4, or 5;
R¹ is —C(O)OR⁹, —C(O)N(R⁹)₂, —C(O)N(R⁹)S(O)₂R⁹, —NR⁹C(O)R⁹, 5- to 10-membered heterocyclyl or 5- to 10-membered heteroaryl, wherein the 5- to 10-membered heteroaryl or 5- to 10-membered heterocyclyl is optionally substituted with 1-4 R¹¹;
R² is C₁₋₉ alkyl optionally substituted with —O—(C₁₋₉ alkyl), —S—(C₁₋₉ alkyl), —S(O)₂—(C₁₋₉ alkyl), C₃₋₆ cycloalkyl, 3- to 6-membered heterocyclyl, phenyl, or 5- to 6-membered heteroaryl; wherein each C₁₋₉ alkyl, —O—(C₁₋₉ alkyl), —S—(C₁₋₉ alkyl), —S(O)₂—(C₁₋₉ alkyl), C₃₋₆ cycloalkyl, 3- to 6-membered heterocyclyl, phenyl, or 5- to 6-membered heteroaryl of R² is further optionally substituted with one to five Z¹;
L is a bond, C₁₋₉ alkylene, C₂₋₉ alkenylene, C₂₋₉ alkynylene, —O—C₁₋₉ alkylene, —NR¹⁰—C₁₋₉ alkylene, —C(O)NR¹⁰—C₁₋₉alkylene, —NR¹⁰C(O)—C₁₋₉ alkylene, 3- to 6-membered heterocyclylene, —O—, —S—, —S(O)—, —S(O)₂—, —NR¹⁰—, —C(O)NR¹⁰—, —NR¹⁰C(O)—, —C(O)—, —OC(O)—, —C(O)O—, —NR¹⁰S(O)—, —S(O)NR¹⁰—, —NR¹⁰S(O)NR¹⁰—, —NR¹⁰S(O)₂NR¹⁰—, —NR¹⁰C(O)NR¹⁰—, —OC(O)NR¹⁰—, or —NR¹⁰C(O)O—; wherein each C₁₋₉ alkylene, C₂₋₉ alkenylene, C₂₋₉ alkynylene, —O—C₁₋₉ alkylene, —NR¹⁰—C₁₋₉ alkylene, —C(O)NR¹⁰—C₁₋₉ alkylene, —NR¹⁰C(O)—C₁₋₉ alkylene, or 3- to 6-membered heterocyclylene of L is independently optionally substituted with one to five Z¹;
each R³ is independently halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹⁰C(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R³ is independently optionally substituted with one to five Z¹;
each R⁴ is independently hydrogen, halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹⁰C(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R⁴ is independently optionally substituted with one to five Z¹;
each R⁵ is independently hydrogen, halo, cyano, nitro, oxo, —OH, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl of R⁵ is independently optionally substituted with one to five substituents independently selected from halo, hydroxy, and cyano;
each R⁶ is independently hydrogen, halo, cyano, nitro, oxo, —OH, —SH, —NH₂, —NH—C₁₋₆ alkyl, —N(C₁₋₆ alkyl)₂, —S—C₁₋₆ alkyl, C₁₋₆ alkoxy, C₁₋₆, alkyl, C₂₋₆ alkenyl, or C₂₋₆ alkynyl; wherein each —NH—C₁₋₆ alkyl, —N(C₁₋₆ alkyl)₂, —S—C₁₋₆ alkyl, C₁₋₆ alkoxy, C₁₋₆, alkyl, C₂₋₆ alkenyl, or C₂₋₆ alkynyl of R⁶ is independently optionally substituted with one to five substituents independently selected from halo, hydroxy, and cyano;
each R⁸ is independently hydrogen or C₁₋₉ alkyl;
each R⁹ is independently hydrogen, C₁₋₉ alkyl, C₂₋₉ alkenyl, C₂₋₉ alkynyl, C₃₋₁₀ cycloalkyl, heterocyclyl, aryl, heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R⁹ is independently optionally substituted with one to five R¹¹;
each R¹⁰ is independently hydrogen, C₁₋₉ alkyl, C₂₋₉ alkenyl, C₂₋₉ alkynyl, C₃₋₁₀ cycloalkyl, heterocyclyl, aryl, heteroaryl, —C(O)R²⁰, —C(O)OR²⁰, —C(O)N(R²⁰)₂, —S(O)R²⁰, —S(O)₂R²⁰, —S(O)N(R²⁰)₂, or —S(O)₂N(R²⁰)₂; wherein each C₁₋₉ alkyl, C₂₋₉ alkenyl, C₂₋₉ alkynyl, C₃₋₁₀ cycloalkyl, heterocyclyl, aryl, or heteroaryl of R¹⁰ is independently optionally substituted with one to five Z^{1a};
each R¹¹ is independently oxo, cyano, halo, hydroxy, C₁₋₉alkyl, C₁₋₉ alkoxy, C₁₋₉haloalkyl, C₁₋₉ haloalkoxy, C₃₋₉ cycloalkyl, —C₁₋₉alkyl-C(O)OR¹², —C(O)OR², —C(O)N(R¹²)₂, —SR², or —S(O)₂R¹²;
each R¹² is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R¹² is independently optionally substituted with one to five Z^{1a};
each Z¹ is independently halo, cyano, nitro, oxo, C₁₋₉ alkyl, C₂₋₉ alkenyl, C₂₋₉ alkynyl, C₃₋₁₀ cycloalkyl, heterocyclyl, aryl, heteroaryl, -L¹-C₁₋₉ alkyl, -L¹-C₂₋₉ alkenyl, -L¹-C₂₋₉ alkynyl, -L¹-C₃₋₁₀ cycloalkyl, -L¹-heterocyclyl, -L¹-aryl, or -L¹-heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of Z¹ is independently optionally substituted with one to five Z^{1a};
each L¹ is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²⁰—, —S(O)—, —S(O)₂—, —S(O)NR²⁰—, —S(O)₂NR²⁰—, —NR²⁰S(O)—, —NR²⁰S(O)₂—, —NR²⁰S(O)NR²⁰—, or —NR²⁰S(O)₂NR²⁰—;
each R²⁰ is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R²⁰ is independently optionally substituted with one to five Z^{1a};
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₃₋₆ cycloalkyl, heterocyclyl, aryl, or heteroaryl; wherein each C₁₋₆ alkyl, C₂₋₆ alkenyl, C₂₋₆ alkynyl, C₃₋₆ cycloalkyl, heterocyclyl, aryl, or heteroaryl of Z^{1a} is independently optionally substituted with one to five substituents selected from C₁₋₉ alkyl, oxo, halo, hydroxy, and cyano.

In certain embodiments, when ring A is

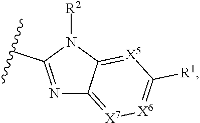

then R¹ is other than —C(O)OH.

In certain embodiments, each R⁸ is independently hydrogen. In certain embodiments, one R⁸ is methyl. In certain embodiments, n is 1. In certain embodiments, n is 1 and R⁸ is hydrogen or methyl. In certain embodiments, n is 1 and R⁸ is hydrogen. In certain embodiments, n is 1 and R⁸ is methyl.

In certain embodiments, provided is a compound of Formula IA:

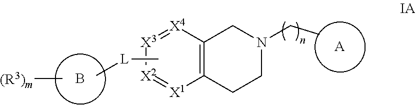

or a pharmaceutically acceptable salt or solvate thereof, wherein each ring A, ring B, X¹, X², X³, X⁴, n, m, L, and R³ are independently as defined herein.

In certain embodiments, provided is a compound of Formula IA:

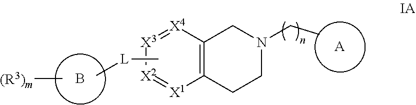

or a pharmaceutically acceptable salt or solvate thereof, wherein:
ring A is

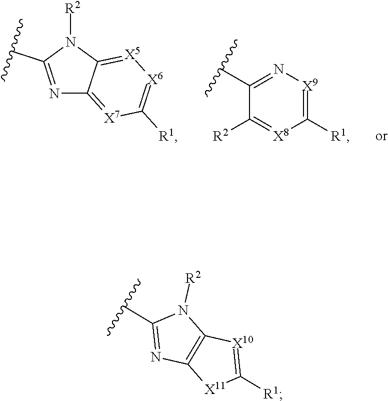

ring B is C₃₋₁₀ cycloalkyl, heterocyclyl, aryl, or heteroaryl;
one of X¹, X², X³, and X⁴ is C covalently bonded to ring B via L; and the remaining of X¹, X², X³, and X⁴ are each independently N or CR⁴; provided that no more than two of X¹, X², X³, and X⁴ are N;
X⁵, X⁶ and X⁷ are each independently N or CR⁵;
X⁸ and X⁹ are each independently N or CR⁶;
X¹⁰ is N or CR⁶ and X¹ is S, O, or NR⁹;
n is 1, 2, or 3;
m is 0, 1, 2, 3, 4, or 5;
R¹ is —C(O)OR⁹, —C(O)N(R⁹)₂, —C(O)N(R⁹)S(O)₂R⁹, —NR⁹C(O)R⁹, 5- to 10-membered heterocyclyl or 5- to 10-membered heteroaryl, wherein the 5- to 10-membered heteroaryl or 5- to 10-membered heterocyclyl is optionally substituted with 1-4 R¹¹;
R² is C₁₋₉ alkyl optionally substituted with —O—(C₁₋₉ alkyl), —S—(C₁₋₉ alkyl), —S(O)₂—(C₁₋₉ alkyl), C₃₋₆ cycloalkyl, 3- to 6-membered heterocyclyl, phenyl, or 5- to 6-membered heteroaryl; wherein each C₁₋₉ alkyl, —O—(C₁₋₉ alkyl), —S—(C₁₋₉alkyl), —S(O)₂—(C₁₋₉ alkyl), C₃₋₆ cycloalkyl, 3- to 6-membered heterocyclyl, phenyl, or 5- to 6-membered heteroaryl of R² is further optionally substituted with one to five Z¹;
L is a bond, C₁₋₉ alkylene, C₂₋₉ alkenylene, C₂₋₉ alkynylene, —O—C₁₋₉ alkylene, —NR¹⁰—C₁₋₉ alkylene, —C(O)NR¹⁴—C₁₋₉ alkylene, —NR¹⁰C(O)—C₁₋₉ alkylene, 3- to 6-membered heterocyclylene, —O—, —S—, —S(O)—, —S(O)₂—, —NR¹⁰—, —C(O)NR¹⁰—, —NR¹⁰C(O)—, —C(O)—, —OC(O)—, —C(O)O—, —NR¹⁰S(O)—, —S(O)NR¹⁰—, —NR¹⁰S(O)NR¹⁰—, —NR¹⁰S(O)₂NR¹⁰—, —NR¹⁰C(O)NR¹⁰—, —OC(O)NR¹⁰—, or —NR¹⁰C(O)O—; wherein each C₁₋₉ alkylene, C₂₋₉ alkenylene, C₂₋₉ alkynylene, —O—C₁₋₉ alkylene, —NR¹⁰—C₁₋₉ alkylene, —C(O)NR¹⁰—C₁₋₉ alkylene, —NR¹⁰C(O)—C₁₋₉ alkylene, or 3- to 6-membered heterocyclylene of L is independently optionally substituted with one to five Z¹;
each R³ is independently halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹⁰C(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R³ is independently optionally substituted with one to five Z¹;
each R⁴ is independently hydrogen, halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹⁰C(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R⁴ is independently optionally substituted with one to five Z¹;
each R⁵ is independently hydrogen, halo, cyano, nitro, oxo, —OH, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl of R is independently optionally substituted with one to five substituents independently selected from halo, hydroxy, and cyano;
each R⁶ is independently hydrogen, halo, cyano, nitro, oxo, —OH, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl of R⁶ is independently optionally substituted with one to five substituents independently selected from halo, hydroxy, and cyano;
each R⁹ is independently hydrogen, C₁₋₉ alkyl, C₂₋₉ alkenyl, C₂₋₉ alkynyl, C₃₋₁₀ cycloalkyl, heterocyclyl, aryl, heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R⁹ is independently optionally substituted with one to five R¹¹;
each R¹⁰ is independently hydrogen, C₁₋₉ alkyl, C₂₋₉ alkenyl, C₂₋₉ alkynyl, C₃₋₁₀ cycloalkyl, heterocyclyl, aryl, heteroaryl, —C(O)R²⁰, —C(O)OR²⁰, —C(O)N(R²⁰)₂, —S(O)R²⁰, —S(O)₂R²⁰, —S(O)N(R²⁰)₂, or —S(O)₂N(R²⁰)₂; wherein each C₁₋₉ alkyl, C₂₋₉ alkenyl, C₂₋₉ alkynyl, C₃₋₁₀ cycloalkyl, heterocyclyl, aryl, or heteroaryl of R¹⁰ is independently optionally substituted with one to five Z^{1a};
each R¹¹ is independently oxo, cyano, halo, hydroxy, C₁₋₉ alkyl, C₁₋₉ alkoxy, C₁₋₉ haloalkyl, C₁₋₉ haloalkoxy, C₃₋₉ cycloalkyl, —C₁₋₉ alkyl-C(O)OR², —C(O)OR², —C(O)N(R¹²)₂, —SR², or —S(O)₂R¹²;
each R¹² is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R¹² is independently optionally substituted with one to five Z^{1a};
each Z¹ is independently halo, cyano, nitro, oxo, C₁₋₉ alkyl, C₂₋₉ alkenyl, C₂₋₉ alkynyl, C₃₋₁₀ cycloalkyl, heterocyclyl, aryl, heteroaryl, -L¹-C₁₋₉ alkyl, -L¹-C₂₋₉ alkenyl, -L¹-C₂₋₉ alkynyl, -L¹-C₃₋₁₀ cycloalkyl, -L¹-heterocyclyl, -L¹-aryl, or -L¹-heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of Z¹ is independently optionally substituted with one to five Z^{1a};
each L¹ is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²⁰—, —S(O)—, —S(O)₂—, —S(O)NR²⁰—, —S(O)₂NR²⁰—, —NR²⁰S(O)—, —NR²⁰S(O)₂—, —NR²⁰S(O)NR²⁰—, or —NR²⁰S(O)₂NR²⁰—;
each R²⁰ is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R²⁰ is independently optionally substituted with one to five Z^{1a};
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₃₋₆ cycloalkyl, heterocyclyl, aryl, or heteroaryl; wherein each C₁₋₆ alkyl, C₂₋₆ alkenyl, C₂₋₆ alkynyl, C₃₋₆ cycloalkyl, heterocyclyl, aryl, or heteroaryl of Z^{1a} is independently optionally substituted with one to five substituents selected from C₁₋₉ alkyl, oxo, halo, hydroxy, and cyano.

Provided herein is a compound of Formula IA:

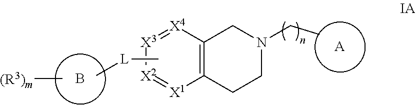

or a pharmaceutically acceptable salt or solvate thereof, wherein:
ring A is

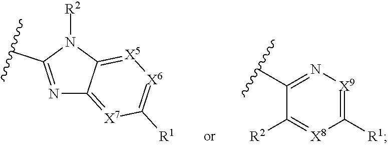

ring B is C₃₋₁₀ cycloalkyl, heterocyclyl, aryl, or heteroaryl;
one of X¹, X², X³, and X⁴ is C covalently bonded to ring B via L; and the remaining of X¹, X², X³, and X⁴ are each independently N or CR⁴; provided that no more than two of X¹, X², X³, and X⁴ are N;
X⁵, X⁶ and X⁷ are each independently N or CR⁵;
X⁸ and X⁹ are each independently N or CR⁶;
n is 1, 2, or 3;
m is 0, 1, 2, 3, 4, or 5;
R¹ is —C(O)OR⁹, —C(O)N(R⁹)₂, —NR⁹C(O)R⁹, 5- to 10-membered heterocyclyl or 5- to 10-membered heteroaryl, wherein the 5- to 10-membered heteroaryl or 5- to 10-membered heterocyclyl is optionally substituted with 1-4 R¹¹;
R² is C₁₋₉ alkyl optionally substituted with —O—(C₁₋₉ alkyl), —S—(C₁₋₉ alkyl), —S(O)₂—(C₁₋₉ alkyl), C₃₋₆ cycloalkyl, 3- to 6-membered heterocyclyl, phenyl, or 5- to 6-membered heteroaryl; wherein each C₁₋₉ alkyl, —O—(C₁₋₉ alkyl), —S—(C₁₋₉ alkyl), —S(O)₂—(C₁₋₉ alkyl), C₃₋₆ cycloalkyl, 3- to 6-membered heterocyclyl, phenyl, or 5- to 6-membered heteroaryl of R² is further optionally substituted with one to five Z¹;
L is a bond, C₁₋₉ alkylene, C₂₋₉ alkenylene, C₂₋₉ alkynylene, —O—C₁₋₉ alkylene, —NR¹⁰—C₁₋₉ alkylene, —C(O)NR¹⁰—C₁₋₉ alkylene, —NR¹⁰C(O)—C₁₋₉ alkylene, 3- to 6-membered heterocyclylene, —O—, —S—, —S(O)—, —S(O)₂—, —NR¹⁰—, —C(O)NR¹⁰—, —NR¹⁰C(O)—, —C(O)—, —OC(O)—, —C(O)O—, —NR¹⁰S(O)—, —S(O)NR¹⁰—, —NR¹⁰S(O)NR¹⁰—, —NR¹⁰S(O)₂NR¹⁰—, —NR¹⁰C(O)NR¹⁰—, —OC(O)NR¹⁰—, or —NR¹⁰C(O)O—; wherein each C₁₋₉ alkylene, C₂₋₉ alkenylene, C₂₋₉ alkynylene, —O—C₁₋₉ alkylene, —NR¹⁰—C₁₋₉ alkylene, —C(O)NR¹⁰—C₁₋₉ alkylene, —NR¹⁰C(O)—C₁₋₉ alkylene, or 3- to 6-membered heterocyclylene of L is independently optionally substituted with one to five Z¹;
each R³ is independently halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹⁰C(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R³ is independently optionally substituted with one to five Z¹;
each R⁴ is independently hydrogen, halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹))₂, —NR¹⁰C(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰—NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R⁴ is independently optionally substituted with one to five Z¹;
each R⁵ is independently hydrogen, halo, cyano, nitro, oxo, —OH, —SH, —NH₂, —NH—C₁₋₆ alkyl, —N(C₁₋₆ alkyl)₂, —S—C₁₋₆ alkyl, C₁₋₆ alkoxy, C₁₋₆, alkyl, C₂₋₆ alkenyl, or C₂₋₆ alkynyl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl of R⁵ is independently optionally substituted with one to five substituents independently selected from halo, hydroxy, and cyano;
each R⁶ is independently hydrogen, halo, cyano, nitro, oxo, —OH, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl of R⁶ is independently optionally substituted with one to five substituents independently selected from halo, hydroxy, and cyano;
each R⁹ is independently hydrogen, C₁₋₉ alkyl, C₂₋₉ alkenyl, C₂₋₉ alkynyl, C₃₋₁₀ cycloalkyl, heterocyclyl, aryl, heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R⁹ is independently optionally substituted with one to five R¹¹;
each R¹⁰ is independently hydrogen, C₁₋₉ alkyl, C₂₋₉ alkenyl, C₂₋₉ alkynyl, C₃₋₁₀ cycloalkyl, heterocyclyl, aryl, heteroaryl, —C(O)R²⁰, —C(O)OR²⁰, —C(O)N(R²⁰)₂, —S(O)R²⁰, —S(O)₂R²⁰, —S(O)N(R²⁰)₂, or —S(O)₂N(R²⁰)₂; wherein each C₁₋₉ alkyl, C₂₋₉ alkenyl, C₂₋₉ alkynyl, C₃₋₁₀ cycloalkyl, heterocyclyl, aryl, or heteroaryl of R¹⁰ is independently optionally substituted with one to five Z^{1a};
each R¹¹ is independently oxo, cyano, halo, hydroxy, C₁₋₉alkyl, C₁₋₉ alkoxy, C₁₋₉haloalkyl, C₁₋₉ haloalkoxy, —C(O)OR¹², —C(O)N(R¹²)₂, or —S(O)₂R¹²;
each R¹² is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R¹² is independently optionally substituted with one to five Z^{1a};
each Z¹ is independently halo, cyano, nitro, oxo, C₁₋₉ alkyl, C₂₋₉ alkenyl, C₂₋₉ alkynyl, C₃₋₁₀ cycloalkyl, heterocyclyl, aryl, heteroaryl, -L¹-C₁₋₉ alkyl, -L¹-C₂₋₉ alkenyl, -L¹-C₂₋₉ alkynyl, -L¹-C₃₋₁₀ cycloalkyl, -L¹-heterocyclyl, -L¹-aryl, or -L¹-heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of Z¹ is independently optionally substituted with one to five Z^{1a};
each L¹ is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²⁰—, —S(O)—, —S(O)₂—, —S(O)NR²⁰—, —S(O)₂NR²⁰—, —NR²⁰S(O)—, —NR²⁰S(O)₂—, —NR²⁰S(O)NR²⁰—, or —NR²⁰S(O)₂NR²⁰—;
each R²⁰ is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R²⁰ is independently optionally substituted with one to five Z^{1a};
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₃₋₆ cycloalkyl, heterocyclyl, aryl, or heteroaryl; wherein each C₁₋₆ alkyl, C₂₋₆ alkenyl, C₂₋₆ alkynyl, C₃₋₆ cycloalkyl, heterocyclyl, aryl, or heteroaryl of Z^{1a} is independently optionally substituted with one to five substituents selected from C₁₋₉ alkyl, oxo, halo, hydroxy, and cyano.

In certain embodiments, provided herein is a compound of Formula II:

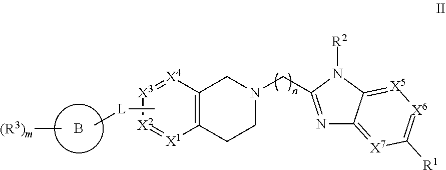

or a pharmaceutically acceptable salt or solvate thereof, wherein R¹, R², R³, X¹, X², X³, X⁴, X⁵, X⁶, X⁷, ring B, m, and n are each independently as defined herein.

In some embodiments, X⁶ is N.

In some embodiments, X⁵ is N.

In some embodiments, X⁵ is N or CR⁵, X⁶ is N, and X⁷ is CR⁵.

In certain embodiments, provided herein is a compound of Formula III:

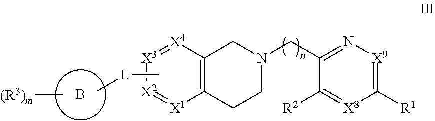

or a pharmaceutically acceptable salt or solvate thereof, wherein R¹, R², R³, X¹, X², X³, X⁴, X⁸, X⁹, ring B, m, and n are each independently as defined herein.

In some embodiments, X⁹ is CR⁶.

In some embodiments, X⁹ is N.

In some embodiments, X⁸ is CR⁶, and X⁹ is N or CR⁶.

In certain embodiments, provided herein is a compound of Formula IV:

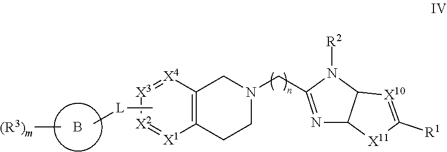

or a pharmaceutically acceptable salt or solvate thereof, wherein R¹, R², R³, X¹, X², X³, X⁴, X¹⁰, X¹¹, ring B, m, and n are each independently as defined herein.

In certain embodiments, provided herein is a compound of Formula V:

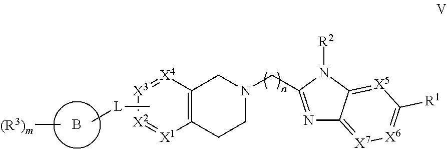

or a pharmaceutically acceptable salt or solvate thereof, wherein R¹, R², R³, X¹, X², X³, X⁴, X⁵, X⁶, X⁷, ring B, m, and n are each independently as defined herein.

In some embodiments, X¹⁰ is CR⁷.

In some embodiments, X¹¹ is S.

In some embodiments, n is 1.

In some embodiments, R¹ is 5-membered heteroaryl, optionally substituted with one to five R¹¹.

In some embodiments, R¹ is

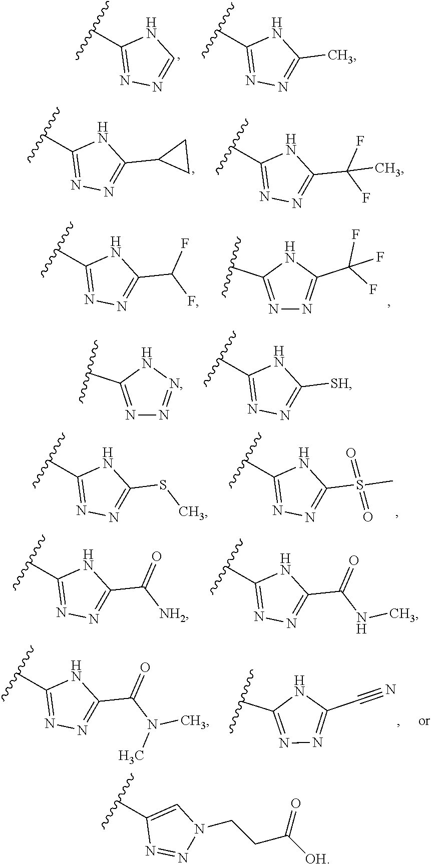

In some embodiments, R¹ is

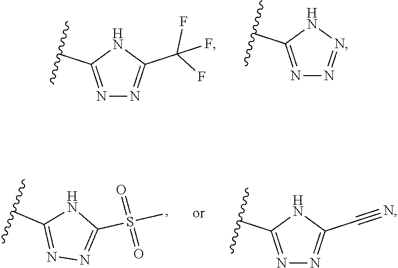

In some embodiments, R¹ is 5- to 10-membered heterocyclyl optionally substituted with one to five R¹¹, wherein the heterocyclyl comprises an endocyclic

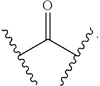 [SMILES: *C(*)=O]

In some embodiments, R¹ is

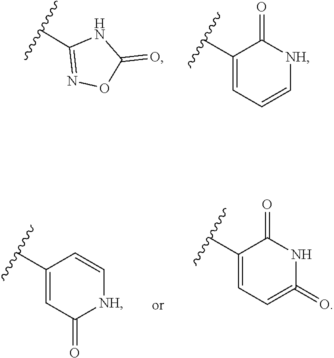

In some embodiments, R¹ is —C(O)OR⁹, —C(O)N(R⁹)₂, —C(O)N(R⁹)S(O)₂R⁹, or —NR⁹C(O)R⁹.

In some embodiments, R¹ is —C(O)OR⁹, —C(O)NHR⁹, —C(O)NHS(O)₂R⁹, or —NHC(O)R⁹.

In some embodiments, R¹ is —C(O)OH.

In some embodiments, R¹ is —C(O)NH₂.

In some embodiments, R¹ is —C(O)NHR⁹, or —NHC(O)R⁹, and R⁹ is Cig alkyl, C₃₋₁₀ cycloalkyl, or heteroaryl; wherein the C₁₋₉ alkyl, C₃₋₁₀ cycloalkyl, or heteroaryl is independently optionally substituted with one to five R¹¹.

In some embodiments, R¹ is —C(O)NHR⁹, or —NHC(O)R⁹, and R⁹ is methyl, 2,2,2-trifluoroethyl, cyclopropyl substituted with cyano, or pyridyl. In some embodiments, R¹ is —C(O)NHR⁹, and R⁹ is methyl, 2,2,2-trifluoroethyl, cyclopropyl substituted with cyano, or pyridyl. In some embodiments, R¹ is —NHC(O)R⁹, and R⁹ is methyl, 2,2,2-trifluoroethyl, cyclopropyl substituted with cyano, or pyridyl.

In some embodiments, R¹ is —C(O)NHS(O)₂R⁹, and R⁹ is methyl.

In some embodiments, one of X¹, X², and X³ is C covalently bonded to ring B via L; X⁴ is N; and the remaining of X¹, X², and X³, are each independently CR⁴.

In some embodiments, one of X¹, X², X³, and X⁴ is C covalently bonded to ring B via L; and the remaining of X¹, X², X³, and X⁴ are each independently CR⁴.

In some embodiments, X³ is C covalently bonded to ring B via L.

In some embodiments, each R⁴ is independently hydrogen, halo, C₁₋₉ haloalkyl, or C₃₋₁₀ cycloalkyl.

In some embodiments, X² is CR⁴, and R⁴ is hydrogen, halo, C₁₋₉ haloalkyl, or C₃₋₁₀ cycloalkyl.

In some embodiments, each R⁴ is independently hydrogen, chloro, —CF₃, or cyclopropyl.

In some embodiments, X¹ is CH.

In some embodiments, L is —O—C₁₋₉ alkylene, —NR¹⁰—C₁₋₉ alkylene, —C(O)NR¹⁰—C₁₋₉ alkylene, or —NR¹⁰C(O)—C₁₋₉ alkylene.

In some embodiments, L is a bond, C₁₋₉ alkylene, —O—C₁₋₉ alkylene, —NH—C₁₋₉ alkylene, —C(O)NH—C₁₋₉ alkylene, 3- to 6-membered heterocyclylene, or —O—.

In some embodiments, L is a bond, —CH₂—, —O—CH₂—, —O—C(CH₃)H—, —NH—CH₂—, —C(O)NH—CH₂—, or pyrrolidinyl.

In some embodiments, L is —O—CH₂—.

In some embodiments, ring B is C₃₋₆ cycloalkyl, phenyl, a 5- or 9-membered heterocyclyl, or a 5- or 9-membered heteroaryl. In some embodiments, ring B is C₃₋₆ cycloalkyl. In some embodiments, ring B is a 5- or 9-membered heterocyclyl. In some embodiments, ring B is a 5- or 9-membered heteroaryl. In some embodiments, ring B is phenyl.

In some embodiments, ring B is phenyl, thiophenyl, thiazolyl, pyridinyl, pyrazinyl, pyrimidinyl, cyclopropyl, 2,3-dihydrobenzofuranyl, benzo[d][1,3]dioxolyl, or benzofuranyl. In some embodiments, ring B is phenyl. In some embodiments, ring B is thiophenyl. In some embodiments, ring B is thiazolyl. In some embodiments, ring B is pyridinyl. In some embodiments, ring B is pyrazinyl. In some embodiments, ring B is pyrimidinyl. In some embodiments, ring B is cyclopropyl. In some embodiments, ring B is 2,3-dihydrobenzofuranyl. In some embodiments, ring B is benzo[d][1,3]dioxolyl. In some embodiments, ring B is benzofuranyl.

In some embodiments, R² is C₁₋₉ alkyl, C₁₋₉ alkyl substituted with a 3- to 6-membered heterocyclyl, or C₁₋₉ alkyl substituted with a C₃₋₆ cycloalkyl which is substituted with cyano.

In some embodiments, R² is C₁₋₉ alkyl.

In some embodiments, R² is methyl.

In some embodiments, R² is C₁₋₉ alkyl substituted with 3- to 6-membered heterocyclyl or C₁₋₉ alkyl substituted with a C₃₋₆ cycloalkyl which is substituted with cyano. In some embodiments, R² is C₁₋₉ alkyl substituted with 3- to 6-membered heterocyclyl. In some embodiments, R² is C₁₋₉ alkyl substituted with a C₃₋₆ cycloalkyl which is substituted with cyano.

In some embodiments, R² is

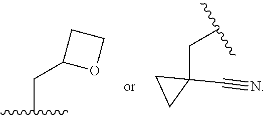 [SMILES: *CC1CCO1 ; *CC1(C#N)CC1]

In some embodiments, R² is

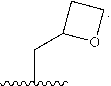 [SMILES: *CC1CCO1]

In some embodiments, R² is

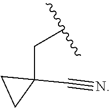 [SMILES: *CC1(C#N)CC1]

In some embodiments, m is 1, 2, or 3.

In some embodiments, each R³ is independently halo, cyano, —OR¹⁰, —C(O)N(R¹⁰)₂, —S(O)₂R¹⁰, C₁₋₉ alkyl, C₃₋₁₀ cycloalkyl, or heteroaryl; wherein each C₁₋₉ alkyl of R³ is independently optionally substituted with one to five halo.

In some embodiments, each R³ is independently halo.

In some embodiments, Ring B is phenyl, m is 2 or 3, and each R³ is independently halo.

In some embodiments, the moiety

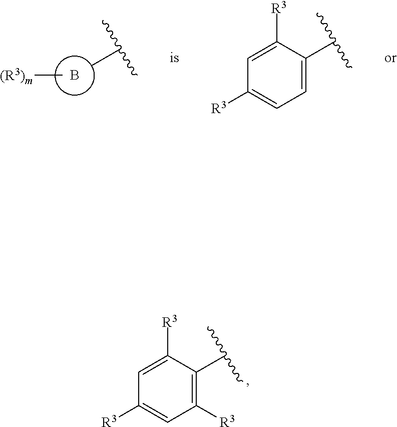

where the wavy line indicates the attachment to L, and each R³ is independently as defined herein. In some embodiments, each R³ is independently halo.

In certain embodiments, provided herein is a compound of Formula IIA:

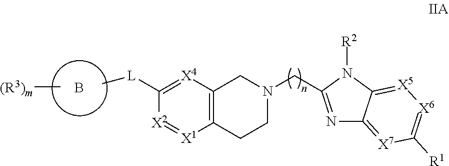

or a pharmaceutically acceptable salt or solvate thereof, wherein Ring B, m, n, L, X¹, X², X⁴, X⁵, X⁶, X⁷, R¹, R², and R³ are each independently as defined herein.

In certain embodiments, provided herein is a compound of Formula IIB:

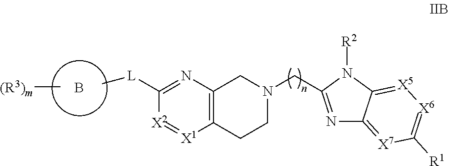

or a pharmaceutically acceptable salt or solvate thereof, wherein Ring B, m, n, L, X¹, X², X⁵, X⁶, X⁷, R¹, R², and R³ are each independently as defined herein.

In certain embodiments, provided herein is a compound of Formula IIC:

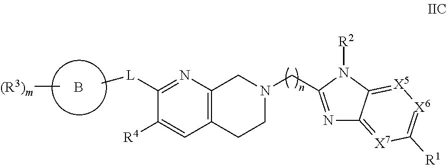

or a pharmaceutically acceptable salt or solvate thereof, wherein Ring B, m, n, L, R⁴, X⁵, X⁶, X⁷, R¹, R², and R³ are each independently as defined herein.

In certain embodiments, provided herein is a compound of Formula IID:

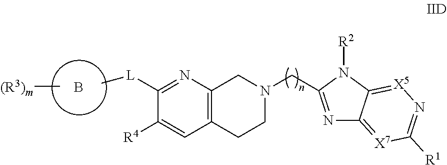

or a pharmaceutically acceptable salt or solvate thereof, wherein Ring B, m, n, L, R⁴, X⁵, X⁷, R¹, R², and R³ are each independently as defined herein.

In certain embodiments, provided herein is a compound of Formula IIE:

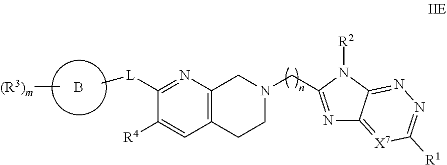

or a pharmaceutically acceptable salt or solvate thereof, wherein Ring B, m, n, L, R⁴, X⁷, R¹, R², and R³ are each independently as defined herein.

In certain embodiments, provided herein is a compound of Formula IIF:

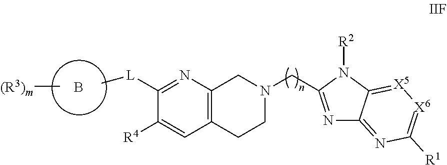

or a pharmaceutically acceptable salt or solvate thereof, wherein Ring B, m, n, L, R⁴, X⁵, X⁶, R¹, R², and R³ are each independently as defined herein.

In certain embodiments, provided herein is a compound of Formula IIG:

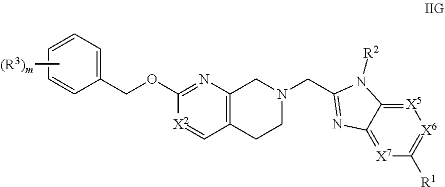

or a pharmaceutically acceptable salt or solvate thereof, wherein m, X², X⁵, X⁶, X⁷, R¹, R², and R³ are each independently as defined herein.

In certain embodiments, provided herein is a compound of Formula IIH:

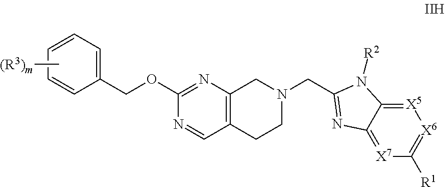

or a pharmaceutically acceptable salt or solvate thereof, wherein m, X⁵, X⁶, X⁷, R¹, R², and R³ are each independently as defined herein.

In certain embodiments, provided herein is a compound of Formula IIJ:

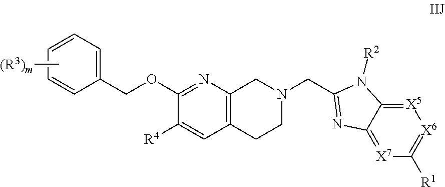

or a pharmaceutically acceptable salt or solvate thereof, wherein m, R⁴, X⁵, X⁶, X⁷, R¹, R², and R³ are each independently as defined herein.

In certain embodiments, provided herein is a compound of Formula IIK:

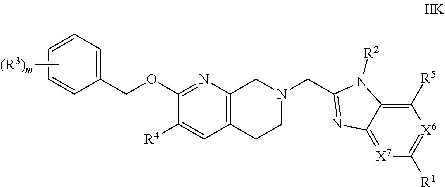

or a pharmaceutically acceptable salt or solvate thereof, wherein each m, R⁴, R⁵, R¹, R², and R³ are independently as defined herein.

In certain embodiments, provided herein is a compound of Formula IIL:

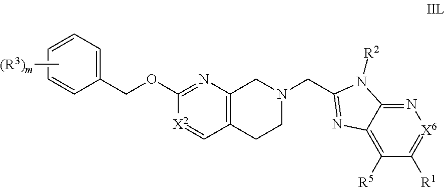

or a pharmaceutically acceptable salt or solvate thereof, wherein m, R⁴, R⁵, R¹, R², and R³ are each independently as defined herein.

In certain embodiments, provided herein is a compound of Formula IIM:

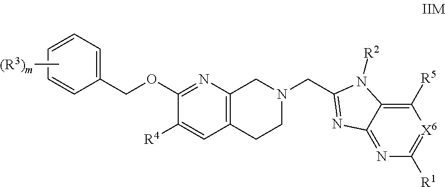

or a pharmaceutically acceptable salt or solvate thereof, wherein m, R⁴, R⁵, R¹, R², and R³ are each independently as defined herein.

In certain embodiments, provided herein is a compound of Formula IIN:

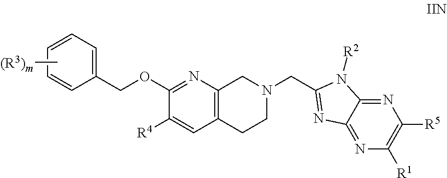

or a pharmaceutically acceptable salt or solvate thereof, wherein m, R⁴, R⁵, R¹, R², and R³ are each independently as defined herein.

In certain embodiments, provided herein is a compound of Formula IIO:

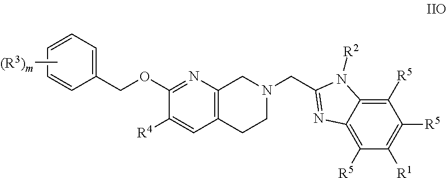

or a pharmaceutically acceptable salt or solvate thereof, wherein each m, R⁴, R⁵, R¹, R², and R³ are independently as defined herein

In certain embodiments, provided herein is a compound of Formula IIP:

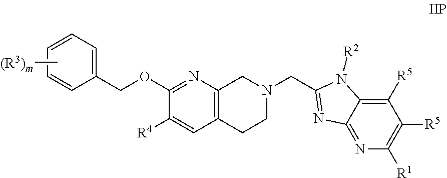

or a pharmaceutically acceptable salt or solvate thereof, wherein each m, R⁴, R⁵, R¹, R², and R³ are independently as defined herein.

In some embodiments, each R⁵ is independently hydrogen or halo.

In certain embodiments, provided herein is a compound of Formula IIIA:

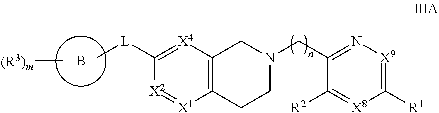

or a pharmaceutically acceptable salt or solvate thereof, wherein Ring B, m, n, L, X¹, X², X⁴, X⁸, X⁹, R¹, R², and R³ are each independently as defined herein.

In certain embodiments, provided herein is a compound of Formula IIIB:

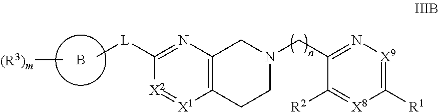

or a pharmaceutically acceptable salt or solvate thereof, wherein each R², R³, R⁴, ring B, m, L, X⁵, and X⁶ are independently as defined herein.

In certain embodiments, provided herein is a compound of Formula IIIC:

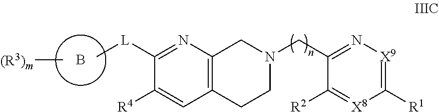

or a pharmaceutically acceptable salt or solvate thereof, wherein Ring B, m, n, L, R⁴, X⁸, X⁹, R¹, R², and R³ are each independently as defined herein.

In certain embodiments, provided herein is a compound of Formula IIID:

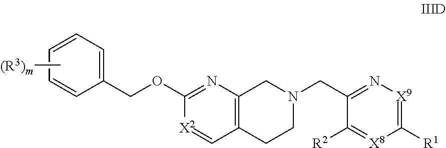

or a pharmaceutically acceptable salt or solvate thereof, wherein m, X², X⁸, X⁹, R¹, R², and R³ are each independently as defined herein.

In certain embodiments, provided herein is a compound of Formula IIIE:

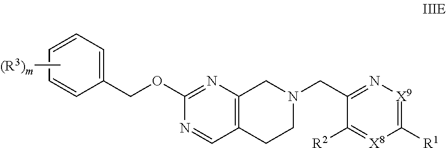

or a pharmaceutically acceptable salt or solvate thereof, wherein m, X⁸, X⁹, R¹, R², and R³ are each independently as defined herein.

In certain embodiments, provided herein is a compound of Formula IIIF:

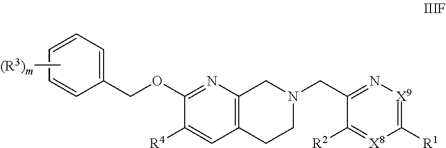

or a pharmaceutically acceptable salt or solvate thereof, wherein m, R⁴, X⁸, X⁹, R¹, R², and R³ are each independently as defined herein.

In certain embodiments, provided herein is a compound of Formula IIIG:

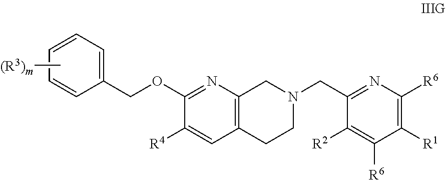

or a pharmaceutically acceptable salt or solvate thereof, wherein each m, R⁴, R⁶, X⁸, X⁹, R¹, R², and R³ are independently as defined herein.

In certain embodiments, provided herein is a compound of Formula IIIH:

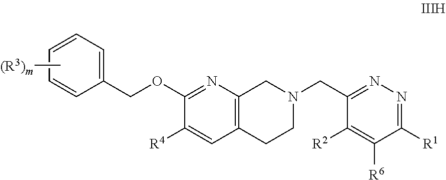

or a pharmaceutically acceptable salt or solvate thereof, wherein m, R⁴, R⁶, R¹, R², and R³ are each independently as defined herein.

In certain embodiments, provided herein is a compound of Formula IIIJ:

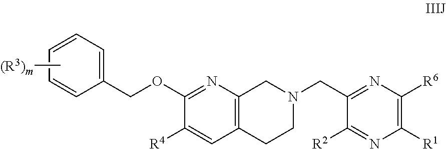

or a pharmaceutically acceptable salt or solvate thereof, wherein m, R⁴, R⁶, R¹, R², and R³ are each independently as defined herein.

In certain embodiments, provided herein is a compound of Formula IVA:

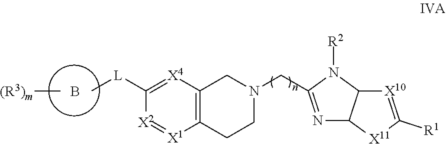

or a pharmaceutically acceptable salt or solvate thereof, wherein Ring B, n, m, L, X¹, X², X⁴, X¹⁰, X¹¹, R¹, R², and R³ are each independently as defined herein.

In certain embodiments, provided herein is a compound of Formula IVB:

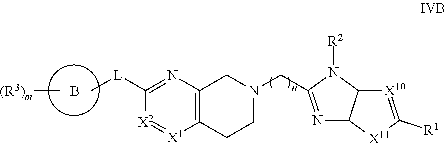

or a pharmaceutically acceptable salt or solvate thereof, wherein Ring B, n, m, L, X¹, X², X¹⁰, X¹¹, R¹, R², and R³ are each independently as defined herein.

In certain embodiments, provided herein is a compound of Formula IVC:

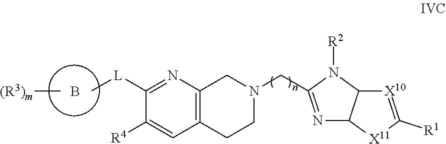

or a pharmaceutically acceptable salt or solvate thereof, wherein Ring B, n, m, L, X¹⁰, X¹¹, R⁴, R¹, R², and R³ are each independently as defined herein.

In certain embodiments, provided herein is a compound of Formula IVD:

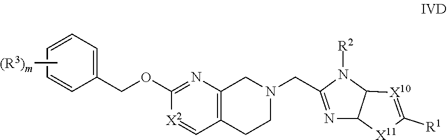

or a pharmaceutically acceptable salt or solvate thereof, wherein m, X², X¹⁰, X¹¹, R¹, R², and R³ are each independently as defined herein.

In certain embodiments, provided herein is a compound of Formula IVE:

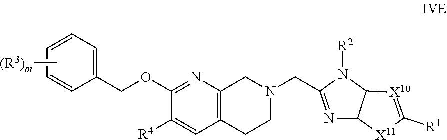

or a pharmaceutically acceptable salt or solvate thereof, wherein m, X¹⁰, X¹¹, R⁴, R¹, R², and R³ are each independently as defined herein.

In certain embodiments, provided herein is a compound of Formula IVF:

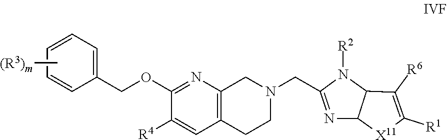

or a pharmaceutically acceptable salt or solvate thereof, wherein m, X¹¹, R⁴, R⁶, R¹, R², and R³ are each independently as defined herein.

In certain embodiments, provided herein is a compound of Formula IVG:

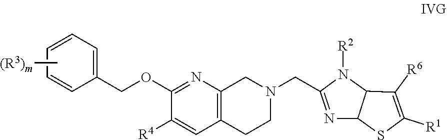

or a pharmaceutically acceptable salt or solvate thereof, wherein m, R⁴, R⁶, R¹, R², and R³ are each independently as defined herein.

In certain embodiments, provided herein is a compound of Formula VA:

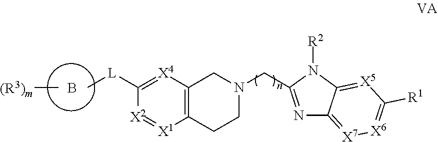

or a pharmaceutically acceptable salt or solvate thereof, wherein Ring B, n, m, L, X¹, X², X⁴, X⁵, X⁶, X⁷, R¹, R², and R³ are each independently as defined herein.

In certain embodiments, provided herein is a compound of Formula VB:

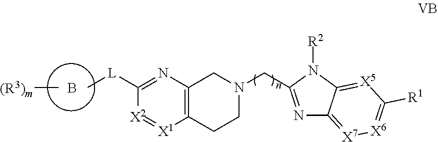

or a pharmaceutically acceptable salt or solvate thereof, wherein Ring B, n, m, L, X¹, X², X⁵, X⁶, X⁷, R¹, R², and R³ are each independently as defined herein.

In certain embodiments, provided herein is a compound of Formula VC:

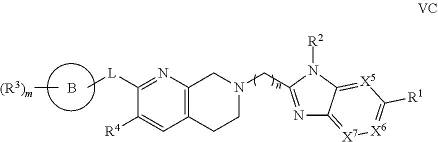

or a pharmaceutically acceptable salt or solvate thereof, wherein Ring B, n, m, L, X⁵, X⁶, X⁷, R⁴, R¹, R², and R³ are each independently as defined herein.

In certain embodiments, provided herein is a compound of Formula VD:

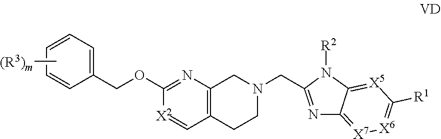

or a pharmaceutically acceptable salt or solvate thereof, wherein m, X², X⁵, X⁶, X⁷, R¹, R², and R³ are each independently as defined herein.

In certain embodiments, provided herein is a compound of Formula VE:

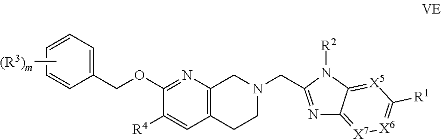

or a pharmaceutically acceptable salt or solvate thereof, wherein m, X⁵, X⁶, X⁷, R⁴, R¹, R², and R³ are each independently as defined herein.

In certain embodiments, provided herein is a compound of Formula VF:

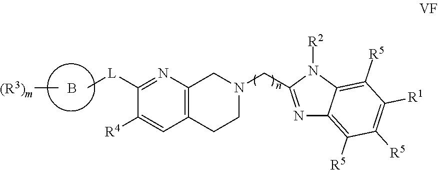

or a pharmaceutically acceptable salt or solvate thereof, wherein m, R⁴, R⁵, R¹, R², and R³ are each independently as defined herein.

In certain embodiments, provided herein is a compound of Formula VG:

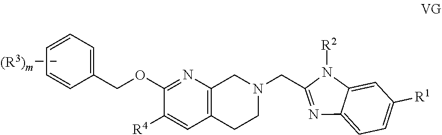

or a pharmaceutically acceptable salt or solvate thereof, wherein m, R⁴, R¹, R², and R³ are each independently as defined herein.

In some embodiments, each R⁶ is independently hydrogen or halo. In some embodiments, each R⁶ is hydrogen.

In certain embodiments, R⁹ is other than hydrogen. In certain embodiments, R¹ is other than —C(O)OH.

In certain embodiments, provided is a compound selected from Table 1, or a pharmaceutically acceptable salt or solvate thereof:

TABLE 1

| Compound No. | Structure |
|---|---|
| 1 | 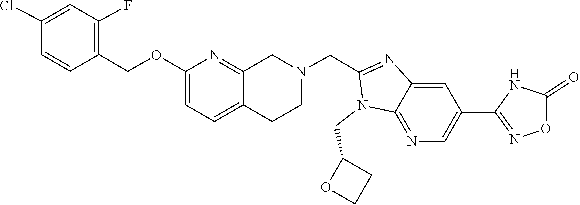 [SMILES: O=c1[nH]c(-c2cnc3c(c2)nc(CN2CCc4ccc(OCc5ccc(Cl)cc5F)nc4C2)n3C[C@@H]2CCO2)no1] |
| 2 | 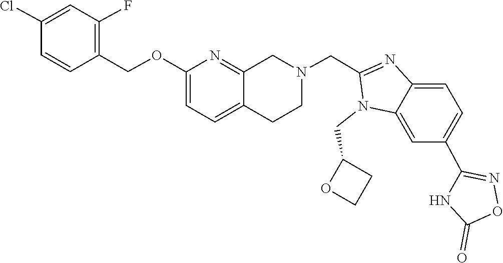 [SMILES: O=c1[nH]c(-c2ccc3nc(CN4CCc5ccc(OCc6ccc(Cl)cc6F)nc5C4)n(C[C@@H]4CCO4)c3c2)no1] |
| 3 | 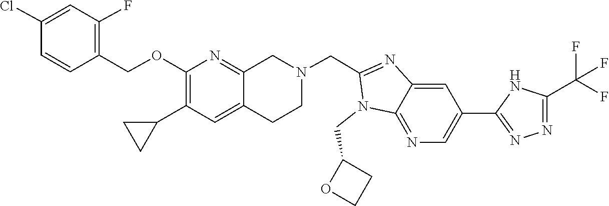 [SMILES: Fc1cc(Cl)ccc1COc1nc2c(cc1C1CC1)CCN(Cc1nc3cc(-c4nnc(C(F)(F)F)[nH]4)cnc3n1C[C@@H]1CCO1)C2] |
| 4 | 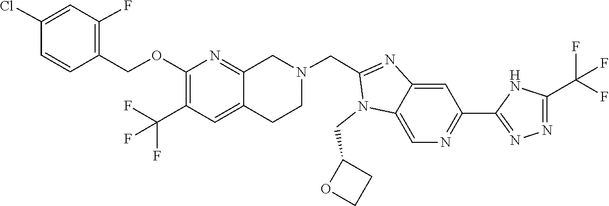 [SMILES: Fc1cc(Cl)ccc1COc1nc2c(cc1C(F)(F)F)CCN(Cc1nc3cc(-c4nnc(C(F)(F)F)[nH]4)ncc3n1C[C@@H]1CCO1)C2] |
| 5 | 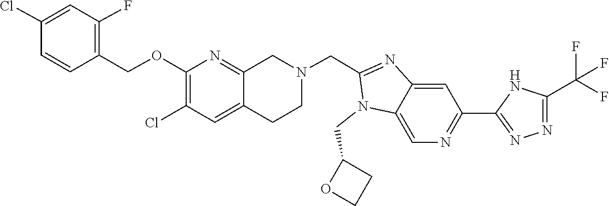 [SMILES: Fc1cc(Cl)ccc1COc1nc2c(cc1Cl)CCN(Cc1nc3cc(-c4nnc(C(F)(F)F)[nH]4)ncc3n1C[C@@H]1CCO1)C2] |
| 6 | 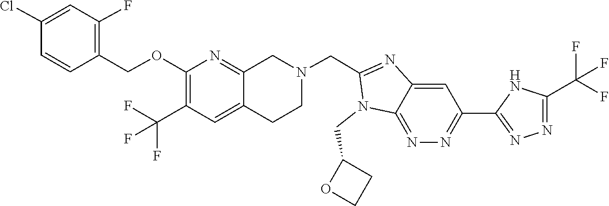 [SMILES: Fc1cc(Cl)ccc1COc1nc2c(cc1C(F)(F)F)CCN(Cc1nc3cc(-c4nnc(C(F)(F)F)[nH]4)nnc3n1C[C@@H]1CCO1)C2] |
| 7 | 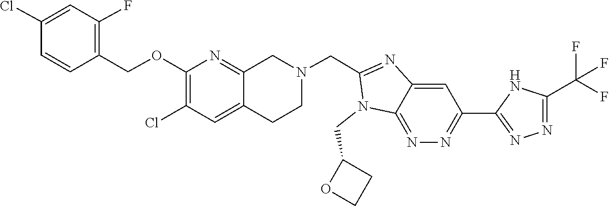 [SMILES: Fc1cc(Cl)ccc1COc1nc2c(cc1Cl)CCN(Cc1nc3cc(-c4nnc(C(F)(F)F)[nH]4)nnc3n1C[C@@H]1CCO1)C2] |
| 8 | 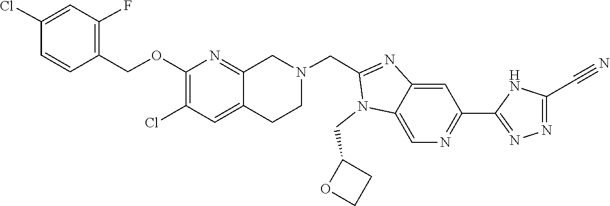 [SMILES: N#Cc1nnc(-c2cc3nc(CN4CCc5cc(Cl)c(OCc6ccc(Cl)cc6F)nc5C4)n(C[C@@H]4CCO4)c3cn2)[nH]1] |
| 9 | 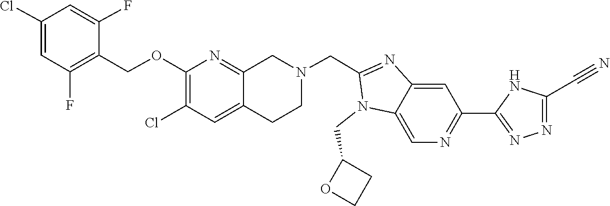 [SMILES: N#Cc1nnc(-c2cc3nc(CN4CCc5cc(Cl)c(OCc6c(F)cc(Cl)cc6F)nc5C4)n(C[C@@H]4CCO4)c3cn2)[nH]1] |
| 10 | 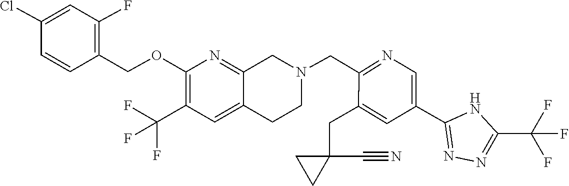 [SMILES: N#CC1(Cc2cc(-c3nnc(C(F)(F)F)[nH]3)cnc2CN2CCc3cc(C(F)(F)F)c(OCc4ccc(Cl)cc4F)nc3C2)CC1] |
| 11 | 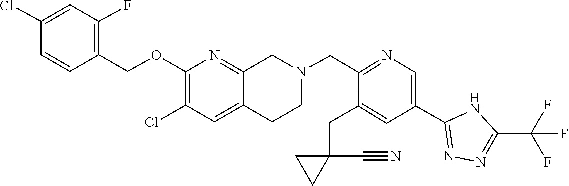 [SMILES: N#CC1(Cc2cc(-c3nnc(C(F)(F)F)[nH]3)cnc2CN2CCc3cc(Cl)c(OCc4ccc(Cl)cc4F)nc3C2)CC1] |
| 12 | 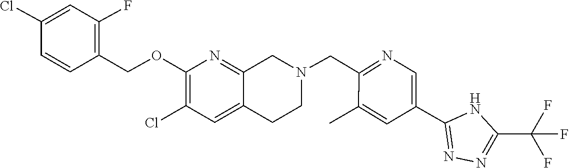 [SMILES: Cc1cc(-c2nnc(C(F)(F)F)[nH]2)cnc1CN1CCc2cc(Cl)c(OCc3ccc(Cl)cc3F)nc2C1] |
| 13 | 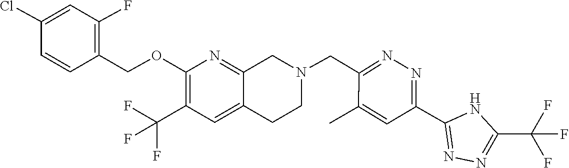 [SMILES: Cc1cc(-c2nnc(C(F)(F)F)[nH]2)nnc1CN1CCc2cc(C(F)(F)F)c(OCc3ccc(Cl)cc3F)nc2C1] |
| 14 | 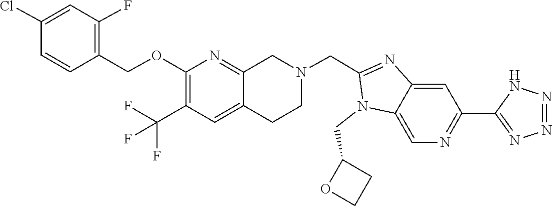 [SMILES: Fc1cc(Cl)ccc1COc1nc2c(cc1C(F)(F)F)CCN(Cc1nc3cc(-c4nnn[nH]4)ncc3n1C[C@@H]1CCO1)C2] |
| 15 | 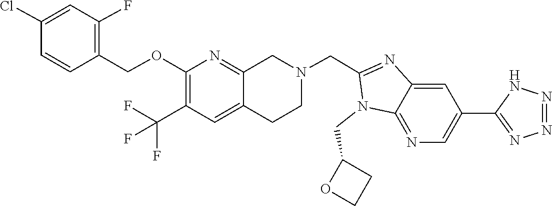 [SMILES: Fc1cc(Cl)ccc1COc1nc2c(cc1C(F)(F)F)CCN(Cc1nc3cc(-c4nnn[nH]4)cnc3n1C[C@@H]1CCO1)C2] |
| 16 | 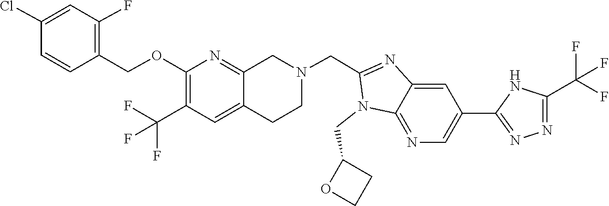 [SMILES: Fc1cc(Cl)ccc1COc1nc2c(cc1C(F)(F)F)CCN(Cc1nc3cc(-c4nnc(C(F)(F)F)[nH]4)cnc3n1C[C@@H]1CCO1)C2] |
| 17 | 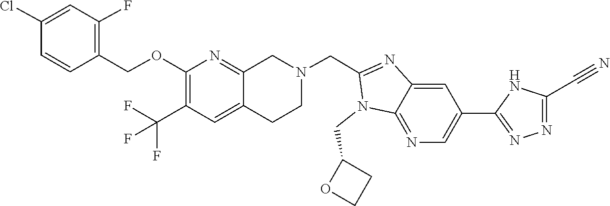 [SMILES: N#Cc1nnc(-c2cnc3c(c2)nc(CN2CCc4cc(C(F)(F)F)c(OCc5ccc(Cl)cc5F)nc4C2)n3C[C@@H]2CCO2)[nH]1] |
| 18 | 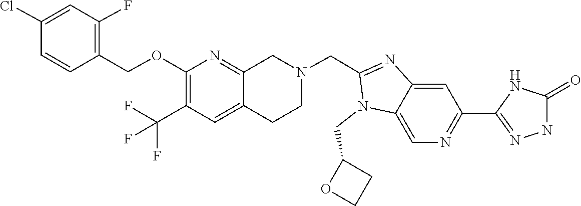 [SMILES: O=c1[nH]nc(-c2cc3nc(CN4CCc5cc(C(F)(F)F)c(OCc6ccc(Cl)cc6F)nc5C4)n(C[C@@H]4CCO4)c3cn2)[nH]1] |
| 19 | 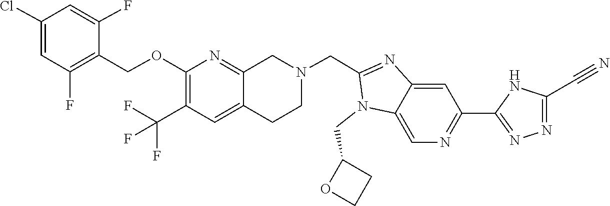 [SMILES: N#Cc1nnc(-c2cc3nc(CN4CCc5cc(C(F)(F)F)c(OCc6c(F)cc(Cl)cc6F)nc5C4)n(C[C@@H]4CCO4)c3cn2)[nH]1] |
| 20 | 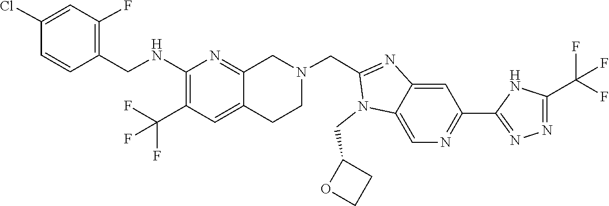 [SMILES: Fc1cc(Cl)ccc1CNc1nc2c(cc1C(F)(F)F)CCN(Cc1nc3cc(-c4nnc(C(F)(F)F)[nH]4)ncc3n1C[C@@H]1CCO1)C2] |
| 21 | 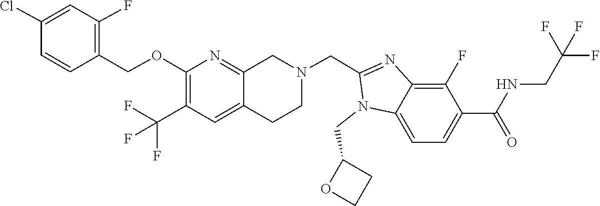 [SMILES: O=C(NCC(F)(F)F)c1ccc2c(nc(CN3CCc4cc(C(F)(F)F)c(OCc5ccc(Cl)cc5F)nc4C3)n2C[C@@H]2CCO2)c1F] |
| 22 | 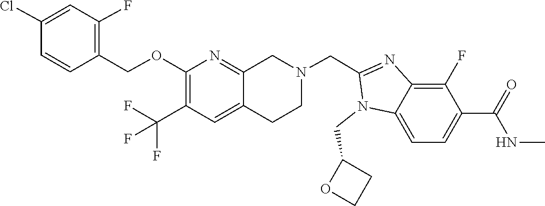 [SMILES: CNC(=O)c1ccc2c(nc(CN3CCc4cc(C(F)(F)F)c(OCc5ccc(Cl)cc5F)nc4C3)n2C[C@@H]2CCO2)c1F] |
| 23 | 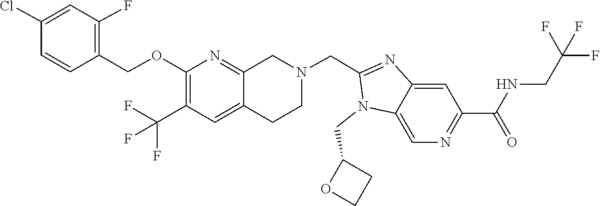 [SMILES: O=C(NCC(F)(F)F)c1cc2nc(CN3CCc4cc(C(F)(F)F)c(OCc5ccc(Cl)cc5F)nc4C3)n(C[C@@H]3CCO3)c2cn1] |
| 24 | 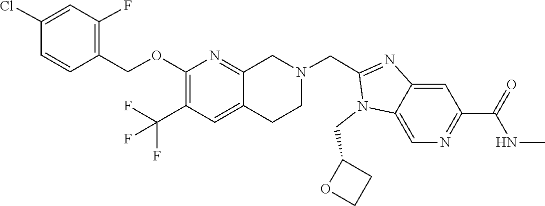 [SMILES: CNC(=O)c1cc2nc(CN3CCc4cc(C(F)(F)F)c(OCc5ccc(Cl)cc5F)nc4C3)n(C[C@@H]3CCO3)c2cn1] |
| 25 | 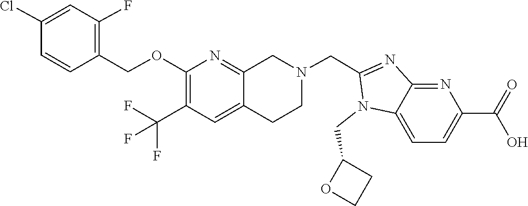 [SMILES: O=C(O)c1ccc2c(n1)nc(CN1CCc3cc(C(F)(F)F)c(OCc4ccc(Cl)cc4F)nc3C1)n2C[C@@H]1CCO1] |
| 26 | 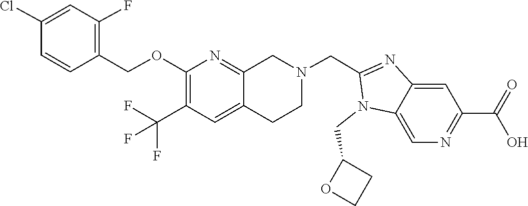 [SMILES: O=C(O)c1cc2nc(CN3CCc4cc(C(F)(F)F)c(OCc5ccc(Cl)cc5F)nc4C3)n(C[C@@H]3CCO3)c2cn1] |
| 27 | 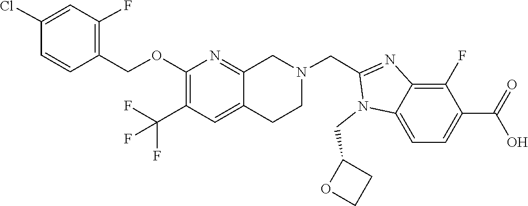 [SMILES: O=C(O)c1ccc2c(nc(CN3CCc4cc(C(F)(F)F)c(OCc5ccc(Cl)cc5F)nc4C3)n2C[C@@H]2CCO2)c1F] |
| 28 | 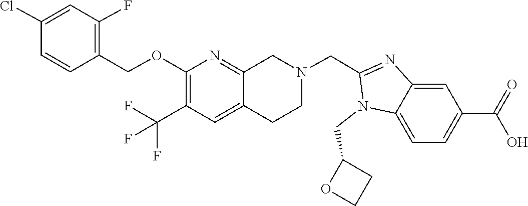 [SMILES: O=C(O)c1ccc2c(c1)nc(CN1CCc3cc(C(F)(F)F)c(OCc4ccc(Cl)cc4F)nc3C1)n2C[C@@H]1CCO1] |
| 29 | 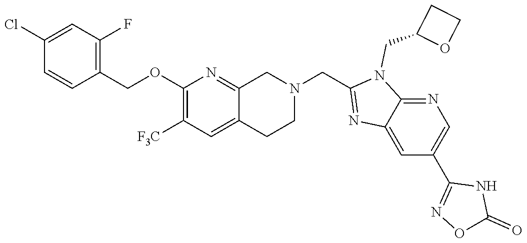 [SMILES: O=c1[nH]c(-c2cnc3c(c2)nc(CN2CCc4cc(C(F)(F)F)c(OCc5ccc(Cl)cc5F)nc4C2)n3C[C@@H]2CCO2)no1] |
| 30 | 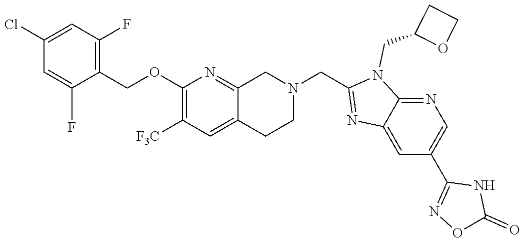 [SMILES: O=c1[nH]c(-c2cnc3c(c2)nc(CN2CCc4cc(C(F)(F)F)c(OCc5c(F)cc(Cl)cc5F)nc4C2)n3C[C@@H]2CCO2)no1] |
| 31 | 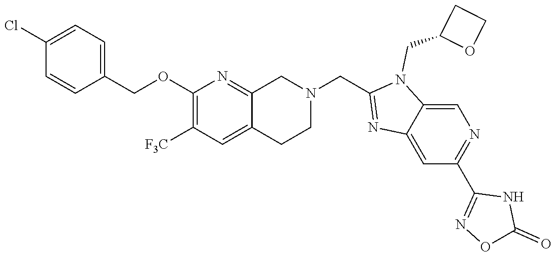 [SMILES: O=c1[nH]c(-c2cc3nc(CN4CCc5cc(C(F)(F)F)c(OCc6ccc(Cl)cc6)nc5C4)n(C[C@@H]4CCO4)c3cn2)no1] |
| 32 | 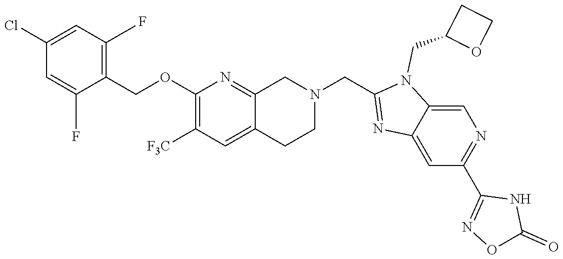 [SMILES: O=c1[nH]c(-c2cc3nc(CN4CCc5cc(C(F)(F)F)c(OCc6c(F)cc(Cl)cc6F)nc5C4)n(C[C@@H]4CCO4)c3cn2)no1] |
| 33 | 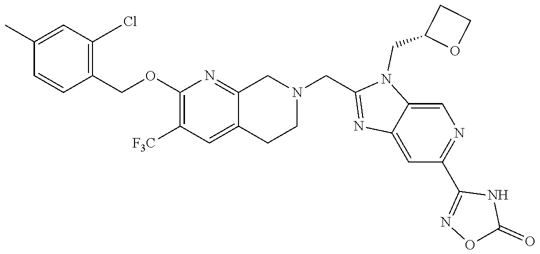 [SMILES: Cc1ccc(COc2nc3c(cc2C(F)(F)F)CCN(Cc2nc4cc(-c5noc(=O)[nH]5)ncc4n2C[C@@H]2CCO2)C3)c(Cl)c1] |
| 34 | 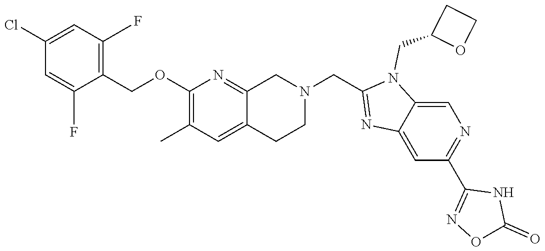 [SMILES: Cc1cc2c(nc1OCc1c(F)cc(Cl)cc1F)CN(Cc1nc3cc(-c4noc(=O)[nH]4)ncc3n1C[C@@H]1CCO1)CC2] |
| 35 | 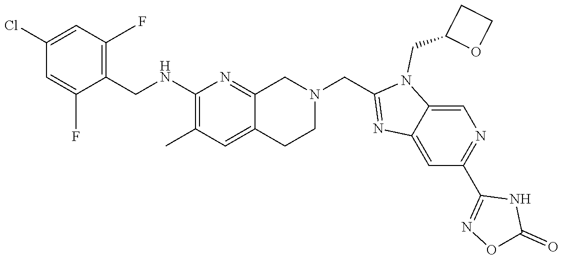 [SMILES: Cc1cc2c(nc1NCc1c(F)cc(Cl)cc1F)CN(Cc1nc3cc(-c4noc(=O)[nH]4)ncc3n1C[C@@H]1CCO1)CC2] |
| 36 | 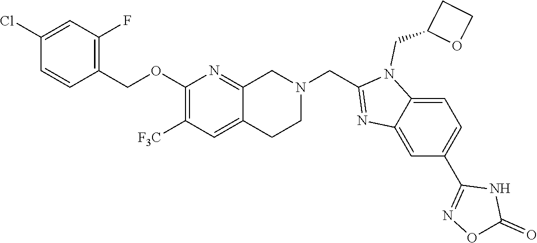 [SMILES: O=c1[nH]c(-c2ccc3c(c2)nc(CN2CCc4cc(C(F)(F)F)c(OCc5ccc(Cl)cc5F)nc4C2)n3C[C@@H]2CCO2)no1] |
| 37 | 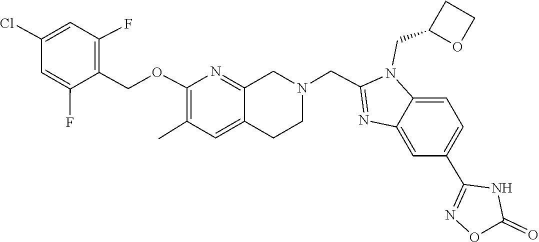 [SMILES: Cc1cc2c(nc1OCc1c(F)cc(Cl)cc1F)CN(Cc1nc3cc(-c4noc(=O)[nH]4)ccc3n1C[C@@H]1CCO1)CC2] |
| 38 | 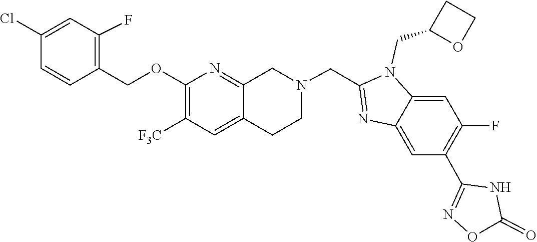 [SMILES: O=c1[nH]c(-c2cc3nc(CN4CCc5cc(C(F)(F)F)c(OCc6ccc(Cl)cc6F)nc5C4)n(C[C@@H]4CCO4)c3cc2F)no1] |
| 39 | 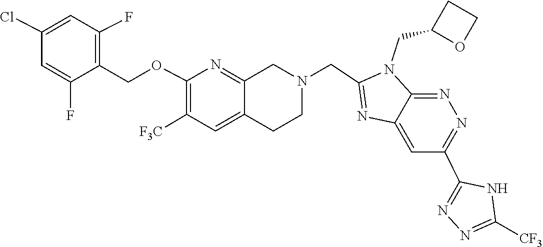 [SMILES: Fc1cc(Cl)cc(F)c1COc1nc2c(cc1C(F)(F)F)CCN(Cc1nc3cc(-c4nnc(C(F)(F)F)[nH]4)nnc3n1C[C@@H]1CCO1)C2] |
| 40 | 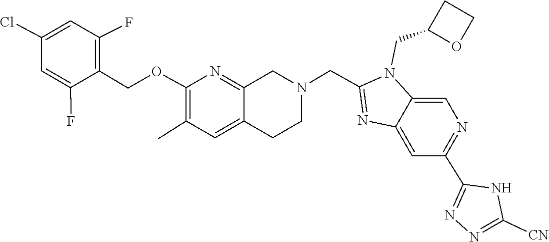 [SMILES: Cc1cc2c(nc1OCc1c(F)cc(Cl)cc1F)CN(Cc1nc3cc(-c4nnc(C#N)[nH]4)ncc3n1C[C@@H]1CCO1)CC2] |
| 41 | 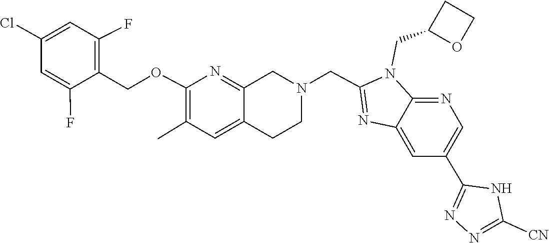 [SMILES: Cc1cc2c(nc1OCc1c(F)cc(Cl)cc1F)CN(Cc1nc3cc(-c4nnc(C#N)[nH]4)cnc3n1C[C@@H]1CCO1)CC2] |
| 42 | 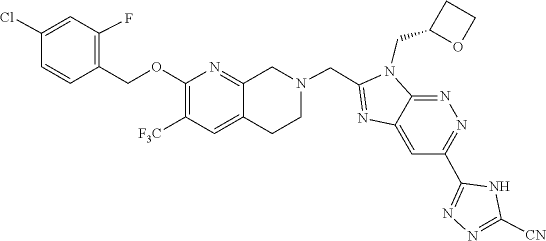 [SMILES: N#Cc1nnc(-c2cc3nc(CN4CCc5cc(C(F)(F)F)c(OCc6ccc(Cl)cc6F)nc5C4)n(C[C@@H]4CCO4)c3nn2)[nH]1] |
| 43 | 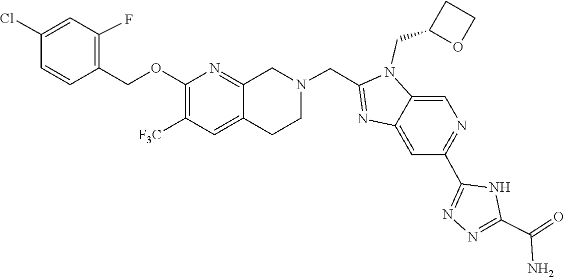 [SMILES: NC(=O)c1nnc(-c2cc3nc(CN4CCc5cc(C(F)(F)F)c(OCc6ccc(Cl)cc6F)nc5C4)n(C[C@@H]4CCO4)c3cn2)[nH]1] |
| 44 | 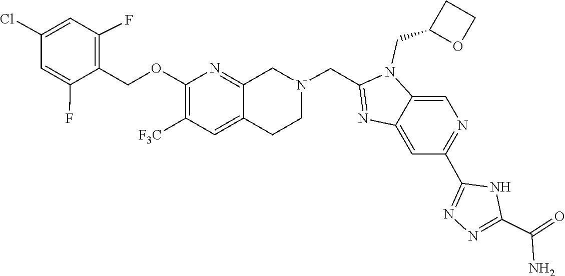 [SMILES: NC(=O)c1nnc(-c2cc3nc(CN4CCc5cc(C(F)(F)F)c(OCc6c(F)cc(Cl)cc6F)nc5C4)n(C[C@@H]4CCO4)c3cn2)[nH]1] |
| 45 | 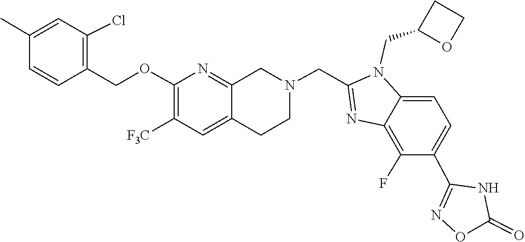 [SMILES: Cc1ccc(COc2nc3c(cc2C(F)(F)F)CCN(Cc2nc4c(F)c(-c5noc(=O)[nH]5)ccc4n2C[C@@H]2CCO2)C3)c(Cl)c1] |
| 46 | 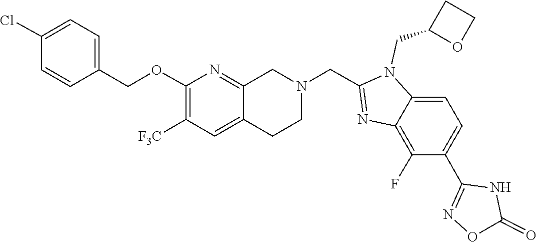 [SMILES: O=c1[nH]c(-c2ccc3c(nc(CN4CCc5cc(C(F)(F)F)c(OCc6ccc(Cl)cc6)nc5C4)n3C[C@@H]3CCO3)c2F)no1] |
| 47 | 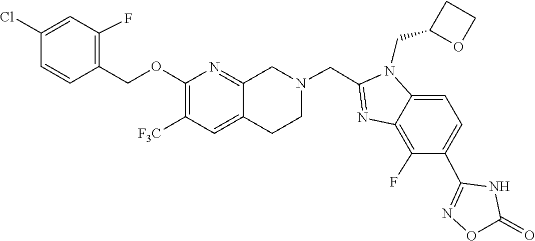 [SMILES: O=c1[nH]c(-c2ccc3c(nc(CN4CCc5cc(C(F)(F)F)c(OCc6ccc(Cl)cc6F)nc5C4)n3C[C@@H]3CCO3)c2F)no1] |
| 48 | 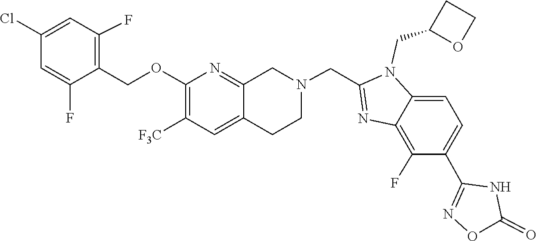 [SMILES: O=c1[nH]c(-c2ccc3c(nc(CN4CCc5cc(C(F)(F)F)c(OCc6c(F)cc(Cl)cc6F)nc5C4)n3C[C@@H]3CCO3)c2F)no1] |
| 49 | 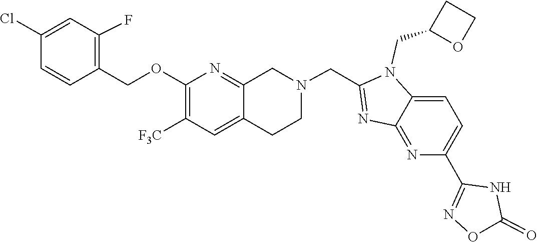 [SMILES: O=c1[nH]c(-c2ccc3c(n2)nc(CN2CCc4cc(C(F)(F)F)c(OCc5ccc(Cl)cc5F)nc4C2)n3C[C@@H]2CCO2)no1] |
| 50 | 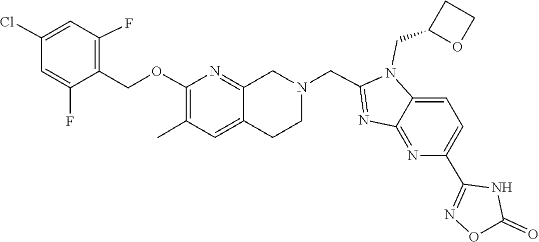 [SMILES: Cc1cc2c(nc1OCc1c(F)cc(Cl)cc1F)CN(Cc1nc3nc(-c4noc(=O)[nH]4)ccc3n1C[C@@H]1CCO1)CC2] |
| 51 | 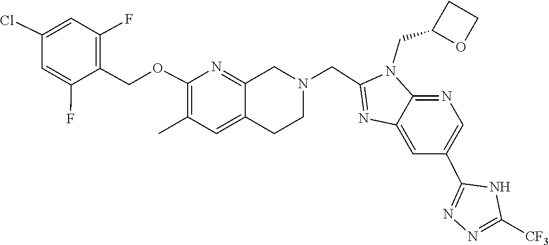 [SMILES: Cc1cc2c(nc1OCc1c(F)cc(Cl)cc1F)CN(Cc1nc3cc(-c4nnc(C(F)(F)F)[nH]4)cnc3n1C[C@@H]1CCO1)CC2] |
| 52 | 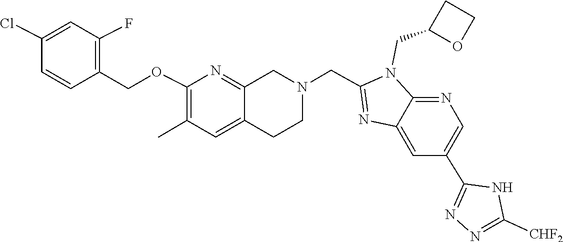 [SMILES: Cc1cc2c(nc1OCc1ccc(Cl)cc1F)CN(Cc1nc3cc(-c4nnc(C(F)F)[nH]4)cnc3n1C[C@@H]1CCO1)CC2] |
| 53 | 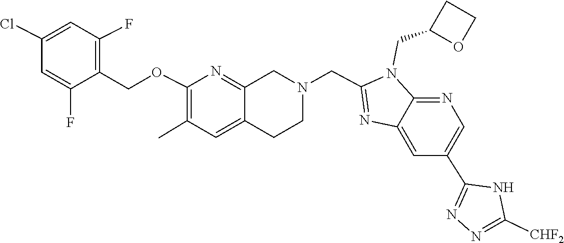 [SMILES: Cc1cc2c(nc1OCc1c(F)cc(Cl)cc1F)CN(Cc1nc3cc(-c4nnc(C(F)F)[nH]4)cnc3n1C[C@@H]1CCO1)CC2] |
| 54 | 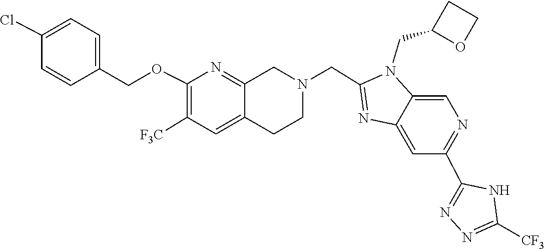 [SMILES: FC(F)(F)c1nnc(-c2cc3nc(CN4CCc5cc(C(F)(F)F)c(OCc6ccc(Cl)cc6)nc5C4)n(C[C@@H]4CCO4)c3cn2)[nH]1] |
| 55 | 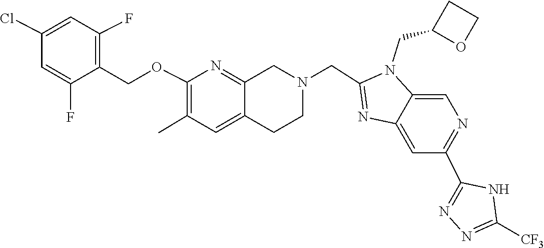 [SMILES: Cc1cc2c(nc1OCc1c(F)cc(Cl)cc1F)CN(Cc1nc3cc(-c4nnc(C(F)(F)F)[nH]4)ncc3n1C[C@@H]1CCO1)CC2] |
| 56 | 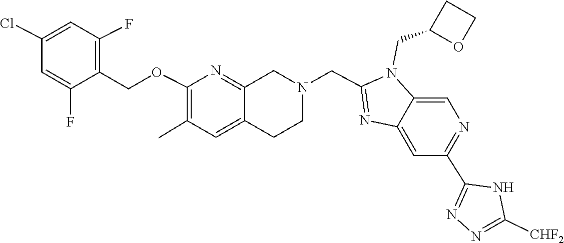 [SMILES: Cc1cc2c(nc1OCc1c(F)cc(Cl)cc1F)CN(Cc1nc3cc(-c4nnc(C(F)F)[nH]4)ncc3n1C[C@@H]1CCO1)CC2] |
| 57 | 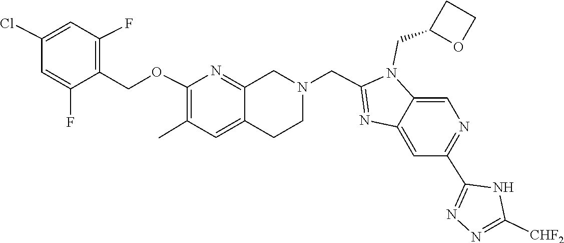 [SMILES: Cc1cc2c(nc1OCc1c(F)cc(Cl)cc1F)CN(Cc1nc3cc(-c4nnc(C(F)F)[nH]4)ncc3n1C[C@@H]1CCO1)CC2] |
| 58 | 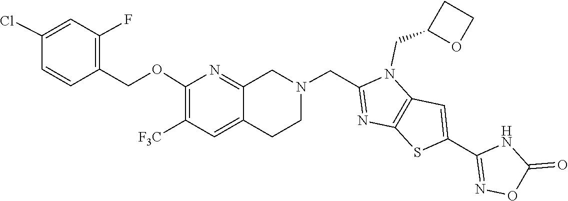 [SMILES: O=c1[nH]c(-c2cc3c(nc(CN4CCc5cc(C(F)(F)F)c(OCc6ccc(Cl)cc6F)nc5C4)n3C[C@@H]3CCO3)s2)no1] |
| 59 | 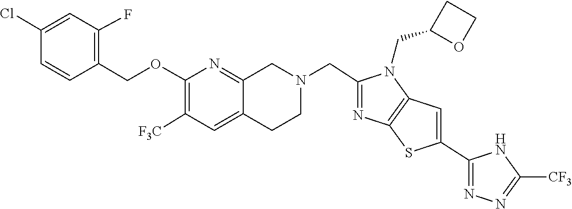 [SMILES: Fc1cc(Cl)ccc1COc1nc2c(cc1C(F)(F)F)CCN(Cc1nc3sc(-c4nnc(C(F)(F)F)[nH]4)cc3n1C[C@@H]1CCO1)C2] |
| 60 | 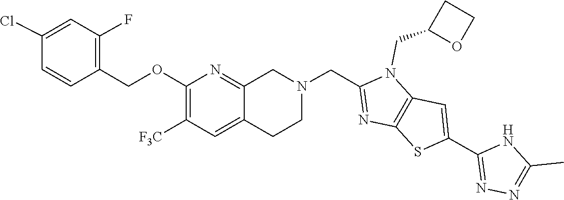 [SMILES: Cc1nnc(-c2cc3c(nc(CN4CCc5cc(C(F)(F)F)c(OCc6ccc(Cl)cc6F)nc5C4)n3C[C@@H]3CCO3)s2)[nH]1] |
| 61 | 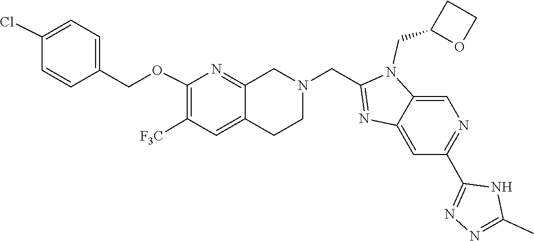 [SMILES: Cc1nnc(-c2cc3nc(CN4CCc5cc(C(F)(F)F)c(OCc6ccc(Cl)cc6)nc5C4)n(C[C@@H]4CCO4)c3cn2)[nH]1] |
| 62 | 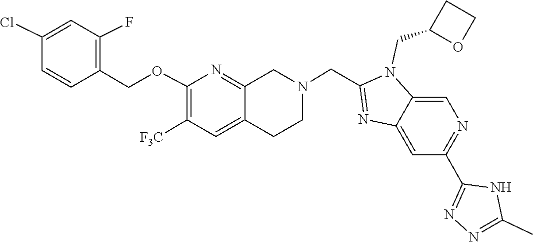 [SMILES: Cc1nnc(-c2cc3nc(CN4CCc5cc(C(F)(F)F)c(OCc6ccc(Cl)cc6F)nc5C4)n(C[C@@H]4CCO4)c3cn2)[nH]1] |
| 63 | 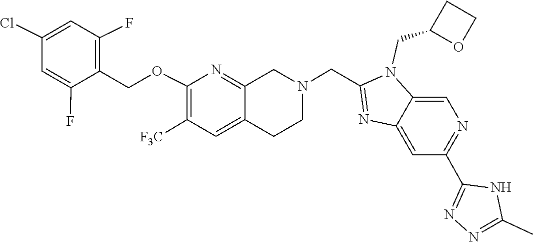 [SMILES: Cc1nnc(-c2cc3nc(CN4CCc5cc(C(F)(F)F)c(OCc6c(F)cc(Cl)cc6F)nc5C4)n(C[C@@H]4CCO4)c3cn2)[nH]1] |
| 64 | 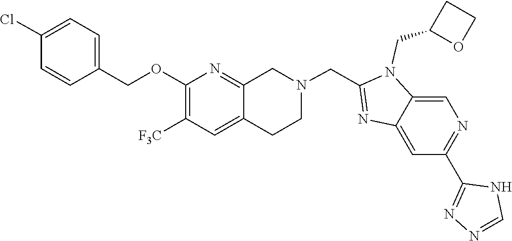 [SMILES: FC(F)(F)c1cc2c(nc1OCc1ccc(Cl)cc1)CN(Cc1nc3cc(-c4nnc[nH]4)ncc3n1C[C@@H]1CCO1)CC2] |
| 65 | 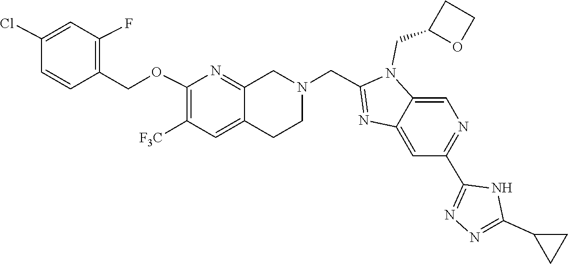 [SMILES: Fc1cc(Cl)ccc1COc1nc2c(cc1C(F)(F)F)CCN(Cc1nc3cc(-c4nnc(C5CC5)[nH]4)ncc3n1C[C@@H]1CCO1)C2] |
| 66 | 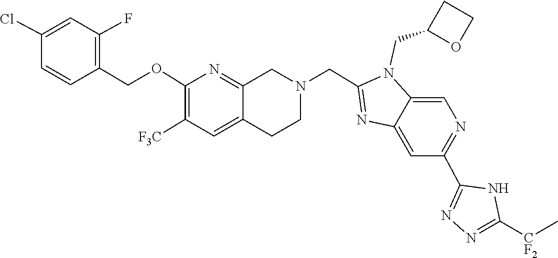 [SMILES: CC(F)(F)c1nnc(-c2cc3nc(CN4CCc5cc(C(F)(F)F)c(OCc6ccc(Cl)cc6F)nc5C4)n(C[C@@H]4CCO4)c3cn2)[nH]1] |
| 67 | 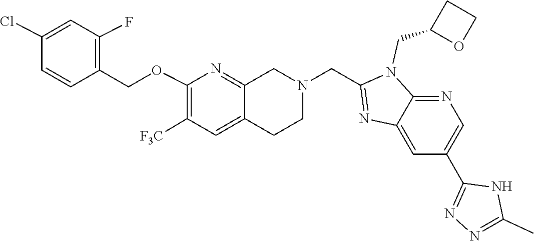 [SMILES: Cc1nnc(-c2cnc3c(c2)nc(CN2CCc4cc(C(F)(F)F)c(OCc5ccc(Cl)cc5F)nc4C2)n3C[C@@H]2CCO2)[nH]1] |
| 68 | 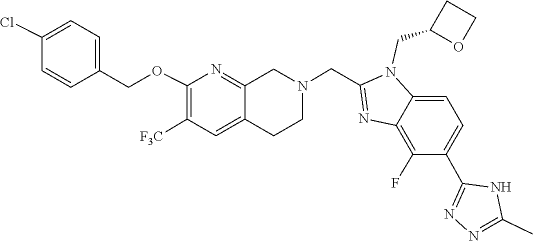 [SMILES: Cc1nnc(-c2ccc3c(nc(CN4CCc5cc(C(F)(F)F)c(OCc6ccc(Cl)cc6)nc5C4)n3C[C@@H]3CCO3)c2F)[nH]1] |
| 69 | 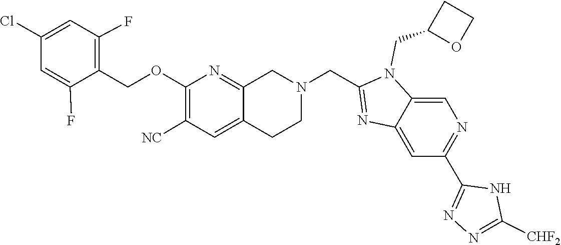 [SMILES: N#Cc1cc2c(nc1OCc1c(F)cc(Cl)cc1F)CN(Cc1nc3cc(-c4nnc(C(F)F)[nH]4)ncc3n1C[C@@H]1CCO1)CC2] |
| 70 | 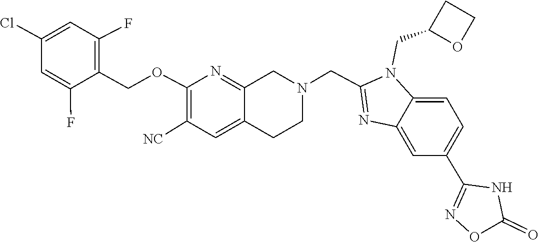 [SMILES: N#Cc1cc2c(nc1OCc1c(F)cc(Cl)cc1F)CN(Cc1nc3cc(-c4noc(=O)[nH]4)ccc3n1C[C@@H]1CCO1)CC2] |
| 71 | 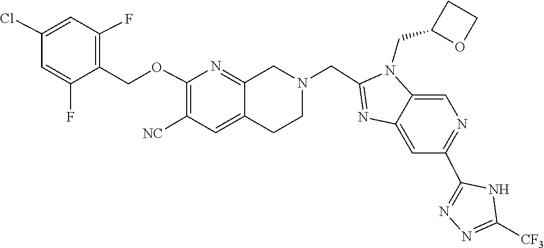 [SMILES: N#Cc1cc2c(nc1OCc1c(F)cc(Cl)cc1F)CN(Cc1nc3cc(-c4nnc(C(F)(F)F)[nH]4)ncc3n1C[C@@H]1CCO1)CC2] |
| 72 | 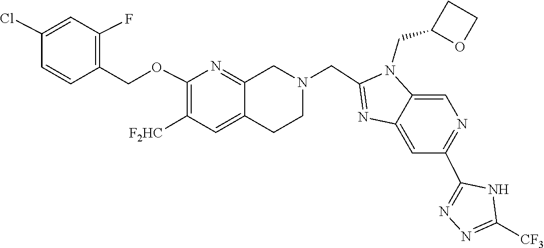 [SMILES: Fc1cc(Cl)ccc1COc1nc2c(cc1C(F)F)CCN(Cc1nc3cc(-c4nnc(C(F)(F)F)[nH]4)ncc3n1C[C@@H]1CCO1)C2] |
| 73 | 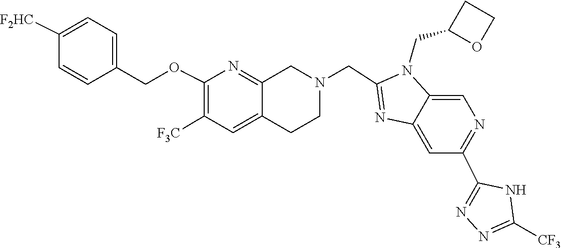 [SMILES: FC(F)c1ccc(COc2nc3c(cc2C(F)(F)F)CCN(Cc2nc4cc(-c5nnc(C(F)(F)F)[nH]5)ncc4n2C[C@@H]2CCO2)C3)cc1] |
| 74 | 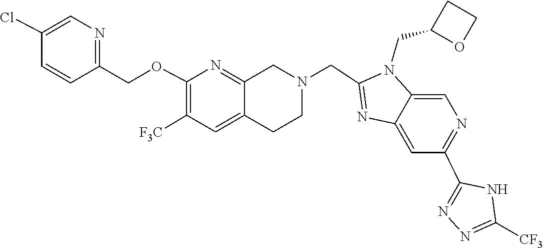 [SMILES: FC(F)(F)c1nnc(-c2cc3nc(CN4CCc5cc(C(F)(F)F)c(OCc6ccc(Cl)cn6)nc5C4)n(C[C@@H]4CCO4)c3cn2)[nH]1] |
| 75 | 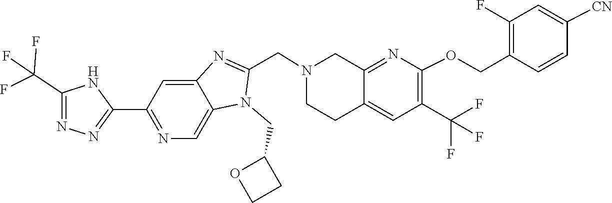 [SMILES: N#Cc1ccc(COc2nc3c(cc2C(F)(F)F)CCN(Cc2nc4cc(-c5nnc(C(F)(F)F)[nH]5)ncc4n2C[C@@H]2CCO2)C3)c(F)c1] |
| 76 | 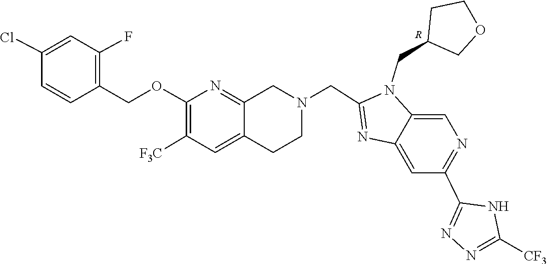 |
| 77 | 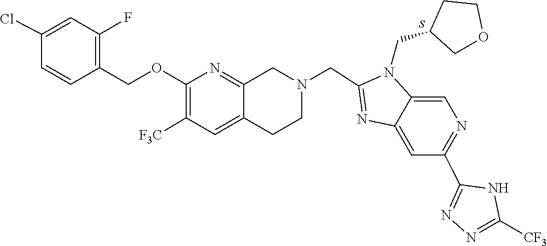 |
| 78 | 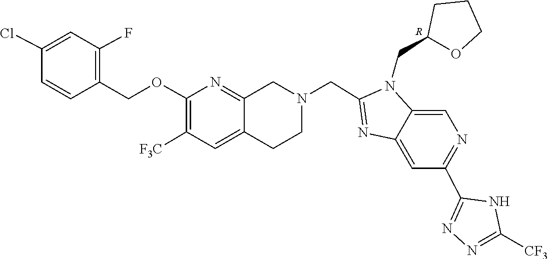 |
| 79 | 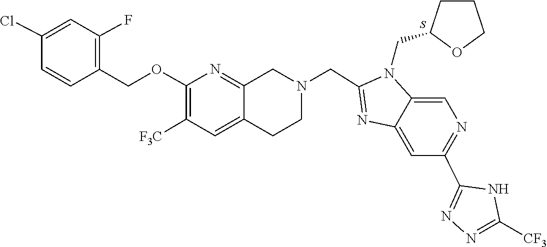 |
| 80 | 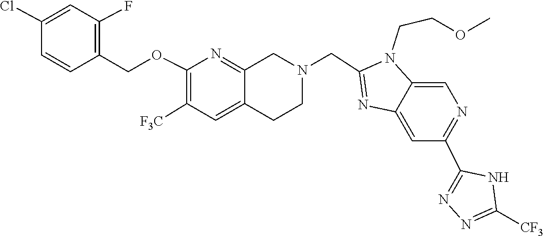 [SMILES: COCCn1c(CN2CCc3cc(C(F)(F)F)c(OCc4ccc(Cl)cc4F)nc3C2)nc2cc(-c3nnc(C(F)(F)F)[nH]3)ncc21] |
| 81 | 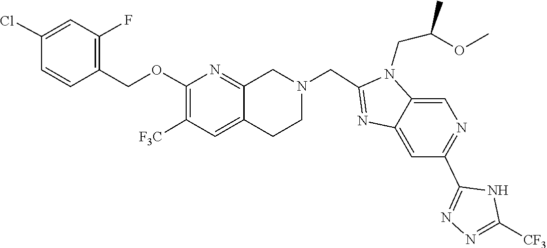 [SMILES: CO[C@H](C)Cn1c(CN2CCc3cc(C(F)(F)F)c(OCc4ccc(Cl)cc4F)nc3C2)nc2cc(-c3nnc(C(F)(F)F)[nH]3)ncc21] |
| 82 | 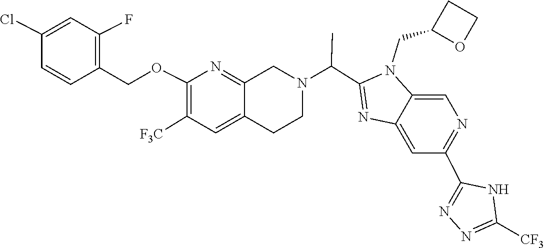 [SMILES: CC(c1nc2cc(-c3nnc(C(F)(F)F)[nH]3)ncc2n1C[C@@H]1CCO1)N1CCc2cc(C(F)(F)F)c(OCc3ccc(Cl)cc3F)nc2C1] |
| 83 | 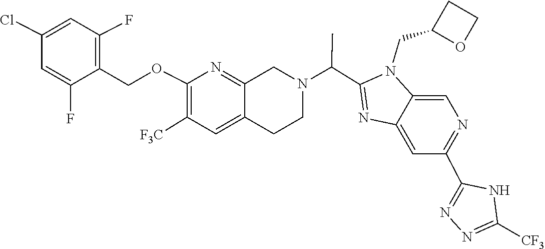 [SMILES: CC(c1nc2cc(-c3nnc(C(F)(F)F)[nH]3)ncc2n1C[C@@H]1CCO1)N1CCc2cc(C(F)(F)F)c(OCc3c(F)cc(Cl)cc3F)nc2C1] |
| 84 | 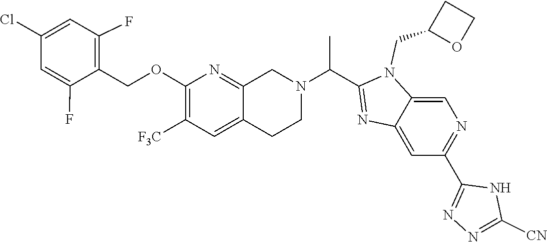 [SMILES: CC(c1nc2cc(-c3nnc(C#N)[nH]3)ncc2n1C[C@@H]1CCO1)N1CCc2cc(C(F)(F)F)c(OCc3c(F)cc(Cl)cc3F)nc2C1] |
| 85 | 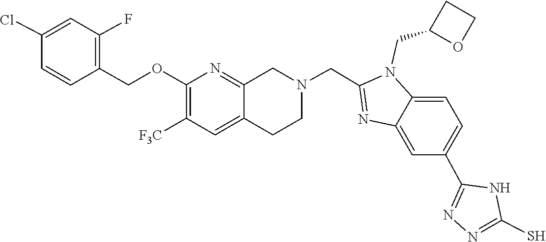 [SMILES: Fc1cc(Cl)ccc1COc1nc2c(cc1C(F)(F)F)CCN(Cc1nc3cc(-c4nnc(S)[nH]4)ccc3n1C[C@@H]1CCO1)C2] |
| 86 | 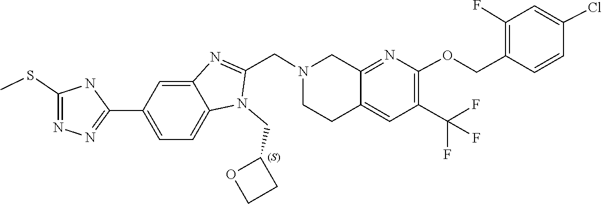 |
| 87 | 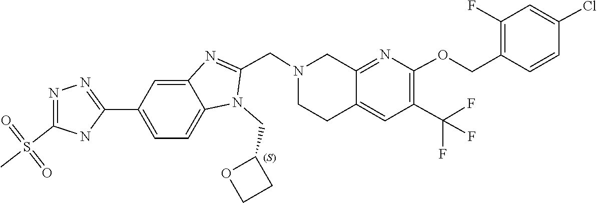 |
| 88 | 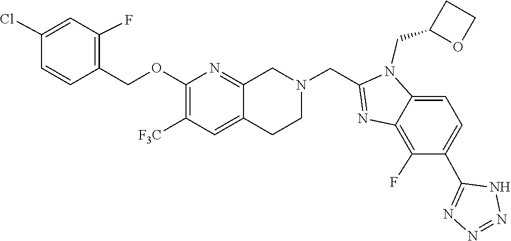 [SMILES: Fc1cc(Cl)ccc1COc1nc2c(cc1C(F)(F)F)CCN(Cc1nc3c(F)c(-c4nnn[nH]4)ccc3n1C[C@@H]1CCO1)C2] |
| 89 | 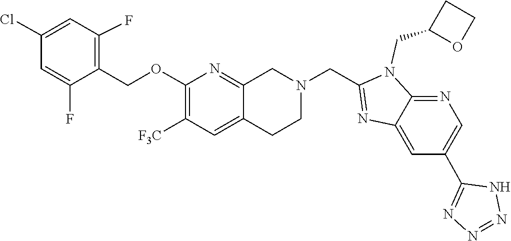 [SMILES: Fc1cc(Cl)cc(F)c1COc1nc2c(cc1C(F)(F)F)CCN(Cc1nc3cc(-c4nnn[nH]4)cnc3n1C[C@@H]1CCO1)C2] |
| 90 | 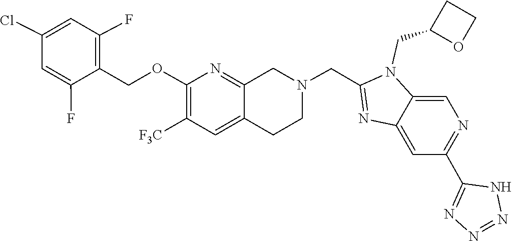 [SMILES: Fc1cc(Cl)cc(F)c1COc1nc2c(cc1C(F)(F)F)CCN(Cc1nc3cc(-c4nnn[nH]4)ncc3n1C[C@@H]1CCO1)C2] |
| 91 | 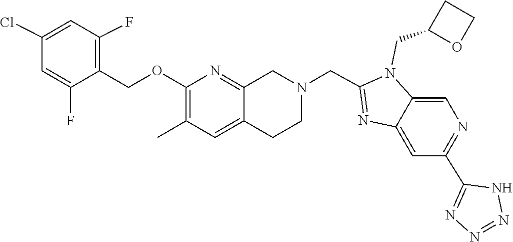 [SMILES: Cc1cc2c(nc1OCc1c(F)cc(Cl)cc1F)CN(Cc1nc3cc(-c4nnn[nH]4)ncc3n1C[C@@H]1CCO1)CC2] |
| 92 | 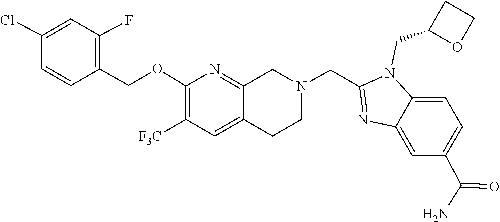 [SMILES: NC(=O)c1ccc2c(c1)nc(CN1CCc3cc(C(F)(F)F)c(OCc4ccc(Cl)cc4F)nc3C1)n2C[C@@H]1CCO1] |
| 93 | 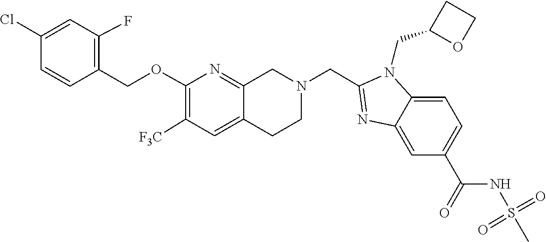 [SMILES: CS(=O)(=O)NC(=O)c1ccc2c(c1)nc(CN1CCc3cc(C(F)(F)F)c(OCc4ccc(Cl)cc4F)nc3C1)n2C[C@@H]1CCO1] |
| 94 | 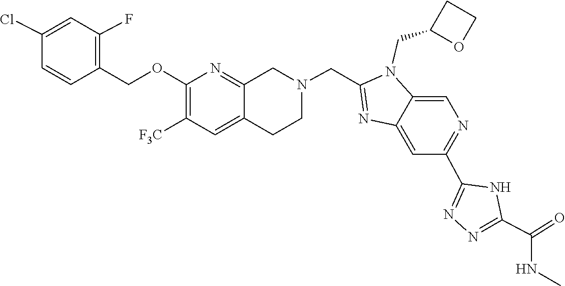 [SMILES: CNC(=O)c1nnc(-c2cc3nc(CN4CCc5cc(C(F)(F)F)c(OCc6ccc(Cl)cc6F)nc5C4)n(C[C@@H]4CCO4)c3cn2)[nH]1] |
| 95 | 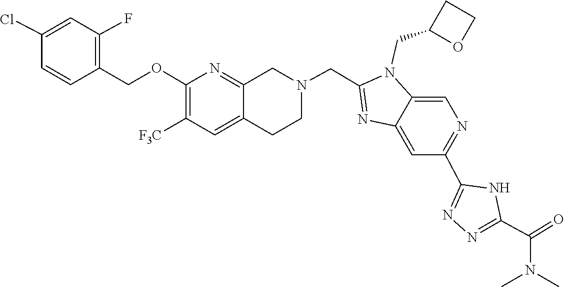 [SMILES: CN(C)C(=O)c1nnc(-c2cc3nc(CN4CCc5cc(C(F)(F)F)c(OCc6ccc(Cl)cc6F)nc5C4)n(C[C@@H]4CCO4)c3cn2)[nH]1] |
| 96 | 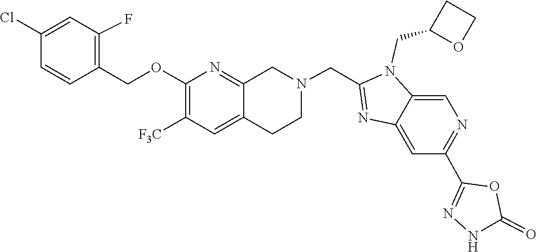 [SMILES: O=c1[nH]nc(-c2cc3nc(CN4CCc5cc(C(F)(F)F)c(OCc6ccc(Cl)cc6F)nc5C4)n(C[C@@H]4CCO4)c3cn2)o1] |
| 97 | 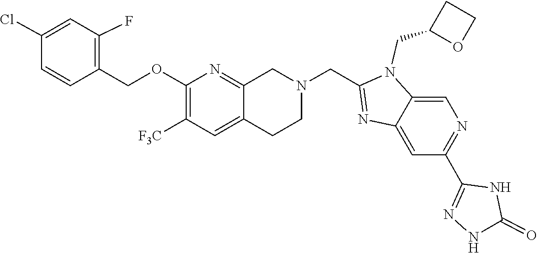 [SMILES: O=c1[nH]nc(-c2cc3nc(CN4CCc5cc(C(F)(F)F)c(OCc6ccc(Cl)cc6F)nc5C4)n(C[C@@H]4CCO4)c3cn2)[nH]1] |
| 98 | 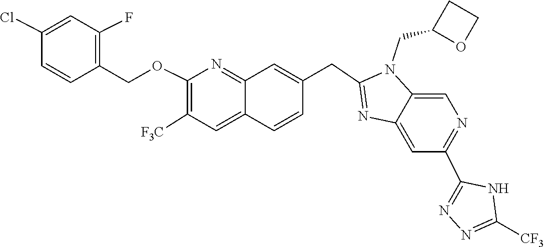 [SMILES: Fc1cc(Cl)ccc1COc1nc2cc(Cc3nc4cc(-c5nnc(C(F)(F)F)[nH]5)ncc4n3C[C@@H]3CCO3)ccc2cc1C(F)(F)F] |
| 99 | 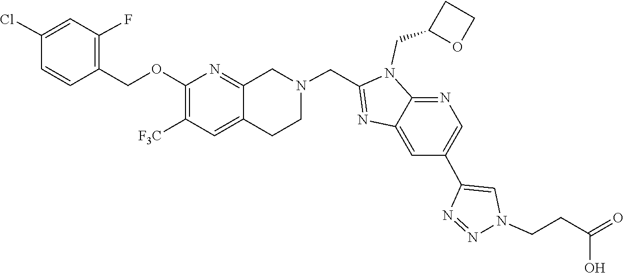 [SMILES: O=C(O)CCn1cc(-c2cnc3c(c2)nc(CN2CCc4cc(C(F)(F)F)c(OCc5ccc(Cl)cc5F)nc4C2)n3C[C@@H]2CCO2)nn1] |
| 100 | 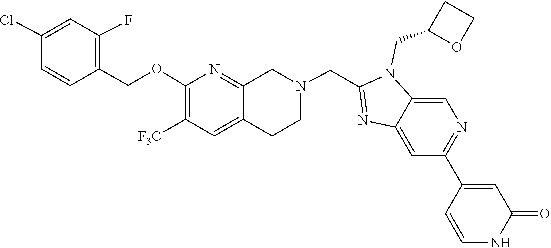 [SMILES: O=c1cc(-c2cc3nc(CN4CCc5cc(C(F)(F)F)c(OCc6ccc(Cl)cc6F)nc5C4)n(C[C@@H]4CCO4)c3cn2)cc[nH]1] |
| 101 | 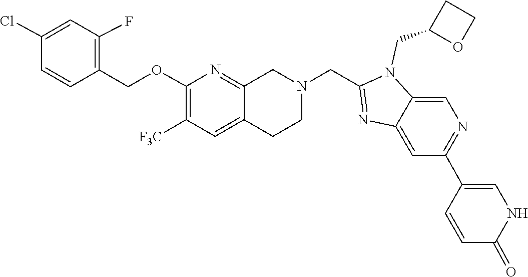 [SMILES: O=c1ccc(-c2cc3nc(CN4CCc5cc(C(F)(F)F)c(OCc6ccc(Cl)cc6F)nc5C4)n(C[C@@H]4CCO4)c3cn2)c[nH]1] |
| 102 | 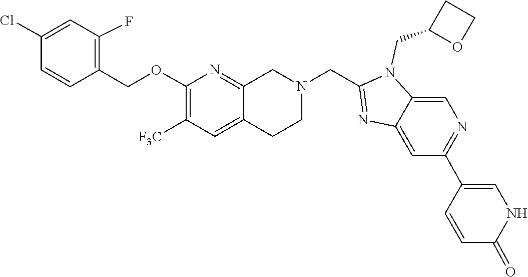 [SMILES: O=c1ccc(-c2cc3nc(CN4CCc5cc(C(F)(F)F)c(OCc6ccc(Cl)cc6F)nc5C4)n(C[C@@H]4CCO4)c3cn2)c[nH]1] |
| 103 | 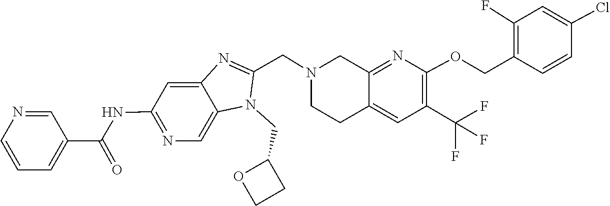 [SMILES: O=C(Nc1cc2nc(CN3CCc4cc(C(F)(F)F)c(OCc5ccc(Cl)cc5F)nc4C3)n(C[C@@H]3CCO3)c2cn1)c1cccnc1] |
| 104 | 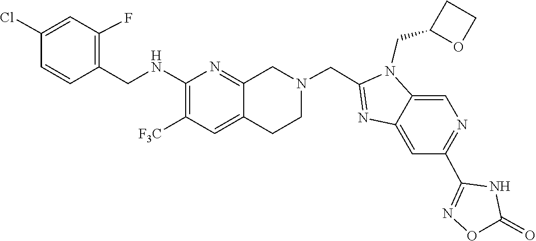 [SMILES: O=c1[nH]c(-c2cc3nc(CN4CCc5cc(C(F)(F)F)c(NCc6ccc(Cl)cc6F)nc5C4)n(C[C@@H]4CCO4)c3cn2)no1] |
| 105 | 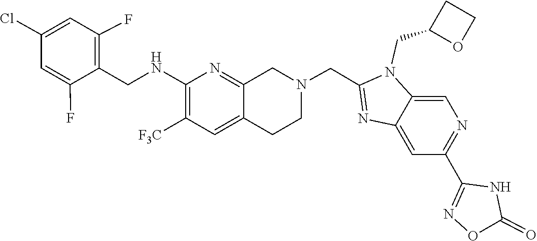 [SMILES: O=c1[nH]c(-c2cc3nc(CN4CCc5cc(C(F)(F)F)c(NCc6c(F)cc(Cl)cc6F)nc5C4)n(C[C@@H]4CCO4)c3cn2)no1] |
| 106 | 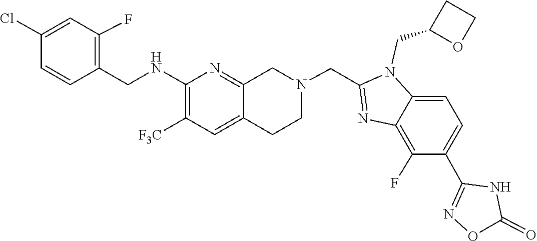 [SMILES: O=c1[nH]c(-c2ccc3c(nc(CN4CCc5cc(C(F)(F)F)c(NCc6ccc(Cl)cc6F)nc5C4)n3C[C@@H]3CCO3)c2F)no1] |
| 107 | 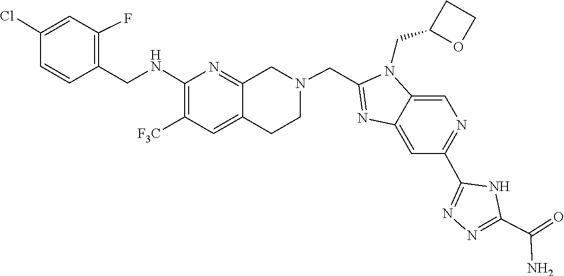 [SMILES: NC(=O)c1nnc(-c2cc3nc(CN4CCc5cc(C(F)(F)F)c(NCc6ccc(Cl)cc6F)nc5C4)n(C[C@@H]4CCO4)c3cn2)[nH]1] |
| 108 | 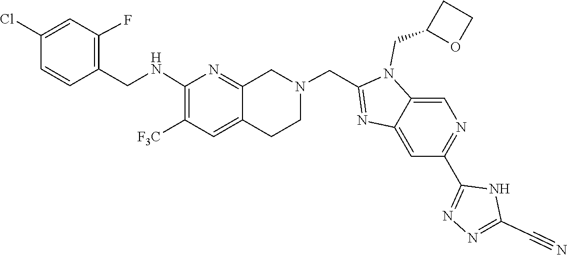 [SMILES: N#Cc1nnc(-c2cc3nc(CN4CCc5cc(C(F)(F)F)c(NCc6ccc(Cl)cc6F)nc5C4)n(C[C@@H]4CCO4)c3cn2)[nH]1] |
| 109 | 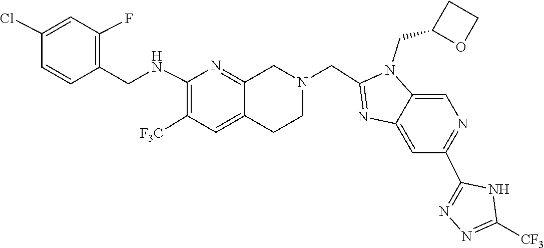 [SMILES: Fc1cc(Cl)ccc1CNc1nc2c(cc1C(F)(F)F)CCN(Cc1nc3cc(-c4nnc(C(F)(F)F)[nH]4)ncc3n1C[C@@H]1CCO1)C2] |
| 110 | 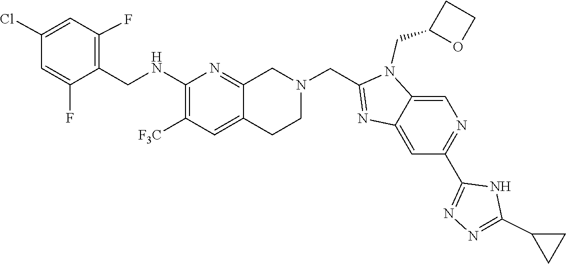 [SMILES: Fc1cc(Cl)cc(F)c1CNc1nc2c(cc1C(F)(F)F)CCN(Cc1nc3cc(-c4nnc(C5CC5)[nH]4)ncc3n1C[C@@H]1CCO1)C2] |

The compounds of Formula I include pharmaceutically acceptable salts thereof. In addition, the compounds of Formula I also include other salts of such compounds which are not necessarily pharmaceutically acceptable salts, and which may be useful as intermediates for preparing and/or purifying compounds of Formula I and/or for separating enantiomers of compounds of Formula I. Non-limiting examples of pharmaceutically acceptable salts of compounds of Formula I include trifluoroacetic acid salts.

It will further be appreciated that the compounds of Formula I or their salts may be isolated in the form of solvates, and accordingly that any such solvate is included within the scope of the present disclosure. For example, compounds of Formula I and salts thereof can exist in unsolvated as well as solvated forms with pharmaceutically acceptable solvents such as water, ethanol, and the like.

### Pharmaceutical Compositions and Administration

When employed as pharmaceuticals, compounds as described herein (e.g., a compound of Formula I, or a pharmaceutically acceptable salt or solvate thereof) can be administered in the form of a pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Formula I, or a pharmaceutically acceptable salt or solvate thereof, in combination with one or more pharmaceutically acceptable excipients (carriers). For example, a pharmaceutical composition prepared using a compound of Formula I, or a pharmaceutically acceptable salt or solvate thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula I, or a pharmaceutically acceptable salt or solvate thereof with a pharmaceutically acceptable excipient. Pharmaceutical compositions containing a compound of Formula I, or a pharmaceutically acceptable salt or solvate thereof as the active ingredient can be prepared by intimately mixing the compound of Formula I, or a pharmaceutically acceptable salt or solvate thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In some embodiments, the compound or pharmaceutical composition can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22nd Edition (Pharmaceutical Press, London, U K. 2012).

In some embodiments, the compounds and pharmaceutical compositions described herein or a pharmaceutical composition thereof can be administered to patient in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal (e.g., intranasal), nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In some embodiments, a route of administration is parenteral (e.g., intratumoral).

In some embodiments, a compound of Formula I, or a pharmaceutically acceptable salt or solvate thereof as described herein or pharmaceutical compositions thereof can be formulated for parenteral administration, e.g., formulated for injection via the intraarterial, intrasternal, intracranial, intravenous, intramuscular, sub-cutaneous, or intraperitoneal routes. For example, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure. In some embodiments, devices are used for parenteral administration. For example, such devices may include needle injectors, microneedle injectors, needle-free injectors, and infusion techniques.

In some embodiments, the pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In some embodiments, the form must be sterile and must be fluid to the extent that it may be easily injected. In some embodiments, the form should be stable under the conditions of manufacture and storage and must be preserved against the contaminating action of microorganisms, such as bacteria and fungi.

In some embodiments, the carrier also can be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity can be maintained, for example, by the use of a coating, such as lecithin, by the maintenance of the required particle size in the case of dispersion, and by the use of surfactants. In some embodiments, the prevention of the action of microorganisms can be brought about by various antibacterial and antifungal agents, for example, parabens, chlorobutanol, phenol, sorbic acid, thimerosal, and the like. In some embodiments, isotonic agents, for example, sugars or sodium chloride are included. In some embodiments, prolonged absorption of the injectable compositions can be brought about by the use in the compositions of agents delaying absorption, for example, aluminum monostearate and gelatin.

In some embodiments, sterile injectable solutions are prepared by incorporating a compound of Formula I, or a pharmaceutically acceptable salt or solvate thereof in the required amount in the appropriate solvent with various of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In some embodiments, sterile powders are used for the preparation of sterile injectable solutions. In some embodiments, the methods of preparation are vacuum-drying and freeze-drying techniques, which yield a powder of the active ingredient, plus any additional desired ingredient from a previously sterile-filtered solution thereof.

In some embodiments, pharmacologically acceptable excipients usable in a rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol, Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In some embodiments, suppositories can be prepared by mixing a compound of Formula I, or a pharmaceutically acceptable salt or solvate thereof, or pharmaceutical compositions as described herein with suitable non-irritating excipients or carriers such as cocoa butter, polyethylene glycol or a suppository wax which are solid at ambient temperature but liquid at body temperature and therefore melt in the rectum and release the active compound. In some embodiments, compositions for rectal administration are in the form of an enema.

In some embodiments, a compound of Formula I, or a pharmaceutically acceptable salt or solvate thereof, as described herein or a pharmaceutical composition thereof is formulated for local delivery to the digestive or GI tract by way of oral administration (e.g., solid or liquid dosage forms).

In some embodiments, solid dosage forms for oral administration include capsules, tablets, pills, powders, and granules. In some embodiments, a compound of Formula I, or a pharmaceutically acceptable salt or solvate thereof, is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For example, in the case of capsules, tablets and pills, the dosage form may also comprise buffering agents. In some embodiments, solid compositions of a similar type may also be employed as fillers in soft and hard-filled gelatin capsules using such excipients as lactose or milk sugar as well as high molecular weight polyethylene glycols and the like.

In some embodiments, the pharmaceutical compositions will take the form of a unit dosage form such as a pill or tablet and thus the composition may contain, along with a compound of Formula I, or a pharmaceutically acceptable salt or solvate thereof as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In some embodiments, another solid dosage form, a powder, marume, solution or suspension (e.g., in propylene carbonate, vegetable oils, PEG's, poloxamer 124 or triglycerides) is encapsulated in a capsule (gelatin or cellulose base capsule). In some embodiments, unit dosage forms in which one or more compounds and pharmaceutical compositions as provided herein or additional active agents are physically separated are also contemplated; e.g., capsules with granules (or tablets in a capsule) of each drug; two-layer tablets; two-compartment gel caps, etc. In some embodiments, enteric coated or delayed release oral dosage forms are also contemplated.

In some embodiments, other physiologically acceptable compounds may include wetting agents, emulsifying agents, dispersing agents or preservatives that are particularly useful for preventing the growth or action of microorganisms. For example, various preservatives are well known and include, for example, phenol and ascorbic acid.

In some embodiments, the excipients are sterile and generally free of undesirable matter. For example, these compositions can be sterilized by conventional, well-known sterilization techniques. In some embodiments, for various oral dosage form excipients such as tablets and capsules, sterility is not required. For example, the United States Pharmacopeia/National Formulary (USP/NF) standard can be sufficient.

In some embodiments, a compound of Formula I, or a pharmaceutically acceptable salt or solvate thereof as described herein or a pharmaceutical composition thereof is formulated for ocular administration. In some embodiments, ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, EDTA, SofZia (boric acid, propylene glycol, sorbitol, and zinc chloride; Alcon Laboratories, Inc.), Purite (stabilized oxychloro complex; Allergan, Inc.)).

In some embodiments, a compound of Formula I, or a pharmaceutically acceptable salt or solvate thereof as described herein or a pharmaceutical composition thereof is formulated for topical administration to the skin or mucosa (e.g., dermally or transdermally). In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and non-sensitizing.

In any of the foregoing embodiments, pharmaceutical compositions as described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

The amount of the compound in a pharmaceutical composition or formulation can vary within the full range employed by those skilled in the art. Typically, the formulation will contain, on a weight percent (wt %) basis, from about 0.01-99.99 wt % of a compound of this disclosure based on the total formulation, with the balance being one or more suitable pharmaceutical excipients. In one embodiment, the compound is present at a level of about 1-80 wt %. Representative pharmaceutical formulations are described below.

### Formulation Example 1—Tablet formulation

The following ingredients are mixed intimately and pressed into single scored tablets.

| Ingredient | Quantity per tablet, mg |
|---|---|
| compound of this disclosure | 400 |
| cornstarch | 50 |
| croscarmellose sodium | 25 |
| lactose | 120 |
| magnesium stearate | 5 |

### Formulation Example 2—Capsule formulation

The following ingredients are mixed intimately and loaded into a hard-shell gelatin capsule

| Ingredient | Quantity per capsule, mg |
|---|---|
| compound of this disclosure | 200 |
| lactose, spray-dried | 148 |
| magnesium stearate | 2 |

### Formulation Example 3—Suspension formulation

The following ingredients are mixed to form a suspension for oral administration.

| Ingredient | Amount | |
|---|---|---|
| compound of this disclosure | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| coloring | 0.5 | mg |

| | |
|---|---|
| distilled water | q.s. to 100 mL |

### Formulation Example 4—Injectable formulation

The following ingredients are mixed to form an injectable formulation.

| Ingredient | Amount |
|---|---|
| compound of this disclosure | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this disclosure with Witepsol® H-15 (triglycerides of saturated vegetable fatty acid; Riches-Nelson, Inc., New York), and has the following composition:

| Ingredient | Amount |
|---|---|
| compound of this disclosure | 500 mg |
| Witepsol ® H-15 | balance |

In some embodiments, the dosage for a compound of Formula I, or a pharmaceutically acceptable salt or solvate thereof, is determined based on a multiple factors including, but not limited to, type, age, weight, sex, medical condition of the patient, severity of the medical condition of the patient, route of administration, and activity of the compound or pharmaceutically acceptable salt or solvate thereof. In some embodiments, proper dosage for a particular situation can be determined by one skilled in the medical arts. In some embodiments, the total daily dosage may be divided and administered in portions throughout the day or by means providing continuous delivery.

In some embodiments, a compound of Formula I, or a pharmaceutically acceptable salt or solvate thereof, is administered at a dose from about 0.01 to about 1000 mg. For example, from about 0.1 to about 30 mg, about 10 to about 80 mg, about 0.5 to about 15 mg, about 50 mg to about 200 mg, about 100 mg to about 300 mg, about 200 to about 400 mg, about 300 mg to about 500 mg, about 400 mg to about 600 mg, about 500 mg to about 800 mg, about 600 mg to about 900 mg, or about 700 mg to about 1000 mg. In some embodiments, the dose is a therapeutically effective amount.

In some embodiments, a compound of Formula I, or a pharmaceutically acceptable salt or solvate thereof as described herein is administered at a dosage of from about 0.0002 mg/Kg to about 100 mg/Kg (e.g., from about 0.0002 mg/Kg to about 50 mg/Kg; from about 0.0002 mg/Kg to about 25 mg/Kg; from about 0.0002 mg/Kg to about 10 mg/Kg; from about 0.0002 mg/Kg to about 5 mg/Kg; from about 0.0002 mg/Kg to about 1 mg/Kg; from about 0.0002 mg/Kg to about 0.5 mg/Kg; from about 0.0002 mg/Kg to about 0.1 mg/Kg; from about 0.001 mg/Kg to about 50 mg/Kg; from about 0.001 mg/Kg to about 25 mg/Kg; from about 0.001 mg/Kg to about 10 mg/Kg; from about 0.001 mg/Kg to about 5 mg/Kg; from about 0.001 mg/Kg to about 1 mg/Kg; from about 0.001 mg/Kg to about 0.5 mg/Kg; from about 0.001 mg/Kg to about 0.1 mg/Kg; from about 0.01 mg/Kg to about 50 mg/Kg; from about 0.01 mg/Kg to about 25 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 50 mg/Kg; from about 0.1 mg/Kg to about 25 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg). In some embodiments, a compound of Formula I, or a pharmaceutically acceptable salt or solvate thereof as described herein is administered as a dosage of about 100 mg/Kg.

In some embodiments, the foregoing dosages of a compound of Formula I, or a pharmaceutically acceptable salt or solvate thereof, can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound of Formula I, or a pharmaceutically acceptable salt or solvate thereof as described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a compound of Formula I, or a pharmaceutically acceptable salt or solvate thereof is administered to a patient for a period of time followed by a separate period of time where administration of the compound of Formula I, or a pharmaceutically acceptable salt or solvate thereof is stopped. In some embodiments, a compound of Formula I, or a pharmaceutically acceptable salt or solvate thereof is administered for a first period and a second period following the first period, with administration stopped during the second period, followed by a third period where administration of the compound of Formula I, or a pharmaceutically acceptable salt or solvate thereof is started and then a fourth period following the third period where administration is stopped. For example, the period of administration of a compound of Formula I, or a pharmaceutically acceptable salt or solvate thereof followed by a period where administration is stopped is repeated for a determined or undetermined period of time. In some embodiments, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, a compound of Formula I, or a pharmaceutically acceptable salt or solvate thereof, is orally administered to the patient one or more times per day (e.g., one time per day, two times per day, three times per day, four times per day per day or a single daily dose).

In some embodiments, a compound of Formula I, or a pharmaceutically acceptable salt or solvate thereof, is administered by parenteral administration to the patient one or more times per day (e.g., 1 to 4 times, one time per day, two times per day, three times per day, four times per day or a single daily dose).

In some embodiments, a compound of Formula I, or a pharmaceutically acceptable salt or solvate thereof, is administered by parenteral administration to the patient weekly.

### Methods of Treatment

In some embodiments, this disclosure features methods for treating a patient (e.g., a human) having a disease, disorder, or condition in which modulation of GLP-1R (e.g., repressed or impaired and/or elevated or unwanted GLP-1R) is beneficial for the treatment of the underlying pathology and/or symptoms and/or progression of the disease, disorder, or condition. In some embodiments, the methods described herein can include or further include treating one or more conditions associated, co-morbid or sequela with any one or more of the conditions described herein.

Provided herein is a method for treating a GLP-1 associated disease, disorder, or condition, the method comprising administering to a patient in need thereof an effective amount of a compound of Formula I, or a pharmaceutically acceptable salt or solvate thereof, or a pharmaceutical composition as disclosed herein.

In some embodiments, the disease, disorder, or condition includes, but is not limited to type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, and Polycystic Ovary Syndrome (PCOS).

In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof.

In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

In some embodiments, the compounds and pharmaceutical compositions and methods for treating a patient described herein induce one or more of blood glucose reduction (e.g., reduce blood glucose levels), reduce blood hemoglobin A1c (HbA1c) levels, promote insulin synthesis, stimulate insulin secretion, increase the mass of β-cells, modulate gastric acid secretion, modulate gastric emptying, decrease the body mass index (BMI), and/or decrease glucagon production (e.g., level). In certain embodiments, the compounds and pharmaceutical compositions and methods for treating a patient described herein stabilize serum glucose and serum insulin levels (e.g., serum glucose and serum insulin concentrations). Also provided herein are methods for modulating glucose or insulin levels in a patient in need of such modulating, the method comprising administering to the patient an effective amount of a compound of Formula I, or a pharmaceutically acceptable salt or solvate thereof, or a pharmaceutical composition as disclosed herein.

In some embodiments, provided herein is a method for reducing the risk (e.g., by about at least 20%, at least 30%, at least 40%, at least 50%, at least 60%, at least 70%, or at least 80%) of major adverse cardiovascular events (MACE) in a patient in need thereof, the method comprising administering to the patient an effective amount of a compound of Formula I, or a pharmaceutically acceptable salt or solvate thereof, or a pharmaceutical composition as disclosed herein. In certain of these embodiments, the patient is an adult that has been diagnosed with type 2 diabetes (T2D). In certain embodiments, the patient is an adult that has been diagnosed with a heart disease. In certain embodiments, the patient is an adult that has been diagnosed with type 2 diabetes (T2D) and a heart disease. In certain embodiments, the patient is an adult that has type 2 diabetes (T2D). In certain embodiments, the patient is an adult that has a heart disease. In certain embodiments, the patient has type 2 diabetes (T2D) and a heart disease.

### Indications

### Obesity

In some embodiments, the condition, disease or disorder is obesity and conditions, diseases or disorders that are associated with or related to obesity. Non-limiting examples of obesity and obesity related conditions include symptomatic obesity, simple obesity, childhood obesity, morbid obesity, and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

In some embodiments, the condition, disease or disorder is associated with obesity. Examples of such conditions, diseases or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, and metabolic syndrome. In some embodiments, the chemical compound and pharmaceutical compositions described herein can be used to treat patients exhibiting symptoms of both obesity and insulin deficiency.

### Diabetes

In some embodiments, the condition, disease or disorder is diabetes. Non-limiting examples of diabetes include type 1 diabetes mellitus, type 2 diabetes mellitus (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes. In some embodiments, the condition, disease or disorder is type 2 diabetes mellitus (e.g., diet-treated type 2-diabetes, sulfonylurea-treated type 2-diabetes, a far-advanced stage type 2-diabetes, long-term insulin-treated type 2-diabetes).

Provided herein is a method of treating a diabetes mellitus in a patient, the method comprising (a) determining that the patient has type 2 diabetes mellitus, and (b) administering to the patient a therapeutically effective amount of a compound of Formula I, or a pharmaceutically acceptable salt or solvate thereof, or a pharmaceutical composition as disclosed herein.

Provided herein is a method for treating type 2 diabetes mellitus in a patient, the method comprising administering to a patient identified or diagnosed as having type 2 diabetes mellitus a therapeutically effective amount of a compound of Formula I, or a pharmaceutically acceptable salt or solvate thereof, or a pharmaceutical composition as disclosed herein.

Also provided herein is a method of treating type 2 diabetes mellitus in a patient in need thereof, the method comprising administering to the patient a therapeutically effective amount of a compound of Formula I, or a pharmaceutically acceptable salt or solvate thereof, or a pharmaceutical composition as disclosed herein.

In some embodiments, the compounds and pharmaceutical compositions and methods for treating a patient with a condition, disease, or disorder (e.g., type 2 diabetes mellitus) described herein reduce fasting plasma glucose levels. In some embodiments, the compounds and pharmaceutical compositions and methods for treating a patient with a condition, disease, or disorder (e.g., type 2 diabetes mellitus) described herein reduce non-fasting plasma glucose levels. In some embodiments, the compounds and pharmaceutical compositions and methods for treating a patient with a condition, disease, or disorder (e.g., type 2 diabetes mellitus) described herein reduce HbA1c levels. In some embodiments, the compounds and pharmaceutical compositions and methods for treating a patient with a condition, disease, or disorder (e.g., type 2 diabetes mellitus) described herein reduce glucagon levels. In some embodiments, the compounds and pharmaceutical compositions and methods for treating a patient with a condition, disease, or disorder (e.g., type 2 diabetes mellitus) described herein increase insulin levels. In some embodiments, the compounds and pharmaceutical compositions and methods for treating a patient with a condition, disease, or disorder (e.g., type 2 diabetes mellitus) described herein reduce BMI.

In some embodiments, a reduction in fasting plasma glucose levels of about 5% to about 95% indicates treatment of type 2 diabetes mellitus. In some embodiments, a reduction in fasting plasma glucose levels of about 15% to about 80% indicates treatment of type 2 diabetes mellitus. In some embodiments, a reduction in fasting plasma glucose levels of about 25% to about 60% indicates treatment of type 2 diabetes mellitus. In some embodiments, a reduction in fasting plasma glucose levels to about or below 126 mg/dL, about or below 110 mg/dL, or about or below 90 mg/dL indicates treatment of the type 2 diabetes mellitus.

In some embodiments, a reduction in non-fasting plasma glucose levels of about 5% to about 95% indicates treatment of type 2 diabetes mellitus. In some embodiments, a reduction in non-fasting plasma glucose levels of about 15% to about 80% indicates treatment of type 2 diabetes mellitus. In some embodiments, a reduction in non-fasting plasma glucose levels of about 25% to about 60% indicates treatment of type 2 diabetes mellitus. In some embodiments, a reduction in non-fasting plasma glucose levels to about or below 200 mg/dL, about or below 150 mg/dL, or about or below 130 mg/dL indicates treatment of type 2 diabetes mellitus.

In some embodiments, a reduction in HbA1c levels of about 5% to about 95% indicates treatment of type 2 diabetes mellitus. In some embodiments, a reduction in HbA1c levels of about 15% to about 80% indicates treatment of type 2 diabetes mellitus. In some embodiments, a reduction in HbA1c levels of about 25% to about 60% indicates treatment of type 2 diabetes mellitus. In some embodiments, reduction in HbA1c levels to about or below 6.5%, about or below 6.0%, or about or below 5.0% indicates treatment of type 2 diabetes mellitus.

In some embodiments, a reduction in glucagon levels of about 5% to about 95% indicates treatment of type 2 diabetes mellitus. In some embodiments, a reduction in glucagon levels of about 15% to about 80% indicates treatment of type 2 diabetes mellitus. In some embodiments, a reduction in glucagon levels of about 25% to about 60% indicates treatment of type 2 diabetes mellitus. In some embodiments, an increase in insulin levels of about 5% to about 95% indicates treatment of type 2 diabetes mellitus. In some embodiments, an increase in insulin levels of about 15% to about 80% indicates treatment of type 2 diabetes mellitus. In some embodiments, an increase in insulin levels of about 25% to about 60% indicates treatment of type 2 diabetes mellitus.

In some embodiments, a reduction in BMI of about 5% to about 95% indicates treatment of type 2 diabetes mellitus. In some embodiments, a reduction in BMI of about 15% to about 80% indicates treatment of the type 2 diabetes mellitus. In some embodiments, a reduction in BMI of about 25% to about 60% indicates treatment of type 2 diabetes mellitus. In some embodiments, a reduction in BMI of about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of type 2 diabetes mellitus. In some embodiments, a reduction in BMI to about or below 40, about or below 30, or about or below 20 indicates treatment of type 2 diabetes mellitus.

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture, and cognitive dysfunction

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity). In some embodiments, the compounds described herein are also useful in improving the therapeutic effectiveness of metformin.

### Disorders of Metabolically Important Tissues

In some embodiments, the condition, disease or disorder is a disorder of a metabolically important tissue. Non-limiting examples of metabolically important tissues include liver, fat, pancreas, kidney, and gut.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, glycogen storage diseases, Weber-Christian disease, Wolman's disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g., metabolic syndrome (including obesity, diabetes and hypertriglyceridemia) and insulin resistance. It can cause liver disease in adults and children and may ultimately lead to cirrhosis (Skelly et al., J Hepatol 2001; 35: 195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl:S186-90). In some embodiments, the patient is a pediatric patient. The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age. In some embodiments, the patient is an adult patient.

Other non-limiting examples of disorders in metabolically important tissues include joint disorders (e.g., osteoarthritis, secondary osteoarthritis), steatosis (e.g. in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or hemodialysis, bone fracture, bone surgery, aging, pregnancy, protection against bone fractures, and malnutrition polycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction), and geriatric syndrome. In some embodiments, the compounds and pharmaceutical compositions described herein can be used for treating surgical trauma by improving recovery after surgery and/or by preventing the catabolic reaction caused by surgical trauma.

### Cardiovascular and Vascular Diseases

In some embodiments, the condition, disease or disorder is a cardiovascular disease. Non-limiting examples of cardiovascular disease include congestive heart failure, atherosclerosis, arteriosclerosis, coronary heart disease, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood).

In some embodiments, the condition, disease or disorder is related to a vascular disease. Non-limiting examples of vascular diseases include peripheral vascular disease, macrovascular complications (e.g., stroke), vascular dysfunction, peripheral artery disease, abdominal aortic aneurysm, carotid artery disease, cerebrovascular disorder (e.g., cerebral infarction), pulmonary embolism, chronic venous insufficiency, critical limb ischemia, retinopathy, nephropathy, and neuropathy.

### Neurological Diseases

In some embodiments, the condition, disease or disorder is a neurological disorder (e.g., neurodegenerative disorder) or a psychiatric disorder. Non-limiting examples of neurological disorders include brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), and chronic wasting syndrome). See, e.g., US2006/0275288A1.

Non-limiting examples of psychiatric disorders include drug dependence/addiction (narcotics and amphetamines and attention deficit/hyperactivity disorder (ADHD). The compounds and pharmaceutical compositions described herein can be useful in improving behavioral response to addictive drugs, decreasing drug dependence, prevention drug abuse relapse, and relieving anxiety caused by the absence of a given addictive substance. See, e.g., US2012/0021979A1.

In some embodiments, the compounds and pharmaceutical compositions described herein are useful in improving learning and memory by enhancing neuronal plasticity and facilitation of cellular differentiation, and also in preserving dopamine neurons and motor function in Morbus Parkinson.

### Insulin-Related Conditions and Disorders

In some embodiments, the condition, disease or disorder is impaired fasting glucose (IFG), impaired fasting glycemia (IFG), hyperglycemia, insulin resistance (impaired glucose homeostasis), hyperinsulinemia, elevated blood levels of fatty acids or glycerol, a hypoglycemic condition, insulin resistant syndrome, paresthesia caused by hyperinsulinemia, hyperlipidemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperprolactinemia, hypoglycemia (e.g., nighttime hypoglycemia), and concomitant comatose endpoint associated with insulin.

In some embodiments, the compounds and pharmaceutical compositions described herein can reduce or slow down the progression of borderline type, impaired fasting glucose or impaired fasting glycemia into diabetes.

### Autoimmune Disorders

In some embodiments, the condition, disease or disorder is an autoimmune disorder. Non-limiting examples of autoimmune disorders include multiple sclerosis, experimental autoimmune encephalomyelitis, autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves' disease. See, e.g., US20120148586A1.

### Stomach and Intestine-Related Disorders

In some embodiments, the condition, disease or disorder is a stomach or intestine related disorder. Non-limiting examples of these disorders include ulcers of any etiology (e.g. peptic ulcers, Zollinger-Ellison syndrome, drug-induced ulcers, ulcers related to infections or other pathogens), digestion disorders, malabsorption, short bowel syndrome, cul-de-sac syndrome, inflammatory bowel diseases (Crohn's disease and ulcerative colitis), celiac sprue, hypogammaglobulinemic sprue, chemotherapy and/or radiation therapy-induced mucositis and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, and cachexia caused by acquired immunodeficiency syndrome).

### Body Weight

In some embodiments, the compounds and pharmaceutical compositions described herein can be used to reduce body weight (e.g., excess body weight), prevent body weight gain, induce weight loss, decrease body fat, or reduce food intake in a patient (e.g., a patient in need thereof). In some embodiments, the weight increase in a patient may be attributed to excessive ingestion of food or unbalanced diets, or may be weight increase derived from a concomitant drug (e.g., insulin sensitizers having a PPARγ agonist-like action, such as troglitazone, rosiglitazone, englitazone, ciglitazone, pioglitazone and the like). In some embodiments, the weight increase may be weight increase before reaching obesity, or may be weight increase in an obese patient. In some embodiments, the weight increase may also be medication-induced weight gain or weight gain subsequent to cessation of smoking.

In some embodiments, the condition, disease or disorder is an eating disorder, such as hyperphagia, binge eating, bulimia, or compulsive eating.

### Inflammatory Diseases

In some embodiments, the condition, disease or disorder is an inflammatory disorder. Non-limiting examples of inflammatory disorders include chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney and gut, and a proinflammatory state (e.g., elevated levels of proinflammatory cytokines or markers of inflammation-like C-reactive protein in the blood).

### Cancer

In some embodiments, the condition, disease or disorder is cancer. Suitable examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

### Hypothalamic-Pituitary Disorders

In some embodiments, the condition, disease or disorder is related to the hypothalamic-pituitary-gonadal axis. For example, the condition, disease or disorder is related to the hypothalamus-pituitary-ovary axis. In another example, the condition, disease or disorder is related to the hypothalamus-pituitary-testis axis. Hypothalamic-pituitary-gonadal axis diseases include, but are not limited to, hypogonadism, polycystic ovary syndrome, hypothyroidism, hypopituitarism, sexual dysfunction, and Cushing's disease.

In some embodiments, the condition, disease or disorder associated with diabetes is related to the hypothalamic-pituitary-gonadal axis.

### Pulmonary Disease

In some embodiments, the condition, disease or disorder is related to a pulmonary disease. Pulmonary diseases include, but are not limited to, asthma, idiopathic pulmonary fibrosis, pulmonary hypertension, obstructive sleep apnoea-hypopnoea syndrome, and chronic obstructive pulmonary disease (COPD) (e.g., emphysema, chronic bronchitis, and refractory (non-reversible) asthma).

In some embodiments, the condition, disease or disorder associated with diabetes is a pulmonary disease.

### Combination Therapy

In some embodiments, this disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In some embodiments, the methods described herein include administering a compound described herein in combination with one or more of a diet therapy (e.g., dietary monitoring, diet therapy for diabetes), an exercise therapy (e.g., physical activity), blood sugar monitoring, gastric electrical stimulation (e.g., TANTALUS®), and diet modifications.

In some embodiments, the compounds of Formula I, or a pharmaceutically acceptable salt or solvate thereof as described herein can be administered in combination with one or more additional therapeutic agents.

Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, therapeutic agents for NASH, therapeutic agents for dysuria and anti-emetic agents.

In some embodiments, the one or more additional therapeutic agents include those useful, for example, as anti-obesity agents. Non-limiting examples include monoamine uptake inhibitors (e.g., tramadol, phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), including GABA receptor agonists (e.g., gabapentin, pregabalin), neuropeptide Y antagonists (e.g., velneperit), cannabinoid receptor antagonists (e.g., rimonabant, taranabant), ghrelin antagonists, ghrelin receptor antagonists, ghrelin acylation enzyme inhibitors, opioid receptor antagonists (e.g., GSK-1521498), orexin receptor antagonists, melanocortin 4 receptor agonists, 11β-hydroxysteroid dehydrogenase inhibitors (e.g., AZD-4017, BVT-3498, INCB-13739), pancreatic lipase inhibitors (e.g., orlistat, cetilistat), P3 agonists (e.g., N-5984), diacylglycerol acyltransferase 1 (DGAT1) inhibitors, acetylCoA carboxylase (ACC) inhibitors, stearoyl-CoA desaturated enzyme inhibitors, microsomal triglyceride transfer protein inhibitors (e.g., R-256918), sodium-glucose cotransporter 2 (SGLT-2) inhibitors (e.g., JNJ-28431754, dapagliflozin, AVE2268, TS-033, YM543, TA-7284, ASP1941, remogliflozin), NFK inhibitors (e.g., HE-3286), PPAR agonists (e.g., GFT-505, DRF-11605, gemfibrozil and fenofibrate), phosphotyrosine phosphatase inhibitors (e.g., sodium vanadate, trodusquemin), GPR119 agonists (e.g., PSN-821, MBX-2982, APD597), glucokinase activators (e.g., piragliatin, AZD-1656, AZD6370, TTP-355, compounds described in W0006/112549, W0007/028135, W0008/047821, W0008/050821, W0008/136428 and W0008/156757), leptin, leptin derivatives (e.g., metreleptin), leptin resistance improving drugs, CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin (OXM) preparations, appetite suppressants (e.g. ephedrine), FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), human proislet peptide (HIP), famesoid X receptor (FXR) agonist, phentermine, zonisamide, norepinephrine/dopamine reuptake inhibitor, GDF-15 analog, methionine aminopeptidase 2 (MetAP2) inhibitor, diethylpropion, phendimetrazine, benzphetamine, fibroblast growth factor receptor (FGFR) modulator, and AMP-activated protein kinase (AMPK) activator.

In some embodiments, the one or more additional therapeutic agents include those useful, for example, as anti-diabetic agents. Non-limiting examples include insulin and insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation, synthetic human insulin), insulin sensitizers (e.g., pioglitazone or a salt thereof), biguanides (e.g., metformin, buformin or a salt thereof (e.g., hydrochloride, fumarate, succinate)), glucagon analogs (e.g., any of glucagon analogs described, e.g., in WO 2010/011439), agents which antagonize the actions of or reduce secretion of glucagon, sulfonylurea agents (e.g., chlorpropamide, tolazamide, gliclazide, glimepiride, tolbutamide, glibenclamide, gliclazide, acetohexamide, glyclopyramide, glybuzole, glyburide), thiazolidinedione agents (e.g. rosiglitazone or pioglitazone), α-glucosidase inhibitors (e.g., voglibose, acarbose, miglitol, emiglitate), insulin secretagogues, such as prandial glucose regulators (sometimes called “short-acting secretagogues”), e.g., meglitinides (e.g. repaglinide and nateglinide), cholinesterase inhibitors (e.g., donepezil, galantamine, rivastigmine, tacrine), NMDA receptor antagonists, dual GLP-1/GIP receptor agonists (e.g., LBT-2000, ZPD1-70), GLP-1R agonists (e.g., exenatide, liraglutide, albiglutide, dulaglutide, abiglutide, taspoglutide, lixisenatide, semaglutide, AVE-0010, S4P and Boc5), and dipeptidyl peptidase IV (DPP-4) inhibitors (e.g., vildagliptin, dutogliptin, gemigliptin, alogliptin, saxagliptin, sitagliptin, linagliptin, berberine, adogliptin, BI1356, GRC8200, MP-513, PF-00734200, PHX1149, SK-0403, ALS2-0426, TA-6666, TS-021, KRP-104, trelagliptin).

In some embodiments, the one or more additional therapeutic agents include those useful, for example, for treating NAFL and NASH. Non-limiting examples include FXR agonists, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21), a niacin analogue, a leukotriene D4 (LTD4) receptor antagonist, an acetyl-CoA carboxylase (ACC) inhibitor, a ketohexokinase (KHK) inhibitor, an apoptosis signal-regulating kinase 1 (ASK1) inhibitor, an ileal bile acid transporter (IBAT) inhibitor, glycyrrhizin, Schisandra extract, ascorbic acid, glutathione, silymarin, lipoic acid, and d-alpha-tocopherol, ascorbic acid, glutathione, vitamin B-complex, glitazones/thiazolidinediones (e.g., troglitazone, rosiglitazone, pioglitazone), metformin, cysteamine, sulfonylureas, alpha-glucosidase inhibitors, meglitinides, vitamin E, tetrahydrolipstatin, milk thistle protein, anti-virals, and anti-oxidants.

In some embodiments, the one or more additional therapeutic agents include those useful, for example, for treating diabetic complications. Non-limiting examples include aldose reductase inhibitors (e.g., tolrestat, epalrestat, zopolrestat, fidarestat, CT-112, ranirestat, lidorestat), neurotrophic factor and increasing agents thereof (e.g., NGF, NT-3, BDNF, neurotrophic production/secretion promoting agents described in WO01/14372 (e.g., 4-(4-chlorophenyl)-2-(2-methyl-1-imidazolyl)-5-[3-(2-methylphenoxyl)propyl]oxazole), compounds described in WO2004/039365), PKC inhibitors (e.g., ruboxistaurin mesylate), AGE inhibitors (e.g., ALT946, N-phenacylthiazolium bromide (ALT766), EXO-226, pyridorin, pyridoxamine), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), and apoptosis signal regulating kinase-1 (ASK-1) inhibitors.

In some embodiments, the one or more additional therapeutic agents include those useful, for example, for treating hyperlipidemia. Non-limiting examples include HMG-COA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, e.g., N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid), fibrate compounds (e.g., bezafibrate, clofibrate, simfibrate, clinofibrate), anion exchange resin (e.g., colestyramine), nicotinic acid drugs (e.g., nicomol, niceritrol, niaspan), phytosterols (e.g., soysterol, gamma oryzanol (γ-oryzanol)), cholesterol absorption inhibitors (e.g., zechia), CETP inhibitors (e.g., dalcetrapib, anacetrapib) and ω-3 fatty acid preparations (e.g., ω-3-fatty acid ethyl esters 90).

In some embodiments, the one or more additional therapeutic agents include those useful, for example, as anti-hypertensive agents. Non-limiting examples include angiotensin converting enzyme inhibitors (e.g., captopril, enalapril, delapril), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil), calcium antagonists (e.g., manidipine, nifedipine, amlodipine, efonidipine, nicardipine, cilnidipine) and β-blockers (e.g., metoprolol, atenolol, propranolol, carvedilol, pindolol).

In some embodiments, the one or more additional therapeutic agents include those useful, for example, as diuretics. Non-limiting examples include xanthine derivatives (e.g., theobromine sodium salicylate, theobromine calcium salicylate), thiazide preparations (e.g., ethiazide, cyclopenthiazide, trichloromethiazide, hydrochlorothiazide, hydroflumethiazide, benzylhydrochlorothiazide, penfluthiazide, polythiazide, methyclothiazide), antialdosterone preparations (e.g., spironolactone, triamterene), carbonic anhydrase inhibitors (e.g., acetazolamide) and chlorobenzenesulfonamide agents (e.g., chlortalidone, mefruside, indapamide).

In some embodiments, the one or more additional therapeutic agents include those useful, for example, as immunotherapeutic agents. Non-limiting examples include microbial or bacterial compounds (e.g., muramyl dipeptide derivative, picibanil), polysaccharides having immunoenhancing activity (e.g., lentinan, sizofiran, krestin), cytokines obtained by genetic engineering approaches (e.g., interferon, interleukin (IL) such as IL-1, IL-2, IL-12), and colony-stimulating factors (e.g., granulocyte colony-stimulating factor, erythropoietin).

In some embodiments, the one or more additional therapeutic agents include those useful, for example, as anti-thrombotic agents. Non-limiting examples include heparins (e.g., heparin sodium, heparin calcium, enoxaparin sodium, dalteparin sodium) warfarin (e.g., warfarin potassium); anti-thrombin drugs (e.g., aragatroban, dabigatran) FXa inhibitors (e.g., rivaroxaban, apixaban, edoxaban, betrixaban, YM150, compounds described in WO02/06234, WO2004/048363, WO2005/030740, WO2005/058823, and WO2005/113504) thrombolytic agents (e.g., urokinase, tisokinase, alteplase, nateplase, monteplase, pamiteplase), and platelet aggregation inhibitors (e.g., ticlopidine hydrochloride, clopidogrel, prasugrel, E5555, SHC530348, cilostazol, ethyl icosapentate, beraprost sodium, and sarpogrelate hydrochloride).

In some embodiments, the one or more additional therapeutic agents include those useful, for example, for treating osteoporosis. Non-limiting examples include alfacalcidol, calcitriol, elcatonin, calcitonin salmon, estriol, ipriflavone, pamidronate disodium, alendronate sodium hydrate, incadronate disodium, and risedronate disodium. Suitable examples of vitamins include vitamin B1 and vitamin B12. Suitable examples of erectile dysfunction drugs include apomorphine and sildenafil citrate. Suitable examples of therapeutic agents for urinary frequency or urinary incontinence include flavorxate hydrochloride, oxybutynin hydrochloride and propiverine hydrochloride. Suitable examples of therapeutic agents for dysuria include acetylcholine esterase inhibitors (e.g., distigmine). Suitable examples of anti-inflammatory agents include nonsteroidal anti-inflammatory drugs such as aspirin, acetaminophen, indomethacin.

Other exemplary additional therapeutic agents include agents that modulate hepatic glucose balance (e.g., fructose 1,6-bisphosphatase inhibitors, glycogen phosphorylase inhibitors, glycogen synthase kinase inhibitors, glucokinase activators), agents designed to treat the complications of prolonged hyperglycemia, such as aldose reductase inhibitors (e.g. epalrestat and ranirestat), agents used to treat complications related to micro-angiopathies, anti-dyslipidemia agents, such as HMG-CoA reductase inhibitors (statins, e.g. rosuvastatin), cholesterol-lowering agents, bile acid sequestrants (e.g., cholestyramine), cholesterol absorption inhibitors (e.g. plant sterols such as phytosterols), cholesteryl ester transfer protein (CETP) inhibitors, inhibitors of the ileal bile acid transport system (IBAT inhibitors), bile acid binding resins, nicotinic acid (niacin) and analogues thereof, anti-oxidants (e.g., probucol), omega-3 fatty acids, antihypertensive agents, including adrenergic receptor antagonists, such as beta blockers (e.g. atenolol), alpha blockers (e.g. doxazosin), and mixed alpha/beta blockers (e.g. labetalol), adrenergic receptor agonists, including alpha-2 agonists (e.g. clonidine), angiotensin converting enzyme (ACE) inhibitors (e.g. lisinopril), calcium channel blockers, such as dihydropridines (e.g. nifedipine), phenylalkylamines (e.g. verapamil), and benzothiazepines (e.g. diltiazem), angiotensin II receptor antagonists (e.g. candesartan), aldosterone receptor antagonists (e.g. eplerenone), centrally acting adrenergic drugs, such as central alpha agonists (e.g. clonidine), diuretic agents (e.g. furosemide), haemostasis modulators, including antithrombotics (e.g., activators of fibrinolysis), thrombin antagonists, factor VIIa inhibitors, anticoagulants (e.g., vitamin K antagonists such as warfarin), heparin and low molecular weight analogues thereof, factor Xa inhibitors, and direct thrombin inhibitors (e.g. argatroban), antiplatelet agents (e.g., cyclooxygenase inhibitors (e.g. aspirin)), adenosine diphosphate (ADP) receptor inhibitors (e.g. clopidogrel), phosphodiesterase inhibitors (e.g. cilostazol), glycoprotein JIB/IIA inhibitors (e.g. tirofiban), adenosine reuptake inhibitors (e.g. dipyridamole), noradrenergic agents (e.g. phentermine), serotonergic agents (e.g. sibutramine), diacyl glycerolacyltransferase (DGAT) inhibitors, feeding behavior modifying agents, pyruvate dehydrogenase kinase (PDK) modulators, serotonin receptor modulators, monoamine transmission-modulating agents, such as selective serotonin reuptake inhibitors (SSRI) (e.g. fluoxetine), noradrenaline reuptake inhibitors (NARI), noradrenaline-serotonin reuptake inhibitors (SNRI), and monoamine oxidase inhibitors (MAOI) (e.g. toloxatone and amiflamine), compounds described in W0007/013694, WO2007/018314, WO2008/093639 and WO2008/099794, GPR40 agonists (e.g., fasiglifam or a hydrate thereof, compounds described in WO2004/041266, WO2004/106276, WO2005/063729, WO2005/063725, WO2005/087710, WO2005/095338, WO2007/013689 and WO2008/001931), SGLT1 inhibitors, adiponectin or agonist thereof, IKK inhibitors (e.g., AS-2868), somatostatin receptor agonists, ACC2 inhibitors, cachexia-ameliorating agents, such as a cyclooxygenase inhibitors (e.g., indomethacin), progesterone derivatives (e.g., megestrol acetate), glucocorticoids (e.g., dexamethasone), metoclopramide agents, tetrahydrocannabinol agents, agents for improving fat metabolism (e.g., eicosapentaenoic acid), growth hormones, IGF-1, antibodies against a cachexia-inducing factor TNF-α, LIF, IL-6, and oncostatin M, metabolism-modifying proteins or peptides such as glucokinase (GK), glucokinase regulatory protein (GKRP), uncoupling proteins 2 and 3 (UCP2 and UCP3), peroxisome proliferator-activated receptor α (PPARα), MC4r agonists, insulin receptor agonist, PDE 5 inhibitors, glycation inhibitors (e.g., ALT-711), nerve regeneration-promoting drugs (e.g., Y-128, VX853, prosaptide), antidepressants (e.g., desipramine, amitriptyline, imipramine), antiepileptic drugs (e.g., lamotrigine, trileptal, keppra, zonegran, pregabalin, harkoseride, carbamazepine), antiarrhythmic drugs (e.g., mexiletine), acetylcholine receptor ligands (e.g., ABT-594), endothelin receptor antagonists (e.g., ABT-627), narcotic analgesics (e.g., morphine), α2 receptor agonists (e.g., clonidine), local analgesics (e.g., capsaicin), antianxiety drugs (e.g., benzothiazepine), phosphodiesterase inhibitors (e.g., sildenafil), dopamine receptor agonists (e.g., apomorphine), cytotoxic antibodies (e.g., T-cell receptor and IL-2 receptor-specific antibodies), B cell depleting therapies (e.g., anti-CD20 antibody (e.g., rituxan), i-BLyS antibody), drugs affecting T cell migration (e.g., anti-integrin alpha 4/beta 1 antibody (e.g., tysabri), drugs that act on immunophilins (e.g., cyclosporine, tacrolimus, sirolimus, rapamicin), interferons (e.g., IFN-β), immunomodulators (e.g., glatiramer), TNF-binding proteins (e.g., circulating receptors), immunosupressants (e.g., mycophenolate), and metaglidasen, AMG-131, balaglitazone, MBX-2044, rivoglitazone, aleglitazar, chiglitazar, lobeglitazone, PLX-204, PN-2034, GFT-505, THR-0921, exenatide, exendin-4, memantine, midazolam, ketoconazole, ethyl icosapentate, clonidine, azosemide, isosorbide, ethacrynic acid, piretanide, bumetanide, etoposide.

In some embodiments, the one or more additional therapeutic agents include those useful, for example, as anti-emetic agents. As used herein, an “anti-emetic” agent refers to any agent that counteracts (e.g., reduces or removes) nausea or emesis (vomiting). While not wishing to be bound by theory, it is believed that administering one or more anti-emetic agents in combination with the Formula I compounds described herein may allow higher dosages of the Formula I compounds to be administered, e.g., because the patient may be able to have a normal food intake and thereby respond faster to the treatment.

Non-limiting examples of anti-emetic agents include 5HT3-receptor antagonists (serotonin receptor antagonists), neuroleptics/anti-psychotics, antihistamines, anticholinergic agents, steroids (e.g., corticosteroids), NK1-receptor antagonists (e.g., Neurokinin 1 substance P receptor antagonists), antidopaminergic agents/dopamine receptor antagonists, benzodiazepines, cannabinoids.

For example, the antiemetic agent can be selected from the group consisting of; neuroleptics, antihistamines, anti-cholinergic agents, steroids, 5HT-3-receptor antagonists, NK1-receptor antagonists, anti-dopaminergic agents/dopamine receptor antagonists, benzodiazepines and non-psychoactive cannabinoids.

In some embodiments, the anti-emetic agent is a 5HT3-receptor antagonist (serotonin receptor antagonist). Non-limiting examples of 5HT3-receptor antagonists (serotonin receptor antagonists) include: Granisetron (Kytril), Dolasetron, Ondansetron (Zofran), Tropisetron, Ramosetron, Palonosetron, Alosetron, azasetron, Bemesetron, Zatisetron, Batanopirde, MDL-73147EF; Metoclopramide, N-3389 (endo-3,9-dimethyl-3,9-diazabicyclo[3,3,1]non-7-yl-1H-indazole-3-carboxamide dihydrochloride), Y-25130 hydrochloride, MDL 72222, Tropanyl-3,5-dimethylbenzoate, 3-(4-Allylpiperazin-1-yl)-2-quinoxalinecarbonitrile maleate, Zacopride hydrochloride, and Mirtazepine. Other non-limiting examples of 5HT3-receptor antagonists (serotonin receptor antagonists) include: cilansetron, clozapine, cyproheptadine, dazopride, hydroxyzine, lerisetron, metoclopramide, mianserin, olanzapine, palonosetron (+ netupitant), quetiapine, qamosetron, ramosteron, ricasetron, risperidone, ziprasidone, and zatosetron.

In certain embodiments, the 5HT-3-receptor antagonist is Granisetron, Dolasetron, Ondansetron hydrochloride, Tropisetron, Ramosetron, Palonosetron, Alosetron, Bemesetron, Zatisetron, Batanopirde, MDL-73147EF, Metoclopramide, N-3389, Y-25130 hydrochloride, MDL 72222, Tropanyl-3,5-dimethylbenzoate 3-(4-AIIyI-piperazin-1-yl)-2-quinoxalinecarbonitrile maleate, Zacopride hydrochloride and Mirtazepine.

In certain embodiments, the 5HT-3-receptor antagonist is Granisetron, Dolasetron, Ondansetron hydrochloride, Tropisetron, Ramosetron, Palonosetron, Alosetron, Bemesetron, and Zatisetron.

In certain embodiments, the 5HT-3-receptor antagonist is Granisetron, Dolasetron and Ondansetron.

In certain embodiments, the 5HT-3-receptor antagonist is Granisetron.

In certain embodiments, the 5HT-3-receptor antagonist is Ondansetron.

In some embodiments, the anti-emetic agent is an antihistamine. Non-limiting examples of antihistamines include: piperazine derivatives (e.g., cyclizine, meclizine, and cinnarizine); Promethazine; Dimenhydrinate (Dramamine, Gravol); Diphenhydramine; Hydroxyzine; Buclizine; and Meclizine hydrochloride (Bonine, Antivert), doxylamine, and mirtazapine.

In some embodiments, the anti-emetic agent is an anticholinergic agent (Inhibitors of the acetylcholine receptors). Non-limiting examples of anticholinergic agents include: atropine, Scopolamine, Glycopyrron, Hyoscine, Artane (Trihexy-5 trihexyphenidyl hydrochloride), Cogentin (benztropine mesylate), Akineton (biperiden hydrochloride), Disipal (Norflex orphenadrine citrate), diphenhydramine, hydroxyzine, hyoscyamine, and Kemadrin (procyclidine hydrochloride).

In some embodiments, the anti-emetic agent is a steroid (e.g., a corticosteroid). Non-limiting examples of steroids include: betamethasone, Dexamethasone, Methylprednisolone, Prednisone®, and Trimethobenzamide (Tigan).

In some embodiments, the anti-emetic agent is an NK1-receptor antagonists (e.g., Neurokinin 1 substance P receptor antagonists). Non-limiting examples of NK1-receptor antagonists include: aprepitant, casopitant, ezlopitant, fosaprepitant, maropitant, netupitant, rolapitant, and vestipitant.

Other non-limiting examples of NK1-receptor antagonists include: MPC-4505, GW597599, MPC-4505, GR205171, L-759274, SR 140333, CP-96,345, BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-1, CJ-11974 j. Benserazide and carbidopa k. TAK-637 [(aR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthyridine-6,13-dione], PD 154075, ([(2-benzofuran)-CH₂OCO]—(R)-alpha-MeTrp-(S)—NHCH(CH₃) Ph), FK888, and (D-Pro4, D-Trp7,9,10, Phe11)SP4-11.

In some embodiments, the anti-emetic agent is an anti-dopaminergic agents/dopamine receptor antagonist (e.g., dopamine receptor antagonist, e.g., D2 or D3 antagonists). Non-limiting examples include phenothiazines (e.g., promethazine, chlorpromazine, prochlorperazine, perphenazine, hydroxyzine, thiethylperazine, metopimazine); benzamides (e.g., Metoclopramide, domperidone), butyrophenones (e.g., haloperidol, droperidol); alizapride, bromopride, clebopride, domperidone, itopride, metoclopramide, trimethobenzamide, and amisulpride.

In some embodiments, the anti-emetic agent is a non-psychoactive cannabinoids (e.g., Cannabidiol (CBD), Cannabidiol dimethylheptyl (CBD-DMH), Tetra-hydro-cannabinol (THC), Cannabinoid agonists such as WIN 55-212 (a CB1 and CB2 receptor agonist), Dronabinol (Marinol®), and Nabilone (Cesamet)).

Other exemplary anti-emetic agents include: c-9280 (Merck); benzodiazepines (diazepam, midazolam, lorazepam); neuroleptics/anti-psychotics (e.g., dixyrazine, haloperidol, and Prochlorperazine (Compazine®)); cerium oxalate; propofol; sodium citrate; dextrose; fructose (Nauzene); orthophosphoric acid; fructose; glucose (Emetrol); bismuth subsalicylate (Pepto Bismol); ephedrine; vitamin B6; peppermint, lavender, and lemon essential oils; and ginger.

Still other exemplary anti-emetic agents include those disclosed in US 20120101089A1; U.S. Pat. No. 10,071,088 B2; U.S. Pat. No. 6,673,792 B1; U.S. Pat. No. 6,197,329 B1; U.S. Pat. No. 10,828,297 B2; U.S. Pat. No. 10,322,106 B2; U.S. Pat. No. 10,525,033 B2; WO 2009080351 A1; WO 2019203753 A2; WO 2002020001 A2; U.S. Pat. No. 8,119,697 B2; U.S. Pat. No. 5,039,528; US20090305964A1; and WO 2006/111169, each of which is incorporated by reference in its entirety.

In some embodiments, the additional therapeutic agent or regimen is administered to the patient prior to contacting with or administering the compounds and pharmaceutical compositions (e.g., about one hour prior, or about 6 hours prior, or about 12 hours prior, or about 24 hours prior, or about 48 hours prior, or about 1 week prior, or about 1 month prior).

In some embodiments, the additional therapeutic agent or regimen is administered to the patient at about the same time as contacting with or administering the compounds and pharmaceutical compositions. By way of example, the additional therapeutic agent or regimen and the compounds and pharmaceutical compositions are provided to the patient simultaneously in the same dosage form. As another example, the additional therapeutic agent or regimen and the compounds and pharmaceutical compositions are provided to the patient concurrently in separate dosage forms.

### Patient Selection

In some embodiments, the methods described herein further include the step of identifying a patient (e.g., a subject) in need of such treatment (e.g., by way of blood assay, body mass index, or other conventional method known in the art).

In some embodiments, the methods described herein further include the step of identifying a patient (e.g., patient) that has a disease, disorder, or condition as provided here (e.g., a GLP-1 associated disease, disorder, or condition).

In some embodiments, the methods described herein further include the step of identifying a patient (e.g., patient) that has type 2 diabetes mellitus. In some embodiments, determining if the patient has type 2 diabetes mellitus includes performing an assay to determine the level of hemoglobin Ale (HbA1c), fasting plasma glucose, non-fasting plasma glucose, or any combination thereof. In some embodiments, the level of HbA1c is about 6.5% to about 24.0%. In some embodiments, the level of HbA1c is greater than or about 6.5%. In some embodiments, the level of HbA1c is greater than or about 8.0%. In some embodiments, the level of HbA1c is greater than or about 10.0%. In some embodiments, the level of HbA1c is greater than or about 12.0%. In some embodiments, the level of HbA1c is greater than or about 14.0%. In some embodiments, the level of HbA1c is greater than or about 16.0%. In some embodiments, the level of HbA1c is greater than or about 18.0%. In some embodiments, the level of HbA1c is greater than or about 20.0%. In some embodiments, the level of HbA1c is greater than or about 22.0%. In some embodiments, the level of HbA1c is greater than or about 24.0%.

In some embodiments, the level of fasting plasma glucose is greater than or about 120 mg/dL to greater than or about 750 mg/dL. In some embodiments, the level of fasting plasma glucose is greater than or about 200 mg/dL to greater than or about 500 mg/dL. In some embodiments, the level of fasting plasma glucose is greater than or about 300 mg/dL to greater than or about 700 mg/dL.

In some embodiments, the level of non-fasting plasma glucose is greater than or about 190 mg/dL to greater than or about 750 mg/dL. In some embodiments, the level of non-fasting plasma glucose is greater than or about 250 mg/dL to greater than or about 450 mg/dL. In some embodiments, the level of non-fasting plasma glucose is greater than or about 400 mg/dL to greater than or about 700 mg/dL.

In some embodiments, determining if the patient has type 2 diabetes mellitus further includes determining the patient's BMI. In some embodiments, the BMI of the patient is greater than or about 22 kg/m2 to greater than or about 100 kg/m2. In some embodiments, the BMI of the patient is greater than or about 30 kg/m2 to greater than or about 90 kg/m2. In some embodiments, the BMI of the patient is greater than or about 40 kg/m2 to greater than or about 80 kg/m2. In some embodiments, the BMI of the patient is greater than or about 50 kg/m2 to greater than or about 70 kg/m2.

In some embodiments, additional factors (e.g. risk factors) used for determining if the patient has type 2 diabetes mellitus further includes age and ethnicity of the patient. In some embodiments, the patient's age is greater than or about 10 years. In some embodiments, the patient's age is greater than or about 15 years. In some embodiments, the patient's age is greater than or about 20 years. In some embodiments, the patient's age is greater than or about 25 years. In some embodiments, the patient's age is greater than or about 30 years. In some embodiments, the patient's age is greater than or about 35 years. In some embodiments, the patient's age is greater than or about 40 years. In some embodiments, the patient's age is greater than or about 42 years. In some embodiments, the patient's age is greater than or about 44 years. In some embodiments, the patient's age is greater than or about 46 years. In some embodiments, the patient's age is greater than or about 48 years. In some embodiments, the patient's age is greater than or about 50 years. In some embodiments, the patient's age is greater than or about 52 years. In some embodiments, the patient's age is greater than or about 54 years. In some embodiments, the patient's age is greater than or about 56 years. In some embodiments, the patient's age is greater than or about 58 years. In some embodiments, the patient's age is greater than or about 60 years. In some embodiments, the patient's age is greater than or about 62 years. In some embodiments, the patient's age is greater than or about 64 years. In some embodiments, the patient's age is greater than or about 66 years. In some embodiments, the patient's age is greater than or about 68 years. In some embodiments, the patient's age is greater than or about 70 years. In some embodiments, the patient's age is greater than or about 72 years. In some embodiments, the patient's age is greater than or about 74 years. In some embodiments, the patient's age is greater than or about 76 years. In some embodiments, the patient's age is greater than or about 78 years. In some embodiments, the patient's age is greater than or about 80 years. In some embodiments, the patient's age is greater than or about 85 years. In some embodiments, the patient's age is greater than or about 90 years. In some embodiments, the patient's age is greater than or about 95 years. In some embodiments, the ethnicity of the patient may be African American, American Indian or Alaska Native, Asian American, Hispanics or Latinos, or Native Hawaiian or Pacific Islander.

### General Synthetic Methods

The compounds of this disclosure can be prepared from readily available starting materials using, for example, the following general methods, and procedures. It will be appreciated that where certain process conditions (i.e., reaction temperatures, times, mole ratios of reactants, solvents, pressures, etc.) are given, other process conditions can also be used unless otherwise stated. Optimum reaction conditions may vary with the reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting certain functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of this disclosure may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance CA USA), EMKA-Chemie Gmbh & Co. KG (Eching Germany), or Millipore Sigma (Burlington MA USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

Scheme I illustrates a general method which can be employed for the synthesis of compounds described herein, where each Ring A, Ring B, m, n, L, R³, R⁸, X¹, X², X³, and X⁴ are defined herein, and LG is a leaving group, such as halo (e.g., Cl, Br, or I) or a suitable electrophilic moiety, such as an aldehyde.

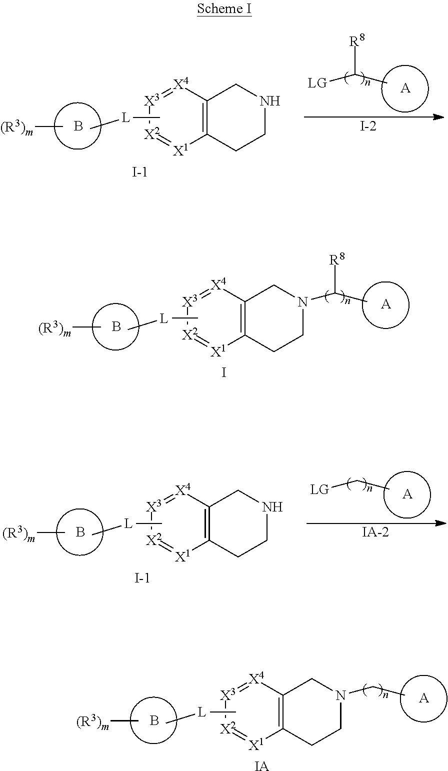

Compounds of Formula I can be provided by coupling compound I-1 with compound I-2 under suitable coupling reaction conditions, such as SN2 reaction conditions. Compounds of Formula IA can be provided by coupling compound I-1 with compound IA-2 under suitable coupling reaction conditions, such as SN2 reaction conditions. Exemplary suitable reaction conditions include, but are not limited to, a polar aprotic solvent (e.g., acetonitrile), optionally in the presence of a base (e.g., potassium carbonate). Further derivatization can be performed of the resulting product via methods and chemical transformations which are known to those of skill in the art can provide alternative compounds of Formula I or IA. For example, when the leaving group is an electrophile, such as an aldehyde, the coupling reaction conditions may comprise reductive amination reaction conditions. Thus, the conversion may comprise more than one reaction or set of reactants.

For any compound shown in Scheme I, it should be understood that various derivatives can be provided by functional group interconversion at any step. For example with R³, various compounds of Formula I or IA can be provided via transesterification or hydrolysis using methods known to one of skill in the art. Likewise, various compounds of Formula I or IA can be prepared by contacting compounds where one or more R⁴ is a leaving group (e.g., halo, such as Cl, Br, or I, or a pseudohalide, such as a triflate, sulfonate, or phosphate), with a compound of Formula (R³)ₘ-(Ring B)-M, wherein M is a suitable functional group such as, but not limited to, a boronic acid or a derivative thereof, such as a boronic ester, zinc or magnesium halide, an organotin compound, such as tributylstannane or trimethylstannane, fluorosulfonyl esters, tin, sodium, hydrogen, and the like. Such reactions are commonly utilized for aromatic functionalization, and are typically conducted in the presence of suitable catalyst such as, but not limited to, a palladium catalyst including [1,1′-bis(diphenylphosphino)ferrocene]palladium(II) dichloride, Pd(OAc)₂, Pd(PPh₃)₄, PdCl₂(PPh₃)₂ or tris(dibenzylideneacetone)dipalladium(0), and the like, or a copper catalyst such as CuCl or CuI, and if required suitable mediator, co-catalyst and/or base known to one skilled in the art using suitable solvents/solvent mixtures. Upon reaction completion, compounds of Formula I can be recovered by conventional techniques such as neutralization, extraction, precipitation, chromatography, filtration and the like. In certain embodiments, when control of stereochemistry is desired, proper control of reaction conditions and selection of substituents for the reagents can at least partially dictate or preserve the formation of the various stereoisomers.

In some embodiments, the various substituents of Formula I-1, I-A, or IA-2 are as defined herein. However, derivatization thereof prior to reacting in any step, and/or further derivatization of the resulting reaction product, provides various compounds of Formula I or IA. Appropriate starting materials and reagents can be purchased or prepared by methods known to one of skill in the art. Upon each reaction completion, each of the intermediate or final compounds can be recovered, and optionally purified, by conventional techniques such as neutralization, extraction, precipitation, chromatography, filtration, and the like. Other modifications to arrive at compounds of this disclosure are within the skill of the art.

In some embodiments, the compound of Formula I-2 or IA-2 for use in Scheme I is represented by Formula II-1, 111-1, IV-1, or V-1:

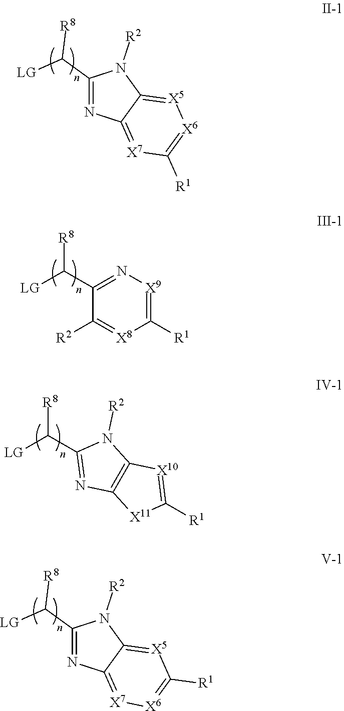

where n, R¹, R², R⁸, X⁵, X⁶, and X⁷ are each independently as defined herein, and LG is a leaving group, such as halo (e.g., Cl, Br, or I) or a suitable electrophilic moiety, such as an aldehyde.

In some embodiments, the compound of Formula I-2 or IA-2 for use in Scheme I is represented by Formula IIA-1:

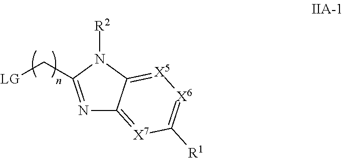

where n, R¹, R², X⁵, X⁶, and X⁷ are each independently as defined herein, and LG is a leaving group, such as halo (e.g., Cl, Br, or I) or a suitable electrophilic moiety, such as an aldehyde.

In some embodiments, the compound of Formula I-2 is represented by Formula IIIA-1:

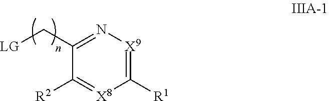

where n, R¹, R², X⁸, and X⁹ are each independently as defined herein, and LG is a leaving group, such as halo (e.g., Cl, Br, or I) or a suitable electrophilic moiety, such as an aldehyde.

In some embodiments, the compound of Formula I-2 is represented by Formula IVA-1:

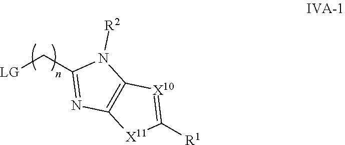

where n, R¹, R², X¹⁰, and X¹¹ are each independently as defined herein, and LG is a leaving group, such as halo (e.g., Cl, Br, or I) or a suitable electrophilic moiety, such as an aldehyde.

In some embodiments, the compound of Formula I-2 is represented by Formula VA-1:

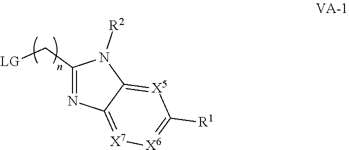

where n, R¹, R², X⁵, X⁶, and X⁷ are each independently as defined herein, and LG is a leaving group, such as halo (e.g., Cl, Br, or I) or a suitable electrophilic moiety, such as an aldehyde.

In some embodiments, provided is a process for preparing a compound of Formula I as described herein, or a pharmaceutically acceptable salt or solvate thereof, comprising contacting a compound of Formula IIA-1 or IIIA-1, IVA-1, or VA-1:

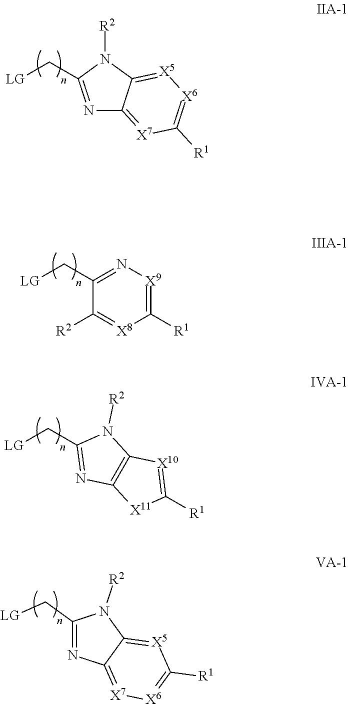

with a compound of Formula I-1:

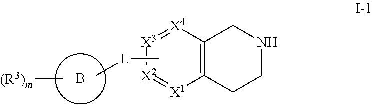

under conditions sufficient to provide the compound of Formula I, or a pharmaceutically acceptable salt or solvate thereof, where each Ring B, m, n, L, R¹, R², R³, X¹, X², X³, X⁴, X⁵, X⁶, X⁷, X⁸, X⁹, X¹⁰, and X¹¹ are each independently as defined herein, and LG is a leaving group, such as halo (e.g., Cl, Br, or I) or a suitable electrophilic moiety, such as an aldehyde. In certain embodiments, the conditions are described in Scheme I or the procedures described below.

In some embodiments, provided is a process for preparing a compound of Formula I as described herein, or a pharmaceutically acceptable salt or solvate thereof, comprising contacting a compound of Formula IIA-1 or IIIA-1:

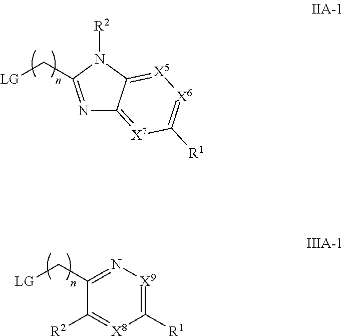

with a compound of Formula I-1:

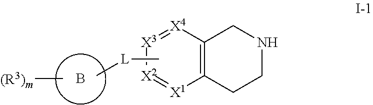

under conditions sufficient to provide the compound of Formula I, or a pharmaceutically acceptable salt or solvate thereof, where each Ring B, m, n, L, R¹, R², R³, X¹, X², X³, X⁴, X⁵, X⁶, X⁷, X⁸, and X⁹ are each independently as defined herein, and LG is a leaving group, such as halo (e.g., Cl, Br, or I) or a suitable electrophilic moiety, such as an aldehyde. In certain embodiments, the conditions are described in Scheme I or the procedures described below.

### EXAMPLES

General information: All evaporations were carried out in vacuo with a rotary evaporator. Analytical samples were dried in vacuo (1-5 mm Hg) at it. Thin layer chromatography (TLC) was performed on silica gel plates, spots were visualized by UV light (214 and 254 nm). Purification by column and flash chromatography was carried out using silica gel (100-200 mesh). Solvent systems were reported as mixtures by volume. NMR spectra were recorded on a Bruker 400 or Varian (400 MHz) spectrometer. 1H chemical shifts are reported in 6 values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on SHIMADZU LC20-MS2020 or Agilent 1260 series 6125B mass spectrometer or Agilent 1200 series, 6110 or 6120 mass spectrometer with electrospray ionization and excepted as otherwise indicated.

This disclosure is further understood by reference to the following procedures, which are intended to be purely exemplary of the disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description and accompanying figures. Such modifications fall within the scope of the appended claims.

### Procedure 1

### (S)-3-(2-((2-((4-chloro-2-fluorobenzyl)oxy)-5,8-dihydro-1,7-naphthyridin-7(6H)-yl)methyl)-3-(oxetan-2-ylmethyl)-3H-imidazo[4,5-b]pyridin-6-yl)-1,2,4-oxadiazol-5(4H)-one (compound 1)

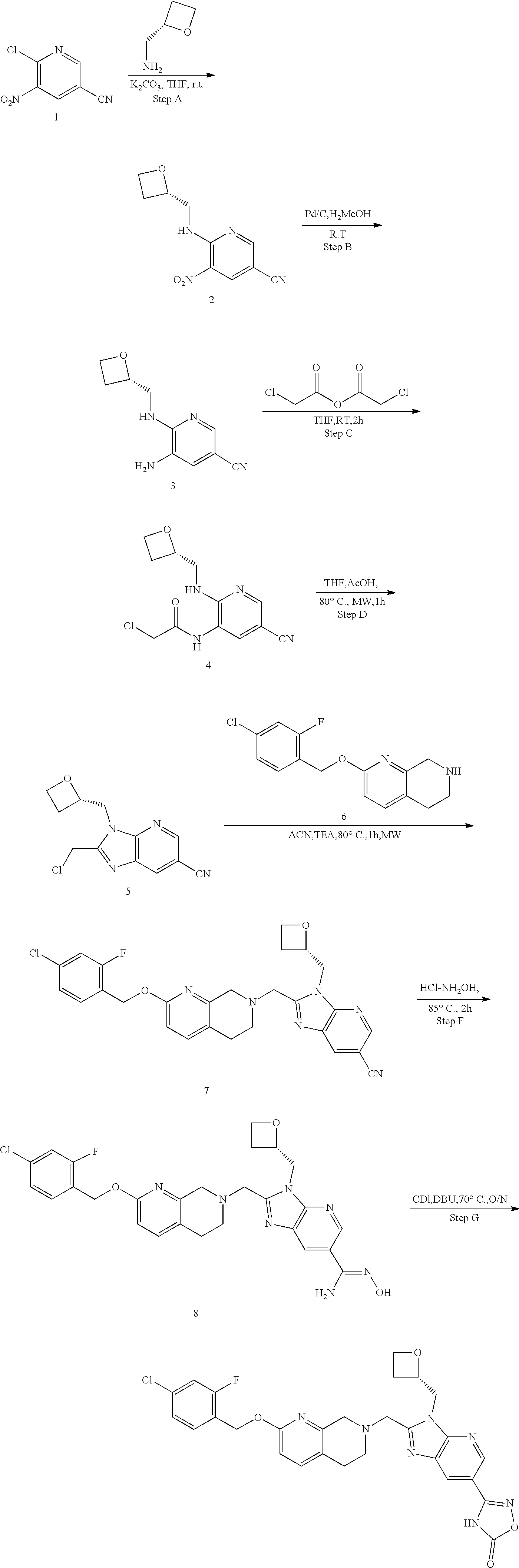

### Step A: (S)-5-nitro-6-((oxetan-2-ylmethyl)amino)nicotinonitrile

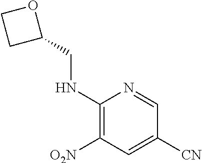 [SMILES: N#Cc1cnc(NC[C@@H]2CCO2)c([N+](=O)[O-])c1]

A mixture of 6-chloro-5-nitronicotinonitrile (1.00 g, 5.45 mmol), (S)-oxetan-2-ylmethanamine 4-methylbenzenesulfonate (1.67 g, 6.54 mmol) and K₂CO₃ (3.20 g, 16.35 mmol) in THF (35 mL) was stirred at room temperature overnight. The mixture was filtered, washed with DCM (30 mL), concentrated to give (S)-5-nitro-6-((oxetan-2-ylmethyl)amino)nicotinonitrile (1.00 g, 79% yield). LC-MS: m/z 235.2 (M+H)⁺.

### Step B: (S)-5-amino-6-((oxetan-2-ylmethyl)amino)nicotinonitrile

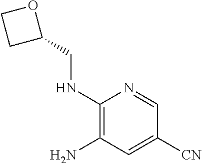 [SMILES: N#Cc1cnc(NC[C@@H]2CCO2)c(N)c1]

To a mixture of (S)-5-nitro-6-((oxetan-2-ylmethyl)amino)nicotinonitrile (1.00 g, 4.27 mmol) in MeOH (30 mL) was added Pd/C (10%) (200 mg) at room temperature. The mixture was stirred at room temperature under H₂ atmosphere overnight. The mixture was filtered, washed with MeOH (20 mL), concentrated to give (S)-5-amino-6-((oxetan-2-ylmethyl)amino)nicotinonitrile (650 mg, 74% yield). LC-MS: m/z 205.1 (M+H)⁺.

### Step C: (S)-2-chloro-N-(5-cyano-2-((oxetan-2-ylmethyl)amino)pyridin-3-yl)acetamide

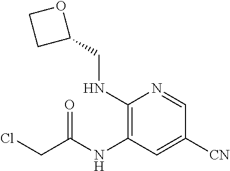 [SMILES: N#Cc1cnc(NC[C@@H]2CCO2)c(NC(=O)CCl)c1]

To a mixture of (S)-5-amino-6-((oxetan-2-ylmethyl)amino)nicotinonitrile (650 mg, 3.18 mmol) in THF (20 mL) was added 2-chloroacetic anhydride (673 mg, 3.97 mmol) at room temperature. The mixture was stirred at room temperature for 2 hours. The mixture was concentrated to give (S)-2-chloro-N-(5-cyano-2-((oxetan-2-ylmethyl)amino)pyridin-3-yl)acetamide (crude), which was used in next step without further purification. LC-MS: m/z 281.1 (M+H)⁺.

### Step D: (S)-2-(chloromethyl)-3-(oxetan-2-ylmethyl)-3H-imidazo[4,5-b]pyridine-6-carbonitrile

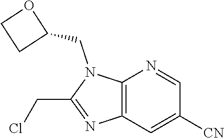 [SMILES: N#Cc1cnc2c(c1)nc(CCl)n2C[C@@H]1CCO1]

A mixture of (S)-2-chloro-N-(5-cyano-2-((oxetan-2-ylmethyl)amino)pyridin-3-yl)acetamide in THF/AcOH (10 mL/0.5 mL) was stirred at 80° C. for 1 hour under microwave irradiation. The mixture was concentrated, diluted with NaHCO₃(aq), extracted with EtOAc (20 mL), dried over Na₂SO₄ and concentrated, the residue was purified by Prep-HPLC (0.10% formic acid in H₂O and MeOH) to give (S)-2-(chloromethyl)-3-(oxetan-2-ylmethyl)-3H-imidazo[4,5-b]pyridine-6-carbonitrile (85.0 mg, 10% yield). LC-MS: m/z 263.1 (M+H)⁺.

### Step E: (S)-2-((2-((4-chloro-2-fluorobenzyl)oxy)-5,8-dihydro-1,7-naphthyridin-7(6H)-yl)methyl)-3-(oxetan-2-ylmethyl)-3H-imidazo[4,5-b]pyridine-6-carbonitrile

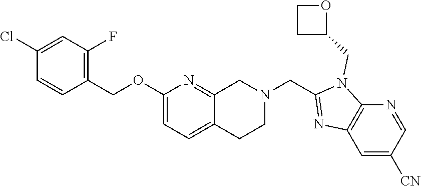 [SMILES: N#Cc1cnc2c(c1)nc(CN1CCc3ccc(OCc4ccc(Cl)cc4F)nc3C1)n2C[C@@H]1CCO1]

To a mixture of (S)-2-(chloromethyl)-3-(oxetan-2-ylmethyl)-3H-imidazo[4,5-b]pyridine-6-carbonitrile (85.0 mg, 0.323 mmol) in ACN (3 mL) were added 2-((4-chloro-2-fluorobenzyl)oxy)-5,6,7,8-tetrahydro-1,7-naphthyridine (94.0 mg, 0.323 mmol), TEA (162 mg, 1.61 mmol) at room temperature. The mixture was stirred at 80° C. for 1 hour under microwave irradiation. The mixture was concentrated and purified by Pre-TLC (PE/EtOAc=1:1) to give (S)-2-((2-((4-chloro-2-fluorobenzyl)oxy)-5,8-dihydro-1,7-naphthyridin-7(6H)-yl)methyl)-3-(oxetan-2-ylmethyl)-3H-imidazo[4,5-b]pyridine-6-carbonitrile (60.0 mg, 36% yield). LC-MS: m/z 519.0 (M+H)⁺.

### Step F: (S,Z)-2-((2-((4-chloro-2-fluorobenzyl)oxy)-5,8-dihydro-1,7-naphthyridin-7(6H)-yl)methyl)-N′-hydroxy-3-(oxetan-2-ylmethyl)-3H-imidazo[4,5-b]pyridine-6-carboximidamide

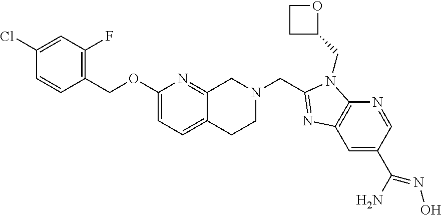 [SMILES: N/C(=N\O)c1cnc2c(c1)nc(CN1CCc3ccc(OCc4ccc(Cl)cc4F)nc3C1)n2C[C@@H]1CCO1]

To a mixture of (S)-2-((2-((4-chloro-2-fluorobenzyl)oxy)-5,8-dihydro-1,7-naphthyridin-7(6H)-yl)methyl)-3-(oxetan-2-ylmethyl)-3H-imidazo[4,5-b]pyridine-6-carbonitrile (60 mg, 0.115 mmol) in EtOH (10 mL) was added HCl·NH₂OH (40.0 mg, 0.575 mmol) and TEA (58.0 mg, 0.575 mmol) at room temperature. The mixture was stirred at 85° C. for 2 hours. The mixture was concentrated, the residue was diluted with H₂O, extracted with DCM (8 mL*3), The organic layer was dried over Na₂SO₄, and concentrated to give (S,Z)-2-((2-((4-chloro-2-fluorobenzyl)oxy)-5,8-dihydro-1,7-naphthyridin-7(6H)-yl)methyl)-N′-hydroxy-3-(oxetan-2-ylmethyl)-3H-imidazo[4,5-b]pyridine-6-carboximidamide (63.4 mg) (crude, product and byproduct same retention time). LC-MS: m/z 537.2, 552 (M+H)⁺

### Step G: (S)-3-(2-((2-((4-chloro-2-fluorobenzyl)oxy)-5,8-dihydro-1,7-naphthyridin-7(6H)-yl)methyl)-3-(oxetan-2-ylmethyl)-3H-imidazo[4,5-b]pyridin-6-yl)-1,2,4-oxadiazol-5(4H)-one

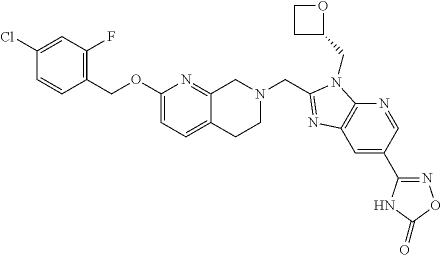 [SMILES: O=c1[nH]c(-c2cnc3c(c2)nc(CN2CCc4ccc(OCc5ccc(Cl)cc5F)nc4C2)n3C[C@@H]2CCO2)no1]

To a mixture of (S,Z)-2-((2-((4-chloro-2-fluorobenzyl)oxy)-5,8-dihydro-1,7-naphthyridin-7(6H)-yl)methyl)-N′-hydroxy-3-(oxetan-2-ylmethyl)-3H-imidazo[4,5-b]pyridine-6-carboximidamide (63.4 mg, 0.115 mmol) in DMSO (6 mL) were added CDI (37.2 mg, 0.230 mmol) and DBU (43.7 mg, 0.287 mmol) at room temperature. The mixture was stirred at 70° C. overnight. The mixture was adjusted pH=4 with diluted HCOOH, then H₂O (6 mL) was added to the mixture, filtered, the filter residue was dissolved with DCM, dried over Na₂SO₄ and concentrated. The residue was purified by Prep-HPLC (0.1% formic acid in H₂O and MeOH) to give (S)-3-(2-((2-((4-chloro-2-fluorobenzyl)oxy)-5,8-dihydro-1,7-naphthyridin-7(6H)-yl)methyl)-3-(oxetan-2-ylmethyl)-3H-imidazo[4,5-b]pyridin-6-yl)-1,2,4-oxadiazol-5(4H)-one (3.00 mg, 4% yield).

¹H NMR (400 MHz, CDCl₃) δ 8.77 (s, 1H), 8.38 (s, 1H), 7.41 (t, J=8.0 Hz, 1H), 7.35 (d, J=8.4 Hz, 1H), 7.09-7.13 (m, 2H), 6.63 (d, J=8.4 Hz, 1H), 5.28 (s, 2H), 5.18-5.24 (m, 1H), 4.88-4.97 (m, 1H), 4.72-4.79 (m, 1H), 4.61 (dd, J=14.0, 7.6 Hz, 1H), 4.44-4.52 (m, 1H), 4.34-4.41 (m, 1H), 3.79-4.00 (m, 2H), 3.00-3.27 (m, 1H), 2.85-2.97 (m, 2H), 2.67-2.81 (m, 2H), 2.39-2.49 (m, 2H). ¹⁹F NMR (376 MHz, CDCl₃) δ −115.57. LC-MS: m/z 578.0 (M+H)⁺.

### Procedure 2

### (S)-3-(2-((2-((4-chloro-2-fluorobenzyl)oxy)-5,8-dihydro-1,7-naphthyridin-7(6H)-yl)methyl)-1-(oxetan-2-ylmethyl)-1H-benzo[d]imidazol-6-yl)-1,2,4-oxadiazol-5(4H)-one (compound 2)

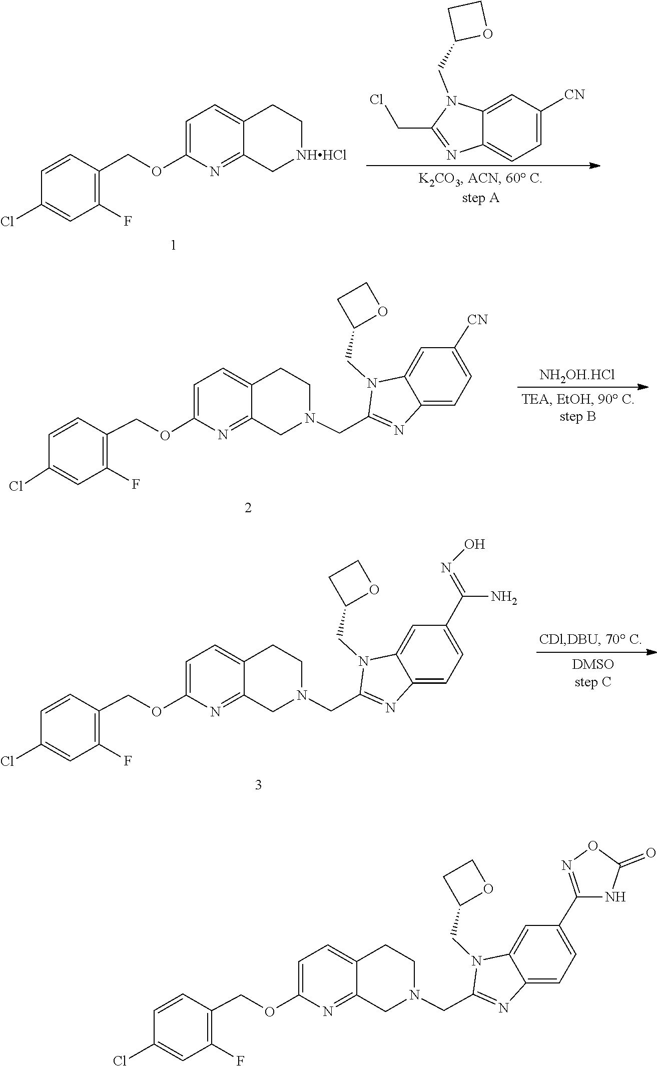

### Step A: (S)-2-((2-((4-chloro-2-fluorobenzyl)oxy)-5,8-dihydro-1,7-naphthyridin-7(6H)-yl)methyl)-1-(oxetan-2-ylmethyl)-1H-benzo[d]imidazole-6-carbonitrile

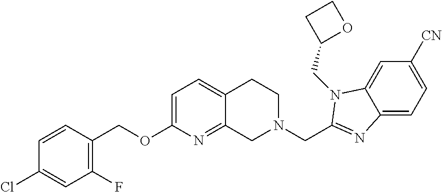 [SMILES: N#Cc1ccc2nc(CN3CCc4ccc(OCc5ccc(Cl)cc5F)nc4C3)n(C[C@@H]3CCO3)c2c1]

A mixture of 2-((4-chloro-2-fluorobenzyl)oxy)-5,6,7,8-tetrahydro-1,7-naphthyridin-7-ium chloride (50.0 mg, 0.152 mmol), (S)-2-(chloromethyl)-1-(oxetan-2-ylmethyl)-1H-benzo[d]imidazole-6-carbonitrile (39.8 mg, 0.152 mmol), K₂CO₃ (62.9 mg, 0.456 mmol) in acetonitrile (4 mL) was stirred at 60° C. overnight. The mixture was diluted with H₂O (20 mL) and extracted with EtOAc (30 mL*3). The combined organic phase was dried over Na₂SO₄ and filtered. The filtrate was concentrated and purified by prep-TLC (PE/EtOAc=1/1) to give (S)-2-((2-((4-chloro-2-fluorobenzyl)oxy)-5,8-dihydro-1,7-naphthyridin-7(6H)-yl)methyl)-1-(oxetan-2-ylmethyl)-1H-benzo[d]imidazole-6-carbonitrile (72.0 mg, 91% yield). LC-MS: m/z 518.4 (M+H)⁺.

### Step B: (S,Z)-2-((2-((4-chloro-2-fluorobenzyl)oxy)-58-dihydro-1,7-naphthyridin-7(6H)-yl)methyl)-N′-hydroxy-1-(oxetan-2-ylmethyl)-1H-benzo[d]imidazole-6-carboximidamide

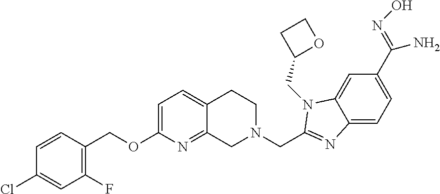 [SMILES: N/C(=N\O)c1ccc2nc(CN3CCc4ccc(OCc5ccc(Cl)cc5F)nc4C3)n(C[C@@H]3CCO3)c2c1]

A solution of (S)-2-((2-((4-chloro-2-fluorobenzyl)oxy)-5,8-dihydro-1,7-naphthyridin-7(6H)-yl)methyl)-1-(oxetan-2-ylmethyl)-1H-benzo[d]imidazole-6-carbonitrile (72.0 mg, 0.139 mmol), hydroxylamine hydrochloride (48.0 mg, 0.695 mmol), triethylamine (78.0 mg, 0.765 mmol) in ethanol (5 mL) was stirred at 90° C. for 3 hours. The mixture was diluted with H₂O (20 mL) and extracted with EtOAc (30 mL*3). The combined organic phase was dried over Na₂SO₄ and filtered. The filtrate was concentrated and purified by prep-TLC (DCM/MeOH=10/1) to give (S,Z)-2-((2-((4-chloro-2-fluorobenzyl)oxy)-5,8-dihydro-1,7-naphthyridin-7(6H)-yl)methyl)-N′-hydroxy-1-(oxetan-2-ylmethyl)-1H-benzo[d]imidazole-6-carboximidamide (62.0 mg, 81% yield). LC-MS: m/z 551.4 (M+H)⁺.

### Step C: (S)-3-(2-((2-((4-chloro-2-fluorobenzyl)oxy)-5,8-dihydro-1,7-naphthyridin-7(6H)-yl)methyl)-1-(oxetan-2-ylmethyl)-1H-benzo[d]imidazol-6-yl)-1,2,4-oxadiazol-5(4H)-one

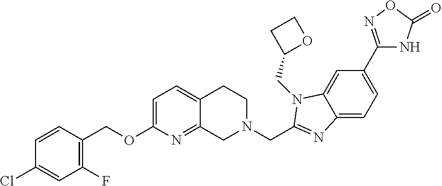 [SMILES: O=c1[nH]c(-c2ccc3nc(CN4CCc5ccc(OCc6ccc(Cl)cc6F)nc5C4)n(C[C@@H]4CCO4)c3c2)no1]

A mixture of (S,Z)-2-((2-((4-chloro-2-fluorobenzyl)oxy)-5,8-dihydro-1,7-naphthyridin-7(6H)-yl)methyl)-N′-hydroxy-1-(oxetan-2-ylmethyl)-1H-benzo[d]imidazole-6-carboximidamide (72.0 mg, 0.139 mmol), 1,1′-Carbonyldiimidazole (36.5 mg, 0.225 mmol), DBU (42.9 mg, 0.283 mmol) in DMSO (3 mL) was stirred at 70° C. for 1 hour. The mixture was diluted with H₂O (20 mL) and extracted with DCM (30 mL*3). The combined organic phase was dried over Na₂SO₄ and filtered. The filtrate was concentrated and purified by prep-HPLC (0.1% HCOOH in water/CH₃CN) to give (S)-3-(2-((2-((4-chloro-2-fluorobenzyl)oxy)-5,8-dihydro-1,7-naphthyridin-7(6H)-yl)methyl)-1-(oxetan-2-ylmethyl)-1H-benzo[d]imidazol-6-yl)-1,2,4-oxadiazol-5(4H)-one (30.0 mg, 38% yield).

¹H NMR (400 MHz, DMSO-d₆) δ 12.95 (br.s, 1H), 8.14 (d, J=1.2 Hz, 1H), 7.80 (d, J=8.8 Hz, 1H), 7.65 (dd, J=8.4, 1.6 Hz, 1H), 7.53 (t, J=8.0 Hz, 1H), 7.49 (d, J=8.4 Hz, 1H), 7.44 (dd, J=10.0, 2.0 Hz, 1H), 7.28 (dd, J=8.4, 2.0 Hz, 1H), 6.67 (d, J=8.0 Hz, 1H), 5.26 (s, 2H), 5.02-5.13 (m, 1H), 4.76 (dd, J=15.2, 7.2 Hz, 1H), 4.63 (dd, J=15.2, 3.2 Hz, 1H), 4.43-4.51 (m, 1H), 4.35-4.42 (m, 1H), 4.15 (d, J=13.6 Hz, 1H), 4.02 (d, J=13.6 Hz, 1H), 3.62 (q, J=16.0 Hz, 2H), 2.70-2.87 (m, 4H), 2.59-2.69 (m, 1H), 2.35-2.45 (m, 1H). ¹⁹F NMR (376 MHz, DMSO-d₆) δ −115.18. LC-MS: m/z 577.1 (M+H)⁺.

### Procedure 3

### 2-[(4-chloro-2-fluorophenyl)methoxy]-3-cyclopropyl-7-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-5,6,7,8-tetrahydro-1,7-naphthyridine (compound 3)

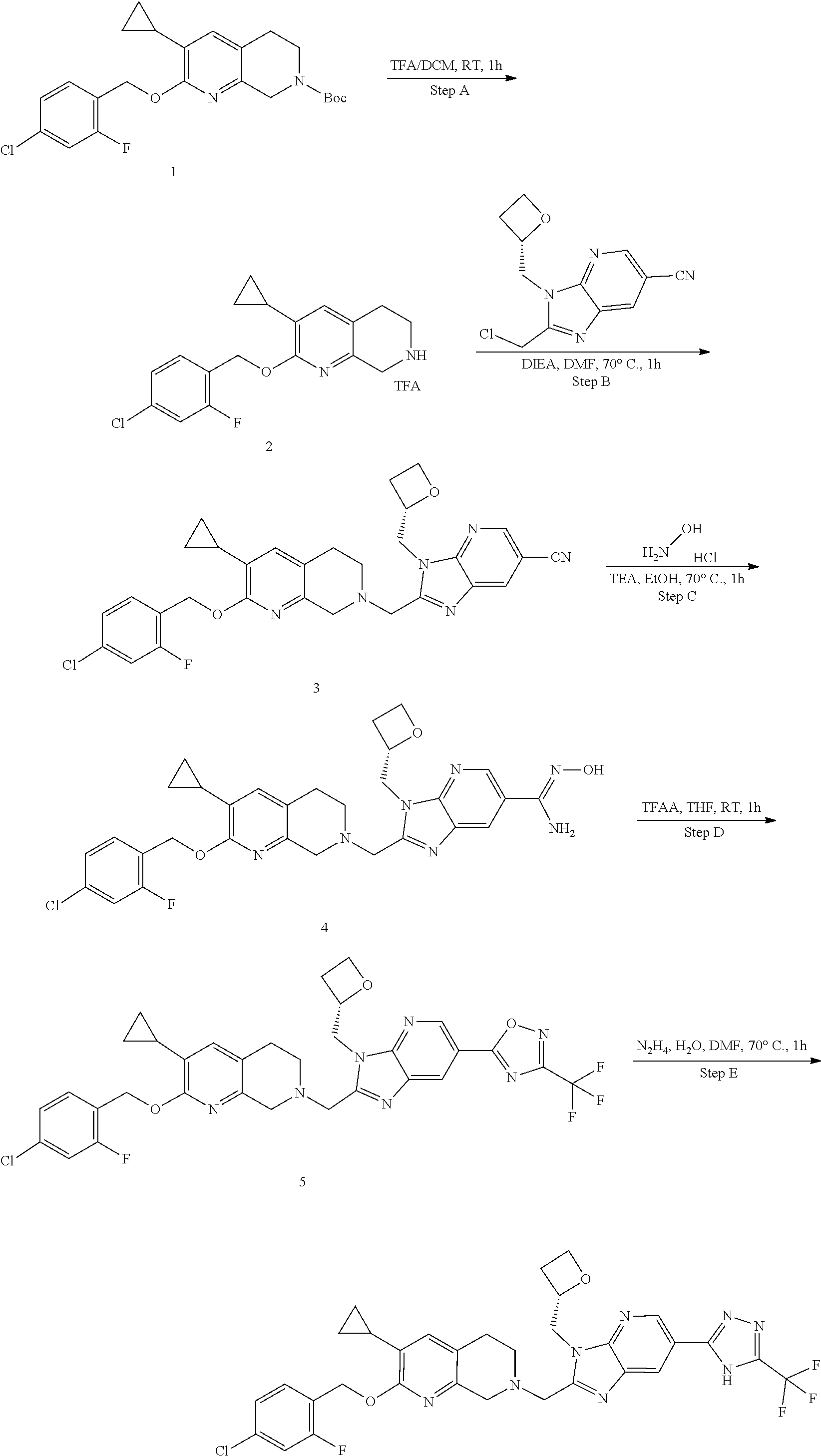

### Step A: 2-((4-chloro-2-fluorobenzyl)oxy)-3-cyclopropyl-5,6,7,8-tetrahydro-1,7-naphthyridine, TFA Salt

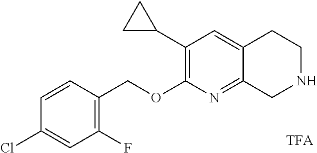 [SMILES: Fc1cc(Cl)ccc1COc1nc2c(cc1C1CC1)CCNC2]

To a solution of tert-butyl 2-((4-chloro-2-fluorobenzyl)oxy)-3-cyclopropyl-5,8-dihydro-1,7-naphthyridine-7(6H)-carboxylate (200 mg, 0.46 mmol) in DCM (5 mL) was added TFA (1 mL). The solution was stirred at room temperature for 1 hour. The reaction mixture was removed in vacuo to give 2-((4-chloro-2-fluorobenzyl)oxy)-3-cyclopropyl-5,6,7,8-tetrahydro-1,7-naphthyridine, TFA Salt (250 mg, crude). MS Calcd.: 332.1; MS Found: 333.1 [M+H−TFA]⁺.

### Step B: (S)-2-((2-((4-chloro-2-fluorobenzyl)oxy)-3-cyclopropyl-5,8-dihydro-1,7-naphthyridin-7(6H)-yl)methyl)-3-(oxetan-2-ylmethyl)-3H-imidazo[4,5-b]pyridine-6-carbonitrile

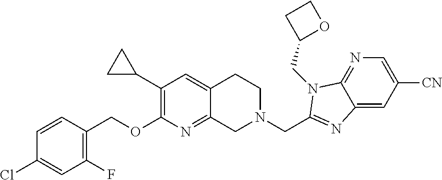 [SMILES: N#Cc1cnc2c(c1)nc(CN1CCc3cc(C4CC4)c(OCc4ccc(Cl)cc4F)nc3C1)n2C[C@@H]1CCO1]

A mixture of 2-((4-chloro-2-fluorobenzyl)oxy)-3-cyclopropyl-5,6,7,8-tetrahydro-1,7-naphthyridine TFA Salt (250 mg, 0.75 mmol), (S)-2-(chloromethyl)-3-(oxetan-2-ylmethyl)-3H-imidazo[4,5-b]pyridine-6-carbonitrile (100 mg, 0.38 mmol), DIEA (492.37 mg, 3.82 mmol) in DMF (5.0 mL) was stirred at 70° C. for 1 hour. The reaction mixture was diluted with H₂O (40 mL), extracted with EA (30 mL×2). The combined organic layer was dried over anhydrous Na₂SO₄, filtered and evaporated to dryness. The residue was purified by column chromatography on silica gel (DCM/MeOH=100/1) to give (S)-2-((2-((4-chloro-2-fluorobenzyl)oxy)-3-cyclopropyl-5,8-dihydro-1,7-naphthyridin-7(6H)-yl)methyl)-3-(oxetan-2-ylmethyl)-3H-imidazo[4,5-b]pyridine-6-carbonitrile (200 mg, yield: 94%). MS Calcd.: 558.19; MS Found: 559.2 [M+H]⁺.

### Step C: (S,Z)-2-((2-((4-chloro-2-fluorobenzyl)oxy)-3-cyclopropyl-5,8-dihydro-1,7-naphthyridin-7(6H)-yl)methyl)-N′-hydroxy-3-(oxetan-2-ylmethyl)-3H-imidazo[4,5-b]pyridine-6-carboximidamide

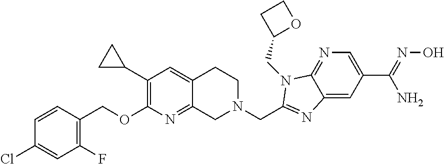 [SMILES: N/C(=N\O)c1cnc2c(c1)nc(CN1CCc3cc(C4CC4)c(OCc4ccc(Cl)cc4F)nc3C1)n2C[C@@H]1CCO1]

A solution of (S)-2-((2-((4-chloro-2-fluorobenzyl)oxy)-3-cyclopropyl-5,8-dihydro-1,7-naphthyridin-7(6H)-yl)methyl)-3-(oxetan-2-ylmethyl)-3H-imidazo[4,5-b]pyridine-6-carbonitrile (200 mg, 0.36 mmol), HONH₂·HCl (149.46 mg, 2.15 mmol), TEA (145.44 mg, 1.44 mmol) in EtOH (4 mL) was stirred at 70° C. for 1 hour. The reaction mixture was filtered and dried to give (S,Z)-2-((2-((4-chloro-2-fluorobenzyl)oxy)-3-cyclopropyl-5,8-dihydro-1,7-naphthyridin-7(6H)-yl)methyl)-N′-hydroxy-3-(oxetan-2-ylmethyl)-3H-imidazo[4,5-b]pyridine-6-carboximidamide (130 mg, yield: 61%). MS Calcd.: 591.22; MS Found: 592.2 [M+H]⁺.

### Step D: (S)-5-(2-((2-((4-chloro-2-fluorobenzyl)oxy)-3-cyclopropyl-5,8-dihydro-1,7-naphthyridin-7(6H)-yl)methyl)-3-(oxetan-2-ylmethyl)-3H-imidazo[4,5-b]pyridin-6-yl)-3-(trifluoromethyl)-1,2,4-oxadiazole

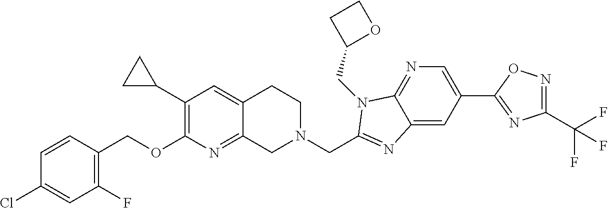 [SMILES: Fc1cc(Cl)ccc1COc1nc2c(cc1C1CC1)CCN(Cc1nc3cc(-c4nc(C(F)(F)F)no4)cnc3n1C[C@@H]1CCO1)C2]

To a solution of (S,Z)-2-((2-((4-chloro-2-fluorobenzyl)oxy)-3-cyclopropyl-5,8-dihydro-1,7-naphthyridin-7(6H)-yl)methyl)-N′-hydroxy-3-(oxetan-2-ylmethyl)-3H-imidazo[4,5-b]pyridine-6-carboximidamide (130 mg, 0.22 mmol) in THF (2 mL) was added TFAA (92.23 mg, 0.44 mmol) and the reaction mixture was stirred at room temperature for 1 hour. The reaction mixture was quenched with sat. sodium bicarbonate aqueous solution (5.0 mL), extracted with EtOAc (15 mL×3). The organic layers were combined, dried over anhydrous Na₂SO₄, filtered and concentrated under vacuum to dryness. The residue was purified by Prep-TLC (DCM/MeOH=30/1) to give (S)-5-(2-((2-((4-chloro-2-fluorobenzyl)oxy)-3-cyclopropyl-5,8-dihydro-1,7-naphthyridin-7(6H)-yl)methyl)-3-(oxetan-2-ylmethyl)-3H-imidazo[4,5-b]pyridin-6-yl)-3-(trifluoromethyl)-1,2,4-oxadiazole (130 mg, yield: 89%). MS Calcd.: 669.19; MS Found: 670.2 [M+H]⁺.

### Step E: 2-[(4-chloro-2-fluorophenyl)methoxy]-3-cyclopropyl-7-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-5,6,7,8-tetrahydro-1,7-naphthyridine

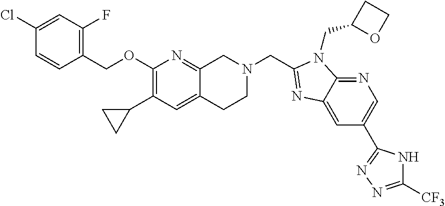 [SMILES: Fc1cc(Cl)ccc1COc1nc2c(cc1C1CC1)CCN(Cc1nc3cc(-c4nnc(C(F)(F)F)[nH]4)cnc3n1C[C@@H]1CCO1)C2]

A mixture of (S)-5-(2-((2-((4-chloro-2-fluorobenzyl)oxy)-3-cyclopropyl-5,8-dihydro-1,7-naphthyridin-7(6H)-yl)methyl)-3-(oxetan-2-ylmethyl)-3H-imidazo[4,5-b]pyridin-6-yl)-3-(trifluoromethyl)-1,2,4-oxadiazole (130 mg, 0.19 mmol) and N₂H₄·H₂O (19.43 mg, 0.39 mmol) in DMF (2 mL) was stirred at 70° C. for 1 hour. The reaction mixture was purified directly by Prep-HPLC (0.1% FA/H₂O/CH₃CN) to give 2-[(4-chloro-2-fluorophenyl)methoxy]-3-cyclopropyl-7-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-5,6,7,8-tetrahydro-1,7-naphthyridine (65.40 mg, yield: 51%).

¹H NMR (400 MHz, DMSO-d₆) δ 9.00 (d, J=1.6 Hz, 1H), 8.61 (d, J=1.6 Hz, 1H), 7.56 (t, J=8.4 Hz, 1H), 7.44 (dd, J=10.0 Hz, 2.0 Hz, 1H), 7.29 (dd, J=8.0 Hz, 1.6 Hz, 1H), 7.06 (s, 1H), 5.32 (s, 2H), 5.09-5.20 (m, 1H), 4.77-4.86 (m, 1H), 4.65-4.74 (m, 1H), 4.42-4.52 (m, 1H), 4.30-4.39 (m, 1H), 4.07-4.21 (m, 2H), 3.61 (s, 2H), 2.76-2.84 (m, 2H), 2.60-2.74 (m, 3H), 2.38-2.50 (m, 1H), 1.91-2.00 (m, 1H), 0.82-0.91 (m, 2H), 0.60-0.68 (m, 2H). ¹⁹F NMR (376 MHz, DMSO-d₆) δ −63.34, −115.25. LC-MS: m/z 669.0 (M+H)⁺.

### 2-[(4-chloro-2-fluorophenyl)methoxy]-7-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridine (compound 4)

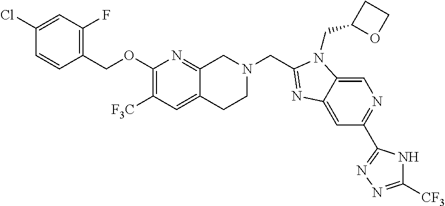 [SMILES: Fc1cc(Cl)ccc1COc1nc2c(cc1C(F)(F)F)CCN(Cc1nc3cc(-c4nnc(C(F)(F)F)[nH]4)ncc3n1C[C@@H]1CCO1)C2]

Compound 4 was synthesized following the similar route of Procedure 3, using tert-butyl 2-((4-chloro-2-fluorobenzyl)oxy)-3-(trifluoromethyl)-5,8-dihydro-1,7-naphthyridine-7(6H)-carboxylate in step A and (S)-2-(chloromethyl)-3-(oxetan-2-ylmethyl)-3H-imidazo[4,5-c]pyridine-6-carbonitrile in step B.

¹H NMR (400 MHz, DMSO-d₆) δ 9.16 (s, 1H), 8.31 (s, 1H), 7.93 (s, 1H), 7.41-7.55 (m, 2H), 7.31 (d, J=8.0 Hz, 1H), 5.42 (s, 2H), 5.05-5.16 (m, 1H), 4.85-4.94 (m, 1H), 4.70-4.79 (m, 1H), 4.45-4.53 (m, 1H), 4.34-4.42 (m, 1H), 4.22, 4.11 (ABq, J=14.0 Hz, 2H), 3.66-3.80 (m, 2H), 2.78-2.90 (m, 4H), 2.61-2.72 (m, 1H), 2.33-2.44 (m, 1H). ¹⁹F NMR (376 MHz, DMSO-d₆) δ −61.69, −63.42, −115.00. LC-MS: m/z 697.0 (M+H)⁺.

### 3-chloro-2-[(4-chloro-2-fluorophenyl)methoxy]-7-[(3-{1[(2S)-oxetan-2-yl]methyl}-6-[5-(trifluoromethyl)-4H-1,2,4-triazol-3-yl]-3H-imidazo[4,5-c]pyridin-2-yl)methyl]-5,6,7,8-tetrahydro-1,7-naphthyridine (compound 5)

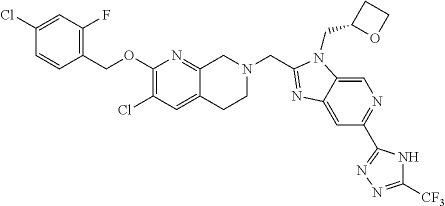 [SMILES: Fc1cc(Cl)ccc1COc1nc2c(cc1Cl)CCN(Cc1nc3cc(-c4nnc(C(F)(F)F)[nH]4)ncc3n1C[C@@H]1CCO1)C2]

Compound 5 was synthesized following the similar route of Procedure 3, using tert-butyl 3-chloro-2-((4-chloro-2-fluorobenzyl)oxy)-5,8-dihydro-1,7-naphthyridine-7(6H)-carboxylate in step A and (S)-2-(chloromethyl)-3-(oxetan-2-ylmethyl)-3H-imidazo[4,5-c]pyridine-6-carbonitrile in step B.

¹H NMR (400 MHz, DMSO-d₆) δ 9.17 (d, J=0.8 Hz, 1H), 8.32 (d, J=0.4 Hz, 1H), 7.73 (s. 1H), 7.55 (t, J=8.0 Hz, 1H), 7.45 (dd, J=10.0 Hz, J=2.0 Hz, 1H), 7.31 (dd, J=8.0 Hz, J=1.6 Hz, 1H), 5.35 (s, 2H), 5.06-5.14 (m. 1H), 4.86-4.95 (m, 1H), 4.70-4.79 (m, 1H), 4.45-4.52 (m, 1H), 4.36-4.41 (m, 1H), 4.20, 4.09 (ABq, J=13.6 Hz, 2H), 3.58-3.73 (m, 2H), 2.72-2.88 (m, 4H), 2.62-2.70 (m, 1H), 2.34-2.44 (m, 1H). ¹⁹F NMR (376 MHz, DMSO-d₆) δ −63.73, −114.99. LC-MS: m/z 663.2 (M+H)⁺.

### Procedure 4

### 2-[(4-chloro-2-fluorophenyl)methoxy]-7-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]-3-(trifluoromethyl)-5,6,7,8-tetrahydro-1,7-naphthyridine compound 6)

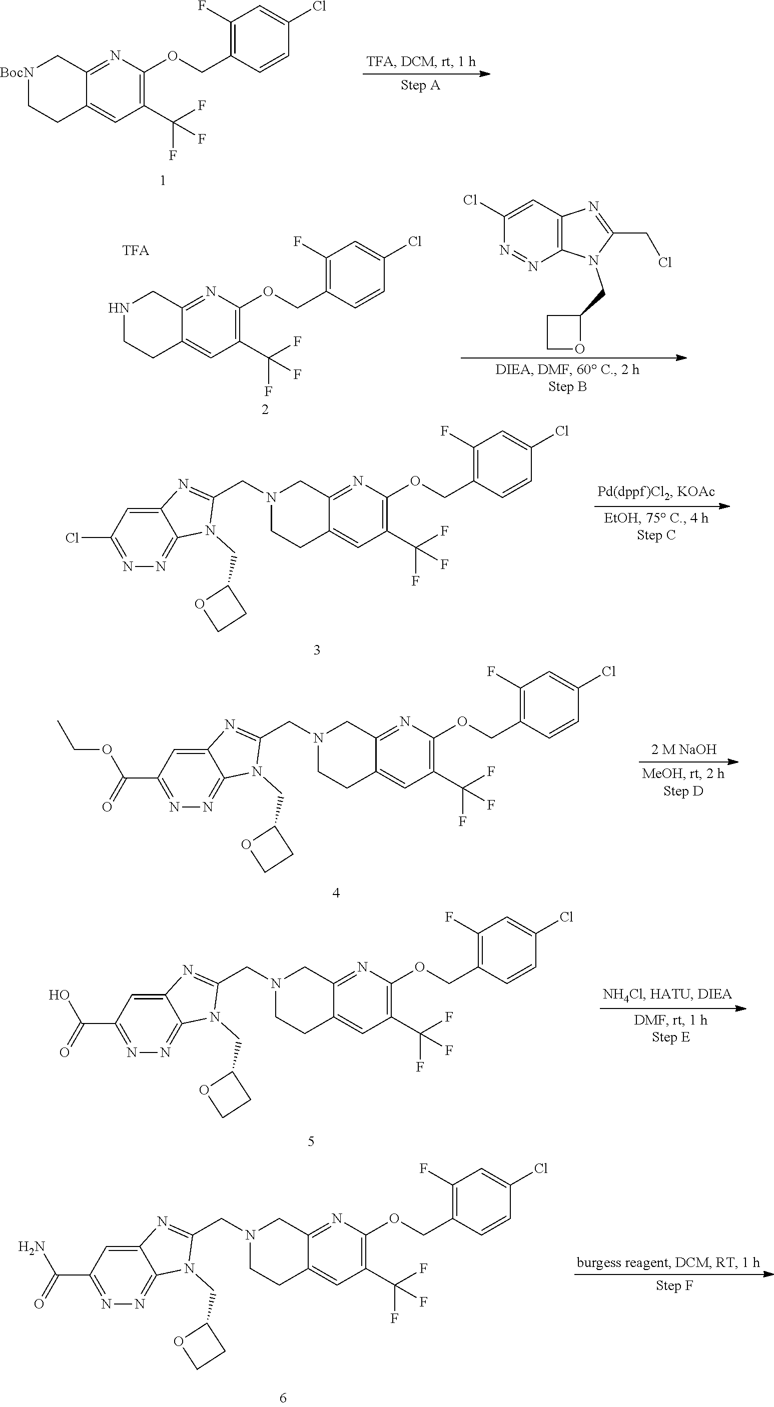

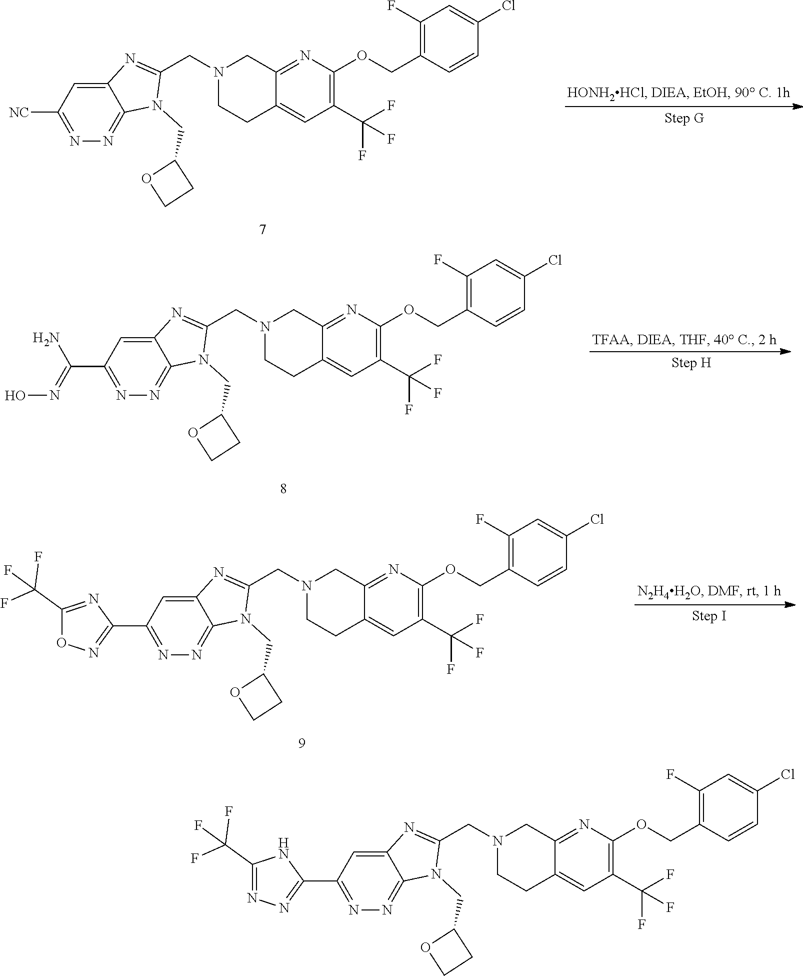 [SMILES: N#Cc1cc2nc(CN3CCc4cc(C(F)(F)F)c(OCc5ccc(Cl)cc5F)nc4C3)n(C[C@@H]3CCO3)c2nn1 ; N/C(=N\O)c1cc2nc(CN3CCc4cc(C(F)(F)F)c(OCc5ccc(Cl)cc5F)nc4C3)n(C[C@@H]3CCO3)c2nn1 ; Fc1cc(Cl)ccc1COc1nc2c(cc1C(F)(F)F)CCN(Cc1nc3cc(-c4noc(C(F)(F)F)n4)nnc3n1C[C@@H]1CCO1)C2 ; Fc1cc(Cl)ccc1COc1nc2c(cc1C(F)(F)F)CCN(Cc1nc3cc(-c4nnc(C(F)(F)F)[nH]4)nnc3n1C[C@@H]1CCO1)C2]

### Step A: 2-((4-chloro-2-fluorobenzyl)oxy)-3-(trifluoromethyl)-5,6,7,8-tetrahydro-1,7-naphthyridine, TFA Salt

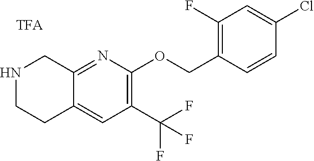 [SMILES: Fc1cc(Cl)ccc1COc1nc2c(cc1C(F)(F)F)CCNC2]

To a solution of tert-butyl 2-((4-chloro-2-fluorobenzyl)oxy)-3-(trifluoromethyl)-5,8-dihydro-1,7-naphthyridine-7(6H)-carboxylate (400 mg, 0.87 mmol) in DCM (6 mL) was added TFA (2 mL). The solution was stirred at room temperature for 1 hour. The reaction mixture was removed in vacuo to give 2-((4-chloro-2-fluorobenzyl)oxy)-3-(trifluoromethyl)-5,6,7,8-tetrahydro-1,7-naphthyridine, TFA Salt (980 mg, crude). MS Calcd.: 360.07; MS Found: 361.0 [M+H−TFA]⁺.

### Step B: (S)-2-((4-chloro-2-fluorobenzyl)oxy)-7-((3-chloro-7-(oxetan-2-ylmethyl)-7H-imidazo[4,5-c]pyridazin-6-yl)methyl)-3-(trifluoromethyl)-5,6,7,8-tetrahydro-1,7-naphthyridine

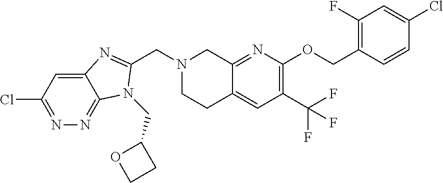 [SMILES: Fc1cc(Cl)ccc1COc1nc2c(cc1C(F)(F)F)CCN(Cc1nc3cc(Cl)nnc3n1C[C@@H]1CCO1)C2]

A mixture of 2-((4-chloro-2-fluorobenzyl)oxy)-3-(trifluoromethyl)-5,6,7,8-tetrahydro-1,7-naphthyridine, TFA salt (480 mg, 1.33 mmol), (S)-3-chloro-6-(chloromethyl)-7-(oxetan-2-ylmethyl)-7H-imidazo[4,5-c]pyridazine (254 mg, 0.93 mmol), DIEA (1.70 g, 13.30 mmol) in DMF (5.0 mL) was stirred at 60° C. for 2 hours. The reaction mixture was diluted with H₂O (60 mL), extracted with EtOAc (40 mL×2). The combined organic layer was dried over anhydrous Na₂SO₄, filtered and evaporated to dryness. The residue was purified by column chromatography on silica gel (DCM/MeOH=60/1) to give (S)-2-((4-chloro-2-fluorobenzyl)oxy)-7-((3-chloro-7-(oxetan-2-ylmethyl)-7H-imidazo[4,5-c]pyridazin-6-yl)methyl)-3-(trifluoromethyl)-5,6,7,8-tetrahydro-1,7-naphthyridine (430 mg, yield: 78%). MS Calcd.: 596.11; MS Found: 597.2 [M+H]⁺.

### Step C: ethyl (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carboxylate

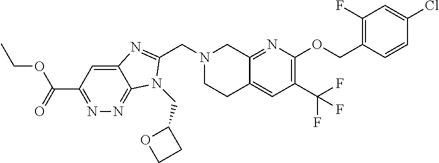 [SMILES: CCOC(=O)c1cc2nc(CN3CCc4cc(C(F)(F)F)c(OCc5ccc(Cl)cc5F)nc4C3)n(C[C@@H]3CCO3)c2nn1]

To a mixture of (S)-2-((4-chloro-2-fluorobenzyl)oxy)-7-((3-chloro-7-(oxetan-2-ylmethyl)-7H-imidazo[4,5-c]pyridazin-6-yl)methyl)-3-(trifluoromethyl)-5,6,7,8-tetrahydro-1,7-naphthyridine (390 mg, 0.65 mmol) in EtOH (10.0 mL) was added PdCl₂(dppf) (48.00 mg, 0.065 mmol) and KOAc (127.00 mg, 1.30 mmol). The mixture was stirred at 75° C. for 4 hours under CO. The reaction mixture was filtered and the filtrate was evaporated to dryness. The residue was purified by column chromatography on silica gel (DCM/MeOH=60/1) to give ethyl (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carboxylate (450 mg, yield: 98.5%). MS Calcd.: 634.17; MS Found: 635.4 [M+H]⁺.

### Step D: (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carboxylic acid

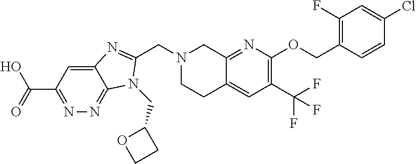 [SMILES: O=C(O)c1cc2nc(CN3CCc4cc(C(F)(F)F)c(OCc5ccc(Cl)cc5F)nc4C3)n(C[C@@H]3CCO3)c2nn1]

To a mixture of ethyl (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carboxylate (420 mg, 0.66 mmol) in MeOH (10.0 mL) was added NaOH (2 M, 0.66 mL, aq.). The mixture was stirred at room temperature for 2 hours. The reaction mixture was adjusted to pH=7 with 1 N HCl (aq.) and extracted with EtOAc (30 mL×2). The combined organic layer was dried over anhydrous sodium sulfate, filtered and evaporated to give (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carboxylic acid (410 mg, crude). MS Calcd.: 606.14; MS Found: 607.0 [M+H]⁺.

### Step E: (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carboxamide

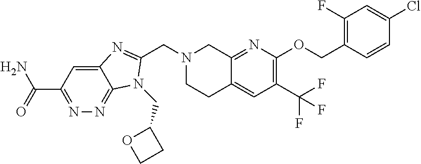 [SMILES: NC(=O)c1cc2nc(CN3CCc4cc(C(F)(F)F)c(OCc5ccc(Cl)cc5F)nc4C3)n(C[C@@H]3CCO3)c2nn1]

A mixture of (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carboxylic acid (380 mg, 0.63 mmol), NH₄Cl (49.85 mg, 0.94 mmol), HATU (357.20 mg, 0.94 mmol), DIEA (162.54 mg, 1.26 mmol) in DMF (4.0 mL) was stirred at room temperature for 1 hour. The reaction mixture was diluted with H₂O (30 mL), extracted with EtOAc (20 mL×2). The combined organic layer was dried over anhydrous Na₂SO₄, filtered and evaporated to dryness. The residue was purified by column chromatography on silica gel (DCM/MeOH=80/1) to give (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carboxamide (530 mg, impure). MS Calcd.: 605.16; MS Found: 606.0 [M+H]⁺.

### Step F: (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carbonitrile

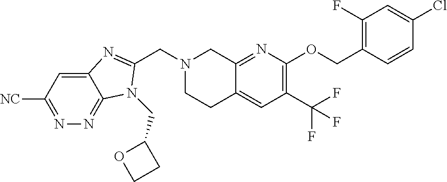 [SMILES: N#Cc1cc2nc(CN3CCc4cc(C(F)(F)F)c(OCc5ccc(Cl)cc5F)nc4C3)n(C[C@@H]3CCO3)c2nn1]

A mixture of (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carboxamide (530 mg, 0.88 mmol), Burgess reagent (833.98 mg, 3.50 mmol) in DCM (4.0 mL) was stirred at room temperature for 1 hour. The reaction mixture was diluted with H₂O (20 mL), extracted with EtOAc (10 mL×2). The combined organic layer was dried over Na₂SO₄, filtered and concentrated in vacuum. The residue was purified by column chromatography on silica gel (DCM/MeOH=80/1) to give (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carbonitrile (380 mg, yield: 74%) (2 steps). MS Calcd.: 587.15; MS Found: 588.2 [M+H]⁺.

### Step G: (S,Z)-6-((2-((4-chloro-2-fluorobenzyl)oxy)-3-(trifluoromethyl)-5,8-dihydro-1,7-naphthyridin-7(6H)-yl)methyl)-N′-hydroxy-7-(oxetan-2-ylmethyl)-7H-imidazo[4,5-c]pyridazine-3-carboximidamide

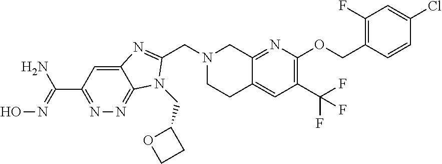 [SMILES: N/C(=N\O)c1cc2nc(CN3CCc4cc(C(F)(F)F)c(OCc5ccc(Cl)cc5F)nc4C3)n(C[C@@H]3CCO3)c2nn1]

A solution of (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carbonitrile (350 mg, 0.60 mmol), HONH₂—HCl (248.64 mg, 3.60 mmol), TEA (242.40 mg, 2.40 mmol) in EtOH (4.0 mL) was stirred at 90° C. for 1 hour. The reaction mixture was filtered and dried to give (S,Z)-6-((2-((4-chloro-2-fluorobenzyl)oxy)-3-(trifluoromethyl)-5,8-dihydro-1,7-naphthyridin-7(6H)-yl)methyl)-N′-hydroxy-7-(oxetan-2-ylmethyl)-7H-imidazo[4,5-c]pyridazine-3-carboximidamide (360 mg, yield: 98%). MS Calcd.: 620.17; MS Found: 620.9 [M+H]⁺.

### Step H: (S)-3-(6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazin-3-yl)-5-(trifluoromethyl)-1,2,4-oxadiazole

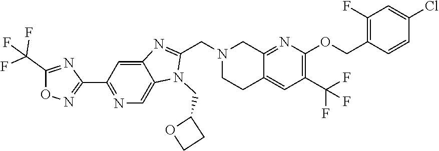 [SMILES: Fc1cc(Cl)ccc1COc1nc2c(cc1C(F)(F)F)CCN(Cc1nc3cc(-c4noc(C(F)(F)F)n4)ncc3n1C[C@@H]1CCO1)C2]

To a solution of (S,Z)-6-((2-((4-chloro-2-fluorobenzyl)oxy)-3-(trifluoromethyl)-5,8-dihydro-1,7-naphthyridin-7(6H)-yl)methyl)-N′-hydroxy-7-(oxetan-2-ylmethyl)-7H-imidazo[4,5-c]pyridazine-3-carboximidamide (200 mg, 0.32 mmol) in THF (4.0 mL) was added DIEA (206.40 mg, 1.60 mmol) and TFAA (135.48 mg, 0.65 mmol) and the mixture stirred at 40° C. for 2 hours. The reaction mixture was quenched with sat. sodium bicarbonate aqueous solution (10.0 mL), extracted with EtOAc (15 mL×3). The organic layers were combined, dried over anhydrous Na₂SO₄, filtered and concentrated under vacuum to dryness. The residue was purified by Prep-TLC (DCM/MeOH=40/1) to give (S)-3-(6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazin-3-yl)-5-(trifluoromethyl)-1,2,4-oxadiazole (80 mg, yield: 36%). MS Calcd.: 698.14; MS Found: 699.1 [M+H]⁺.

### Step I: 2-[(4-chloro-2-fluorophenyl)methoxy]-7-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]-3-(trifluoromethyl)-5,6,7,8-tetrahydro-1,7-naphthyridine

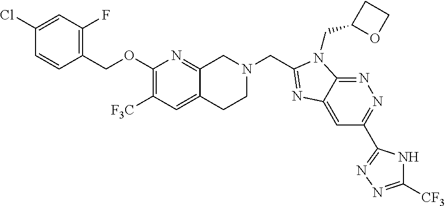 [SMILES: Fc1cc(Cl)ccc1COc1nc2c(cc1C(F)(F)F)CCN(Cc1nc3cc(-c4nnc(C(F)(F)F)[nH]4)nnc3n1C[C@@H]1CCO1)C2]

A mixture of (S)-3-(6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazin-3-yl)-5-(trifluoromethyl)-1,2,4-oxadiazole (80 mg, 0.11 mmol) and N₂H₄·H₂O (11.46 mg, 0.23 mmol) in DMF (1.0 mL) was stirred at room temperature for 1 hour. The reaction mixture was purified by Prep-HPLC (0.1% FA/H₂O/CH₃CN) to give 2-[(4-chloro-2-fluorophenyl)methoxy]-7-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]-3-(trifluoromethyl)-5,6,7,8-tetrahydro-1,7-naphthyridine (37.18 mg, yield: 46%).

¹H NMR (400 MHz, DMSO-d₆) δ 8.53 (s, 1H), 7.94 (s, 1H), 7.43-7.57 (m, 2H), 7.32 (d, J=8.4 Hz, 1H), 5.43 (s, 2H), 5.18-5.29 (m, 1H), 4.94-5.04 (m, 1H), 4.81-4.91 (m, 1H), 4.45-4.54 (m, 1H), 4.33-4.42 (m, 1H), 4.22-4.32 (m, 2H), 3.80 (s, 2H), 2.81-2.98 (m, 4H), 2.65-2.77 (m, 2H). ¹⁹F NMR (376 MHz, DMSO-d₆) δ −61.67, −63.08, −115.03. LC-MS: m/z 698.0 (M+H)⁺.

### 3-chloro-2-[(4-chloro-2-fluorophenyl)methoxy]-7-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]-5,6,7,8-tetrahydro-1,7-naphthyridine (compound 7)

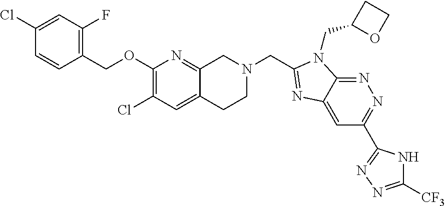 [SMILES: Fc1cc(Cl)ccc1COc1nc2c(cc1Cl)CCN(Cc1nc3cc(-c4nnc(C(F)(F)F)[nH]4)nnc3n1C[C@@H]1CCO1)C2]

Compound 7 was synthesized following the similar route of Procedure 4, using tert-butyl 3-chloro-2-((4-chloro-2-fluorobenzyl)oxy)-5,8-dihydro-1,7-naphthyridine-7(6H)-carboxylate in step A.

¹H NMR (400 MHz, DMSO-d₆) δ 8.50 (s, 1H), 8.23 (s, 1H), 7.74 (s, 1H), 7.56 (t, J=8.0 Hz, 1H), 7.46 (dd, J=10.0 Hz, J=1.6 Hz, 1H), 7.32 (dd, J=8.0 Hz, J=1.6 Hz, 1H), 5.37 (s, 2H), 5.30-5.34 (m, 1H), 5.19-5.27 (m, 1H), 4.93-5.01 (m, 1H), 4.81-4.89 (m, 1H), 4.45-4.52 (m, 1H), 4.33-4.40 (m, 1H), 4.23-4.27 (m, 2H), 3.72 (s, 2H), 2.84-2.90 (m, 2H), 2.77-2.82 (m, 2H), 2.69-2.76 (m, 1H). ¹⁹F NMR (376 MHz, DMSO-d₆) δ −62.81, −115.00. LC-MS: m/z 664.0 (M+H)⁺.

### Procedure 5

### (S)-3-(2-((2-((4-chloro-2-fluorobenzyl)oxy)-5,8-dihydro-1,7-naphthyridin-7(6H)-yl)methyl)-1-(oxetan-2-ylmethyl)-1H-benzo[d]imidazol-6-yl)-1,2,4-oxadiazol-5(4H)-one (compound 8)

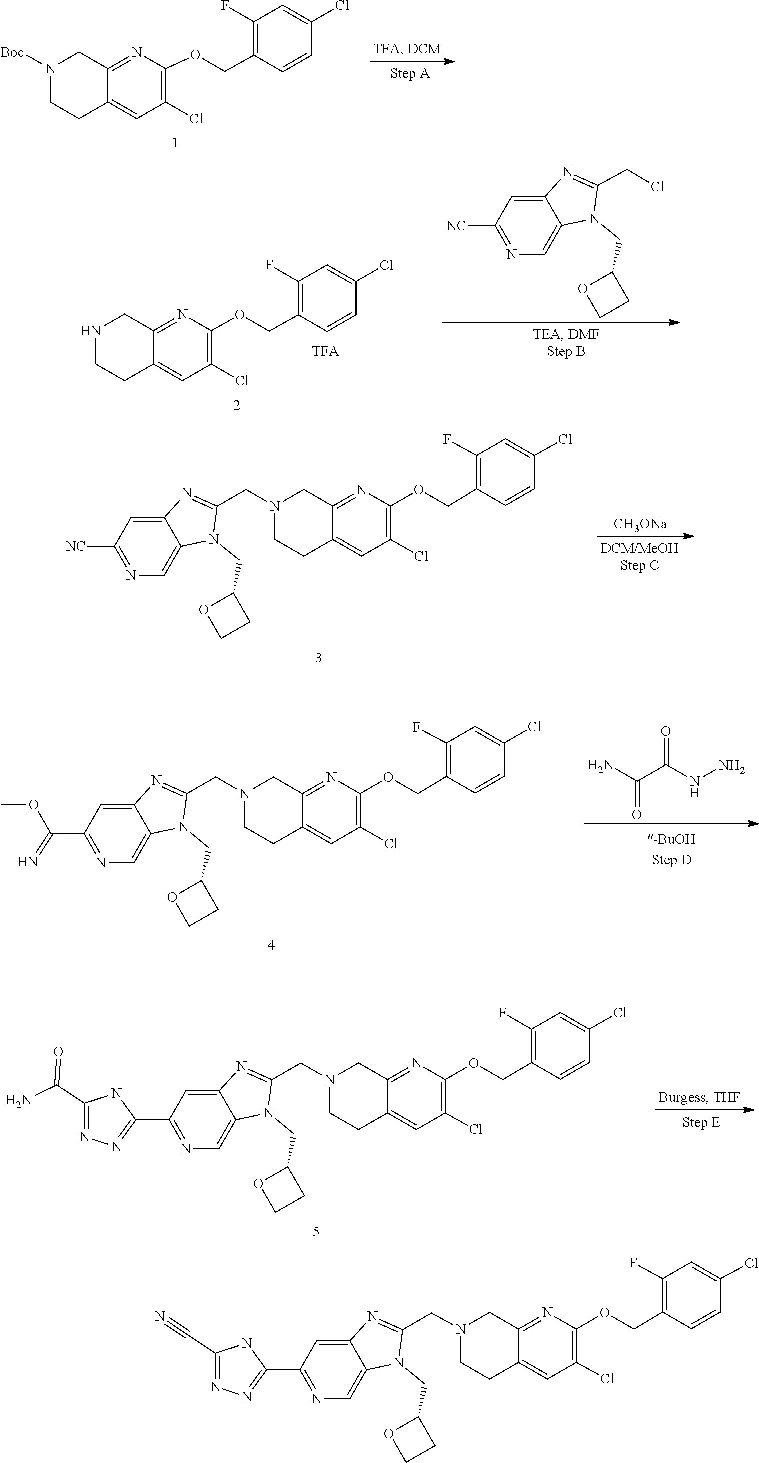

### Step A: 3-chloro-2-((4-chloro-2-fluorobenzyl)oxy)-5,6,7,8-tetrahydro-1,7-naphthyridine, TFA salt

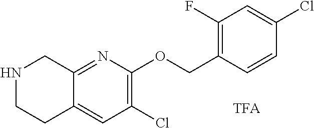 [SMILES: Fc1cc(Cl)ccc1COc1nc2c(cc1Cl)CCNC2 ; "TFA"]

To a solution of tert-butyl 3-chloro-2-((4-chloro-2-fluorobenzyl)oxy)-5,8-dihydro-1,7-naphthyridine-7(6H)-carboxylate (200 mg, 0.47 mmol) in DCM (3 mL) was added TFA (1 mL) at room temperature. The reaction mixture was stirred at room temperature for 1 hour. The reaction mixture was concentrated in vacuum to give 3-chloro-2-((4-chloro-2-fluorobenzyl)oxy)-5,6,7,8-tetrahydro-1,7-naphthyridine, TFA salt (206 mg, crude). MS Calcd.: 326.04; MS Found: 327.2 [M+H−TFA]⁺.

### Step B: (S)-2-((3-chloro-2-((4-chloro-2-fluorobenzyl)oxy)-5,8-dihydro-1,7-naphthyridin-7(6H)-yl)methyl)-3-(oxetan-2-ylmethyl)-3H-imidazo[4,5-c]pyridine-6-carbonitrile

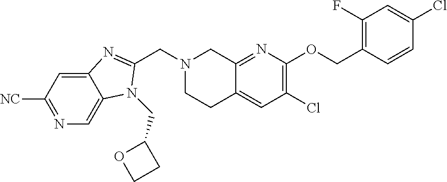 [SMILES: N#Cc1cc2nc(CN3CCc4cc(Cl)c(OCc5ccc(Cl)cc5F)nc4C3)n(C[C@@H]3CCO3)c2cn1]

A mixture of 3-chloro-2-((4-chloro-2-fluorobenzyl)oxy)-5,6,7,8-tetrahydro-1,7-naphthyridine, TFA salt (206 mg, 0.47 mmol), (S)-2-(chloromethyl)-3-(oxetan-2-ylmethyl)-3H-imidazo[4,5-c]pyridine-6-carbonitrile (123 mg, 0.47 mmol) and TEA (142 mg, 1.41 mmol) in DMF (4 mL) was stirred at 50° C. for 2 hours. The reaction mixture was filtered, and the filtrate was concentrated in vacuum. The residue was purified by column chromatography (DCM:MeOH=95:5) to give (S)-2-((3-chloro-2-((4-chloro-2-fluorobenzyl)oxy)-5,8-dihydro-1,7-naphthyridin-7(6H)-yl)methyl)-3-(oxetan-2-ylmethyl)-3H-imidazo[4,5-c]pyridine-6-carbonitrile (250 mg, 97% yield). MS Calcd.: 552.12; MS Found: 553.4 [M+H]⁺.

### Step C: methyl (S)-2-((3-chloro-2-((4-chloro-2-fluorobenzyl)oxy)-5,8-dihydro-1,7-naphthyridin-7(6H)-yl)methyl)-3-(oxetan-2-ylmethyl)-3H-imidazo[4,5-c]pyridine-6-carbimidate

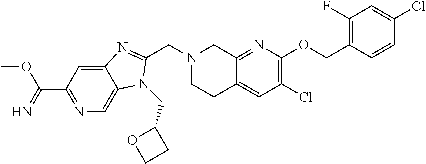 [SMILES: COC(=N)c1cc2nc(CN3CCc4cc(Cl)c(OCc5ccc(Cl)cc5F)nc4C3)n(C[C@@H]3CCO3)c2cn1]

To a solution of (S)-2-((3-chloro-2-((4-chloro-2-fluorobenzyl)oxy)-5,8-dihydro-1,7-naphthyridin-7(6H)-yl)methyl)-3-(oxetan-2-ylmethyl)-3H-imidazo[4,5-c]pyridine-6-carbonitrile (250 mg, 0.45 mmol) in DCM (2 mL)/MeOH (2 mL) was added MeONa (254 mg, 4.5 mmol) at room temperature. The reaction mixture was stirred at room temperature for 4 hours. The reaction mixture was quenched with H₂O (20 mL) and extracted with DCM (20 mL×3). The organic layers were combined and washed with brine (30 mL×2), dried over anhydrous Na₂SO₄, filtered and concentrated in vacuum. The residue was purified by column chromatography on silica gel (DCM:MeOH=95:5) to give methyl (S)-2-((3-chloro-2-((4-chloro-2-fluorobenzyl)oxy)-5,8-dihydro-1,7-naphthyridin-7(6H)-yl)methyl)-3-(oxetan-2-ylmethyl)-3H-imidazo[4,5-c]pyridine-6-carbimidate (270 mg, crude). MS Calcd.: 584.15; MS Found: 585.4 [M+H]⁺.

### Step D: (S)-5-(2-((3-chloro-2-((4-chloro-2-fluorobenzyl)oxy)-5,8-dihydro-1,7-naphthyridin-7(6H)-yl)methyl)-3-(oxetan-2-ylmethyl)-3H-imidazo[4,5-c]pyridin-6-yl)-4H-1,2,4-triazole-3-carboxamide

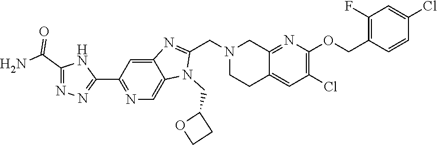 [SMILES: NC(=O)c1nnc(-c2cc3nc(CN4CCc5cc(Cl)c(OCc6ccc(Cl)cc6F)nc5C4)n(C[C@@H]4CCO4)c3cn2)[nH]1]

### Step E: 5-[2-({3-chloro-2-[(4-chloro-2-fluorophenyl)methoxy]-5,6,7,8-tetrahydro-1,7-naphthyridin-7-yl}methyl)-3-((2S)-oxetan-2-yl]methyl}-3H-imidazo[4,5-c]pyridin-6-yl)-4H-1,2,4-triazole-3-carbonitrile

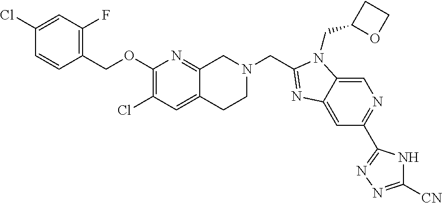 [SMILES: N#Cc1nnc(-c2cc3nc(CN4CCc5cc(Cl)c(OCc6ccc(Cl)cc6F)nc5C4)n(C[C@@H]4CCO4)c3cn2)[nH]1]

To a solution of (S)-5-(2-((3-chloro-2-((4-chloro-2-fluorobenzyl)oxy)-5,8-dihydro-1,7-naphthyridin-7(6H)-yl)methyl)-3-(oxetan-2-ylmethyl)-3H-imidazo[4,5-c]pyridin-6-yl)-4H-1,2,4-triazole-3-carboxamide (150 mg, 0.24 mmol) in THF (2 mL) was added burgess reagent (112 mg, 0.48 mmol) at room temperature. The reaction mixture was stirred at 70° C. for 2 hours. The reaction mixture was filtered and the filtrate purified by Prep-HPLC to give 5-[2-({3-chloro-2-[(4-chloro-2-fluorophenyl)methoxy]-5,6,7,8-tetrahydro-1,7-naphthyridin-7-yl}methyl)-3-{[(2S)-oxetan-2-yl]methyl}-3H-imidazo[4,5-c]pyridin-6-yl]-4H-1,2,4-triazole-3-carbonitrile (2.45 mg, 2% yield).

¹H NMR (400 MHz, CD₃OD) δ 9.03 (s, 1H), 8.34 (s, 1H), 7.47-7.54 (m, 2H), 7.13-7.21 (m, 2H), 5.37 (s, 2H), 5.15-5.25 (m, 1H), 4.69-4.81 (m, 2H), 4.55-4.63 (m, 1H), 4.39-4.46 (m, 1H), 4.21, 4.12 (ABq, J=13.6 Hz, 2H), 3.58-3.71 (m, 2H), 2.77-2.89 (m, 4H), 2.66-2.77 (m, 1H), 2.40-2.52 (m, 1H). ¹⁹F NMR (376 MHz, CD₃OD) δ −117.40. LC-MS: m/z 620.1 (M+H)⁺.

### 5-[2-({3-chloro-2-[(4-chloro-2,6-difluorophenyl)methoxy]-5,6,7,8-tetrahydro-1,7-naphthyridin-7-yl}methyl)-3-{[(2S)-oxetan-2-yl]methyl}-3H-imidazo[4,5-c]pyridin-6-yl]-4H-1,2,4-triazole-3-carbonitrile (compound 9)

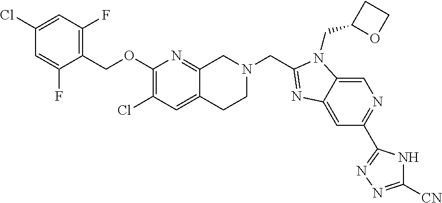 [SMILES: N#Cc1nnc(-c2cc3nc(CN4CCc5cc(Cl)c(OCc6c(F)cc(Cl)cc6F)nc5C4)n(C[C@@H]4CCO4)c3cn2)[nH]1]

Compound 9 was synthesized following the similar route of Procedure 5, using tert-butyl 3-chloro-2-((4-chloro-2,6-difluorobenzyl)oxy)-5,8-dihydro-1,7-naphthyridine-7(6H)-carboxylate in step A.

¹H NMR (400 MHz, DMSO-d₆) δ 9.22 (s, 1H), 8.37 (s, 1H), 7.78 (s, 1H), 7.40 (d, J=7.6 Hz, 2H), 5.34 (s, 2H), 5.06-5.17 (m, 1H), 4.87-4.96 (m, 1H), 4.72-4.80 (m, 1H), 4.30-4.54 (m, 4H), 3.87-3.96 (m, 2H), 3.03-3.14 (m, 2H), 2.80-2.89 (m, 2H), 2.63-2.75 (m, 1H), 2.32-2.43 (m, 1H). ¹⁹F NMR (376 MHz, DMSO-d₆) δ −62.81, −112.02. LC-MS: m/z 638.3 (M+H)⁺.

### Procedure 6

### 1-{[2-({2-[(4-chloro-2-fluorophenyl)methoxy]-3-(trifluoromethyl)-5,6,7,8-tetrahydro-1,7-naphthyridin-7-yl}methyl)-5-[5-(trifluoromethyl)-4H-1,2,4-triazol-3-yl]pyridin-3-yl]methyl}cyclopropane-1-carbonitrile (10)

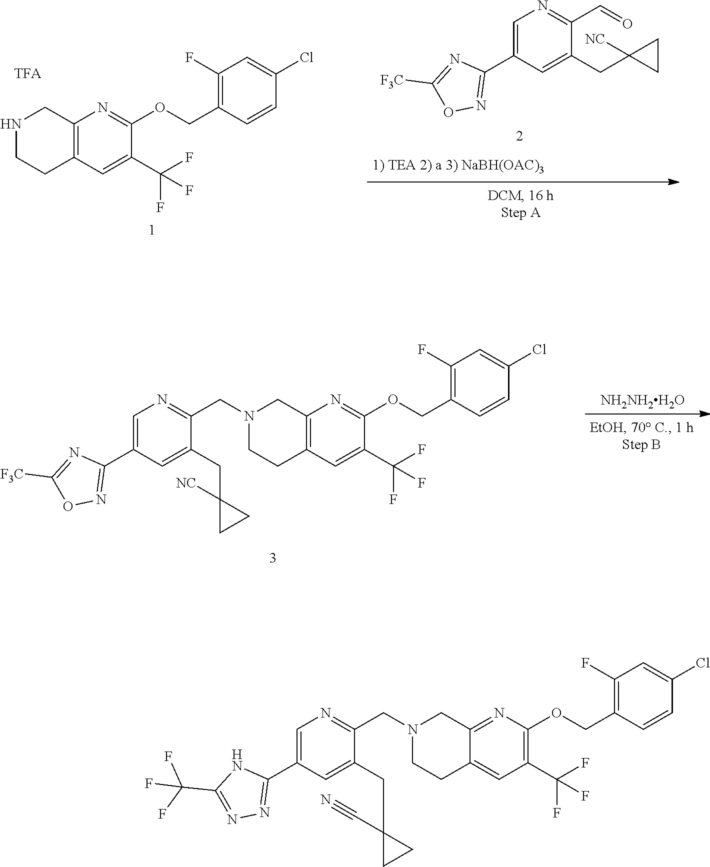 [SMILES: Fc1cc(Cl)ccc1COc1nc2c(cc1C(F)(F)F)CCNC2 ; N#CC1(Cc2cc(-c3noc(C(F)(F)F)n3)cnc2C=O)CC1 ; N#CC1(Cc2cc(-c3noc(C(F)(F)F)n3)cnc2CN2CCc3cc(C(F)(F)F)c(OCc4ccc(Cl)cc4F)nc3C2)CC1 ; "NH2NH2·H2O" ; N#CC1(Cc2cc(-c3nnc(C(F)(F)F)[nH]3)cnc2CN2CCc3cc(C(F)(F)F)c(OCc4ccc(Cl)cc4F)nc3C2)CC1]

### Step A: 1-((2-((2-((4-chloro-2-fluorobenzyl)oxy)-3-(trifluoromethyl)-5,8-dihydro-1,7-naphthyridin-7(6H)-yl)methyl)-5-(5-(trifluoromethyl)-1,2,4-oxadiazol-3-yl)pyridin-3-yl)methyl)cyclopropane-1-carbonitrile

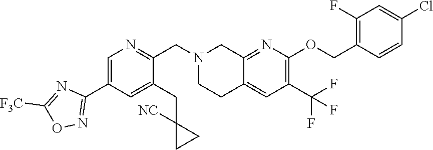 [SMILES: N#CC1(Cc2cc(-c3noc(C(F)(F)F)n3)cnc2CN2CCc3cc(C(F)(F)F)c(OCc4ccc(Cl)cc4F)nc3C2)CC1]

A mixture of 2-((4-chloro-2-fluorobenzyl)oxy)-3-(trifluoromethyl)-5,6,7,8-tetrahydro-1,7-naphthyridine, TFA salt (34 mg, 0.09 mmol) and TEA (48 mg, 0.48 mmol) in DCM (5 mL) was stirred at room temperature for 0.5 hour. 1-((2-formyl-5-(5-(trifluoromethyl)-1,2,4-oxadiazol-3-yl)pyridin-3-yl)methyl)cyclopropane-1-carbonitrile (30 mg, 0.09 mmol) was added and the mixture was stirred for 2 hours. Then NaBH(OAC)₃ (80 mg, 0.38 mmol) was added and the resulting mixture was stirred at room temperature for another 16 hours. The mixture was poured into water (50 mL) and extracted with DCM (2×50 mL), the combined organic layer was washed with brine, dried over anhydrous Na₂SO₄, filtered and the residue was concentrated to give 1-((2-((2-((4-chloro-2-fluorobenzyl)oxy)-3-(trifluoromethyl)-5,8-dihydro-1,7-naphthyridin-7(6H)-yl)methyl)-5-(5-(trifluoromethyl)-1,2,4-oxadiazol-3-yl)pyridin-3-yl)methyl)cyclopropane-1-carbonitrile (70 mg, yield: >99.9%). MS Calcd: 666.1; MS Found: 667.2 [M+H]⁺.

### Step B: 1-{[2-({2-[(4-chloro-2-fluorophenyl)methoxy]-3-(trifluoromethyl)-5,6,7,8-tetrahydro-1,7-naphthyridin-7-yl}methyl)-5-[5-(trifluoromethyl)-4H-1,2,4-triazol-3-yl]pyridin-3-yl]methyl}cyclopropane-1-carbonitrile

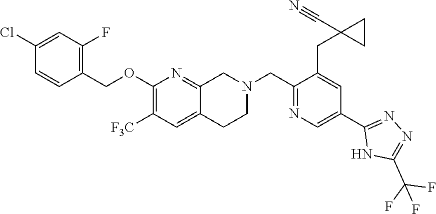 [SMILES: N#CC1(Cc2cc(-c3nnc(C(F)(F)F)[nH]3)cnc2CN2CCc3cc(C(F)(F)F)c(OCc4ccc(Cl)cc4F)nc3C2)CC1]

A mixture of 1-((2-((2-((4-chloro-2-fluorobenzyl)oxy)-3-(trifluoromethyl)-5,8-dihydro-1,7-naphthyridin-7(6H)-yl)methyl)-5-(5-(trifluoromethyl)-1,2,4-oxadiazol-3-yl)pyridin-3-yl)methyl)cyclopropane-1-carbonitrile (70 mg, 0.11 mmol) and NH₂NH₂·H₂O (0.15 mL) in EtOH (2 mL) was stirred at 70° C. for 1 hour. The reaction mixture was purified by prep-HPLC to give 1-{[2-({2-[(4-chloro-2-fluorophenyl)methoxy]-3-(trifluoromethyl)-5,6,7,8-tetrahydro-1,7-naphthyridin-7-yl}methyl)-5-[5-(trifluoromethyl)-4H-1,2,4-triazol-3-yl]pyridin-3-yl]methyl}cyclopropane-1-carbonitrile (10.32 mg, yield: 14.7%).

¹H NMR (400 MHz, CD₃OD) δ 9.11 (d, J=2.4 Hz, 1H), 8.54 (d, J=2.0 Hz, 1H), 7.75 (s, 1H), 7.47 (t, J=8.4 Hz, 1H), 7.22-7.16 (m, 2H), 5.44 (s, 2H), 4.05 (s, 2H), 3.70 (s, 2H), 3.23 (s, 2H), 2.83-2.90 (m, 4H), 1.37-1.42 (m, 2H), 1.18-1.23 (m, 2H). ¹⁹F NMR (376 MHz, CD₃OD) δ −64.52, −66.01, −117.38. LC-MS: m/z 666.2 (M+H)⁺.

### 1-{[2-({3-chloro-2-[(4-chloro-2-fluorophenyl)methoxy]-5,6,7,8-tetrahydro-1,7-naphthyridin-7-yl}methyl)-5-[5-(trifluoromethyl)-4H-1,2,4-triazol-3-yl]pyridin-3-yl]methyl}cyclopropane-1-carbonitrile (compound 11)

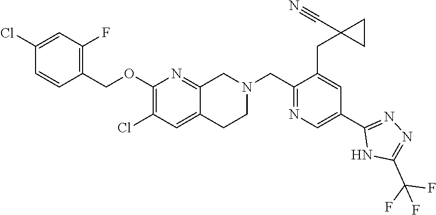 [SMILES: N#CC1(Cc2cc(-c3nnc(C(F)(F)F)[nH]3)cnc2CN2CCc3cc(Cl)c(OCc4ccc(Cl)cc4F)nc3C2)CC1]

Compound 11 was synthesized following the similar route of Procedure 6, using 3-chloro-2-((4-chloro-2-fluorobenzyl)oxy)-5,6,7,8-tetrahydro-1,7-naphthyridine, TFA salt in step A.

¹H NMR (400 MHz, CD₃OD) δ 9.11 (s, 1H), 8.54 (s, 1H), 7.54-7.48 (m, 2H), 7.20-7.16 (m, 2H), 5.38 (s, 2H), 4.10 (s, 2H), 3.70 (s, 2H), 3.22 (s, 2H), 2.92-2.83 (m, 4H), 1.41-1.38 (m, 2H), 1.23-1.19 (m, 2H). ¹⁹F NMR (376 MHz, CD₃OD) δ −66.36, −117.42. LC-MS: m/z 632.2 (M+H)⁺.

### Procedure 7

### 3-chloro-2-[(4-chloro-2-fluorophenyl)methoxy]-7-({3-methyl-5-[5-(trifluoromethyl)-4H-1,2,4-triazol-3-yl]pyridin-2-yl}methyl)-5,6,7,8-tetrahydro-1,7-naphthyridine (compound 12)

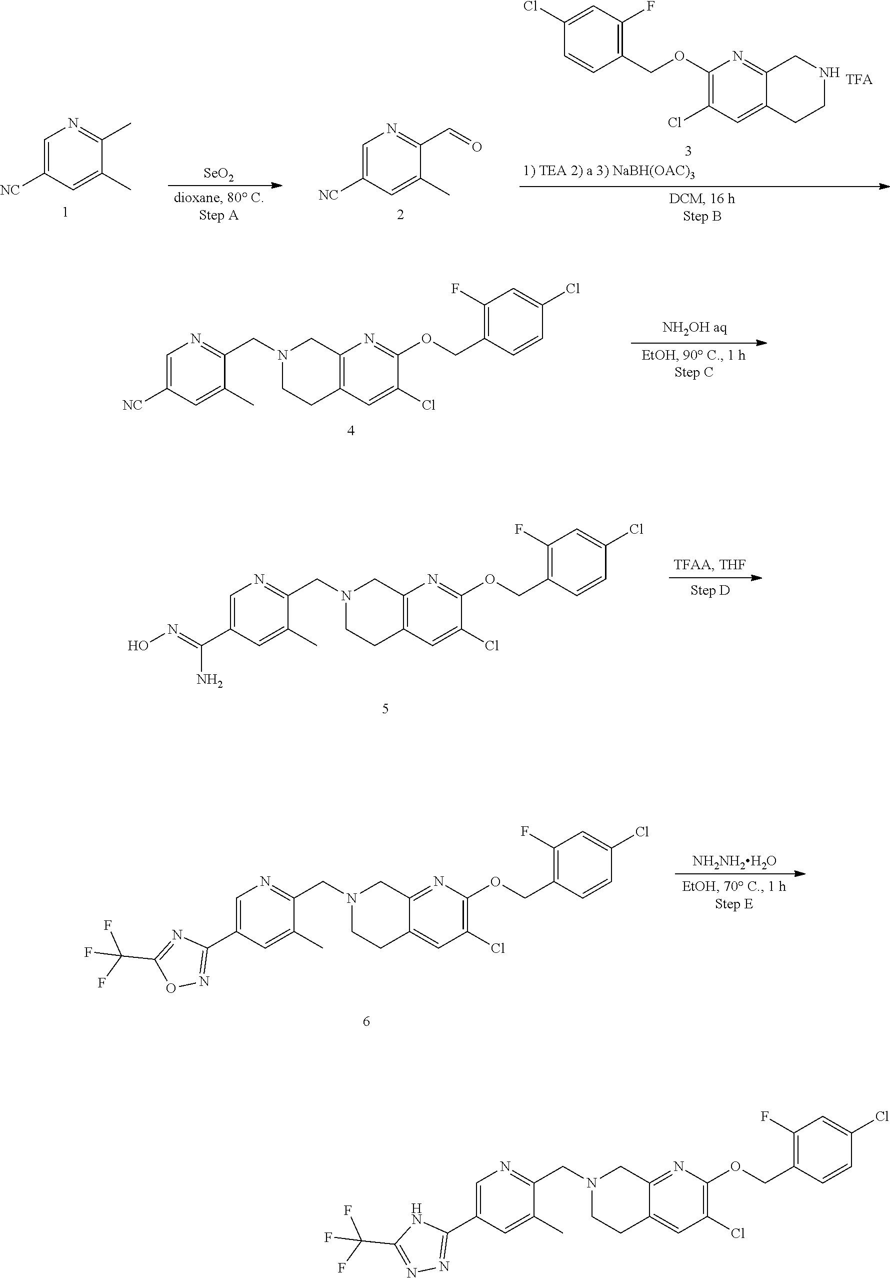

### Step A: 6-formyl-5-methylnicotinonitrile

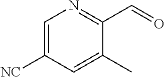

A mixture of 5,6-dimethylnicotinonitrile (400 mg, 3.03 mmol) and SeO₂ (3.36 g, 30.30 mmol) in dioxane (15 mL) was stirred at 80° C. for 16 hours. The reaction mixture was filtered to give 6-formyl-5-methylnicotinonitrile (320 mg, yield: 72.40%, yield). H NMR (400 MHz, CDCl₃) δ 10.21 (s, 1H), 8.90 (s, 1H), 7.93 (s, 1H), 2.72 (s, 3H).

### Step B: 6-((3-chloro-2-((4-chloro-2-fluorobenzyl)oxy)-5,8-dihydro-1,7-naphthyridin-7(6H)-yl)methyl)-5-methylnicotinonitrile

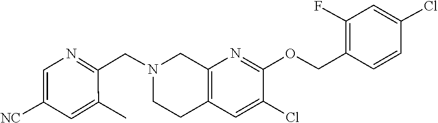 [SMILES: Cc1cc(C#N)cnc1CN1CCc2cc(Cl)c(OCc3ccc(Cl)cc3F)nc2C1]

A mixture of 3-chloro-2-((4-chloro-2-fluorobenzyl)oxy)-5,6,7,8-tetrahydro-1,7-naphthyridine, TFA salt (46 mg, 0.141 mmol) and TEA (71 mg, 0.703 mmol) in DCM (5 mL) was stirred at room temperature for 0.5 hour. To the mixture 6-formyl-5-methylnicotinonitrile (21 mg, 0.141 mmol) was added and the mixture was stirred for 2 hours. Then to the mixture NaBH(OAC)₃ (119 mg, 0.563 mmol) was added and the resulting mixture was stirred at room temperature for another 16 hours. The mixture was poured into water (50 mL) and extracted with DCM (2×50 mL), the combined organic layer was washed with brine, dried over anhydrous Na₂SO₄, filtered and the residue was concentrated and purified by TLC to give 6-((3-chloro-2-((4-chloro-2-fluorobenzyl)oxy)-5,8-dihydro-1,7-naphthyridin-7(6H)-yl)methyl)-5-methylnicotinonitrile (20 mg, yield: 31.25%). MS Calcd: 456.1; MS Found: 457.3 [M+H]⁺.

### Step C: (Z)-6-((3-chloro-2-((4-chloro-2-fluorobenzyl)oxy)-5,8-dihydro-1,7-naphthyridin-7(6H)-yl)methyl)-N′-hydroxy-5-methylnicotinimidamide

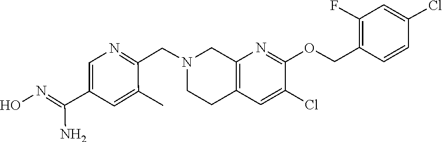 [SMILES: Cc1cc(/C(N)=N/O)cnc1CN1CCc2cc(Cl)c(OCc3ccc(Cl)cc3F)nc2C1]

A mixture of 6-((3-chloro-2-((4-chloro-2-fluorobenzyl)oxy)-5,8-dihydro-1,7-naphthyridin-7(6H)-yl)methyl)-5-methylnicotinonitrile (20 mg, 0.044 mmol) and NH₂OH·aq (50% in water, 3.47 mg, 0.053 mmol) in EtOH (2 mL) was stirred at 90° C. for 0.5 hour. The mixture was concentrated to give (Z)-6-((3-chloro-2-((4-chloro-2-fluorobenzyl)oxy)-5,8-dihydro-1,7-naphthyridin-7(6H)-yl)methyl)-N′-hydroxy-5-methylnicotinimidamide (22 mg, crude). MS Calcd.: 489.1; MS Found: 490.2 [M+H]⁺.

### Step D: 3-(6-((3-chloro-2-((4-chloro-2-fluorobenzyl)oxy)-5,8-dihydro-1,7-naphthyridin-7(6H)-yl)methyl)-5-methylpyridin-3-yl)-5-(trifluoromethyl)-1,2,4-oxadiazole

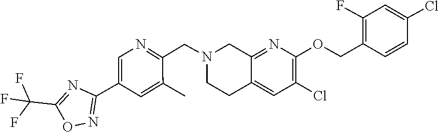 [SMILES: Cc1cc(-c2noc(C(F)(F)F)n2)cnc1CN1CCc2cc(Cl)c(OCc3ccc(Cl)cc3F)nc2C1]

To a solution of (Z)-6-((3-chloro-2-((4-chloro-2-fluorobenzyl)oxy)-5,8-dihydro-1,7-naphthyridin-7(6H)-yl)methyl)-N′-hydroxy-5-methylnicotinimidamide (22 mg, 0.045 mmol) in THE (3 ml) was added TFAA (47 mg, 0.225 mmol) at 0° C., then the mixture was stirred at 25° C. for 16 hours. The reaction mixture was poured into NaHCO₃.aq (20 mL) and extracted with EtOAc (2×20 mL), the combined organic layer was washed with brine, dried over anhydrous Na2SO4, filtered and concentrated under reduced pressure to give 3-(6-((3-chloro-2-((4-chloro-2-fluorobenzyl)oxy)-5,8-dihydro-1,7-naphthyridin-7(6H)-yl)methyl)-5-methylpyridin-3-yl)-5-(trifluoromethyl)-1,2,4-oxadiazole (43 mg, crude). MS Calcd.: 567.1; MS Found: 568.4 [M+H]⁺.

### Step E: 3-chloro-2-[(4-chloro-2-fluorophenyl)methoxy]-7-({3-methyl-5-[5-(trifluoromethyl)-4H-1,2,4-triazol-3-yl]pyridin-2-yl}methyl)-5,6,7,8-tetrahydro-1,7-naphthyridine

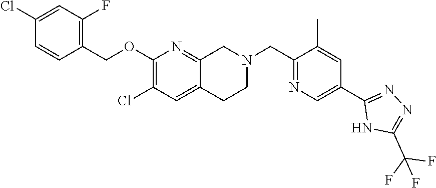 [SMILES: Cc1cc(-c2nnc(C(F)(F)F)[nH]2)cnc1CN1CCc2cc(Cl)c(OCc3ccc(Cl)cc3F)nc2C1]

A mixture of 3-(6-((3-chloro-2-((4-chloro-2-fluorobenzyl)oxy)-5,8-dihydro-1,7-naphthyridin-7(6H)-yl)methyl)-5-methylpyridin-3-yl)-5-(trifluoromethyl)-1,2,4-oxadiazole (43 mg, 0.076 mmol) and NH2NH2·H2O (0.47 ml) in EtOH (2 ml) was stirred at 70° C. for 1 hour under N2. The reaction mixture was purified by prep-HPLC to give 3-chloro-2-[(4-chloro-2-fluorophenyl)methoxy]-7-({3-methyl-5-[5-(trifluoromethyl)-4H-1,2,4-triazol-3-yl]pyridin-2-yl}methyl)-5,6,7,8-tetrahydro-1,7-naphthyridine (19.01 mg, yield: 44.19%).

¹H NMR (400 MHz, CD₃OD) δ 9.00 (s, 1H), 8.23 (s, 1H), 7.56 (s, 1H), 7.49 (t, J=8.0 Hz, 1H), 7.23-7.15 (m, 2H), 5.39 (s, 2H), 4.06 (s, 2H), 3.75 (s, 2H), 2.97-2.95 (m, 2H), 2.86-2.83 (m, 2H), 2.54 (s, 3H). ¹⁹F NMR (376 MHz, CD₃OD) δ −66.47, −117.43. LC-MS: m/z 567.2 (M+H)⁺.

### 2-[(4-chloro-2-fluorophenyl)methoxy]-7-({4-methyl-6-[5-(trifluoromethyl)-4H-1,2,4-triazol-3-yl]pyridazin-3-yl}methyl)-3-(trifluoromethyl)-5,6,7,8-tetrahydro-1,7-naphthyridine (compound 13)

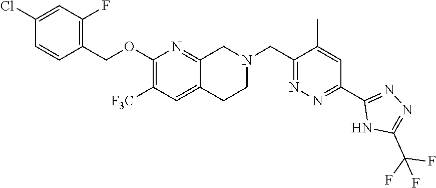 [SMILES: Cc1cc(-c2nnc(C(F)(F)F)[nH]2)nnc1CN1CCc2cc(C(F)(F)F)c(OCc3ccc(Cl)cc3F)nc2C1]

Compound 13 was synthesized following the similar route of Procedure 7, using 2-((4-chloro-2-fluorobenzyl)oxy)-3-(trifluoromethyl)-5,6,7,8-tetrahydro-1,7-naphthyridine, TFA salt and 6-formyl-5-methylpyridazine-3-carbonitrile in step B.

¹H NMR (400 MHz, CD3OD) δ 8.24 (s, 1H), 7.78 (s, 1H), 7.49 (t, J=8.0 Hz, 1H), 7.24-7.18 (m, 2H), 5.47 (s, 2H), 4.18 (s, 2H), 3.75 (s, 2H), 2.97-2.90 (m, 2H), 2.89-2.84 (m, 2H), 2.64 (s, 3H). ¹⁹F NMR (376 MHz, CD₃OD) δ −64.53, −66.46, −117.43. LC-MS: m/z 602.3 (M+H)⁺.

### 2-[(4-chloro-2-fluorophenyl)methoxy]-7-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]-3-(trifluoromethyl)-5,6,7,8-tetrahydro-1,7-naphthyridine (compound 16)

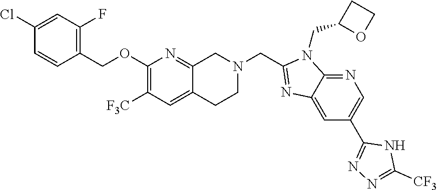 [SMILES: Fc1cc(Cl)ccc1COc1nc2c(cc1C(F)(F)F)CCN(Cc1nc3cc(-c4nnc(C(F)(F)F)[nH]4)cnc3n1C[C@@H]1CCO1)C2]

Compound 16 was synthesized following the similar route of Procedure 3, using tert-butyl 2-((4-chloro-2-fluorobenzyl)oxy)-3-(trifluoromethyl)-5,8-dihydro-1,7-naphthyridine-7(6H)-carboxylate in step A.

¹H NMR (400 MHz, DMSO-d₆) δ 9.00 (d, J=1.6 Hz, 1H), 8.62 (d, J=1.6 Hz, 1H), 7.93 (s, 1H), 7.52 (t, J=8.0 Hz, 1H), 7.46 (dd, J=10.0, 2.0 Hz, 1H), 7.31 (dd, J=8.0, 2.0 Hz, 1H), 5.43 (s, 2H), 5.11-5.19 (m, 1H), 4.77-4.86 (m, 1H), 4.64-4.72 (m, 1H), 4.43-4.51 (m, 1H), 4.30-4.38 (m, 1H), 4.13-4.24 (m, 2H), 3.74 (s, 2H), 2.80-2.91 (m, 4H), 2.61-2.70 (m, 1H), 2.36-2.46 (m, 1H). ¹⁹F NMR (376 MHz, DMSO-d₆) δ −61.67, −63.33, −115.00. LC-MS: m/z 697.5 (M+H)⁺.

### 5-[2-({2-[(4-chlorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-4H-1,2,4-triazole-3-carbonitrile (compound 19)

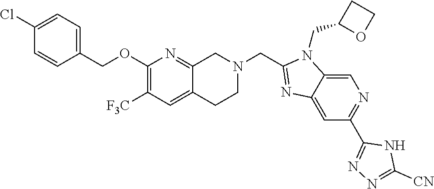 [SMILES: N#Cc1nnc(-c2cc3nc(CN4CCc5cc(C(F)(F)F)c(OCc6ccc(Cl)cc6)nc5C4)n(C[C@@H]4CCO4)c3cn2)[nH]1]

Compound 19 was synthesized following the similar route of Example 5, using tert-butyl 2-((4-chlorobenzyl)oxy)-3-(trifluoromethyl)-5,8-dihydro-1,7-naphthyridine-7(6H)-carboxylate in step A.

¹H NMR (400 MHz, DMSO-d₆) δ 15.75 (br s, 1H), 9.18 (s, 1H), 8.34 (s, 1H), 7.93 (s, 1H), 7.35-7.50 (m, 4H), 5.39 (s, 2H), 5.05-5.15 (m, 1H), 4.85-4.96 (m, 1H), 4.70-4.80 (m, 1H), 4.43-4.52 (m, 1H), 4.33-4.42 (m, 1H), 4.08-4.28 (m, 2H), 3.66-3.80 (m, 2H), 2.78-2.95 (m, 4H), 2.61-2.72 (m, 1H), 2.31-2.36 (m, 1H). ¹⁹F NMR (376 MHz, DMSO-d₆) δ −61.65. LC-MS: m/z 636.6 (M+H)⁺.

### 5-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-4H-1,2,4-triazole-3-carbonitrile (compound 20)

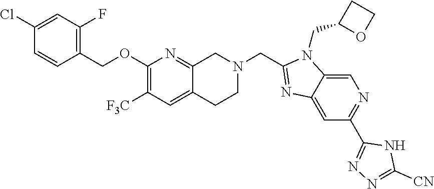 [SMILES: N#Cc1nnc(-c2cc3nc(CN4CCc5cc(C(F)(F)F)c(OCc6ccc(Cl)cc6F)nc5C4)n(C[C@@H]4CCO4)c3cn2)[nH]1]

Compound 20 was synthesized following the similar route of Procedure 5, using tert-butyl 2-((4-chloro-2-fluorobenzyl)oxy)-3-(trifluoromethyl)-5,8-dihydro-1,7-naphthyridine-7(6H)-carboxylate in step A.

¹H NMR (400 MHz, DMSO-d₆) δ 9.20 (s, 1H), 8.36 (s, 1H), 7.97 (s, 1H), 7.52 (t, J=8.0 Hz, 1H), 7.46 (dd, J=10.0, 2.0 Hz, 1H), 7.31 (dd, J=8.0, 1.2 Hz, 1H), 5.43 (s, 2H), 5.06-5.15 (m, 1H), 4.87-4.96 (m, 1H), 4.71-4.79 (m, 1H), 4.43-4.52 (m, 1H), 4.31-4.42 (m, 1H), 3.80-3.95 (m, 2H), 2.95-3.08 (m, 2H), 2.81-2.92 (m, 2H), 2.62-2.74 (m, 2H), 2.29-2.42 (m, 2H). ¹⁹F NMR (376 MHz, DMSO-d₆) δ −61.73, −114.99. LC-MS: m/z 654.2 (M+H)⁺.

### Procedure 8

### 3-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-4,5-dihydro-1,2,4-oxadiazol-5-one (compound 18)

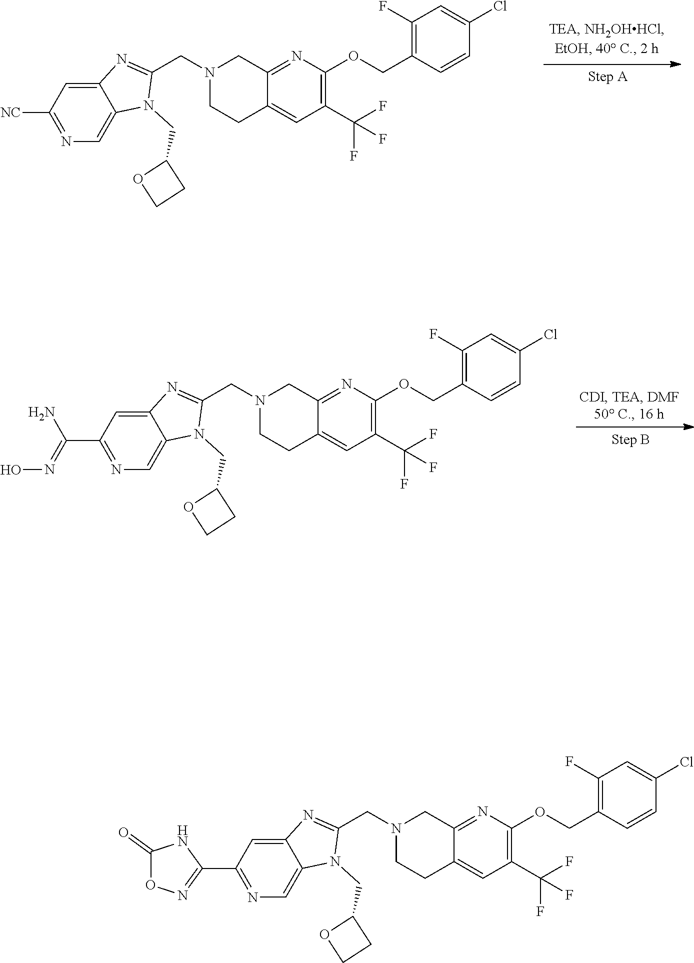 [SMILES: N#Cc1cc2nc(CN3CCc4cc(C(F)(F)F)c(OCc5ccc(Cl)cc5F)nc4C3)n(C[C@@H]3CCO3)c2cn1 ; N/C(=N\O)c1cc2nc(CN3CCc4cc(C(F)(F)F)c(OCc5ccc(Cl)cc5F)nc4C3)n(C[C@@H]3CCO3)c2cn1 ; O=c1[nH]c(-c2cc3nc(CN4CCc5cc(C(F)(F)F)c(OCc6ccc(Cl)cc6F)nc5C4)n(C[C@@H]4CCO4)c3cn2)no1]

### Step A: (S,Z)-2-((2-((4-chloro-2-fluorobenzyl)oxy)-3-(trifluoromethyl)-58-dihydro-1,7-naphthyridin-7(6H)-yl)methyl)-N′-hydroxy-3-(oxetan-2-ylmethyl)-3H-imidazo[4,5-c]pyridine-6-carboximidamide

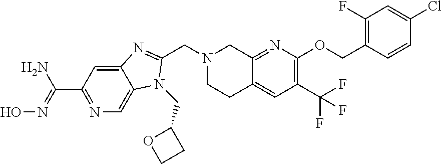 [SMILES: N/C(=N\O)c1cc2nc(CN3CCc4cc(C(F)(F)F)c(OCc5ccc(Cl)cc5F)nc4C3)n(C[C@@H]3CCO3)c2cn1]

A solution of (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbonitrile (80 mg, 0.14 mmol), HONH₂—HCl (37.95 mg, 0.55 mmol), TEA (84.99 mg, 0.84 mmol) in EtOH (4.0 mL) was stirred at 40° C. for 2 hours. The reaction mixture was diluted with H₂O (20 mL) and extracted with EtOAc (15 mL×2). The combined organic layer was dried over anhydrous Na₂SO₄, filtered and evaporated to give (S,Z)-2-((2-((4-chloro-2-fluorobenzyl)oxy)-3-(trifluoromethyl)-5,8-dihydro-1,7-naphthyridin-7(6H)-yl)methyl)-N′-hydroxy-3-(oxetan-2-ylmethyl)-3H-imidazo[4,5-c]pyridine-6-carboximidamide (65 mg, yield: 76.9%). LC-MS: m/z 620.3 (M+H)⁺.

### Step B: 3-[2-({2-[(4-chloro-2-fluorophenyl)methoxy]-3-(trifluoromethyl)-5,6,7,8-tetrahydro-1,7-naphthyridin-7-yl/methyl)-3-{[(2S)-oxetan-2-yl]methyl}-3H-imidazo[4,5-c]pyridin-6-yl]-4,5-dihydro-1,2,4-oxadiazol-5-one

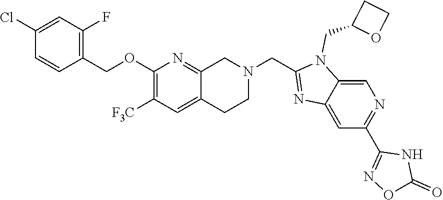 [SMILES: O=c1[nH]c(-c2cc3nc(CN4CCc5cc(C(F)(F)F)c(OCc6ccc(Cl)cc6F)nc5C4)n(C[C@@H]4CCO4)c3cn2)no1]

A mixture of (S,Z)-2-((2-((4-chloro-2-fluorobenzyl)oxy)-3-(trifluoromethyl)-5,8-dihydro-1,7-naphthyridin-7(6H)-yl)methyl)-N′-hydroxy-3-(oxetan-2-ylmethyl)-3H-imidazo[4,5-c]pyridine-6-carboximidamide (45 mg, 0.073 mmol), TEA (22.12 mg, 0.22 mmol) and CDI (17.67 mg, 0.11 mmol) in DMF (2.0 mL) was stirred at 50° C. for 16 hours. The reaction mixture was purified directly by Prep-HPLC (0.1% NH₃H₂O/H₂O/CH₃CN) to give 3-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-4,5-dihydro-1,2,4-oxadiazol-5-one (5.46 mg, yield: 11.6%).

¹H NMR (400 MHz, DMSO-d₆) δ 9.13 (s, 1H), 8.18 (s, 1H), 7.93 (s, 1H), 7.52 (t, J=8.0 Hz, 1H), 7.46 (dd, J=10.0, 2.0 Hz, 1H), 7.32 (dd, J=8.4, 2.0 Hz, 1H), 5.43 (s, 2H), 5.05-5.14 (m, 1H), 4.84-4.93 (m, 1H), 4.70-4.77 (m, 1H), 4.43-4.51 (m, 1H), 4.33-4.41 (m, 1H), 4.21 (d, J=13.6 Hz, 1H), 4.11 (d, J=13.6 Hz, 1H), 3.67-3.80 (m, 2H), 2.77-2.93 (m, 4H), 2.61-2.71 (m, 1H), 2.31-2.43 (m, 1H). ¹⁹F NMR (376 MHz, DMSO-d₆) δ −1.68, −114.99. LC-MS: m/z 646.1 (M+H)⁺.

### Procedure 9

### 2-[(4-chloro-2-fluorophenyl)methoxy]-7-[(3-{[(2S)-oxetan-2-yl]methyl}-6-(1H-1,2,3,4-tetrazol-5-yl)-3H-imidazo[4,5-c]pyridin-2-yl)methyl]-3-(trifluoromethyl)-5,6,7,8-tetrahydro-1,7-naphthyridine (compound 14)

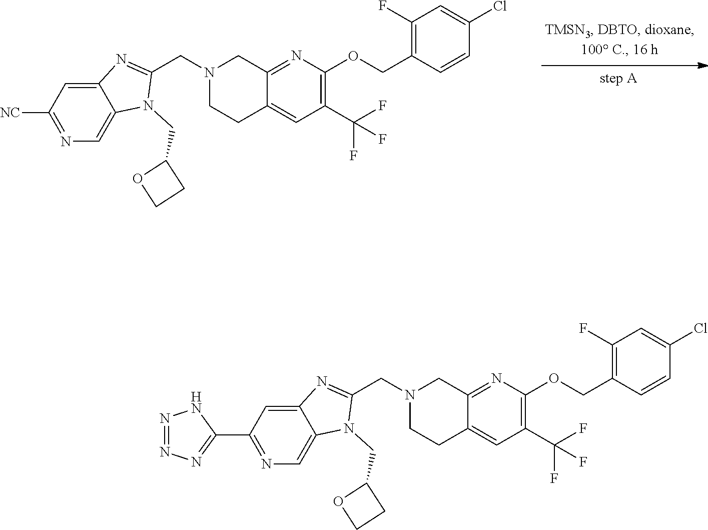 [SMILES: N#Cc1cc2nc(CN3CCc4cc(C(F)(F)F)c(OCc5ccc(Cl)cc5F)nc4C3)n(C[C@@H]3CCO3)c2cn1 ; Fc1cc(Cl)ccc1COc1nc2c(cc1C(F)(F)F)CCN(Cc1nc3cc(-c4nnn[nH]4)ncc3n1C[C@@H]1CCO1)C2]

### Step A: 2-[(4-chloro-2-fluorophenyl)methoxy]-7-[(3-{[(2S)-oxetan-2-yl]methyl)-6-(1H-1,2,3,4-tetrazol-5-yl)-3H-imidazo[4,5-c]pyridin-2-yl)methyl]-3-(trifluoromethyl)-5,6,7,8-tetrahydro-1,7-naphthyridine

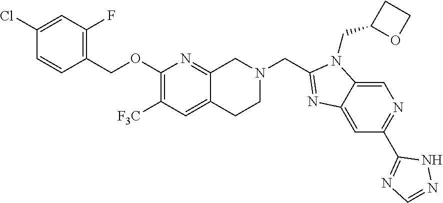 [SMILES: Fc1cc(Cl)ccc1COc1nc2c(cc1C(F)(F)F)CCN(Cc1nc3cc(-c4ncn[nH]4)ncc3n1C[C@@H]1CCO1)C2]

A mixture of (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbonitrile (50 mg, 0.085 mmol), TMSN₃ (49.06 mg, 0.43 mmol) and DBTO (42.31 mg, 0.17 mmol) in dioxane (2.0 mL) was stirred at 100° C. for 16 h. The mixture cooled down to room temperature and then purified directly by Prep-HPLC (0.1% FA/H₂O/CH₃CN) to give 2-[(4-chloro-2-fluorophenyl)methoxy]-7-[(3-{[(2S)-oxetan-2-yl]methyl}-6-(1H-1,2,3,4-tetrazol-5-yl)-3H-imidazo[4,5-c]pyridin-2-yl)methyl]-3-(trifluoromethyl)-5,6,7,8-tetrahydro-1,7-naphthyridine (4.41 mg, yield: 7.6%).

¹H NMR (400 MHz, DMSO-d₆) δ 9.20 (s, 1H), 8.42 (s, 1H), 7.93 (s, 1H), 7.51 (t, J=8.0 Hz, 1H), 7.46 (dd, J=10.0, 2.0 Hz, 1H), 7.31 (dd, J=8.4, 1.6 Hz, 1H), 5.42 (s, 2H), 5.06-5.16 (m, 1H), 4.86-4.96 (m, 1H), 4.71-4.79 (m, 1H), 4.34-4.52 (m, 2H), 4.22 (d, J=14.0 Hz, 1H), 4.12 (d, J=14.0 Hz, 1H), 3.67-3.81 (m, 2H), 2.77-2.93 (m, 4H), 2.61-2.73 (m, 1H), 2.30-2.45 (m, 1H). ¹⁹F NMR (376 MHz, DMSO-d₆) δ −61.68, −114.99. LC-MS: m/z 630.2 (M+H)⁺.

### 2-[(4-chloro-2-fluorophenyl)methoxy]-7-[(3-{[(2S)-oxetan-2-yl]methyl}-6-(1H-1,2,3,4-tetrazol-5-yl)-3H-imidazo[4,5-b]pyridin-2-yl)methyl]-3-(trifluoromethyl)-5,6,7,8-tetrahydro-1,7-naphthyridine (compound 15)

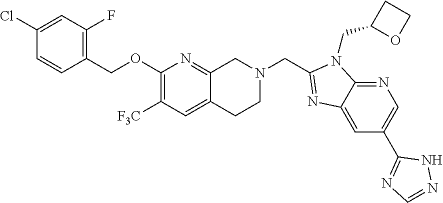 [SMILES: Fc1cc(Cl)ccc1COc1nc2c(cc1C(F)(F)F)CCN(Cc1nc3cc(-c4ncn[nH]4)cnc3n1C[C@@H]1CCO1)C2]

Compound 15 was synthesized following the similar route of Procedure 9, using (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-b]pyridine-6-carbonitrile in step A.

¹H NMR (400 MHz, DMSO-d₆) δ 9.02 (s, 1H), 8.59 (s, 1H), 7.93 (s, 1H), 7.52 (t, J=8.4 Hz, 1H), 7.45 (d, J=9.6 Hz, 1H), 7.31 (d, J=7.6 Hz, 1H), 5.43 (s, 2H), 5.12-5.20 (m, 1H), 4.76-4.86 (m, 1H), 4.63-4.73 (m, 1H), 4.42-4.52 (m, 1H), 4.30-4.38 (m, 1H), 4.13-4.24 (m, 2H), 3.75 (s, 2H), 2.79-2.94 (m, 4H), 2.60-2.71 (m, 1H), 2.36-2.49 (m, 1H). ¹⁹F NMR (376 MHz, DMSO-d₆) δ −61.67, −114.99. LC-MS: m/z 630.2 (M+H)⁺.

### 3-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-b]pyridin-6-yl]-4,5-dihydro-1,2,4-oxadiazol-5-one (compound 29)

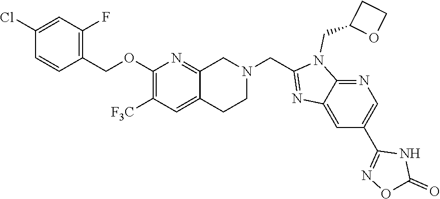 [SMILES: O=c1[nH]c(-c2cnc3c(c2)nc(CN2CCc4cc(C(F)(F)F)c(OCc5ccc(Cl)cc5F)nc4C2)n3C[C@@H]2CCO2)no1]

Compound 29 was synthesized following the similar route of Procedure 1, using 2-((4-chloro-2-fluorobenzyl)oxy)-3-(trifluoromethyl)-5,6,7,8-tetrahydro-1,7-naphthyridine, TFA Salt.

¹H NMR (400 MHz, DMSO-d₆) δ 13.09 (s, 1H), 8.79 (d, J=2.0 Hz, 1H), 8.44 (s, 1H), 7.92 (s, 1H), 7.53-7.44 (m, 2H), 7.32-7.30 (m, 1H), 5.42 (s, 2H), 5.15-5.12 (m, 1H), 4.83-4.77 (m, 1H), 4.70-4.65 (m, 1H), 4.48-4.43 (m, 1H), 4.35-4.29 (m, 1H), 4.19-4.18 (m, 2H), 3.71-3.76 (m, 2H), 2.86-2.83 (m, 4H), 2.67-2.62 (m, 1H), 2.39-2.31 (m, 1H). ¹⁹F NMR (376 MHz, DMSO-d₆) δ −61.84, −115.01. LC-MS: m/z 646.1 (M+H)⁺.

### 3-[2-({2-[(4-chloro-2,6-difluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-b]pyridin-6-yl]-4,5-dihydro-1,2,4-oxadiazol-5-one (compound 30)

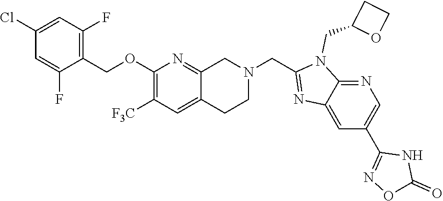 [SMILES: O=c1[nH]c(-c2cnc3c(c2)nc(CN2CCc4cc(C(F)(F)F)c(OCc5c(F)cc(Cl)cc5F)nc4C2)n3C[C@@H]2CCO2)no1]

Compound 30 was synthesized following the similar route of Procedure 1, using 2-((4-chloro-2,6-difluorobenzyl)oxy)-3-(trifluoromethyl)-5,6,7,8-tetrahydro-1,7-naphthyridine, TFA Salt in step E.

¹H NMR (400 MHz, DMSO-d₆) δ 13.11 (s, 1H), 8.80 (s, 1H), 8.46 (s, 1H), 7.92-7.88 (m, 1H), 7.38-7.31 (m, 2H), 5.39 (s, 2H), 5.12-5.15 (m, 1H), 4.69-4.79 (m, 2H), 4.28-4.46 (m, 4H), 3.82-3.89 (m, 2H), 2.85-2.95 (m, 4H), 2.61-2.70 (m, 1H), 2.39-2.43 (m, 1H). ¹⁹F NMR (376 MHz, DMSO-d₆) δ −61.82, −112.04. LC-MS: m/z 666.2 (M+H)⁺.

### 3-[2-({2-[(4-chlorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-4,5-dihydro-1,2,4-oxadiazol-5-one (compound 31)

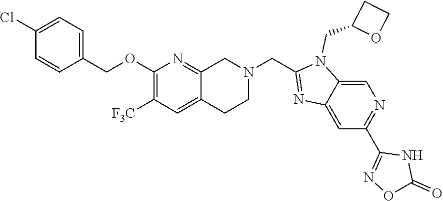 [SMILES: O=c1[nH]c(-c2cc3nc(CN4CCc5cc(C(F)(F)F)c(OCc6ccc(Cl)cc6)nc5C4)n(C[C@@H]4CCO4)c3cn2)no1]

Compound 31 was synthesized following the similar route of Procedure 1, using 2-((4-chlorobenzyl)oxy)-3-(trifluoromethyl)-5,6,7,8-tetrahydro-1,7-naphthyridine, TFA Salt and (S)-2-(chloromethyl)-3-(oxetan-2-ylmethyl)-3H-imidazo[4,5-c]pyridine-6-carbonitrile in step E.

¹H NMR (400 MHz, DMSO-d₆) δ 9.25 (s, 1H), 8.27 (s, 1H), 8.05 (s, 1H), 7.44-7.48 (m, 4H), 5.42 (s, 2H), 5.05-5.13 (m, 1H), 4.88-4.94 (m, 1H), 4.58-4.76 (m, 4H), 4.45-4.50 (m, 1H), 4.34-4.39 (m, 1H), 4.17-4.24 (m, 2H), 3.31-3.39 (m, 2H), 2.94-3.03 (m, 1H), 2.67-2.75 (m, 1H), 2.32-2.41 (m, 1H). ¹⁹F NMR (376 MHz, DMSO-d₆) δ −61.84, −74.36. LC-MS: m/z 628.1 (M+H)⁺.

### 3-[2-({2-[(4-chloro-2,6-difluorophenyl)methoxy]-3-(trifluoromethyl)-5,6,7,8-tetrahydro-1,7-naphthyridin-7-yl}methyl)-3-{1[(2S)-oxetan-2-yl]methyl}-3H-imidazo[4,5-c]pyridin-6-yl]-4,5-dihydro-1,2,4-oxadiazol-5-one (compound 32)

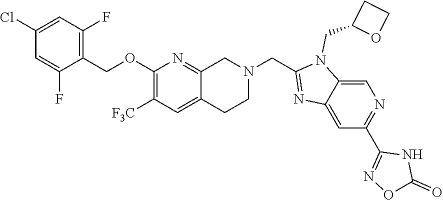 [SMILES: O=c1[nH]c(-c2cc3nc(CN4CCc5cc(C(F)(F)F)c(OCc6c(F)cc(Cl)cc6F)nc5C4)n(C[C@@H]4CCO4)c3cn2)no1]

Compound 32 was synthesized following the similar route of Procedure 1, using 2-((4-chloro-2,6-difluorobenzyl)oxy)-3-(trifluoromethyl)-5,6,7,8-tetrahydro-1,7-naphthyridine, HCl Salt and (S)-2-(chloromethyl)-3-(oxetan-2-ylmethyl)-3H-imidazo[4,5-c]pyridine-6-carbonitrile in step E.

¹H NMR (400 MHz, DMSO-d₆) δ 13.02 (br s, 1H), 9.24 (s, 1H), 8.24 (s, 1H), 7.99 (s, 1H), 7.31-7.40 (m, 2H), 5.42 (s, 2H), 5.04-5.14 (m, 1H), 4.84-4.97 (m, 1H), 4.71-4.79 (m, 1H), 4.45-4.53 (m, 3H), 4.32-4.42 (m, 1H), 3.96-4.03 (m, 2H), 3.11-3.16 (m, 2H), 2.88-2.96 (m, 2H), 2.65-2.72 (m, 1H), 2.32-2.43 (m, 1H). ¹⁹F NMR (376 MHz, DMSO-d₆) δ −61.86, −112.07. LC-MS: m/z 664.4 (M+H)⁺.

### 3-[2-({2-[(2-chloro-4-methylphenyl)methoxy]-3-(trifluoromethyl)-5,6,7,8-tetrahydro-1,7-naphthyridin-7-yl}methyl)-3-{1[(2S)-oxetan-2-yl]methyl}-3H-imidazo[4,5-c]pyridin-6-yl]-4,5-dihydro-1,2,4-oxadiazol-5-one (compound 33)

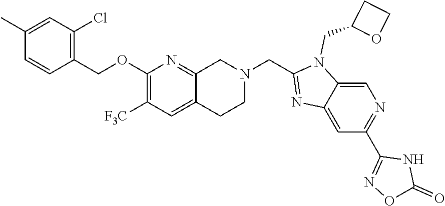 [SMILES: Cc1ccc(COc2nc3c(cc2C(F)(F)F)CCN(Cc2nc4cc(-c5noc(=O)[nH]5)ncc4n2C[C@@H]2CCO2)C3)c(Cl)c1]

Compound 33 was synthesized following the similar route of Procedure 1, using 2-((2-chloro-4-methylbenzyl)oxy)-3-(trifluoromethyl)-5,6,7,8-tetrahydro-1,7-naphthyridine and (S)-2-(chloromethyl)-3-(oxetan-2-ylmethyl)-3H-imidazo[4,5-c]pyridine-6-carbonitrile in step E.

¹H NMR (400 MHz, DMSO-d₆) δ 13.00 (s, 1H), 9.17 (s, 1H), 8.21 (s, 1H), 7.93 (s 1H), 7.41 (d, J=7.6 Hz, 1H), 7.32 (s, 1H), 7.16 (d, J=8.4 Hz, 1H), 5.40 (s, 2H), 5.06-5.11 (m, 1H), 4.87-4.93 (m, 1H), 4.72-4.77 (m, 1H), 4.44-4.49 (m, 1H), 4.34-4.39 (m, 1H), 4.20-4.23 (m, 1H), 4.09-4.13 (m, 1H), 3.67-3.79 (m, 2H), 2.82-2.86 (m, 4H), 2.62-2.68 (m, 1H), 2.33-2.42 (m, 1H), 2.29 (s, 3H). ¹⁹F NMR (376 MHz, DMSO-d₆) δ −61.62. LC-MS: m/z 642.2 (M+H)⁺.

### 3-[2-({2-[(4-chloro-2,6-difluorophenyl)methoxy]-3-methyl-5,6,7,8-tetrahydro-1,7-naphthyridin-7-yl}methyl)-3-{[(2S)-oxetan-2-yl]methyl}-3H-imidazo[4,5-c]pyridin-6-yl]-4,5-dihydro-1,2,4-oxadiazol-5-one (compound 34)

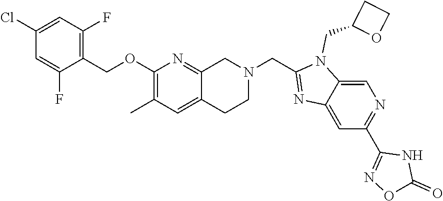 [SMILES: Cc1cc2c(nc1OCc1c(F)cc(Cl)cc1F)CN(Cc1nc3cc(-c4noc(=O)[nH]4)ncc3n1C[C@@H]1CCO1)CC2]

Compound 34 was synthesized following the similar route of Procedure 1, using 2-((4-chloro-2,6-difluorobenzyl)oxy)-3-methyl-5,6,7,8-tetrahydro-1,7-naphthyridine and (S)-2-(chloromethyl)-3-(oxetan-2-ylmethyl)-3H-imidazo[4,5-c]pyridine-6-carbonitrile in step E.

¹H NMR (400 MHz, DMSO-d₆) δ 13.06 (s, 1H), 9.25 (s, 1H), 8.28 (s, 1H), 7.38-7.43 (m, 3H), 5.29 (s, 2H), 5.08-5.12 (m, 1H), 4.90-4.94 (m, 1H), 4.73-4.78 (m, 1H), 4.50 (dd, J=12.4 Hz, 6.4 Hz, 1H), 4.33-4.39 (m, 1H), 4.10-4.30 (m, 2H), 3.60-3.85 (m, 2H), 2.87-2.96 (m, 2H), 2.67-2.73 (m, 2H), 2.57-2.66 (m, 1H), 2.32-2.40 (m, 1H), 2.06 (s, 3H). ¹⁹F NMR (376 MHz, DMSO-d₆) δ −112.28. LC-MS: m/z 610.3 (M+H)⁺.

### 3-[2-({2-[(4-chloro-2-fluorophenyl)methoxy]-3-(trifluoromethyl)-5,6,7,8-tetrahydro-1,7-naphthyridin-7-yl}methyl)-1-{1[(2S)-oxetan-2-yl]methyl}-1H-1,3-benzodiazol-5-yl]-4,5-dihydro-1,2,4-oxadiazol-5-one (compound 36)

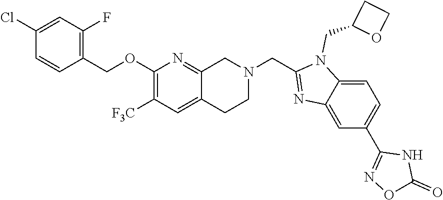 [SMILES: O=c1[nH]c(-c2ccc3c(c2)nc(CN2CCc4cc(C(F)(F)F)c(OCc5ccc(Cl)cc5F)nc4C2)n3C[C@@H]2CCO2)no1]

Compound 36 was synthesized following the similar route of Procedure 1, using 4-fluoro-3-nitrobenzonitrile in step A and 2-((4-chloro-2-fluorobenzyl)oxy)-3-(trifluoromethyl)-5,6,7,8-tetrahydro-1,7-naphthyridine in step E.

¹H NMR (400 MHz, DMSO-d₆) δ 12.91 (s, 1H), 8.09 (s, 1H), 7.92 (s, 1H), 7.82 (d, J=8.4 Hz, 1H), 7.71 (d, J=8.4 Hz, 1H), 7.51 (t, J=8.4 Hz, 1H), 7.46 (dd, J=10.0 Hz, 1.2 Hz, 1H), 7.31 (d, J=8.0 Hz, 1H), 5.42 (s, 2H), 5.03-5.09 (m, 1H), 4.73-4.79 (m, 1H), 4.62 (dd, J=15.2 Hz, 2.4 Hz, 1H), 4.42-4.48 (m, 1H), 4.32-4.37 (m, 1H), 4.15 (d, J=13.6 Hz, 1H), 4.04 (d, J=13.6 Hz, 1H), 3.64-3.74 (m, 2H), 2.82-2.83 (m, 4H), 2.59-2.67 (m, 1H), 2.32-2.40 (m, 1H). ¹⁹F NMR (376 MHz, DMSO-d₆) δ −61.68, −114.99. LC-MS: m/z 645.2 (M+H)⁺.

### 3-[2-({2-[(4-chloro-2,6-difluorophenyl)methoxy]-3-methyl-5,6,7,8-tetrahydro-1,7-naphthyridin-7-yl}methyl)-1-{[(2S)-oxetan-2-yl]methyl}-1H-1,3-benzodiazol-5-yl]-4,5-dihydro-1,2,4-oxadiazol-5-one (compound 37)

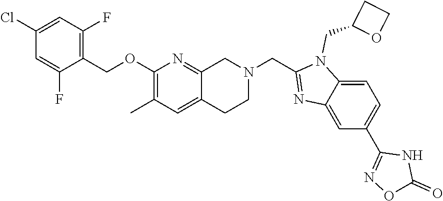 [SMILES: Cc1cc2c(nc1OCc1c(F)cc(Cl)cc1F)CN(Cc1nc3cc(-c4noc(=O)[nH]4)ccc3n1C[C@@H]1CCO1)CC2]

Compound 37 was synthesized following the similar route of Procedure 1, using 4-fluoro-3-nitrobenzonitrile in step A and 2-((4-chloro-2,6-difluorobenzyl)oxy)-3-methyl-5,6,7,8-tetrahydro-1,7-naphthyridine in step E.

¹H NMR (400 MHz, DMSO-d₆) δ 12.84 (s, 1H), 8.07 (s, 1H), 7.78 (d, J=8.4 Hz, 1H), 7.71 (dd, J=8.4 Hz, J=1.2 Hz, 1H), 7.35-7.39 (m, 2H), 7.31 (s, 1H), 5.26-5.29 (m, 2H), 5.03-5.09 (m, 1H), 4.72-4.78 (m, 1H), 4.62 (dd, J=14.8 Hz, J=2.8 Hz, 1H), 4.42-4.48 (m, 1H), 4.32-4.37 (m, 1H), 4.11 (d, J=13.6 Hz, 1H), 4.00 (d, J=13.6 Hz, 1H), 3.51-3.60 (m, 2H), 2.73-2.80 (m, 2H), 2.69-2.70 (m, 2H), 2.57-2.66 (m, 1H), 2.32-2.40 (m, 1H), 2.02 (s, 3H). ¹⁹F NMR (376 MHz, DMSO-d₆) δ −112.29. LC-MS: m/z 609.3 (M+H)⁺.

### 3-[2-({2-[(4-chloro-2-fluorophenyl)methoxy]-3-(trifluoromethyl)-5,6,7,8-tetrahydro-1,7-naphthyridin-7-yl}methyl)-6-fluoro-1-{[(2S)-oxetan-2-yl]methyl}-1H-1,3-benzodiazol-5-yl]-4,5-dihydro-1,2,4-oxadiazol-5-one (compound 38)

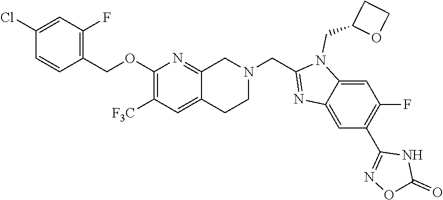 [SMILES: O=c1[nH]c(-c2cc3nc(CN4CCc5cc(C(F)(F)F)c(OCc6ccc(Cl)cc6F)nc5C4)n(C[C@@H]4CCO4)c3cc2F)no1]

Compound 38 was synthesized following the similar route of Procedure 1, using 2,4-difluoro-5-nitrobenzonitrile in step A and 2-((4-chloro-2-fluorobenzyl)oxy)-3-(trifluoromethyl)-5,6,7,8-tetrahydro-1,7-naphthyridine in step E.

¹H NMR (400 MHz, DMSO-d₆) δ 12.85 (br s, 1H), 8.08 (d, J=6.0 Hz, 1H), 8.03 (s, 1H), 7.90 (d, J=10.8 Hz, 1H), 7.52 (t, J=8.0 Hz, 1H), 7.47 (dd, J=10.0 Hz, 2.0 Hz, 1H), 7.32 (dd, J=8.0 Hz, 1.6 Hz, 1H), 5.45 (s, 2H), 5.00-5.08 (m, 1H), 4.71-4.80 (m, 1H), 4.58-4.64 (m, 1H), 4.38-4.55 (m, 3H), 4.32-4.37 (m, 1H), 4.06-4.20 (m, 2H), 3.21-3.35 (m, 2H), 2.93-3.02 (m, 2H), 2.62-2.71 (m, 1H), 2.28-2.40 (m, 1H). ¹⁹F NMR (376 MHz, DMSO-d₆) δ −61.83, −114.97, −116.74. LC-MS: m/z 663.1 (M+H)⁺.

### 2-[(4-chloro-2,6-difluorophenyl)methoxy]-7-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]-3-(trifluoromethyl)-5,6,7,8-tetrahydro-1,7-naphthyridine (compound 39)

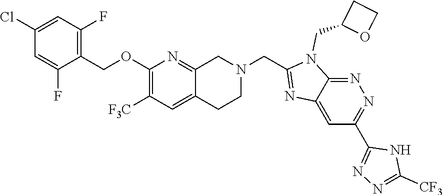 [SMILES: Fc1cc(Cl)cc(F)c1COc1nc2c(cc1C(F)(F)F)CCN(Cc1nc3cc(-c4nnc(C(F)(F)F)[nH]4)nnc3n1C[C@@H]1CCO1)C2]

Compound 39 was synthesized following the similar route of Procedure 4, using 2-((4-chloro-2,6-difluorobenzyl)oxy)-3-(trifluoromethyl)-5,6,7,8-tetrahydro-1,7-naphthyridine in step B.

¹H NMR (400 MHz, DMSO-d₆) δ 15.98 (s, 1H), 8.55 (s, 1H), 7.92 (s, 1H), 7.41-7.38 (m, 2H), 5.39 (s, 2H), 5.23-5.22 (m, 1H), 4.98-4.96 (m, 1H), 4.88-4.84 (m, 1H), 4.49-4.48 (m, 1H), 4.38-4.36 (m, 1H), 4.29 (s, 2H), 3.79 (s, 2H), 2.91-2.88 (m, 2H), 2.84-2.82 (m, 2H), 2.71-2.66 (m, 1H), 2.32 (m, 1H). ¹⁹F NMR (376 MHz, DMSO-d₆) δ −61.77, −63.57, −112.10. LC-MS: m/z 716.2 (M+H)⁺.

### 5-[2-({2-[(4-chloro-2,6-difluorophenyl)methoxy]-3-methyl-5,6,7,8-tetrahydro-1,7-naphthyridin-7-yl}methyl)-3-{[(2S)-oxetan-2-yl]methyl}-3H-imidazo[4,5-c]pyridin-6-yl]-4H-1,2,4-triazole-3-carbonitrile (compound 40)

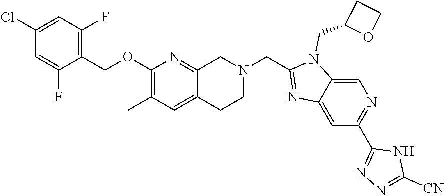 [SMILES: Cc1cc2c(nc1OCc1c(F)cc(Cl)cc1F)CN(Cc1nc3cc(-c4nnc(C#N)[nH]4)ncc3n1C[C@@H]1CCO1)CC2]

Compound 40 was synthesized following the similar route of Procedure 5, using tert-butyl 2-((4-chloro-2,6-di-fluorobenzyl)oxy)-3-methyl-5,8-dihydro-1,7-naphthyridine-7(6H)-carboxylate in step A.

¹H NMR (400 MHz, DMSO-d₆) δ 15.80 (s, 1H), 9.25 (s, 1H), 8.39 (s, 1H), 7.41-7.37 (m, 3H), 5.28 (s, 2H), 5.15-5.07 (m, 1H), 4.94-4.88 (m, 1H), 4.78 (m, 1H), 4.52-4.47 (m, 1H), 4.38-4.34 (m, 1H), 4.15-3.99 (m, 2H), 2.90-2.84 (m, 2H), 2.71-2.63 (m, 2H), 2.41-2.32 (m, 4H), 2.11 (s, 3H). ¹⁹F NMR (376 MHz, DMSO-d₆) δ −112.27. LC-MS: m/z 618.4 (M+H)⁺.

### 5-[2-({2-[(4-chloro-2,6-difluorophenyl)methoxy]-3-methyl-5,6,7,8-tetrahydro-1,7-naphthyridin-7-yl}methyl)-3-{1[(2S)-oxetan-2-yl]methyl}-3H-imidazo[4,5-b]pyridin-6-yl]-4H-1,2,4-triazole-3-carbonitrile (compound 41)

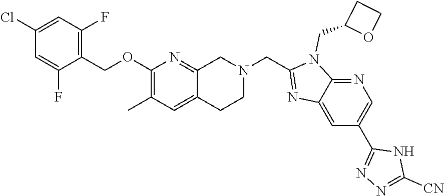 [SMILES: Cc1cc2c(nc1OCc1c(F)cc(Cl)cc1F)CN(Cc1nc3cc(-c4nnc(C#N)[nH]4)cnc3n1C[C@@H]1CCO1)CC2]

Compound 41 was synthesized following the similar route of Procedure 5, using tert-butyl 2-((4-chloro-2,6-di-fluorobenzyl)oxy)-3-methyl-5,8-dihydro-1,7-naphthyridine-7(6H)-carboxylate in step A and (S)-2-(chloromethyl)-3-(oxetan-2-ylmethyl)-3H-imidazo[4,5-b]pyridine-6-carbonitrile in step B.

¹H NMR (400 MHz, DMSO-d₆) δ 8.98-9.06 (m, 1H), 8.64 (s, 1H), 7.37 (d, J=7.2 Hz, 2H), 7.32 (s, 1H), 5.26 (s, 2H), 5.09-5.20 (m, 1H), 4.77-4.86 (m, 1H), 4.65-4.74 (m, 1H), 4.42-4.51 (m, 1H), 4.33-4.38 (m, 1H), 4.11-4.29 (m, 2H), 3.58-3.71 (m, 1H), 3.47 (s, 2H), 2.79-2.91 (m, 1H), 2.60-2.78 (m, 3H), 2.36-2.47 (m, 1H), 2.03 (s, 3H). ¹⁹F NMR (376 MHz, DMSO-d₆) δ −112.29. LC-MS: m/z 618.3 (M+H)⁺.

### 5-[6-({2-[(4-chloro-2-fluorophenyl)methoxy]-3-(trifluoromethyl)-5,6,7,8-tetrahydro-1,7-naphthyridin-7-yl}methyl)-7-{1[(2S)-oxetan-2-yl]methyl}-7H-imidazo[4,5-c]pyridazin-3-yl]-4H-1,2,4-triazole-3-carbonitrile (compound 42)

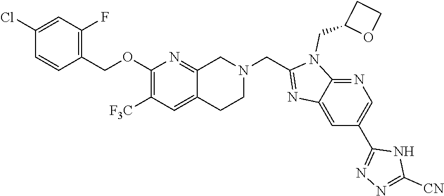 [SMILES: N#Cc1nnc(-c2cnc3c(c2)nc(CN2CCc4cc(C(F)(F)F)c(OCc5ccc(Cl)cc5F)nc4C2)n3C[C@@H]2CCO2)[nH]1]

Compound 42 was synthesized following the similar route of Procedure 5, using (S)-6-((2-((4-chloro-2-fluorobenzyl)oxy)-3-(trifluoromethyl)-5,8-dihydro-1,7-naphthyridin-7(6H)-yl)methyl)-7-(oxetan-2-ylmethyl)-7H-imidazo[4,5-c]pyridazine-3-carbonitrile in step C.

¹H NMR (400 MHz, DMSO-d₆) δ 8.57 (s, 1H), 7.95 (s, 1H), 7.52 (t, J=8.0 Hz, 1H), 7.47 (d, J=10.0 Hz, 1H), 7.32 (d, J=8.0 Hz, 1H), 5.43 (s, 2H), 5.18-5.28 (m, 1H), 4.94-5.04 (m, 1H), 4.81-4.91 (m, 1H), 4.45-4.53 (m, 1H), 4.33-4.41 (m, 1H), 4.24-4.32 (m, 2H), 3.81 (s, 2H), 2.89-2.97 (m, 2H), 2.80-2.88 (m, 2H), 2.65-2.77 (m, 2H). ¹⁹F NMR (376 MHz, DMSO-d₆) δ −61.67, −115.04. LC-MS: m/z 655.4 (M+H)⁺.

### 5-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-4H-1,2,4-triazole-3-carboxamide (compound 43)

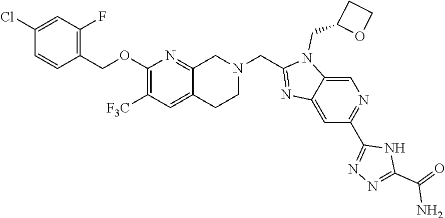 [SMILES: NC(=O)c1nnc(-c2cc3nc(CN4CCc5cc(C(F)(F)F)c(OCc6ccc(Cl)cc6F)nc5C4)n(C[C@@H]4CCO4)c3cn2)[nH]1]

Compound 43 was synthesized following the similar route of Procedure 5, using tert-butyl 2-((4-chloro-2-fluorobenzyl)oxy)-3-(trifluoromethyl)-5,8-dihydro-1,7-naphthyridine-7(6H)-carboxylate in step A.

¹H NMR (400 MHz, DMSO-d₆) δ 9.12 (s, 1H), 8.31 (s, 1H), 7.92 (s, 1H), 7.83 (s, 1H), 7.58 (s, 1H), 7.51 (t, J=10.8 Hz, 1H), 7.44 (d, J=9.6 Hz, 1H), 7.30 (d, J=8.0 Hz, 1H), 5.42 (s, 2H), 5.05-5.17 (m, 1H), 4.82-4.93 (m, 1H), 4.69-4.78 (m, 1H), 4.43-4.53 (m, 1H), 4.33-4.42 (m, 1H), 4.20 (d, J=13.6 Hz, 1H), 4.10 (d, J=13.6 Hz, 1H), 3.64-3.82 (m, 2H), 2.78-2.93 (m, 4H), 2.67-2.71 (m, 1H), 2.35-2.44 (m, 1H). ¹⁹F NMR (376 MHz, DMSO-d₆) δ −61.66, −115.00. LC-MS: m/z 672.3 (M+H)⁺.

### 5-[2-({2-[(4-chloro-2,6-difluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-4H-1,2,4-triazole-3-carboxamide (compound 44)

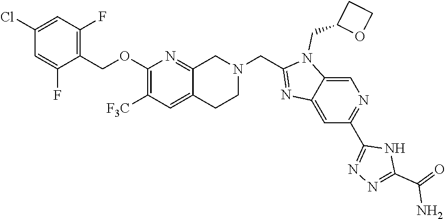 [SMILES: NC(=O)c1nnc(-c2cc3nc(CN4CCc5cc(C(F)(F)F)c(OCc6c(F)cc(Cl)cc6F)nc5C4)n(C[C@@H]4CCO4)c3cn2)[nH]1]

Compound 44 was synthesized following the similar route of Procedure 5, using tert-butyl 2-((4-chloro-2,6-di-fluorobenzyl)oxy)-3-(trifluoromethyl)-5,8-dihydro-1,7-naphthyridine-7(6H)-carboxylate in step A.

¹H NMR (400 MHz, DMSO-d₆) δ 9.18 (s, 1H), 8.35 (s, 1H), 7.97-7.89 (m, 2H), 7.64 (s, 1H), 7.39 (d, J=7.2 Hz, 2H), 5.41 (s, 2H), 5.11-5.09 (m, 1H), 4.92-4.87 (m, 1H), 4.76-4.72 (m, 1H), 4.48-4.45 (m, 1H), 4.40-4.36 (m, 1H), 3.91-4.03 (m, 2H), 3.00-3.11 (m, 2H), 2.89-2.87 (m, 2H), 2.67-2.66 (m, 2H), 2.40-2.36 (m, 1H), 2.33-2.32 (m, 1H). ¹⁹F NMR (376 MHz, DMSO-d₆) δ −61.85, −112.07. LC-MS: m/z 691.0 (M+H)⁺.

### Procedure 10

### 3-[2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-11H-imidazo[4,5-b]pyridin-5-yl]-4,5-dihydro-1,2,4-oxadiazol-5-one (compound 45)

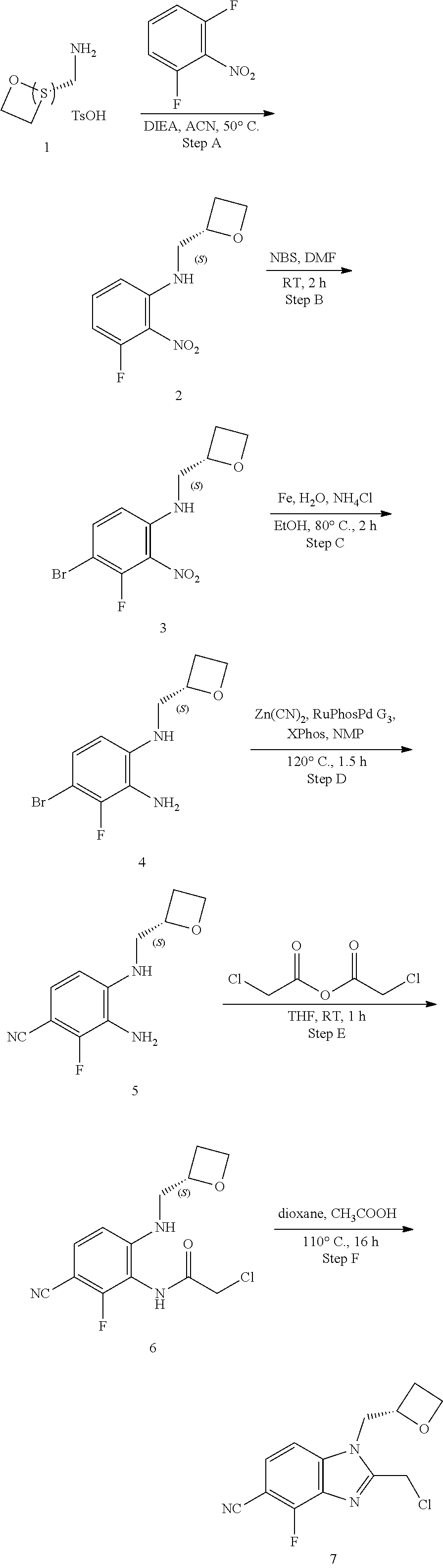

### Step A: (S)-3-fluoro-2-nitro-N-(oxetan-2-ylmethyl)aniline

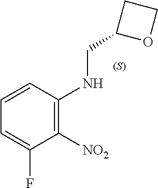

A mixture of (S)-oxetan-2-ylmethanamine, TsOH salt (4.7 g, 54.0 mmol), 1,3-difluoro-2-nitrobenzene (12.6 g, 79.2 mmol), and DIEA (34.4 g, 267.0 mmol) in CH₃CN (120 mL) was stirred at 50° C. for 5 hours. The resulting mixture was concentrated and then purified by column chromatography on silica gel (PE/EtOAc=1/1) to give (S)-3-fluoro-2-nitro-N-(oxetan-2-ylmethyl)aniline (4.1 g, yield: 36.3%). MS Calcd.: 226.08; MS Found: 227.3 [M+H]⁺.

### Step B: (S)-4-bromo-3-fluoro-2-nitro-N-(oxetan-2-ylmethyl)aniline

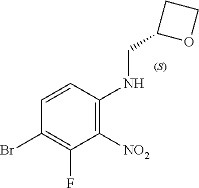

A solution of (S)-3-fluoro-2-nitro-N-(oxetan-2-ylmethyl)aniline (4.1 g, 18.1 mmol) and NBS (3.8 g, 21.3 mmol) in DMF (80 mL) was stirred at room temperature for 2 hours. The mixture was poured into sat. Na₂SO₃ and extracted with EtOAc (100 mL) and washed with brine (100 mL*3). The organic layer was separated and dried over Na₂SO₄, filtered, and purified by reversed phase column (PE/EtOAc=1/1) to give (S)-4-bromo-3-fluoro-2-nitro-N-(oxetan-2-ylmethyl)aniline (6.5 g, yield: 97.0%). MS Calcd.: 303.99; MS Found: 348.3 [M+H+41]⁺.

### Step C: (S)-4-bromo-3-fluoro-NJ-(oxetan-2-ylmethyl)benzene-1,2-diamine

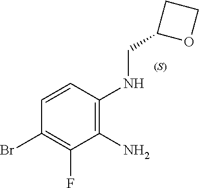

A solution of (S)-4-bromo-3-fluoro-2-nitro-N-(oxetan-2-ylmethyl)aniline (6.5 g, 21.4 mmol), Fe (6.0 g, 107.1 mmol), and NH₄Cl (5.72 g, 106.0 mmol) in EtOH/H₂O (50 mL/10 mL) was stirred at 80° C. for 2 hours. The mixture was diluted with DCM (50 mL) and washed with brine (50 mL*3). The organic layer was separated and dried over Na₂SO₄, filtered, and purified by column chromatography on silica gel (PE/EtOAc=1/1) to give (S)-4-bromo-3-fluoro-N1-(oxetan-2-ylmethyl)benzene-1,2-diamine (5.3 g, yield: 90.4%). MS Calcd.: 274.01; MS Found: 274.9 [M+H]⁺.

### Step D: (S)-3-amino-2-fluoro-4-((oxetan-2-ylmethyl)amino)benzonitrile

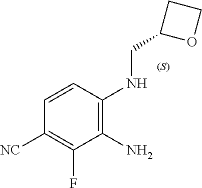

A mixture of (S)-4-bromo-3-fluoro-N1-(oxetan-2-ylmethyl)benzene-1,2-diamine (2.2 g, 8.03 mmol), Zn(CN)₂ (1.41 g, 12.05 mmol), RuPhos Pd G3 (0.67 g, 0.80 mmol) and XPhos (383 mg, 0.80 mmol) in NMP (10 mL) was stirred at 120° C. for 1.5 hours under Ar. The mixture was diluted with EtOAc (100 mL) and washed with sat. NaCl (100 mL*3), dried over Na₂SO₄, filtered, concentrated, and then purified by column chromatography on silica gel (PE/EtOAc=1/1) to give (S)-3-amino-2-fluoro-4-((oxetan-2-ylmethyl)amino)benzonitrile (1.13 g, yield: 63.8%). MS Calcd.: 221.10; MS Found: 222.4 [M+H]⁺.

### Step E: (S)-2-chloro-N-(3-cyano-2-fluoro-6-((oxetan-2-ylmethyl)amino)phenyl)acetamide

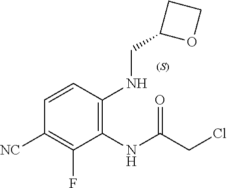

A mixture of (S)-3-amino-2-fluoro-4-((oxetan-2-ylmethyl)amino)benzonitrile (1.13 g, 5.11 mmol) and 2-chloroacetic anhydride (875 mg, 5.12 mmol) in THF (15 mL) was stirred at room temperature for 1 hour. The mixture was poured into sat. Na₂CO₃ and extracted with EtOAc (100 mL) and was washed with brine (100 mL*3). The organic layer was separated and dried over Na₂SO₄, filtered, and purified by column chromatography on silica gel (PE/EtOAc=1/1) to give (S)-2-chloro-N-(3-cyano-2-fluoro-6-((oxetan-2-ylmethyl)amino)phenyl)acetamide (1.2 g, yield: 79.0%). MS Calcd.: 297.07; MS Found: 298.1 [M+H]⁺.

### Step F: (S)-2-(chloromethyl)-4-fluoro-1-(oxetan-2-ylmethyl)-1H-benzo[d]imidazole-5-carbonitrile

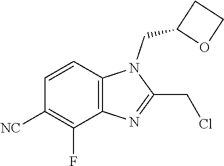 [SMILES: N#Cc1ccc2c(nc(CCl)n2C[C@@H]2CCO2)c1F]

A mixture of(S)-2-chloro-N-(3-cyano-2-fluoro-6-((oxetan-2-ylmethyl)amino)phenyl)-acetamide (1.2 g, 4.04 mmol) in AcOH/dioxane (2 mL/20 mL) was stirred at 110° C. for 16 hours. The mixture was poured into sat. Na₂CO₃ and extracted with EtOAc (100 mL) and was washed with brine (100 mL*3). The organic layer was filtered and purified by column chromatography on silica gel (PE/EtOAc=1/1) to give (S)-2-(chloromethyl)-4-fluoro-1-(oxetan-2-ylmethyl)-1H-benzo[d]imidazole-5-carbonitrile (354 mg, yield: 31%). MS Calcd.: 279.06; MS Found: 280.1 [M+H]⁺.

### 3-[2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-1H-imidazo[4,5-b]pyridin-5-yl]-4,5-dihydro-1,2,4-oxadiazol-5-one (Compound 45)

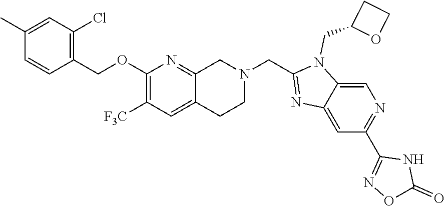 [SMILES: Cc1ccc(COc2nc3c(cc2C(F)(F)F)CCN(Cc2nc4cc(-c5noc(=O)[nH]5)ncc4n2C[C@@H]2CCO2)C3)c(Cl)c1]

Compound 45 was then synthesized following the similar route of Procedure 1, using ((S)-2-(chloromethyl)-4-fluoro-1-(oxetan-2-ylmethyl)-1H-benzo[d]imidazole-5-carbonitrile and (2-((2-chloro-4-methylbenzyl)oxy)-3-(trifluoromethyl)-5,6,7,8-tetrahydro-1,7-naphthyridine in step E.

¹H NMR (400 MHz, DMSO-d₆) δ 12.82 (s, 1H), 7.92 (s, 1H), 7.57-7.66 (m, 2H), 7.42 (d, J=8.0 Hz, 1H), 7.31 (s, 1H), 7.16 (d, J=8.0 Hz, 1H), 5.41 (s, 2H), 5.05-5.08 (m, 1H), 4.76 (dd, J=15.2 Hz, 6.8 Hz, 1H), 4.73 (dd, J=15.2 Hz, 2.8 Hz, 1H), 4.42-4.48 (m, 1H), 4.32-4.38 (m, 1H), 4.16 (d, J=13.6 Hz, 1H), 4.06 (d, J=13.6 Hz, 1H), 3.67-3.77 (m, 2H), 2.82-2.84 (m, 4H), 2.57-2.67 (m, 1H), 2.33-2.45 (m, 1H), 2.23 (s, 3H). 19F NMR (376 MHz, DMSO-d₆) δ −61.60, −126.09. LC-MS: m/z 659.3 (M+H)⁺.

### 3-[2-({2-[(4-chlorophenyl)methoxy]-3-(trifluoromethyl)-5,6,7,8-tetrahydro-1,7-naphthyridin-7-yl}methyl)-4-fluoro-1-{[(2S)-oxetan-2-yl]methyl}-1H-1,3-benzodiazol-5-yl]-4,5-dihydro-1,2,4-oxadiazol-5-one (compound 46)

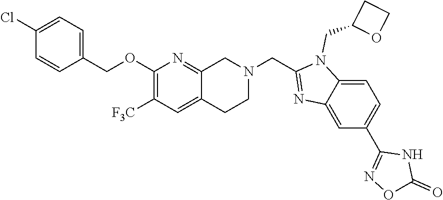 [SMILES: O=c1[nH]c(-c2ccc3c(c2)nc(CN2CCc4cc(C(F)(F)F)c(OCc5ccc(Cl)cc5)nc4C2)n3C[C@@H]2CCO2)no1]

Compound 46 was synthesized following the similar route of Procedure 1, using ((S)-2-(chloromethyl)-4-fluoro-1-(oxetan-2-ylmethyl)-1H-benzo[d]imidazole-5-carbonitrile and 2-((4-chlorobenzyl)oxy)-3-(trifluoromethyl)-5,6,7,8-tetrahydro-1,7-naphthyridine in step E.

¹H NMR (400 MHz, DMSO-d₆) δ 12.86 (br s, 1H), 8.03 (s, 1H), 7.76 (d, J=8.4 Hz, 1H), 7.59-7.66 (m, 1H), 7.40-7.47 (m, 4H), 5.42 (s, 2H), 5.01-5.10 (m, 1H), 4.74-4.84 (m, 1H), 4.61-4.69 (m, 1H), 4.41-4.50 (m, 2H), 4.29-4.38 (m, 2H), 4.02-4.17 (m, 2H), 3.12-3.35 (m, 2H), 2.91-3.01 (m, 2H), 2.62-2.73 (m, 1H), 2.28-2.39 (m, 1H). ¹⁹F NMR (376 MHz, DMSO-d₆) δ −61.79, −125.63. LC-MS: m/z 663.0 (M+H)⁺.

### 3-[2-({2-[(4-chloro-2-fluorophenyl)methoxy]-3-(trifluoromethyl)-5,6,7,8-tetrahydro-1,7-naphthyridin-7-yl}methyl)-4-fluoro-1-{[(2S)-oxetan-2-yl]methyl}-1H-1,3-benzodiazol-5-yl]-4,5-dihydro-1,2,4-oxadiazol-5-one (compound 47)

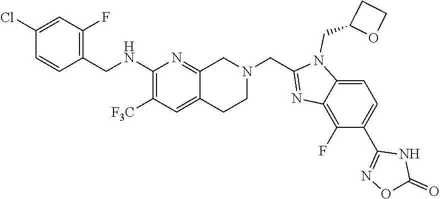 [SMILES: O=c1[nH]c(-c2ccc3c(nc(CN4CCc5cc(C(F)(F)F)c(NCc6ccc(Cl)cc6F)nc5C4)n3C[C@@H]3CCO3)c2F)no1]

Compound 47 was synthesized following the similar route of Procedure 1, using ((S)-2-(chloromethyl)-4-fluoro-1-(oxetan-2-ylmethyl)-1H-benzo[d]imidazole-5-carbonitrile and 2-((4-chloro-2-fluorobenzyl)oxy)-3-(trifluoromethyl)-5,6,7,8-tetrahydro-1,7-naphthyridine in step E.

¹H NMR (400 MHz, DMSO-d₆) δ 12.85 (br s, 1H), 8.03 (s, 1H), 7.77 (d, J=8.8 Hz, 1H), 7.59-7.66 (m, 1H), 7.53 (t, J=8.0 Hz, 1H), 7.46 (dd, J=10.0 Hz, 2.0 Hz, 1H), 7.32 (dd, J=8.4 Hz, 1.6 Hz, 1H), 5.45 (s, 2H), 5.01-5.10 (m, 1H), 4.74-4.84 (m, 1H), 4.61-4.70 (m, 1H), 4.38-4.59 (m, 3H), 4.29-4.38 (m, 1H), 4.06-4.26 (m, 2H), 3.19-3.45 (m, 2H), 2.92-3.06 (m, 2H), 2.61-2.73 (m, 1H), 2.27-2.40 (m, 1H). ¹⁹F NMR (376 MHz, DMSO-d₆) δ −61.84, −114.96, −125.47. LC-MS: m/z 663.0 (M+H)⁺.

### 3-[2-({2-[(4-chloro-2,6-difluorophenyl)methoxy]-3-(trifluoromethyl)-5,6,7,8-tetrahydro-1,7-naphthyridin-7-yl}methyl)-4-fluoro-1-{[(2S)-oxetan-2-yl]methyl}-1H-1,3-benzodiazol-5-yl]-4,5-dihydro-1,2,4-oxadiazol-5-one (compound 48)

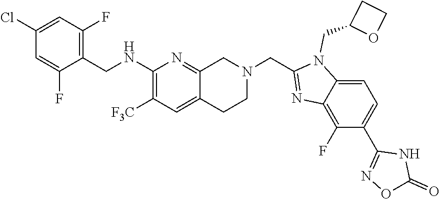 [SMILES: O=c1[nH]c(-c2ccc3c(nc(CN4CCc5cc(C(F)(F)F)c(NCc6c(F)cc(Cl)cc6F)nc5C4)n3C[C@@H]3CCO3)c2F)no1]

Compound 48 was synthesized following the similar route of Procedure 1, using ((S)-2-(chloromethyl)-4-fluoro-1-(oxetan-2-ylmethyl)-1H-benzo[d]imidazole-5-carbonitrile and 2-((4-chloro-2,6-difluorobenzyl)oxy)-3-(trifluoromethyl)-5,6,7,8-tetrahydro-1,7-naphthyridine in step E.

¹H NMR (400 MHz, CD₃OD) δ 7.78 (s, 1H), 7.72-7.68 (m, 1H), 7.64-7.54 (m, 1H), 7.20-7.16 (m, 2H), 5.74-5.64 (m, 2H), 5.50 (m, 1H), 4.91 (m, 4H), 4.48-4.40 (m, 2H), 4.07-4.02 (m, 2H), 3.46-3.33 (m, 2H), 3.22-3.11 (m, 2H), 2.98-2.82 (m, 2H). ¹⁹F NMR (376 MHz, CD₃OD) δ −64.64, −114.33, −129.63. LC-MS: m/z 681.3 (M+H)⁺.

### Procedure 11

### 3-[2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-11H-imidazo[4,5-b]pyridin-5-yl]-4,5-dihydro-1,2,4-oxadiazol-5-one (compound 49)

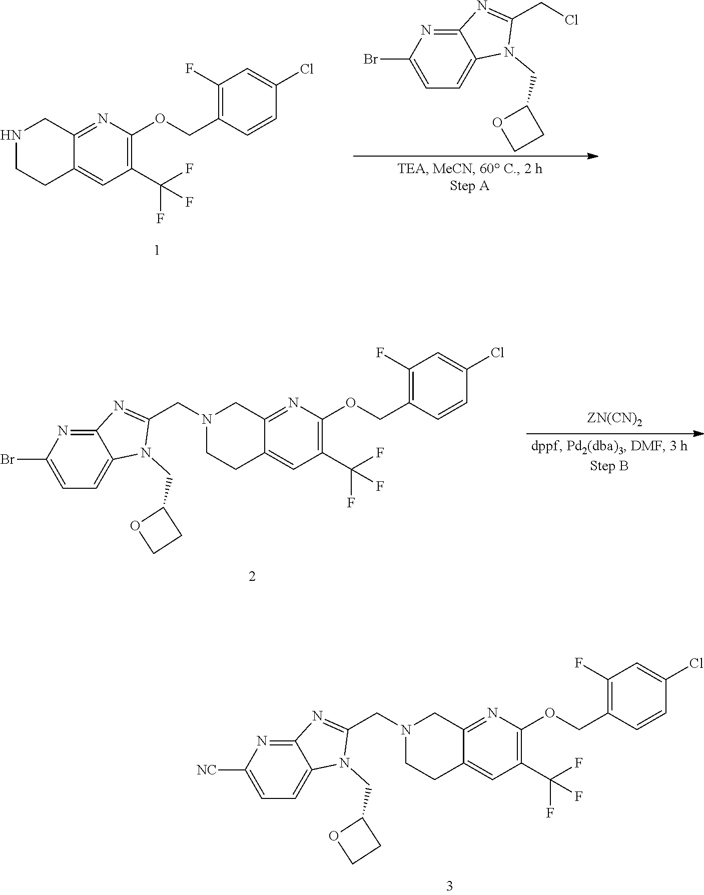 [SMILES: Fc1cc(Cl)ccc1COc1nc2c(cc1C(F)(F)F)CCNC2 ; ClCc1nc2nc(Br)ccc2n1C[C@@H]1CCO1 ; Fc1cc(Cl)ccc1COc1nc2c(cc1C(F)(F)F)CCN(Cc1nc3nc(Br)ccc3n1C[C@@H]1CCO1)C2 ; N#Cc1ccc2c(n1)nc(CN1CCc3cc(C(F)(F)F)c(OCc4ccc(Cl)cc4F)nc3C1)n2C[C@@H]1CCO1]

### Step A: (S)-7-((5-bromo-1-(oxetan-2-ylmethyl)-1H-imidazo[4,5-b]pyridin-2-yl)methyl)-2-((4-chloro-2-fluorobenzyl)oxy)-3-(trifluoromethyl)-5,6,7,8-tetrahydro-1,7-naphthyridine

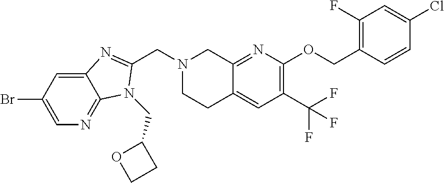 [SMILES: Fc1cc(Cl)ccc1COc1nc2c(cc1C(F)(F)F)CCN(Cc1nc3cc(Br)cnc3n1C[C@@H]1CCO1)C2]

A mixture of 2-((4-chloro-2-fluorobenzyl)oxy)-3-(trifluoromethyl)-5,6,7,8-tetrahydro-1,7-naphthyridine (114 mg, 0.317 mmol), (S)-5-bromo-2-(chloromethyl)-1-(oxetan-2-ylmethyl)-1H-imidazo[4,5-b]pyridine (110 mg, 0.439 mmol), and TEA (96 mg, 0.951 mmol) in MeCN (3.0 mL) was stirred at 60° C. for 2 hours. The mixture was diluted with DCM (50 mL) and washed with brine (50 mL*3). The organic phase was dried over Na₂SO₄, and concentrated to give (S)-7-((5-bromo-1-(oxetan-2-ylmethyl)-1H-imidazo[4,5-b]pyridin-2-yl)methyl)-2-((4-chloro-2-fluorobenzyl)oxy)-3-(trifluoromethyl)-5,6,7,8-tetrahydro-1,7-naphthyridine (208 mg, yield: 102%). MS Calcd.: 639.1; MS Found: 640.1 [M+H]⁺.

### Step B: S)-2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-imidazo[4,5-b]pyridine-5-carbonitrile

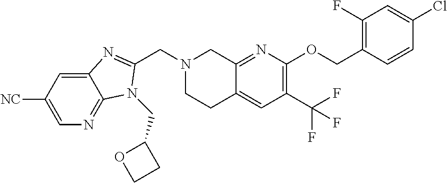 [SMILES: N#Cc1cnc2c(c1)nc(CN1CCc3cc(C(F)(F)F)c(OCc4ccc(Cl)cc4F)nc3C1)n2C[C@@H]1CCO1]

A solution of (S)-7-((5-bromo-1-(oxetan-2-ylmethyl)-1H-imidazo[4,5-b]pyridin-2-yl)methyl)-2-((4-chloro-2-fluorobenzyl)oxy)-3-(trifluoromethyl)-5,6,7,8-tetrahydro-1,7-naphthyridine (208 mg, 0.326 mmol), Zn(CN)₂ (114 mg, 0.977 mmol), dppf (9 mg, 0.016 mmol), and Pd₂(dba)₃ (30 mg, 0.026 mmol) in DMF (3 mL) was stirred at 125° C. for 3 hours under Ar. The mixture was diluted with DCM (50 mL) and washed with brine (50 mL*3). The organic phase was dried over Na₂SO₄, filtered, and concentrated. The mixture was purified by reversed phase column to give (S)-2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-imidazo[4,5-b]pyridine-5-carbonitrile (160 mg, yield: 84%). MS Calcd.: 586.2; MS Found: 587.3 [M+H]⁺.

### tert-butyl 3-[2-({2-[(4-chloro-2-fluorophenyl)methoxy]-3-(trifluoromethyl)-5,6,7,8-tetrahydro-1,7-naphthyridin-7-yl}methyl)-]—{[(2S)-oxetan-2-yl]methyl}-1H-imidazo[4,5-b]pyridin-5-yl]-4,5-dihydro-1,2,4-oxadiazol-5-one

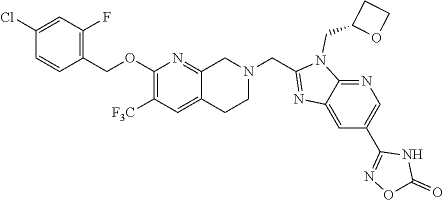 [SMILES: O=c1[nH]c(-c2cnc3c(c2)nc(CN2CCc4cc(C(F)(F)F)c(OCc5ccc(Cl)cc5F)nc4C2)n3C[C@@H]2CCO2)no1]

Compound 49 was then synthesized following the similar route of Procedure 8, using ((S)-2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-imidazo[4,5-b]pyridine-5-carbonitrile in step A.

¹H NMR (400 MHz, DMSO-d₆) δ 13.17 (s, 1H), 8.27 (d, J=8.4 Hz, 1H), 7.87-7.92 (m, 2H), 7.49-7.54 (m, 1H), 7.46 (dd, J=10.0 Hz, 2 Hz, 1H), 7.30-7.33 (m, 1H), 5.43 (s, 2H), 5.04-5.10 (m, 1H), 4.77-4.82 (m, 1H), 4.64-4.68 (m, 1H), 4.43-4.48 (m, 1H), 4.34-4.38 (m, 1H), 4.21 (d, J=14.0 Hz, 1H), 4.12 (d, J=14.0 Hz, 1H), 3.70-3.80 (m, 2H), 2.81-2.88 (m, 4H), 2.56-2.69 (m, 1H), 2.36-2.40 (m, 1H). ¹⁹F NMR (376 MHz, DMSO-d₆) δ −61.67, −115.00. LC-MS: m/z 646.5 (M+H)⁺.

### 3-[2-({2-[(4-chloro-2,6-difluorophenyl)methoxy]-3-methyl-5,6,7,8-tetrahydro-1,7-naphthyridin-7-yl}methyl)-1-{1[(2S)-oxetan-2-yl]methyl}-1H-imidazo[4,5-b]pyridin-5-yl]-4,5-dihydro-1,2,4-oxadiazol-5-one (compound 50)

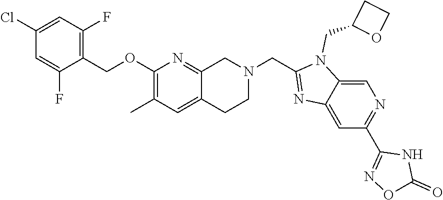 [SMILES: Cc1cc2c(nc1OCc1c(F)cc(Cl)cc1F)CN(Cc1nc3cc(-c4noc(=O)[nH]4)ncc3n1C[C@@H]1CCO1)CC2]

Compound 50 was synthesized following the similar route of Procedure 11, using 2-((4-chloro-2,6-difluorobenzyl)oxy)-3-methyl-5,6,7,8-tetrahydro-1,7-naphthyridine in step A.

¹H NMR (400 MHz, DMSO-d₆) δ 13.12 (s, 1H), 8.26 (d, J=8.4 Hz, 1H), 7.87 (d, J=8.4 Hz, 1H), 7.38 (d, J=7.2 Hz, 2H), 7.31 (s, 1H), 5.26 (s, 2H), 5.06-5.07 (m, 1H), 4.77-4.79 (m, 1H), 4.65-4.69 (m, 1H), 4.45-4.46 (m, 1H), 4.34-4.36 (m, 1H), 4.17 (d, J=13.6 Hz, 1H), 4.07 (d, J=13.6 Hz, 1H), 3.58-3.62 (m, 2H), 2.79-2.82 (m, 2H), 2.67-2.73 (m, 2H), 2.58-2.65 (m, 1H), 2.32-2.43 (m, 1H), 1.99-2.03 (m, 3H). ¹⁹F NMR (376 MHz, DMSO-d₆) δ −112.29. LC-MS: m/z 610.3 (M+H)⁺.

### 5-[2-({2-[(4-chloro-2-fluorophenyl)methoxy]-3-(trifluoromethyl)-5,6,7,8-tetrahydro-1,7-naphthyridin-7-yl}methyl)-3-{1[(2S)-oxetan-2-yl]methyl}-3H-imidazo[4,5-b]pyridin-6-yl]-4H-1,2,4-triazole-3-carbonitrile (compound 17)

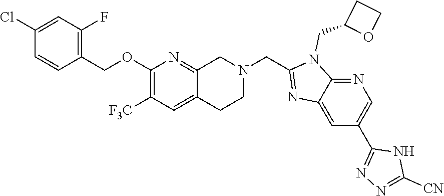 [SMILES: N#Cc1nnc(-c2cnc3c(c2)nc(CN2CCc4cc(C(F)(F)F)c(OCc5ccc(Cl)cc5F)nc4C2)n3C[C@@H]2CCO2)[nH]1]

Compound 17 was synthesized following the similar route of Procedure 5, using tert-butyl 2-((4-chloro-2-fluorobenzyl)oxy)-3-(trifluoromethyl)-5,8-dihydro-1,7-naphthyridine-7(6H)-carboxylate in step A and (S)-2-(chloromethyl)-3-(oxetan-2-ylmethyl)-3H-imidazo[4,5-b]pyridine-6-carbonitrile in step B.

¹H NMR (400 MHz, DMSO-d₆) δ 9.01 (d, J=1.6 Hz, 1H), 8.65 (d, J=1.6 Hz, 1H), 7.95 (s, 1H), 7.52 (t, J=8.4 Hz, 1H), 7.46 (dd, J=1.6 Hz, J=8.8 Hz, 1H), 7.31 (dd, J=2.0 Hz, J=8.4 Hz, 1H), 5.43 (s, 2H), 5.11-5.17 (m, 1H), 4.79-4.84 (m, 1H), 4.66-4.71 (m, 1H), 4.44-4.49 (m, 1H), 4.30-4.36 (m, 2H), 4.21-4.28 (m, 1H), 3.75-3.88 (m, 1H), 3.45-3.48 (m, 1H), 2.91-3.01 (m, 1H), 2.84-2.90 (m, 2H), 2.63-2.71 (m, 1H), 2.38-2.45 (m, 2H). ¹⁹F NMR (376 MHz, DMSO-d₆) δ −61.71, −115.00. LC-MS: m/z 654.2 (M+H)⁺. 2-[(4-chloro-2,6-difluorophenyl)methoxy]-3-methyl-7-[(3-{1[(2S)-oxetan-2-yl]methyl}-6-[5-(trifluoromethyl)-4H-1,2,4-triazol-3-yl]-3H-imidazo[4,5-b]pyridin-2-yl)methyl]-5,6,7,8-tetrahydro-1,7-naphthyridine (compound 51)

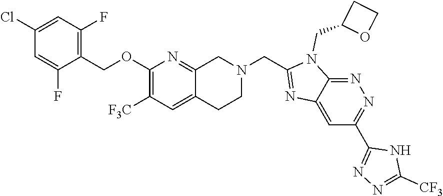 [SMILES: Fc1cc(Cl)cc(F)c1COc1nc2c(cc1C(F)(F)F)CCN(Cc1nc3cc(-c4nnc(C(F)(F)F)[nH]4)nnc3n1C[C@@H]1CCO1)C2]

Compound 51 was synthesized following the similar route of Procedure 3, using tert-butyl 2-((4-chloro-2,6-di-fluorobenzyl)oxy)-3-methyl-5,8-dihydro-1,7-naphthyridine-7(6H)-carboxylate in step A.

¹H NMR (400 MHz, DMSO-d₆) δ 15.42 (s, 1H), 9.00 (d, J₁=1.6 Hz, 1H), 8.62 (s, 1H), 7.37 (d, J=7.2 Hz, 2H), 7.31 (s, 1H), 5.26 (s, 2H), 5.16-5.13 (m, 1H), 4.84-4.79 (dd, J₁=6.0 Hz, J₂-6.4 Hz, 1H), 4.71-4.67 (dd, J₁=4.0 Hz, J₂=3.6 Hz, 1H), 4.48-4.44 (m, 1H), 4.37-4.31 (m, 1H), 4.19-4.10 (m, 2H), 3.61 (s, 2H), 2.81-2.80 (m, 2H), 2.72-2.71 (m, 2H), 2.63-2.54 (m, 1H), 2.44-2.32 (m, 1H), 2.02 (s, 3H). ¹⁹F NMR (376 MHz, DMSO-d₆) δ −63.51, −112.29. LC-MS: m/z 661.3 (M+H)⁺.

### 2-[(4-chloro-2-fluorophenyl)methoxy]-7-({6-[5-(difluoromethyl)-4H-1,2,4-triazol-3-yl]-3-{[(2S)-oxetan-2-yl]methyl}-3H-imidazo[4,5-b]pyridin-2-yl}methyl)-3-methyl-5,6,7,8-tetrahydro-1,7-naphthyridine (compound 52)

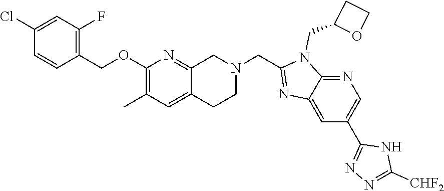 [SMILES: Cc1cc2c(nc1OCc1ccc(Cl)cc1F)CN(Cc1nc3cc(-c4nnc(C(F)F)[nH]4)cnc3n1C[C@@H]1CCO1)CC2]

Compound 52 was synthesized following the similar route of Procedure 3, using tert-butyl 2-((4-chloro-2-fluorobenzyl)oxy)-3-methyl-5,8-dihydro-1,7-naphthyridine-7(6H)-carboxylate in step A and 2,2-difluoroacetic anhydride in step D.

¹H NMR (400 MHz, DMSO-d₆) δ 15.04 (s, 0.8H), 9.00 (d, J=2 Hz, 1H), 8.60 (d, J=2 Hz, 1H), 7.54 (t, J=16.4 Hz, 1H), 7.43 (dd, J₁=2 Hz, J₂=2 Hz, 1H), 7.33 (s, 1H), 7.29-7.04 (m, 2H), 5.29 (s, 2H), 5.16-5.13 (m, 1H), 4.86-4.78 (m, 1H), 4.71-4.66 (m, 1H), 4.49-4.44 (m, 1H), 4.36-4.31 (m, 1H), 4.18-4.09 (m, 2H), 3.60 (s, 2H), 2.80-2.79 (m, 2H), 2.73-2.70 (m, 2H), 2.67-2.66 (m, 1H), 2.33-2.28 (m, 1H), 2.10 (s, 3H). ¹⁹F NMR (376 MHz, DMSO-d₆) δ −115.33, −115.84. LC-MS: m/z 625.4 (M+H)⁺.

### 2-[(4-chloro-2,6-difluorophenyl)methoxy]-7-({6-[5-(difluoromethyl)-4H-1,2,4-triazol-3-yl]-3-{1[(2S)-oxetan-2-yl]methyl}-3H-imidazo[4,5-b]pyridin-2-yl}methyl)-3-methyl-5,6,7,8-tetrahydro-1,7-naphthyridine (compound 53)

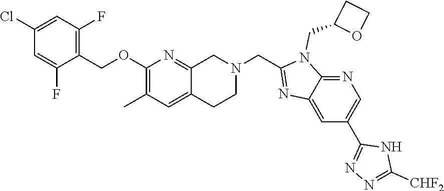 [SMILES: Cc1cc2c(nc1OCc1c(F)cc(Cl)cc1F)CN(Cc1nc3cc(-c4nnc(C(F)F)[nH]4)cnc3n1C[C@@H]1CCO1)CC2]

Compound 53 was synthesized following the similar route of Procedure 3, using tert-butyl 2-((4-chloro-2,6-di-fluorobenzyl)oxy)-3-methyl-5,8-dihydro-1,7-naphthyridine-7(6H)-carboxylate in step A and 2,2-difluoroacetic anhydride in step D.

¹H NMR (400 MHz, CD₃OD) δ 9.06 (d, J=2 Hz, 1H), 8.65 (d, J=2 Hz, 1H), 7.23 (s, 1H), 7.08 (d, J=7.2 Hz, 2H), 6.91 (t, J=107.2 Hz, 1H), 5.30-5.27 (m, 3H), 4.98-4.92 (m, 2H), 4.62-4.60 (m, 1H), 4.43-4.40 (m, 1H), 4.27-4.18 (m, 2H), 3.65 (s, 2H), 2.89-2.86 (m, 2H), 2.81-2.78 (m, 2H), 2.71-2.77 (m, 1H), 2.54-2.51 (m, 1H), 2.06 (s, 3H). ¹⁹F NMR (376 MHz, CD₃OD) δ −114.24, −117.86. LC-MS: m/z 643.4 (M+H)⁺.

### 2-[(4-chlorophenyl)methoxy]-7-[(3-{1[(2S)-oxetan-2-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridine (compound 54)

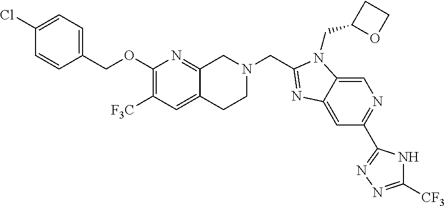 [SMILES: FC(F)(F)c1nnc(-c2cc3nc(CN4CCc5cc(C(F)(F)F)c(OCc6ccc(Cl)cc6)nc5C4)n(C[C@@H]4CCO4)c3cn2)[nH]1]

Compound 54 was synthesized following the similar route of Procedure 3, using 2-((4-chlorobenzyl)oxy)-3-(trifluoromethyl)-5,6,7,8-tetrahydro-1,7-naphthyridine, TFA Salt and (S)-2-(chloromethyl)-3-(oxetan-2-ylmethyl)-3H-imidazo[4,5-c]pyridine-6-carbonitrile in step B.

¹H NMR (400 MHz, DMSO-d₆) δ 15.42 (br s, 1H), 9.17 (s, 1H), 8.33 (s, 1H), 7.93 (s, 1H), 7.40-7.46 (m, 4H), 5.39 (s, 2H), 5.05-5.16 (m, 1H), 4.85-4.97 (m, 1H), 4.69-4.80 (m, 1H), 4.42-4.52 (m, 1H), 4.33-4.42 (m, 1H), 4.18-4.26 (m, 1H), 4.06-4.15 (m, 1H), 3.64-3.80 (m, 2H), 2.78-2.90 (m, 2H), 2.61-2.72 (m, 2H), 2.31-2.43 (m, 2H). ¹⁹F NMR (376 MHz, DMSO-d₆) δ −61.63, −63.74. LC-MS: m/z 679.4 (M+H)⁺.

### 2-[(4-chloro-2,6-difluorophenyl)methoxy]-3-methyl-7-[(3-{1[(2S)-oxetan-2-yl]methyl}-6-[5-(trifluoromethyl)-4H-1,2,4-triazol-3-yl]-3H-imidazo[4,5-c]pyridin-2-yl)methyl]-5,6,7,8-tetrahydro-1,7-naphthyridine (compound 55)

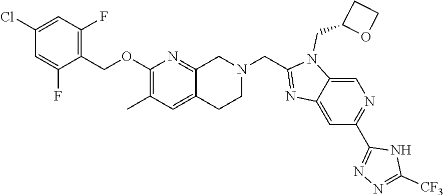 [SMILES: Cc1cc2c(nc1OCc1c(F)cc(Cl)cc1F)CN(Cc1nc3cc(-c4nnc(C(F)(F)F)[nH]4)ncc3n1C[C@@H]1CCO1)CC2]

Compound 55 was synthesized following the similar route of Procedure 3, using tert-butyl 2-((4-chloro-2,6-di-fluorobenzyl)oxy)-3-methyl-5,8-dihydro-1,7-naphthyridine-7(6H)-carboxylate in step A and (S)-2-(chloromethyl)-3-(oxetan-2-ylmethyl)-3H-imidazo[4,5-c]pyridine-6-carbonitrile in step B.

¹H NMR (400 MHz, DMSO-d₆) δ 15.43 (s, 1H), 9.18 (s, 1H), 8.33 (s, 1H), 7.38-7.35 (m, 2H), 7.32 (s, 1H), 5.25 (s, 2H), 5.13-5.07 (m, 1H), 4.94-4.88 (m, 1H), 4.78-4.73 (m, 1H), 4.52-4.45 (m, 1H), 4.40-4.35 (m, 1H), 4.24-4.04 (m, 2H), 3.64-3.54 (m, 2H), 2.91-2.79 (m, 2H), 2.72-2.64 (m, 3H), 2.43-2.37 (m, 1H), 2.03 (s, 3H). ¹⁹F NMR (376 MHz, DMSO-d₆) δ −61.73, −112.29. LC-MS: m/z 661.3 (M+H)⁺.

### 2-[(4-chloro-2,6-difluorophenyl)methoxy]-7-({6-[5-(difluoromethyl)-4H-1,2,4-triazol-3-yl]-3-{[(2S)-oxetan-2-yl]methyl}-3H-imidazo[4,5-c]pyridin-2-yl}methyl)-3-methyl-5,6,7,8-tetrahydro-1,7-naphthyridine (compound 56)

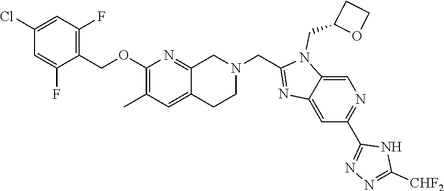 [SMILES: Cc1cc2c(nc1OCc1c(F)cc(Cl)cc1F)CN(Cc1nc3cc(-c4nnc(C(F)F)[nH]4)ncc3n1C[C@@H]1CCO1)CC2]

Compound 56 was synthesized following the similar route of Procedure 3, using tert-butyl 2-((4-chloro-2,6-di-fluorobenzyl)oxy)-3-methyl-5,8-dihydro-1,7-naphthyridine-7(6H)-carboxylate in step A, (S)-2-(chloromethyl)-3-(oxetan-2-ylmethyl)-3H-imidazo[4,5-c]pyridine-6-carbonitrile in step B and 2,2-difluoroacetic anhydride in step D.

¹H NMR (400 MHz, DMSO-d₆) δ 9.25 (s, 1H), 8.41 (s, 1H), 7.45 (s, 1H), 7.35 (d, J=7.6 Hz, 2H), 7.01-7.27 (m, 1H), 5.30 (s, 2H), 5.06-5.15 (m, 1H), 4.86-4.95 (m, 1H), 4.69-4.79 (m, 1H), 4.45-4.54 (m, 1H), 4.33-4.42 (m, 1H), 4.19-4.32 (m, 2H), 3.39-3.53 (m, 2H), 2.91-3.02 (m, 2H), 2.66-2.81 (m, 1H), 2.52-2.55 (m, 2H), 2.31-2.44 (m, 1H), 2.08 (s, 3H). ¹⁹F NMR (376 MHz, DMSO-d₆) δ −112.13, −115.79. LC-MS: m/z 643.1 (M+H)⁺.

### 2-[(4-chloro-2-fluorophenyl)methoxy]-7-({6-[5-(difluoromethyl)-4H-1,2,4-triazol-3-yl]-3-{[(2S)-oxetan-2-yl]methyl}-3H-imidazo[4,5-c]pyridin-2-yl}methyl)-3-(trifluoromethyl)-5,6,7,8-tetrahydro-1,7-naphthyridine (compound 57)

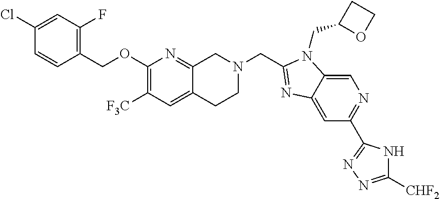 [SMILES: Fc1cc(Cl)ccc1COc1nc2c(cc1C(F)(F)F)CCN(Cc1nc3cc(-c4nnc(C(F)F)[nH]4)ncc3n1C[C@@H]1CCO1)C2]

Compound 57 was synthesized following the similar route of Procedure 3, using tert-butyl 2-((4-chloro-2-fluorobenzyl)oxy)-3-(trifluoromethyl)-5,8-dihydro-1,7-naphthyridine-7(6H)-carboxylate in step A, (S)-2-(chloromethyl)-3-(oxetan-2-ylmethyl)-3H-imidazo[4,5-c]pyridine-6-carbonitrile in step B and 2,2-difluoroacetic anhydride in step D.

¹H NMR (400 MHz, DMSO-d₆) δ 15.02 (s, 1H), 9.15 (s, 1H), 8.31 (s, 1H), 7.92 (s, 1H), 7.49-7.53 (m, 1H), 7.45 (d, J=10.4 Hz, 1H), 7.30 (d, J=8.0 Hz, 1H), 7.13 (t, J=52.0 Hz, 1H), 5.42 (s, 2H), 5.05-5.16 (m, 1H), 4.84-4.95 (m, 1H), 4.67-4.80 (m, 1H), 4.43-4.52 (m, 1H), 4.33-4.41 (m, 1H), 4.21 (d, J=13.6 Hz, 1H), 4.11 (d, J=13.6 Hz, 1H), 3.66-3.82 (m, 2H), 2.77-2.93 (m, 4H), 2.62-2.70 (m, 1H), 2.35-2.44 (m, 1H). ¹⁹F NMR (376 MHz, DMSO-d₆) δ −61.67, −115.00, −115.76. LC-MS: m/z 679.2 (M+H)⁺.

### Procedure 12

### 3-[2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-1H-thieno[2,3-d]imidazol-5-yl]-4,5-dihydro-1,2,4-oxadiazol-5-one (compound 58)

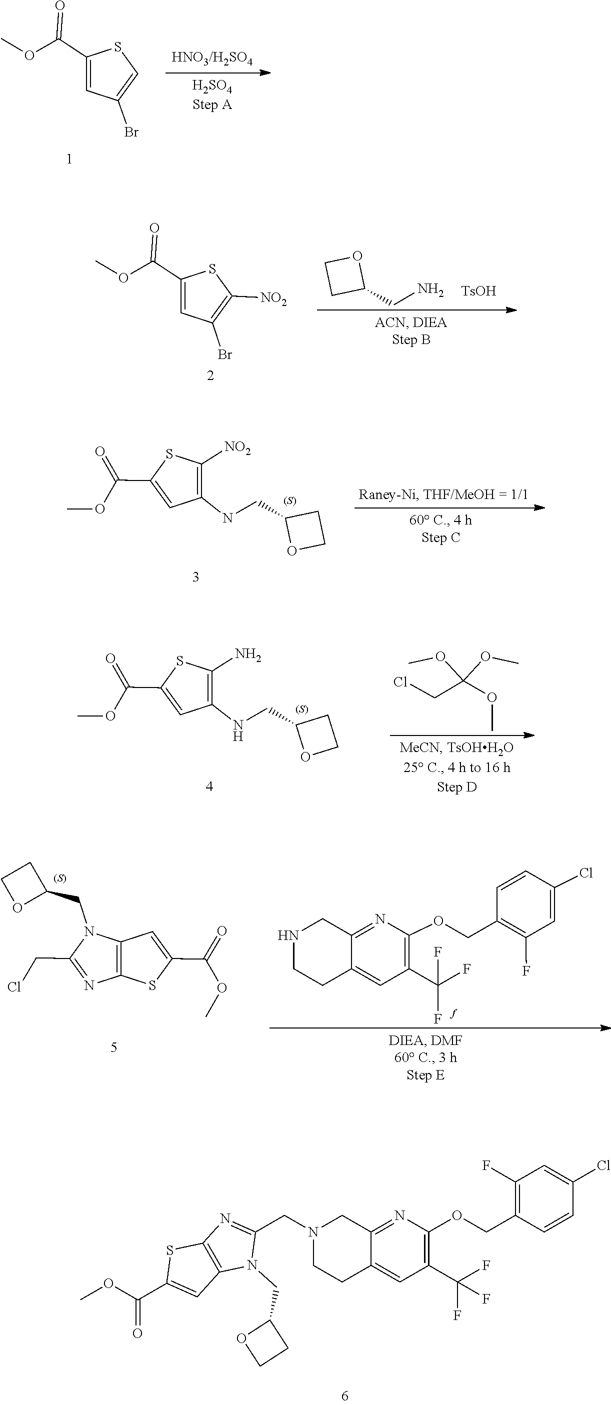

### Step A: methyl 4-bromo-5-nitrothiophene-2-carboxylate

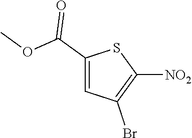

To a solution of methyl 4-bromothiophene-2-carboxylate (10.0 g, 45.25 mmol, 1.0 eq) in conc. H₂SO₄ (33 mL) was added a solution of fuming HNO₃ (6.67 mL, 3.5 eq) in conc. H₂SO₄ (20 mL) at −5˜10° C., and the reaction mixture was stirred at −10° C. for 1 hour under N₂. After the reaction was completed, the mixture was added to ice (500 g), extracted with EtOAc (300 mL*2), and the combined organic layers were washed with brine (200 mL), dried over Na₂SO₄, filtered, and concentrated in vacuum. The residue was purified by flash column chromatography (eluting PE/EA=10/1) to give methyl 4-bromo-5-nitrothiophene-2-carboxylate (9.625 g, 80.27% yield). MS Calcd.: 264.9; MS Found: 235.9 [M+H−30]⁺.

### Step B: methyl (S)-5-nitro-4-((oxetan-2-ylmethyl) amino) thiophene-2-carboxylate

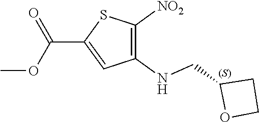

To a solution of methyl 4-bromo-5-nitrothiophene-2-carboxylate (6 g, 22.64 mmol) in MeCN (200 mL) was added (S)-oxetan-2-ylmethanamine, p-toluenesulfonate (11.7 g, 45.3 mmol) and DIEA (8.76 g, 67.9 mmol), and the reaction mixture was stirred at 60° C. for 18 hrs. The reaction mixture was concentrated in vacuum, and the residue was purified by flash column chromatography (eluting DCM/MeOH=50/1) to give methyl (S)-5-nitro-4-((oxetan-2-ylmethyl)amino) thiophene-2-carboxylate (5.15 g, 83.63% yield). MS Calcd.: 272.05; MS Found: 273.1 [M+H]⁺.

### Step C: methyl (S)-5-amino-4-((oxetan-2-ylmethyl) amino) thiophene-2-carboxylate

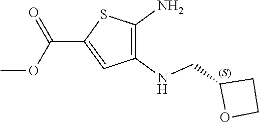

To a solution of methyl (S)-5-nitro-4-((oxetan-2-ylmethyl)amino)thiophene-2-carboxylate (500 mg, 1.84 mmol) in MeOH (40 mL) and THF (40 mL) was added Raney-Ni (200 mg), and the reaction mixture was stirred at 60° C. for 4 hrs under an H₂ balloon. The reaction mixture was filtered by Celite after cooling to room temperature, and the filtrate was concentrated in vacuum to give crude methyl (S)-5-amino-4-((oxetan-2-ylmethyl)amino)thiophene-2-carboxylate (445 mg, crude), which was used for the next step directly without further purification. MS Calcd.: 242.07; MS Found: 243.1 [M+H]⁺.

### Step D: methyl (S)-2-(chloromethyl)-1-(oxetan-2-ylmethyl)-1H-thieno[2,3-d]imidazole-5-carboxylate

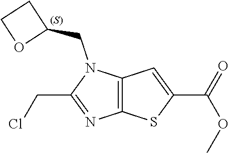

To a solution of crude methyl (S)-5-amino-4-((oxetan-2-ylmethyl)amino)thiophene-2-carboxylate (445 mg, 1.84 mmol) in MeCN (50 mL) was added 1,1,1-trimethoxy-2-chloroethane (426 mg, 2.76 mmol), followed by TsOH·H₂O (70 mg, 0.368 mmol), and the reaction mixture was stirred at 25° C. for 6 hrs under N₂. The reaction mixture was concentrated in vacuum, and the residue was purified by flash column chromatography (eluting PE/EA=1/1) to give methyl (S)-2-(chloromethyl)-1-(oxetan-2-ylmethyl)-1H-thieno[2,3-d]imidazole-5-carboxylate (170 mg, 25.7% yield). MS Calcd.: 300.03; MS Found: 301.0 [M+H]⁺.

¹H NMR (400 MHz, DMSO-d₆) δ 7.96 (s, 1H), 5.08-5.02 (m, 3H), 4.67-4.63 (m, 1H), 4.62-4.46 (m, 2H), 4.37-4.31 (m, 1H), 3.84 (s, 3H), 2.71-2.66 (m, 1H), 2.38-2.32 (m, 1H).

### Step E: methyl (S)-2-((2-((4-chloro-2-fluorobenzyl) oxy)-3-(trifluoromethyl)-5,8-dihydro-1,7-naphthyridin-7(6H)-yl) methyl)-1-(oxetan-2-ylmethyl)-1H-thieno[2,3-d]imidazole-5-carboxylate

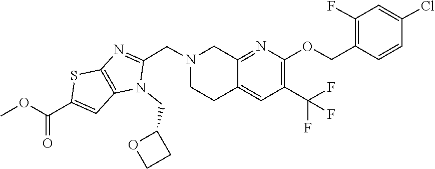 [SMILES: COC(=O)c1cc2c(nc(CN3CCc4cc(C(F)(F)F)c(OCc5ccc(Cl)cc5F)nc4C3)n2C[C@@H]2CCO2)s1]

To a solution of methyl (S)-2-(chloromethyl)-1-(oxetan-2-ylmethyl)-1H-thieno[2,3-d]imidazole-5-carboxylate (170 mg, 0.567 mmol) in DMF (5 mL) was added 2-((4-chloro-2-fluorobenzyl) oxy)-3-(trifluoromethyl)-5,6,7,8-tetrahydro-1,7-naphthyridine (295 mg, 0.623 mmol) and DIEA (366 mg, 2.835 mmol), and the reaction mixture was stirred at 60° C. for 3 hrs under N₂. The reaction mixture was added to water (20 mL), extracted with EtOAc (20 mL*2), and the combined organic layers were washed with brine (20 mL*2), dried over Na₂SO₄, filtered, and concentrated in vacuum. The residue was purified by flash column chromatography (eluting PE/EA=2/1) to give methyl (S)-2-((2-((4-chloro-2-fluorobenzyl) oxy)-3-(trifluoromethyl)-5,8-dihydro-1,7-naphthyridin-7(6H)-yl)methyl)-1-(oxetan-2-ylmethyl)-1H-thieno[2,3-d]imidazole-5-carboxylate (160 mg, 45.2% yield). MS Calcd.: 624.12; MS Found: 625.2 [M+H]⁺.

### 3-[2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-1H-thieno[2,3-d]imidazol-5-yl]-4,5-dihydro-1,2,4-oxadiazol-5-one (compound 58)

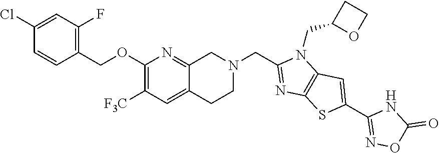 [SMILES: O=c1[nH]c(-c2cc3c(nc(CN4CCc5cc(C(F)(F)F)c(OCc6ccc(Cl)cc6F)nc5C4)n3C[C@@H]3CCO3)s2)no1]

Compound 58 was then synthesized following the similar route of Procedure 4, using methyl (S)-2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-thieno[2,3-d]imidazole-5-carboxylate in step D and CDI in step H.

¹H NMR (400 MHz, DMSO-d₆) δ 13.18 (s, 1H), 8.02 (s, 1H), 7.82 (s, 1H), 7.54-7.50 (t, J=16.4 Hz, 1H), 7.49-7.46 (dd, J1=2 Hz, J2=2 Hz, 1H), 7.33-7.31 (dd, J1=2 Hz, J2=1.6 Hz, 1H), 5.46 (s, 2H), 5.05-5.02 (m, 1H), 4.68-4.62 (m, 1H), 4.58-4.57 (m, 1H), 4.54-4.48 (m, 2H), 4.46-4.43 (m, 2H), 4.34-4.31 (m, 1H), 4.05-4.13 (m, 2H), 3.21-3.34 (m, 2H), 2.91-3.01 (m, 2H), 2.70-2.65 (m, 1H), 2.36-2.32 (m, 1H). ¹⁹F NMR (376 MHz, DMSO-d₆) δ −61.84, −114.95. LC-MS: m/z 651.1 (M+H)⁺.

### 2-[(4-chloro-2-fluorophenyl)methoxy]-7-[(1-{[(2S)-oxetan-2-yl]methyl}-5-[5-(trifluoromethyl)-4H-1,2,4-triazol-3-yl]-1H-thieno[2,3-d]imidazol-2-yl)methyl]-3-(trifluoromethyl)-5,6,7,8-tetrahydro-1,7-naphthyridine (Compound 59)

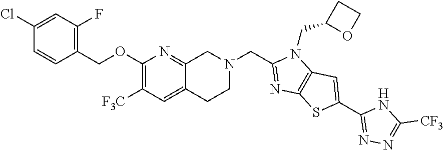 [SMILES: Fc1cc(Cl)ccc1COc1nc2c(cc1C(F)(F)F)CCN(Cc1nc3sc(-c4nnc(C(F)(F)F)[nH]4)cc3n1C[C@@H]1CCO1)C2]

Compound 59 was synthesized following the similar route of Procedure 3, using (S)-2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-thieno[2,3-d]imidazole-5-carbonitrile in step C.

¹H NMR (400 MHz, DMSO-d₆) δ 15.31 (s, 1H), 7.90 (d, J=8.8 Hz, 2H), 7.53-7.45 (m, 2H), 7.31 (dd, J₁=2 Hz, J₂=1.6 Hz, 1H), 5.43 (s, 2H), 5.07-5.04 (m, 1H), 4.66-4.60 (m, 1H), 4.55-4.45 (m, 2H), 4.34-4.31 (m, 1H), 4.03-3.93 (m, 2H), 3.67 (s, 2H), 2.80-2.73 (m, 4H), 2.62-2.60 (m, 1H), 2.37-2.32 (m, 1H). ¹⁹F NMR (376 MHz, DMSO-d₆) δ −61.68, −63.82, −114.98. LC-MS: m/z 702.2 (M+H)⁺.

### Procedure 13

### 2-[(4-chloro-2-fluorophenyl)methoxy]-7-{[5-(5-methyl-4H-1,2,4-triazol-3-yl)-1-{[(2S)-oxetan-2-yl]methyl}-1H-thieno[2,3-d]imidazol-2-yl]methyl}-3-(trifluoromethyl)-5,6,7,8-tetrahydro-1,7-naphthyridine (compound 60)

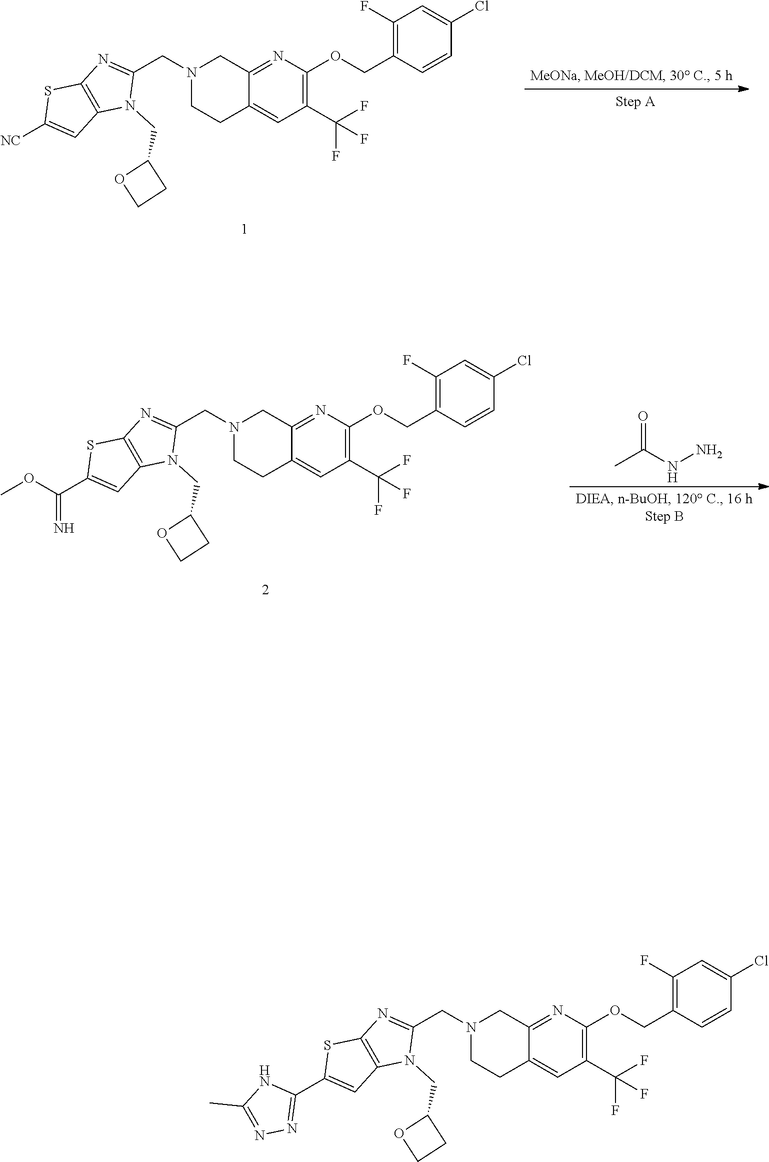

### Step A: methyl (S)-2-((2-((4-chloro-2-fluorobenzyl)oxy)-3-(trifluoromethyl)-5,8-dihydro-1,7-naphthyridin-7(6FH)-yl)methyl)-1-(oxetan-2-ylmethyl)-1H-thieno[2,3-d]imidazole-5-carbimidate

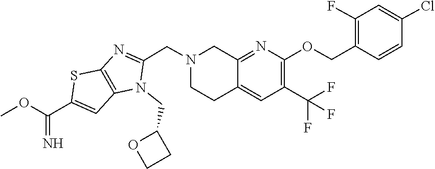 [SMILES: COC(=N)c1cc2c(nc(CN3CCc4cc(C(F)(F)F)c(OCc5ccc(Cl)cc5F)nc4C3)n2C[C@@H]2CCO2)s1]

A mixture of (S)-2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-thieno[2,3-d]imidazole-5-carbonitrile (250 mg, 0.42 mmol) and MeONa (228.43 mg, 4.23 mmol) in MeOH/DCM (2.0 mL/2.0 mL) was stirred at 30° C. for 5 hrs. The reaction mixture was concentrated and purified by column chromatography (DCM/MeOH=100/1) to give methyl (S)-2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-thieno[2,3-d]imidazole-5-carbimidate (198 mg, yield: 75.10%). MS Calcd.: 623.14; MS Found: 624.1 [M+H]⁺.

### Step B: 2-[(4-chloro-2-fluorophenyl)methoxy]-7-{[5-(5-methyl-4H-1,2,4-triazol-3-yl)-1-{[(2S)-oxetan-2-yl]methyl}-1H-thieno[2,3-d]imidazol-2-yl]methyl-3-(trifluoromethyl)-5,6,7,8-tetrahydro-1,7-naphthyridine (Compound 60)

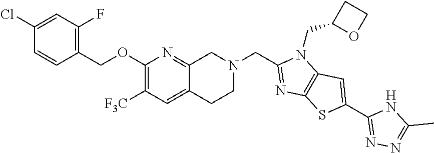 [SMILES: Cc1nnc(-c2cc3c(nc(CN4CCc5cc(C(F)(F)F)c(OCc6ccc(Cl)cc6F)nc5C4)n3C[C@@H]3CCO3)s2)[nH]1]

A mixture of methyl (S)-2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-thieno[2,3-d]imidazole-5-carbimidate (188 mg, 0.30 mmol), acetohydrazide (4.69 mg, 0.60 mmol) and DIEA (116.10 mg, 0.90 mmol) in n-BuOH (4.0 mL) was stirred at 120° C. for 16 hrs. The reaction mixture was diluted with H₂O (20 mL) and extracted with DCM/MeOH (15 mL×2). The combined organic layer was dried over anhydrous sodium sulfate, filtered and evaporated to dryness. The mixture was purified directly by Prep-HPLC (0.10% FA/H₂O/CH₃CN) to give 2-[(4-chloro-2-fluorophenyl)methoxy]-7-{[5-(5-methyl-4H-1,2,4-triazol-3-yl)-1-{[(2S)-oxetan-2-yl]methyl}-1H-thieno[2,3-d]imidazol-2-yl]methyl}-3-(trifluoromethyl)-5,6,7,8-tetrahydro-1,7-naphthyridine (95.24 mg, yield: 48.8%).

¹H NMR (400 MHz, DMSO-d₆) δ 13.67 (br s, 1H), 7.91 (s, 1H), 7.61 (s, 1H), 7.52 (t, J=8.0 Hz, 1H), 7.46 (dd, J=10.0 Hz, 1.6 Hz, 1H), 7.32 (dd, J=8.0 Hz, J=1.2 Hz, 1H), 5.43 (s, 2H), 4.99-5.08 (m, 1H), 4.56-4.65 (m, 1H), 4.42-4.54 (m, 2H), 4.30-4.38 (m, 1H), 3.97 (d, J=13.6 Hz, 1H), 3.91 (d, J=13.6 Hz, 1H), 3.60-3.72 (m, 2H), 2.76-2.83 (m, 4H), 2.56-2.69 (m, 1H), 2.29-2.41 (m, 4H). ¹⁹F NMR (376 MHz, DMSO-d₆) δ −61.67, −114.96. LC-MS: m/z 648.3 (M+H)⁺.

### 2-[(4-chlorophenyl)methoxy]-7-{[6-(5-methyl-4H-1,2,4-triazol-3-yl)-3-{1[(2S)-oxetan-2-yl]methyl}-3H-imidazo[4,5-c]pyridin-2-yl]methyl}-3-(trifluoromethyl)-5,6,7,8-tetrahydro-1,7-naphthyridine (compound 61)

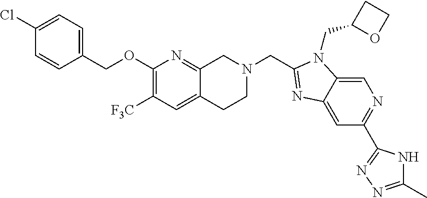 [SMILES: Cc1nnc(-c2cc3nc(CN4CCc5cc(C(F)(F)F)c(OCc6ccc(Cl)cc6)nc5C4)n(C[C@@H]4CCO4)c3cn2)[nH]1]

Compound 61 was synthesized following the similar route of Procedure 13, using (S)-2-((2-((4-chlorophenyl)methoxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbonitrile in step A.

¹H NMR (400 MHz, DMSO-d₆) δ 9.26 (s, 1H), 8.41 (s, 1H), 8.02 (s, 1H), 7.42-7.46 (m, 4H), 5.42 (s, 2H), 5.05-5.15 (m, 1H), 4.90-4.99 (m, 1H), 4.74-4.83 (m, 1H), 4.57-4.65 (m, 1H), 4.45-4.55 (m, 2H), 4.35-4.43 (m, 1H), 4.06-4.15 (m, 2H), 3.21-3.30 (m, 2H), 2.92-3.01 (m, 2H), 2.65-2.75 (m, 1H), 2.46 (s, 3H), 2.33-2.43 (m, 1H). ¹⁹F NMR (376 MHz, DMSO-d₆) δ −61.79. LC-MS: m/z 625.2 (M+H)⁺.

### 2-[(4-chloro-2-fluorophenyl)methoxy]-7-{[6-(5-methyl-4H-1,2,4-triazol-3-yl)-3-{[(2S)-oxetan-2-yl]methyl}-3H-imidazo[4,5-c]pyridin-2-yl]methyl}-3-(trifluoromethyl)-5,6,7,8-tetrahydro-1,7-naphthyridine (compound 62)

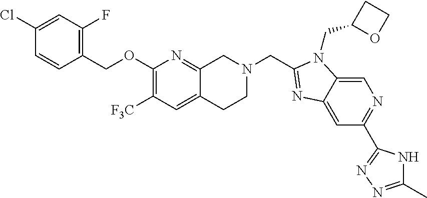 [SMILES: Cc1nnc(-c2cc3nc(CN4CCc5cc(C(F)(F)F)c(OCc6ccc(Cl)cc6F)nc5C4)n(C[C@@H]4CCO4)c3cn2)[nH]1]

Compound 62 was synthesized following the similar route of Procedure 13, using (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbonitrile in step A.

¹H NMR (400 MHz, DMSO-d₆) δ 9.08 (s, 1H), 8.21 (s, 1H), 7.93 (s, 1H), 7.51 (t, J=8.0 Hz, 1H), 7.46 (dd, J=10.0 Hz, 2.0 Hz, 1H), 7.31 (dd, J=8.0 Hz, 1.6 Hz, 1H), 5.42 (s, 2H), 5.07-5.12 (m, 1H), 4.84-4.89 (m, 1H), 4.69-4.73 (m, 1H), 4.44-4.50 (m, 1H), 4.35-4.40 (m, 1H), 4.19 (d, J=13.6 Hz, 1H), 4.09 (d, J=13.6 Hz, 1H), 3.68-3.77 (m, 2H), 2.82-2.88 (m, 4H), 2.64-2.68 (m, 1H), 2.33-2.41 (m, 4H). ¹⁹F NMR (376 MHz, DMSO-d₆) δ −61.68, −114.99. LC-MS: m/z 643.2 (M+H)⁺.

### 2-[(4-chloro-2,6-difluorophenyl)methoxy]-7-{[6-(5-methyl-4H-1,2,4-triazol-3-yl)-3-{[(2S)-oxetan-2-yl]methyl}-3H-imidazo[4,5-c]pyridin-2-yl]methyl}-3-(trifluoromethyl)-5,6,7,8-tetrahydro-1,7-naphthyridine (compound 63)

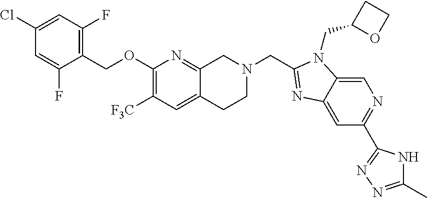 [SMILES: Cc1nnc(-c2cc3nc(CN4CCc5cc(C(F)(F)F)c(OCc6c(F)cc(Cl)cc6F)nc5C4)n(C[C@@H]4CCO4)c3cn2)[nH]1]

Compound 63 was synthesized following the similar route of Procedure 13, using (S)-2-((2-((4-chloro-2,6-difluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbonitrile in step A.

¹H NMR (400 MHz, DMSO-d₆) δ 14.10 (s, 1H), 9.09 (s, 1H), 8.21 (s, 1H), 7.90 (s, 1H), 7.38 (d, J=7.6 Hz, 2H), 5.39 (s, 2H), 5.10-5.09 (m, 1H), 4.90-4.84 (m, 1H), 4.73-4.69 (m, 1H), 4.50-4.44 (m, 1H), 4.40-4.34 (m, 1H), 4.21-4.17 (m, 1H), 4.10-4.07 (m, 1H), 3.76-3.66 (m, 2H), 2.85-2.82 (m, 4H), 2.68-2.63 (m, 1H), 2.43-2.38 (m, 1H), 2.32 (s, 3H). ¹⁹F NMR (376 MHz, DMSO-d₆) δ −61.77, −112.08. LC-MS: m/z 661.4 (M+H)⁺.

### 2-[(4-chlorophenyl)methoxy]-7-[(3-{[(2S)-oxetan-2-yl]methyl}-6-(4H-1,2,4-triazol-3-yl)-3H-imidazo[4,5-c]pyridin-2-yl)methyl]-3-(trifluoromethyl)-5,6,7,8-tetrahydro-1,7-naphthyridine (compound 64)

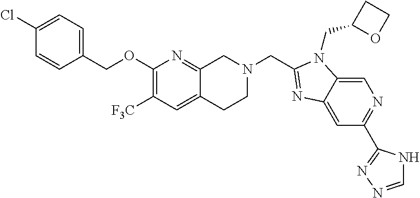 [SMILES: FC(F)(F)c1cc2c(nc1OCc1ccc(Cl)cc1)CN(Cc1nc3cc(-c4nnc[nH]4)ncc3n1C[C@@H]1CCO1)CC2]

Compound 64 was synthesized following the similar route of Procedure 13, using (S)-2-((2-((4-chlorophenyl)methoxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbonitrile in step A and formohydrazide in step B.

¹H NMR (400 MHz, DMSO-d₆) δ 9.26 (s, 1H), 8.42 (s, 1H), 8.37 (s, 1H), 8.05 (s, 1H), 7.41-7.48 (m, 4H), 5.43 (s, 2H), 5.07-5.15 (m, 1H), 4.88-4.98 (m, 1H), 4.74-4.82 (m, 1H), 4.53-4.72 (m, 2H), 4.43-4.50 (m, 1H), 4.34-4.40 (m, 1H), 4.16-4.26 (m, 2H), 3.31-3.42 (m, 2H), 2.95-3.06 (m, 2H), 2.66-2.77 (m, 1H), 2.33-2.44 (m, 1H). ¹⁹F NMR (376 MHz, DMSO-d₆) δ −61.83. LC-MS: m/z 611.1 (M+H)⁺.

### 2-[(4-chloro-2-fluorophenyl)methoxy]-7-{[6-(5-cyclopropyl-4H-1,2,4-triazol-3-yl)-3-{1[(2S)-oxetan-2-yl]methyl}-3H-imidazo[4,5-c]pyridin-2-yl]methyl}-3-(trifluoromethyl)-5,6,7,8-tetrahydro-1,7-naphthyridine (Compound 65)

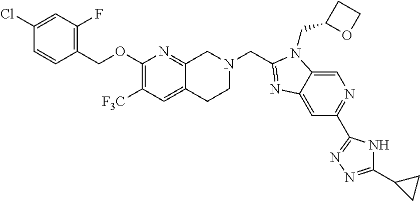 [SMILES: Fc1cc(Cl)ccc1COc1nc2c(cc1C(F)(F)F)CCN(Cc1nc3cc(-c4nnc(C5CC5)[nH]4)ncc3n1C[C@@H]1CCO1)C2]

Compound 65 was synthesized following the similar route of Procedure 13, using (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbonitrile in step A and cyclopropane carbohydrazide in step B.

¹H NMR (400 MHz, CD₃OD) δ 9.06 (s, 1H), 8.49 (s, 0.7H), 8.35 (s, 1H), 7.77 (s, 1H), 7.464 (t, J=16 Hz, 1H), 7.18-7.12 (m, 2H), 5.43 (s, 2H), 5.22-5.20 (m, 1H), 4.93-4.91 (m, 1H), 4.76-4.73 (m, 1H), 4.61-4.58 (m, 1H), 4.44-4.41 (m, 1H), 4.26-4.22 (d, J=13.2 Hz, 1H), 4.15-4.12 (d, J=13.2 Hz, 1H), 3.73-3.71 (m, 2H), 2.89-2.87 (m, 4H), 2.72 (m, 1H), 2.48-2.46 (m, 1H), 2.11 (m, 1H), 1.08-1.02 (m, 4H). ¹⁹F NMR (376 MHz, CD₃OD) δ −64.55, −117.35. LC-MS: m/z 669.2 (M+H)⁺.

### 2-[(4-chloro-2-fluorophenyl)methoxy]-7-({6-[5-(1,1-difluoroethyl)-4H-1,2,4-triazol-3-yl]-3-{[(2S)-oxetan-2-yl]methyl}-3H-imidazo[4,5-c]pyridin-2-yl}methyl)-3-(trifluoromethyl)-5,6,7,8-tetrahydro-1,7-naphthyridine (compound 66)

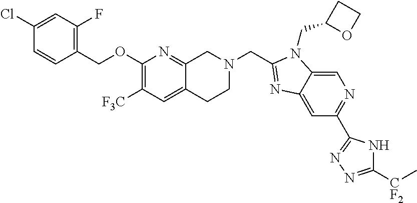 [SMILES: CC(F)(F)c1nnc(-c2cc3nc(CN4CCc5cc(C(F)(F)F)c(OCc6ccc(Cl)cc6F)nc5C4)n(C[C@@H]4CCO4)c3cn2)[nH]1]

Compound 66 was synthesized following the similar route of Procedure 13, using (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbonitrile in step A and 2,2-difluoropropanehydrazide in step B.

¹H NMR (400 MHz, DMSO-d₆) δ 14.93 (br s, 1H), 9.15 (s, 1H), 8.30 (s, 1H), 7.93 (s, 1H), 7.51 (t, J=8.0 Hz, 1H), 7.45 (dd, J=10.0 Hz, 2.0 Hz, 1H), 7.31 (dd, J=8.4 Hz, 2.0 Hz, 1H), 5.42 (s, 2H), 5.06-5.16 (m, 1H), 4.86-4.94 (m, 1H), 4.70-4.79 (m, 1H), 4.44-4.51 (m, 1H), 4.35-4.42 (m, 1H), 4.21 (d, J=14.0 Hz, 1H), 4.10 (d, J=14.0 Hz, 1H), 3.67-3.80 (m, 2H), 2.78-2.91 (m, 4H), 2.61-2.72 (m, 1H), 2.34-2.45 (m, 1H), 2.09 (t, J=18.8 Hz, 3H). ¹⁹F NMR (376 MHz, DMSO-d₆) δ −61.68, −86.46, −115.00. LC-MS: m/z 693.3 (M+H)⁺.

### Procedure 14

### 2-[(4-chloro-2-fluorophenyl)methoxy]-7-{[6-(5-methyl-4H-1,2,4-triazol-3-yl)-3-{[(2S)-oxetan-2-yl]methyl}-3H-imidazo[4,5-b]pyridin-2-yl]methyl}-3-(trifluoromethyl)-5,6,7,8-tetrahydro-1,7-naphthyridine (compound 67)

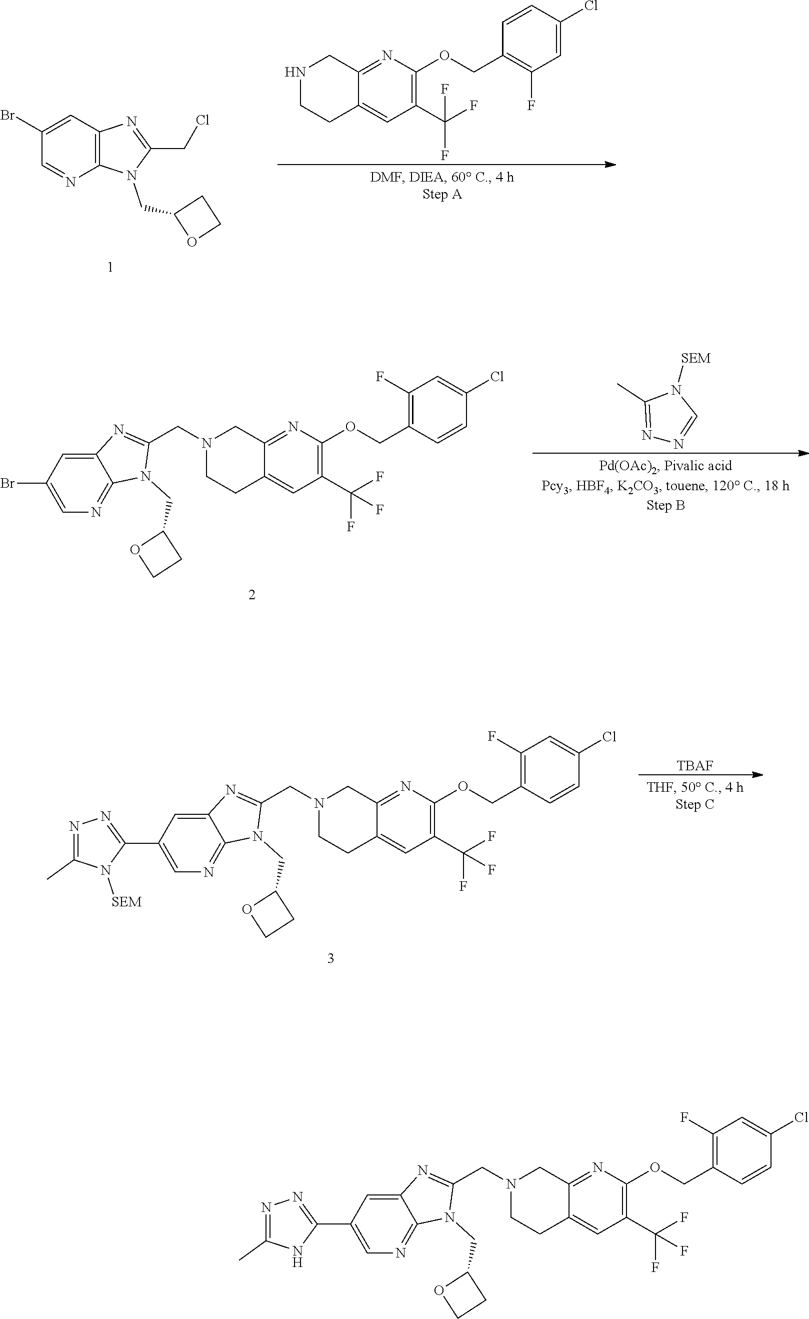

### Step A: (S)-7-((6-bromo-3-(oxetan-2-ylmethyl)-3H-imidazo[4,5-b]pyridin-2-yl)methyl)-2-((4-chloro-2-fluorobenzyl)oxy)-3-(trifluoromethyl)-5,6,7,8-tetrahydro-1,7-naphthyridine

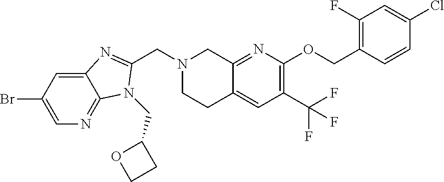 [SMILES: Fc1cc(Cl)ccc1COc1nc2c(cc1C(F)(F)F)CCN(Cc1nc3cc(Br)cnc3n1C[C@@H]1CCO1)C2]

A solution of (S)-6-bromo-2-(chloromethyl)-3-(oxetan-2-ylmethyl)-3H-imidazo[4,5-b]pyridine (500 mg, 1.587 mmol), 2-((4-chloro-2-fluorobenzyl)oxy)-3-(trifluoromethyl)-5,6,7,8-tetrahydro-1,7-naphthyridine (628 mg, 1.746 mmol) and DIEA (1.023 g, 7.935 mmol) in DMF (10 mL) was stirred at 60° C. for 4 hrs under N₂. After the reaction was completed, the mixture was added to water (50 mL), and the mixture was extracted with EtOAc (30 mL*2). The combined organic layers were washed with brine (20 mL*2), dried over Na₂SO₄, filtered, and concentrated in vacuum. The residue was purified by flash column chromatography (eluting PE/EA=1/1) to give (S)-7-((6-bromo-3-(oxetan-2-ylmethyl)-3H-imidazo[4,5-b]pyridin-2-yl)methyl)-2-((4-chloro-2-fluorobenzyl)oxy)-3-(trifluoromethyl)-5,6,7,8-tetrahydro-1,7-naphthyridine (800 mg, 78.89% yield). MS Calcd: 639.07; MS Found: 640.1 [M+H]⁺.

### Step B: (S)-2-((4-chloro-2-fluorobenzyl)oxy)-7-((6-(5-methyl-4-((2-(trimethylsilyl)ethoxy)methyl)-4H-1,2,4-triazol-3-yl)-3-(oxetan-2-ylmethyl)-3H-imidazo[4,5-b]pyridin-2-yl)methyl)-3-(trifluoromethyl)-5,6,7,8-tetrahydro-1,7-naphthyridine

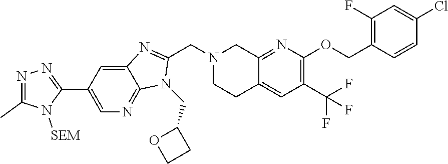 [SMILES: Cc1nnc(-c2cnc3c(c2)nc(CN2CCc4cc(C(F)(F)F)c(OCc5ccc(Cl)cc5F)nc4C2)n3C[C@@H]2CCO2)n1COCC[Si](C)(C)C]

To a solution of (S)-7-((6-bromo-3-(oxetan-2-ylmethyl)-3H-imidazo[4,5-b]pyridin-2-yl)methyl)-2-((4-chloro-2-fluorobenzyl)oxy)-3-(trifluoromethyl)-5,6,7,8-tetrahydro-1,7-naphthyridine (200 mg, 0.313 mmol) in toluene (10 mL) was added 3-methyl-4-((2-(trimethylsilyl)ethoxy) methyl)-4H-1,2,4-triazole (200 mg, 0.939 mmol), pivalic acid (2 mg, 0.019 mmol), PCy₃HBF₄ (23 mg, 0.0626 mmol), Pd (II) acetate (7 mg, 0.03 mmol), and K₂CO₃ (259 mg, 1.878 mmol), and the reaction mixture was stirred at 120° C. for 18 hrs under N₂. After the reaction was completed, the mixture was concentrated in vacuum, and the residue was purified by flash column chromatography (eluting DCM/MeOH=25/1) to give (S)-2-((4-chloro-2-fluorobenzyl)oxy)-7-((6-(5-methyl-4-((2-(trimethylsilyl)ethoxy)methyl)-4H-1,2,4-triazol-3-yl)-3-(oxetan-2-ylmethyl)-3H-imidazo[4,5-b]pyridin-2-yl)methyl)-3-(trifluoromethyl)-5,6,7,8-tetrahydro-1,7-naphthyridine (43 mg, 17.8% yield). MS Calcd.: 772.27; MS Found: 773.4 [M+H]⁺.

### Step C: 2-[(4-chloro-2-fluorophenyl)methoxy]-7-{[6-(5-methyl-4H-1,2,4-triazol-3-yl)-3-{[(2S)-oxetan-2-yl]methyl}-3H-imidazo[4,5-b]pyridin-2-yl]methyl)-3-(trifluoromethyl)-5,6,7,8-tetrahydro-1,7-naphthyridine (Compound 67)

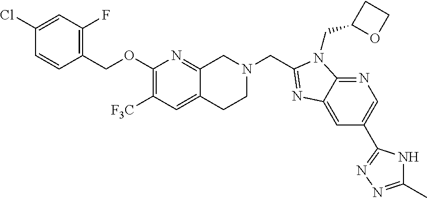 [SMILES: Cc1nnc(-c2cnc3c(c2)nc(CN2CCc4cc(C(F)(F)F)c(OCc5ccc(Cl)cc5F)nc4C2)n3C[C@@H]2CCO2)[nH]1]

To a solution of (S)-2-((4-chloro-2-fluorobenzyl)oxy)-7-((6-(5-methyl-4-((2-(trimethylsilyl)ethoxy)methyl)-4H-1,2,4-triazol-3-yl)-3-(oxetan-2-ylmethyl)-3H-imidazo[4,5-b]pyridin-2-yl)methyl)-3-(trifluoromethyl)-5,6,7,8-tetrahydro-1,7-naphthyridine (43 mg, 0.056 mmol) in THF (10 mL) was added TBAF (1M in THF, 0.17 mL), and the reaction mixture was stirred at 50° C. for 4 hrs under N₂. After the reaction was completed, the mixture was added to EtOAc (50 mL) and washed with aq. NH₄Cl (20 mL*5), dried over Na₂SO₄, filtered, and concentrated in vacuum. The filtrate was purified by prep-HPLC (eluting MeCN in water, 0.1% TFA) to give 2-[(4-chloro-2-fluorophenyl)methoxy]-7-{[6-(5-methyl-4H-1,2,4-triazol-3-yl)-3-{[(2S)-oxetan-2-yl]methyl}-3H-imidazo[4,5-b]pyridin-2-yl]methyl}-3-(trifluoromethyl)-5,6,7,8-tetrahydro-1,7-naphthyridine (1.1 mg, 2.6% yield).

¹H NMR (400 MHz, CD₃OD) δ 9.05 (s, 1H), 8.62 (s, 1H), 7.77 (s, 1H), 7.47 (t, J=16 Hz, 1H), 7.18-7.16 (m, 2H), 5.44 (s, 2H), 4.57 (m, 2H), 4.21-4.29 (m, 2H), 3.74 (s, 2H), 3.55-3.53 (m, 1H), 3.48-3.47 (m, 2H), 3.13-3.12 (m, 2H), 2.91-2.90 (m, 4H), 2.53 (s, 3H). ¹⁹F NMR (376 MHz, CD₃OD) δ −64.54, −117.64. LC-MS: m/z 643.2 (M+H)⁺.

### 2-[(4-chlorophenyl)methoxy]-7-{[4-fluoro-5-(5-methyl-4H-1,2,4-triazol-3-yl)-1-{[(2S)-oxetan-2-yl]methyl}-1H-1,3-benzodiazol-2-yl]methyl}-3-(trifluoromethyl)-5,6,7,8-tetrahydro-1,7-naphthyridine (compound 68)

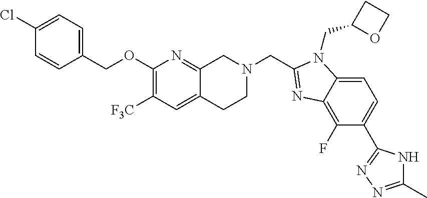 [SMILES: Cc1nnc(-c2ccc3c(nc(CN4CCc5cc(C(F)(F)F)c(OCc6ccc(Cl)cc6)nc5C4)n3C[C@@H]3CCO3)c2F)[nH]1]

Compound 68 was synthesized following the similar route of Procedure 14, using (S)-5-bromo-2-(chloromethyl)-4-fluoro-1-(oxetan-2-ylmethyl)-1H-benzo[d]imidazole in step A.

¹H NMR (400 MHz, DMSO-d₆) δ 8.06 (s, 1H), 7.87 (dd, J=8.4 Hz, J=6.8 Hz, 1H), 7.63 (d, J=8.8 Hz 1H), 7.41-7.46 (m, 4H), 5.43 (s, 2H), 5.01-5.09 (m, 1H), 4.72-4.79 (m, 1H), 4.59-4.66 (m, 2H), 4.42-4.49 (m, 1H), 4.29-4.36 (m, 1H), 4.13-4.25 (m, 3H), 3.32-3.43 (m, 2H), 2.97-3.05 (m, 2H), 2.62-2.72 (m, 1H), 2.41 (s, 3H), 2.29-2.38 (m, 1H). 19F NMR (376 MHz, DMSO-d₆) δ −61.83, −128.26. LC-MS: m/z 642.2 (M+H)⁺.

### Procedure 15

### 2-[(4-chloro-2,6-difluorophenyl)methoxy]-7-({6-[5-(difluoromethyl)-4H-1,2,4-triazol-3-yl]-3-{[(2S)-oxetan-2-yl]methyl}-3H-imidazo[4,5-c]pyridin-2-yl}methyl)-5,6,7,8-tetrahydro-1,7-naphthyridine-3-carbonitrile (compound 69)

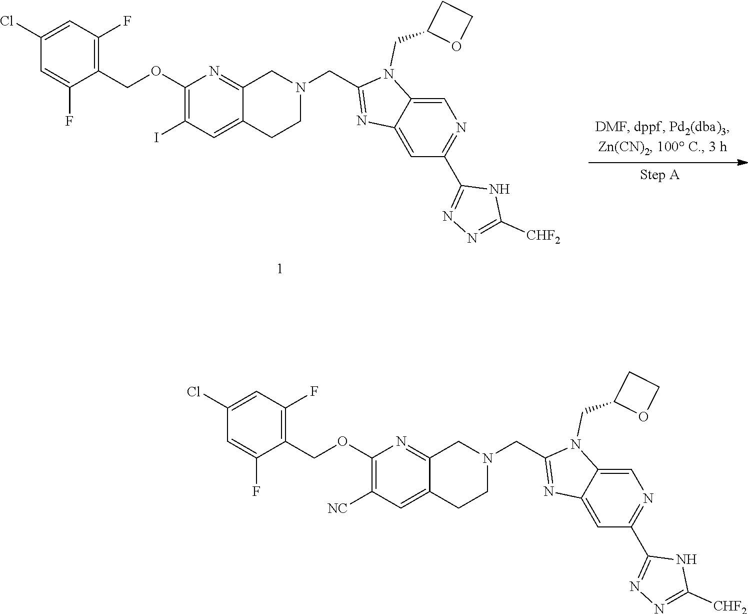 [SMILES: Fc1cc(Cl)cc(F)c1COc1nc2c(cc1I)CCN(Cc1nc3cc(-c4nnc(C(F)F)[nH]4)ncc3n1C[C@@H]1CCO1)C2 ; N#Cc1cc2c(nc1OCc1c(F)cc(Cl)cc1F)CN(Cc1nc3cc(-c4nnc(C(F)F)[nH]4)ncc3n1C[C@@H]1CCO1)CC2]

A mixture of (S)-2-((4-chloro-2,6-difluorobenzyl)oxy)-7-((6-(5-(difluoromethyl)-4H-1,2,4-triazol-3-yl)-3-(oxetan-2-ylmethyl)-3H-imidazo[4,5-c]pyridin-2-yl)methyl)-3-iodo-5,6,7,8-tetrahydro-1,7-naphthyridine which was synthesized following the similar route of Procedure 3, using 2-((4-chloro-2,6-difluorobenzyl)oxy)-3-iodo-5,6,7,8-tetrahydro-1,7-naphthyridine and (S)-2-(chloromethyl)-3-(oxetan-2-ylmethyl)-3H-imidazo[4,5-c]pyridine-6-carbonitrile in step B and 2,2-difluoroacetic anhydride in step D (140 mg, 0.185 mmol) in DMF (6 mL) were added Zn(CN)₂ (43.55 mg, 0.371 mmol), Pd₂(dba)₃ (16.98 mg, 0.019 mmol) and dppf (10.47 mg, 0.019 mmol) under N₂, and the reaction mixture was stirred at 100° C. for 3 hrs. After the reaction was completed, the reaction mixture was quenched with H₂O (20 mL) and extracted with ethyl acetate (20 mL×3). The organic layer was combined and washed with brine (20 mL×2), dried over sodium sulfate, filtered, and purified by column chromatography on silica gel (DCM/MeOH/NH₃·H₂O=8/1/0.1) to give 2-[(4-chloro-2,6-difluorophenyl)methoxy]-7-({6-[5-(difluoromethyl)-4H-1,2,4-triazol-3-yl]-3-{[(2S)-oxetan-2-yl]methyl}-3H-imidazo[4,5-c]pyridin-2-yl}methyl)-5,6,7,8-tetrahydro-1,7-naphthyridine-3-carbonitrile (18.46 mg, 15.22% yield).

¹H NMR (400 MHz, DMSO-d₆) δ 15.02 (s, 1H), 9.15 (s, 1H), 8.31 (s, 1H), 8.09 (s 1H), 7.43 (d, J=7.2 Hz, 2H), 7.14 (t, J=53.6, 1H), 5.41-5.43 (m, 2H), 5.09-5.11 (m, 1H), 4.86-4.92 (m, 1H), 4.72-4.76 (m, 1H), 4.45-4.50 (m, 1H), 4.35-4.40 (m, 1H), 4.21 (d, J=13.6 Hz, 1H), 4.11 (d, J=13.6 Hz, 1H), 3.69-3.79 (m, 2H), 2.81-2.90 (m, 2H), 2.77-2.79 (m, 2H), 2.64-2.69 (m, 1H), 2.38-2.42 (m, 1H). ¹⁹F NMR (376 MHz, DMSO-d₆) δ −111.93, −115-75. LC-MS: m/z 654.6 (M+H)⁺.

### 2-[(4-chloro-2,6-difluorophenyl)methoxy]-7-[(1-{[(2S)-oxetan-2-yl]methyl}-5-(5-oxo-4,5-dihydro-1,2,4-oxadiazol-3-yl)-1H-1,3-benzodiazol-2-yl)methyl]-5,6,7,8-tetrahydro-1,7-naphthyridine-3-carbonitrile (compound 70)

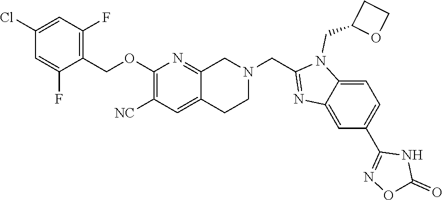 [SMILES: N#Cc1cc2c(nc1OCc1c(F)cc(Cl)cc1F)CN(Cc1nc3cc(-c4noc(=O)[nH]4)ccc3n1C[C@@H]1CCO1)CC2]

Compound 70 was synthesized following the similar route of Procedure 15, using (S)-2-((4-chloro-2,6-difluorobenzyl)oxy)-7-((6-(5-(difluoromethyl)-4H-1,2,4-triazol-3-yl)-3-(oxetan-2-ylmethyl)-3H-imidazo[4,5-c]pyridin-2-yl)methyl)-3-iodo-5,6,7,8-tetrahydro-1,7-naphthyridine in step A.

¹H NMR (400 MHz, DMSO-d₆) δ 12.91 (s, 1H), 8.08-8.09 (m, 1H), 7.83 (d, J=8.8 Hz, 1H), 7.72 (dd, J=8.4 Hz, 1.6 Hz, 1H), 7.40-7.44 (m, 2H), 5.40-5.43 (m, 2H), 5.04-5.07 (m, 1H), 4.72-4.78 (m, 1H), 4.63 (dd, J=14.8 Hz, 2.4 Hz, 1H), 4.42-4.48 (m, 1H), 4.31-4.36 (m, 1H), 4.15 (d, J=13.6 Hz, 1H), 4.04 (d, J=13.6 Hz, 1H), 3.71 (dd, J=24.4 Hz, 17.2 Hz, 1H), 2.76-2.85 (m, 4H), 2.60-2.68 (m, 1H), 2.33-2.40 (m, 1H). ¹⁹F NMR (376 MHz, DMSO-d₆) δ −111.93. LC-MS: m/z 620.5 (M+H)⁺.

### 2-[(4-chloro-2,6-difluorophenyl)methoxy]-7-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-5,6,7,8-tetrahydro-1,7-naphthyridine-3-carbonitrile (compound 71)

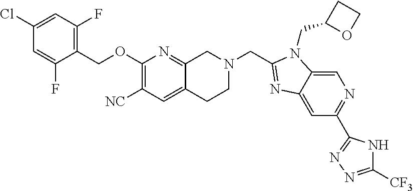 [SMILES: N#Cc1cc2c(nc1OCc1c(F)cc(Cl)cc1F)CN(Cc1nc3cc(-c4nnc(C(F)(F)F)[nH]4)ncc3n1C[C@@H]1CCO1)CC2]

Compound 71 was synthesized following the similar route of Procedure 15, using (S)-2-((4-chloro-2,6-difluorobenzyl)oxy)-3-iodo-7-((3-(oxetan-2-ylmethyl)-6-(5-(trifluoromethyl)-4H-1,2,4-triazol-3-yl)-3H-imidazo[4,5-c]pyridin-2-yl)methyl)-5,6,7,8-tetrahydro-1,7-naphthyridine in step A.

¹H NMR (400 MHz, DMSO-d₆) δ 9.15 (s, 1H), 8.31 (s, 1H), 8.09 (s, 1H), 7.43 (dd, J=10.8 Hz, 3.2 Hz, 2H), 5.41-5.43 (m, 2H), 5.09-5.11 (m, 1H), 4.86-4.92 (m, 1H), 4.71-4.76 (m, 1H), 4.45-4.50 (m, 1H), 4.36-4.38 (m, 1H), 4.23 (d, J=14 Hz, 1H), 4.12 (d, J=14 Hz, 1H), 3.69-3.81 (m, 2H), 2.85-2.89 (m, 2H), 2.77-2.79 (m, 2H), 2.64-2.69 (m, 1H), 2.38-2.41 (m, 1H). ¹⁹F NMR (376 MHz, DMSO-d₆) δ −63.38, −111.93. LC-MS: m/z 672.6 (M+H)⁺.

### Procedure 16

### 2-[(4-chloro-2-fluorophenyl)methoxy]-3-(difluoromethyl)-7-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-5,6,7,8-tetrahydro-1,7-naphthyridine (compound 72)

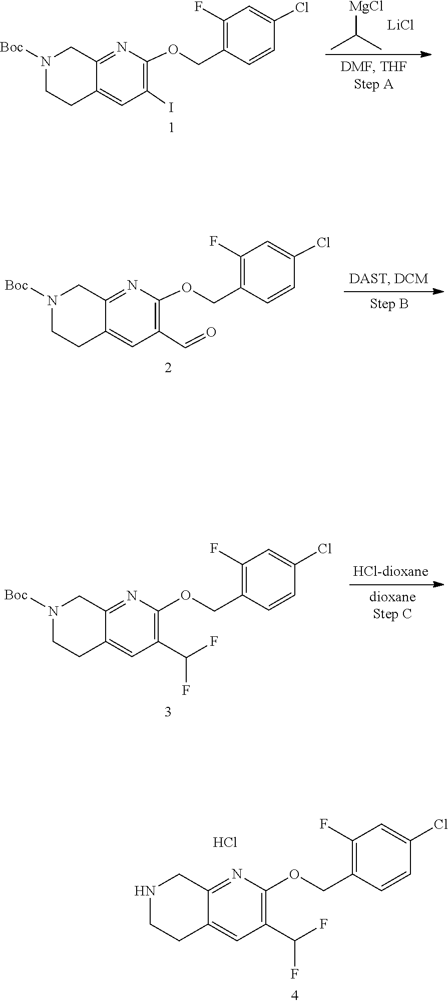

### Step A: tert-butyl 2-((4-chloro-2-fluorobenzyl)oxy)-3-formyl-58-dihydro-1,7-naphthyridine-7(6H)-carboxylate

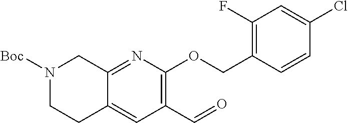 [SMILES: CC(C)(C)OC(=O)N1CCc2cc(C=O)c(OCc3ccc(Cl)cc3F)nc2C1]

To a mixture of tert-butyl 2-((4-chloro-2-fluorobenzyl)oxy)-3-iodo-5,8-dihydro-1,7-naphthyridine-7(6H)-carboxylate (300 mg, 0.580 mmol) in THF (5 mL) was added ⁱPrMgCl·LiCl (1.3 M in THF, 0.6 mL) slowly at 0° C. under N₂. After 10 minutes, DMF (84 mg, 1.16 mmol) was added. The reaction mixture was stirred at 0° C. for 2 hours. The reaction mixture was quenched with H₂O (10 mL) and extracted with EtOAc (30 mL×3). The organic layers were combined and washed with brine (20 mL×5), dried over anhydrous Na₂SO₄, filtered, and concentrated under reduced pressure. The residue was purified by column chromatography (PE:EtOAc=10:1) to give tert-butyl 2-((4-chloro-2-fluorobenzyl)oxy)-3-formyl-5,8-dihydro-1,7-naphthyridine-7(6H)-carboxylate (200 mg, 82.3% yield).

¹H NMR (400 MHz, DMSO-d₆) δ 10.21 (s, 1H), 7.95 (s, 1H), 7.68 (t, J=8.4 Hz, 1H), 7.50 (dd, J=1.6 Hz, 10.0 Hz, 1H), 7.33 (dd, J=2.0 Hz, 8.4 Hz, 1H), 5.76 (s, 2H), 4.51 (s, 2H), 3.59 (t, J=5.6 Hz, 2H), 2.78 (t, J=5.6 Hz, 2H), 1.44 (s, 9H). ¹⁹F NMR (376 MHz, DMSO-d₆) δ −115.03. LC-MS: m/z 295.2 (M+H)⁺.

### Step B: tert-butyl 2-((4-chloro-2-fluorobenzyl)oxy)-3-(difluoromethyl)-5,8-dihydro-1,7-naphthyridine-7(6H)-carboxylate

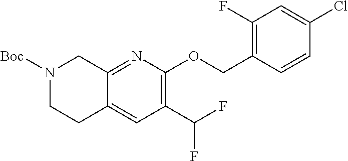 [SMILES: CC(C)(C)OC(=O)N1CCc2cc(C(F)F)c(OCc3ccc(Cl)cc3F)nc2C1]

To a solution of tert-butyl 2-((4-chloro-2-fluorobenzyl)oxy)-3-formyl-5,8-dihydro-1,7-naphthyridine-7(6H)-carboxylate (200 mg, 0.476 mmol) in DCM (5 mL) was added DAST (153 mg, 0.952 mmol) at room temperature. The reaction mixture was stirred at room temperature for 2 hours. The reaction mixture was quenched with sat. NaHCO₃ solution (15 mL) and extracted with EtOAc (30 mL×3). The organic layers were combined and washed with brine (20 mL), dried over anhydrous Na₂SO₄, filtered, and concentrated under reduced pressure. The reside was purified by column chromatography (PE:EtOAc=10:1) to give tert-butyl 2-((4-chloro-2-fluorobenzyl)oxy)-3-(difluoromethyl)-5,8-dihydro-1,7-naphthyridine-7(6H)-carboxylate (150 mg, 71.4% yield). LC-MS: m/z 443.2 (M+H)⁺.

### Step C: 2-((4-chloro-2-fluorobenzyl)oxy)-3-(difluoromethyl)-5,6,7,8-tetrahydro-1,7-naphthyridine, HCl salt

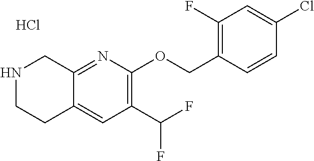 [SMILES: Fc1cc(Cl)ccc1COc1nc2c(cc1C(F)F)CCNC2 ; Cl]

To a solution of tert-butyl 2-((4-chloro-2-fluorobenzyl)oxy)-3-(difluoromethyl)-5,8-dihydro-1,7-naphthyridine-7(6H)-carboxylate (150 mg, 0.339 mmol) in dioxane (2 mL) was added HCl/dioxane (2 mL, 4 M) at room temperature. The reaction mixture was stirred at room temperature for 2 hours. After the reaction was completed, the reaction mixture was concentrated in vacuum to give 2-((4-chloro-2-fluorobenzyl)oxy)-3-(difluoromethyl)-5,6,7,8-tetrahydro-1,7-naphthyridine, HCl salt (200 mg, crude). LC-MS: m/z 343.5 (M+H−HCl)⁺.

### 2-[(4-chloro-2-fluorophenyl)methoxy]-3-(difluoromethyl)-7-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-5,6,7,8-tetrahydro-1,7-naphthyridine (compound 72)

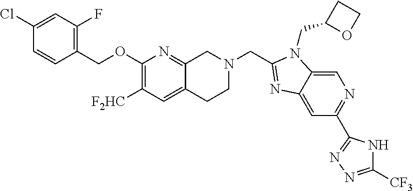 [SMILES: Fc1cc(Cl)ccc1COc1nc2c(cc1C(F)F)CCN(Cc1nc3cc(-c4nnc(C(F)(F)F)[nH]4)ncc3n1C[C@@H]1CCO1)C2]

Compound 72 was then synthesized following the similar route of Procedure 3, using 2-((4-chloro-2-fluorobenzyl)oxy)-3-(difluoromethyl)-5,6,7,8-tetrahydro-1,7-naphthyridine, HCl salt and (S)-2-(chloromethyl)-3-(oxetan-2-ylmethyl)-3H-imidazo[4,5-c]pyridine-6-carbonitrile in step B.

¹H NMR (400 MHz, DMSO-d₆) δ 15.48 (br s, 1H), 9.26 (s, 1H), 8.38 (s, 1H), 7.87 (s, 1H), 7.57 (t, J=8.0 Hz, 1H), 7.46 (d, J=9.6 Hz, 1H), 7.31 (d, J=8.0 Hz, 1H), 7.08 (t, J=55.2 Hz, 1H), 5.41 (s, 2H), 5.05-5.17 (m, 1H), 4.86-4.98 (m, 1H), 4.64-4.80 (m, 3H), 4.43-4.57 (m, 1H), 4.34-4.42 (m, 1H), 4.11-4.31 (m, 2H), 3.26-3.48 (m, 2H), 2.91-3.05 (m, 2H), 2.67-2.77 (m, 1H), 2.35-2.43 (m, 1H). ¹⁹F NMR (376 MHz, DMSO-d₆) δ −63.74, 74.29, −115.06. LC-MS: m/z 654.2 (M+H)⁺.

### Procedure 17

### 2-{[4-(difluoromethyl)phenyl]methoxy}-7-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridine (compound 73)

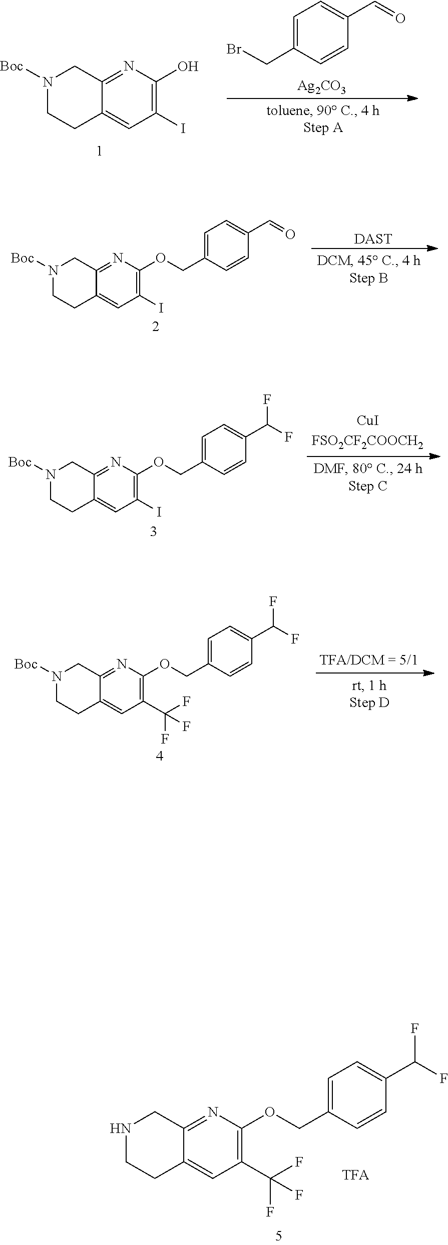

### Step A: tert-butyl 2-((4-formylbenzyl)oxy)-3-iodo-5,8-dihydro-1,7-naphthyridine-7(6H)-carboxylate

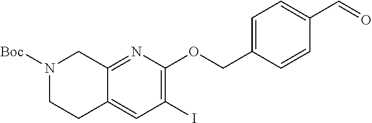 [SMILES: CC(C)(C)OC(=O)N1CCc2cc(I)c(OCc3ccc(C=O)cc3)nc2C1]

To a mixture of tert-butyl 2-hydroxy-3-iodo-5,8-dihydro-1,7-naphthyridine-7(6H)-carboxylate (1.0 g, 2.66 mmol) and 4-(bromomethyl)benzaldehyde (688 mg, 3.46 mmol) in toluene (30 mL) was added Ag₂CO₃ (1.47 g, 5.32 mmol), and the mixture was stirred at room 90° C. for 4 hours. The reaction mixture was quenched with H₂O (80 mL) and extracted with ethyl acetate (60 mL×3). The organic layer was combined and washed with brine (60 mL×2), dried over sodium sulfate, filtered, and concentrated in vacuum. The reaction was purified by column chromatography (EA: PE=0 to 15%) to give tert-butyl 2-((4-formylbenzyl)oxy)-3-iodo-5,8-dihydro-1,7-naphthyridine-7(6H)-carboxylate (880 mg, yield: 66.9%). MS Calcd.: 494.07; MS Found: 495.4 [M+H]⁺.

### Step B: tert-butyl 2-((4-(difluoromethyl)benzyl)oxy)-3-iodo-5,8-dihydro-1,7-naphthyridine-7(6H)-carboxylate

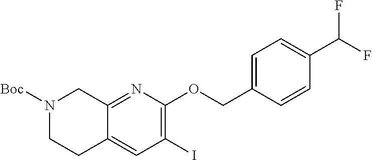 [SMILES: CC(C)(C)OC(=O)N1CCc2cc(I)c(OCc3ccc(C(F)F)cc3)nc2C1]

A mixture of tert-butyl 2-((4-formylbenzyl)oxy)-3-iodo-5,8-dihydro-1,7-naphthyridine-7(6H)-carboxylate (770 mg, 1.56 mmol) and DAST (1.25 g, 7.78 mmol) in DCM (30 mL) was stirred at 45° C. for 4 hours. After the reaction was completed, the mixture was added to H₂O (20 mL) at 0° C. and extracted with ethyl acetate (20 mL×3). The organic layer was combined and washed with brine (20 mL), dried over sodium sulfate, filtered, and concentrated in vacuum. The reaction was purified by column chromatography (EA: PE=0 to 17%) to give tert-butyl 2-((4-(difluoromethyl)benzyl)oxy)-3-iodo-5,8-dihydro-1,7-naphthyridine-7(6H)-carboxylate (520 mg, yield: 64.6%). MS Calcd.: 516.07; MS Found: 517.4 [M+H]⁺.

### Step C: tert-butyl 2-((4-(difluoromethyl)benzyl)oxy)-3-(trifluoromethyl)-5,8-dihydro-1,7-naphthyridine-7(6H)-carboxylate

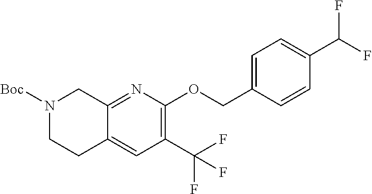 [SMILES: CC(C)(C)OC(=O)N1CCc2cc(C(F)(F)F)c(OCc3ccc(C(F)F)cc3)nc2C1]

A solution of tert-butyl 2-((4-(difluoromethyl)benzyl)oxy)-3-iodo-5,8-dihydro-1,7-naphthyridine-7(6H)-carboxylate (290 mg, 0.56 mmol), FSO₂CF₂COOCH₃ (324 mg, 1.68 mmol), and CuI (11 mg, 0.06 mmol) in DMF (3.0 mL) was stirred at 80° C. for 24 hrs under N₂. After the reaction was completed, the mixture was filtered and evaporated to remove the DMF. The residue was purified by reverse-phase column chromatography (ACN: 0.1% FA/H₂O=0 to 68.2%) give tert-butyl 2-((4-(difluoromethyl)benzyl)oxy)-3-(trifluoromethyl)-5,8-dihydro-1,7-naphthyridine-7(6H)-carboxylate (120 mg, yield: 46.7%). MS Calcd.: 458.16; MS Found: 403.4 [M+H−56]⁺.

### Step D: 2-((4-(difluoromethyl)benzyl)oxy)-3-(trifluoromethyl)-5,6,7,8-tetrahydro-1,7-naphthyridine, TFA Salt

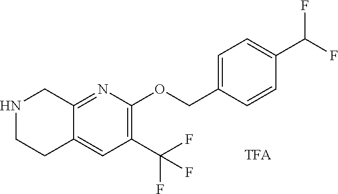 [SMILES: FC(F)c1ccc(COc2nc3c(cc2C(F)(F)F)CCNC3)cc1]

To a solution of tert-butyl 2-((4-(difluoromethyl)benzyl)oxy)-3-(trifluoromethyl)-5,8-dihydro-1,7-naphthyridine-7(6H)-carboxylate (100 mg, 0.26 mmol) in DCM/TFA (5:1, 3 mL) was stirred at 25° C. for 1 hour. After the reaction was completed, The reaction was concentrated in vacuum to give 2-((4-(difluoromethyl)benzyl)oxy)-3-(trifluoromethyl)-5,6,7,8-tetrahydro-1,7-naphthyridine, TFA Salt (200 mg, crude) as a yellow solid. MS Calcd.: 358.11; MS Found: 400.4 [M+H+41−TFA]⁺.

### 2-{{4-(difluoromethyl)phenyl]methoxy}-7-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridine (compound 73)

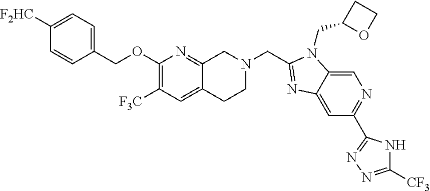 [SMILES: FC(F)c1ccc(COc2nc3c(cc2C(F)(F)F)CCN(Cc2nc4cc(-c5nnc(C(F)(F)F)[nH]5)ncc4n2C[C@@H]2CCO2)C3)cc1]

Compound 73 was then synthesized following the similar route of Procedure 3, using 2-((4-(difluoromethyl)benzyl)oxy)-3-(trifluoromethyl)-5,6,7,8-tetrahydro-1,7-naphthyridine, TFA Salt and (S)-2-(chloromethyl)-3-(oxetan-2-ylmethyl)-3H-imidazo[4,5-c]pyridine-6-carbonitrile in step B.

¹H NMR (400 MHz, DMSO-d₆) δ 15.44 (s, 1H), 9.17 (s, 1H), 8.33 (s, 1H), 7.94 (s, 1H), 7.52-7.58 (m, 4H), 7.01 (t, J=56.0 Hz, 1H), 5.46 (s, 2H), 5.09-5.11 (m, 1H), 4.88-4.94 (m, 1H), 4.77 (d, J=12.8 Hz, 1H), 4.50 (dd, J=12.8 Hz, 7.6 Hz, 1H), 4.40 (dd, J=11.6 Hz, 5.6 Hz, 1H), 4.24 (d, J=13.6 Hz, 1H), 4.13 (d, J=14.0 Hz, 1H), 3.67-3.77 (m, 2H), 2.83-2.87 (m, 4H), 2.65-2.69 (m, 1H), 2.37-2.46 (m, 1H). ¹⁹F NMR (376 MHz, DMSO-d₆) δ −61.64, −63.73, −109.41. LC-MS: m/z 695.5 (M+H)⁺.

### Procedure 18

### 2-[(5-chloropyridin-2-yl)methoxy]-7-[(3-{1[(2S)-oxetan-2-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridine (compound 74)

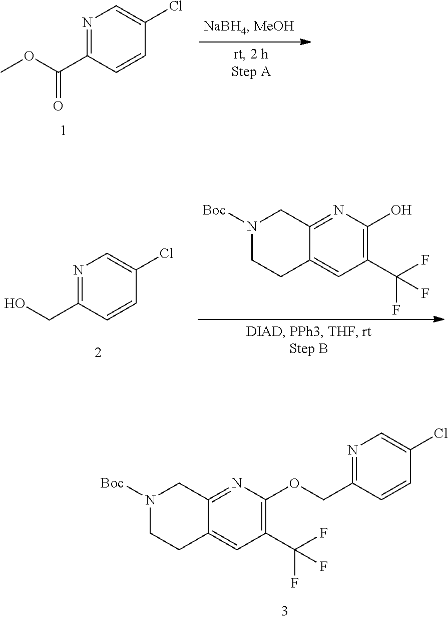 [SMILES: COC(=O)c1ccc(Cl)cn1 ; OCc1ccc(Cl)cn1 ; CC(C)(C)OC(=O)N1CCc2cc(C(F)(F)F)c(O)nc2C1 ; CC(C)(C)OC(=O)N1CCc2cc(C(F)(F)F)c(OCc3ccc(Cl)cn3)nc2C1]

### Step A: (5-chloropyridin-2-yl)methanol

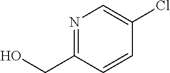

A mixture of methyl 5-chloropicolinate (500 mg, 2.92 mmol) and NaBH₄ (333 mg, 8.76 mmol) in MeOH (10 mL) was stirred at room temperature for 2 hours. The resulting mixture were concentrated and then purified by column chromatography on silica gel (PE/EtOAc=1/1) to give (5-chloropyridin-2-yl)methanol (343 mg, yield: 82%). MS Calcd.: 143.01; MS Found: 144.1 [M+H]⁺.

### Step B: tert-butyl 2-((5-chloropyridin-2-yl)methoxy)-3-(trifluoromethyl)-5,8-dihydro-1,7-naphthyridine-7(6H)-carboxylate

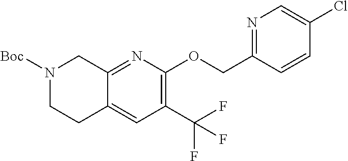 [SMILES: CC(C)(C)OC(=O)N1CCc2cc(C(F)(F)F)c(OCc3ccc(Cl)cn3)nc2C1]

To a solution of (5-chloropyridin-2-yl)methanol (107 mg, 0.75 mmol), tert-butyl 2-hydroxy-3-(trifluoromethyl)-5,8-dihydro-1,7-naphthyridine-7(6H)-carboxylate (250 mg, 0.79 mmol) and PPh₃ (235 mg, 0.90 mmol) in THF (6 mL) was added DIAD (181 mg, 0.90 mmol) at 0° C., and the mixture was stirred at room temperature for 2 hrs under N₂. The mixture was diluted with EtOAc (100 mL) and was washed with brine (100 mL*3). The organic layer was separated and dried over Na₂SO₄, filtered, and concentrated to give tert-butyl 2-((5-chloropyridin-2-yl)methoxy)-3-(trifluoromethyl)-5,8-dihydro-1,7-naphthyridine-7(6H)-carboxylate (157 mg, yield: 47%). MS Calcd.: 443.12; MS Found: 444.1 [M+H]⁺.

### 2-[(5-chloropyridin-2-yl)methoxy]-7-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridine (compound 74)

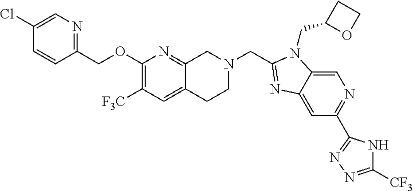 [SMILES: FC(F)(F)c1nnc(-c2cc3nc(CN4CCc5cc(C(F)(F)F)c(OCc6ccc(Cl)cn6)nc5C4)n(C[C@@H]4CCO4)c3cn2)[nH]1]

Compound 74 was then synthesized following the similar route of Procedure 3, using tert-butyl 2-((5-chloropyridin-2-yl)methoxy)-3-(trifluoromethyl)-5,8-dihydro-1,7-naphthyridine-7(6H)-carboxylate in step A and (S)-2-(chloromethyl)-3-(oxetan-2-ylmethyl)-3H-imidazo[4,5-c]pyridine-6-carbonitrile in step B.

¹H NMR (400 MHz, DMSO-d₆) δ 15.42 (s, 1H), 9.17 (d, J=0.8 Hz, 1H), 8.60 (d, J=2.4 Hz, 1H), 8.32 (d, J=0.8 Hz, 1H), 7.95-7.98 (m, 2H), 7.42 (d, J=8.8 Hz, 1H), 5.48 (s, 2H), 5.07-5.13 (m, 1H), 4.87-4.92 (m, 1H), 4.72-4.76 (m, 1H), 4.44-4.49 (m, 1H), 4.34-4.40 (m, 1H), 4.20 (d, J=13.6 Hz, 1H), 4.10 (d, J=13.6 Hz, 1H), 3.65-3.75 (m, 2H), 2.83-2.90 (m, 4H), 2.66-2.70 (m, 1H), 2.35-2.40 (m, 1H). ¹⁹F NMR (376 MHz, DMSO-d₆) δ −61.53-63.73. LC-MS: m/z 680.1 (M+H)⁺.

### Procedure 19

### 3-fluoro-4-[({7-[(3-{1[(2S)-oxetan-2-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridin-2-yl}oxy)methyl]benzonitrile (compound 75)

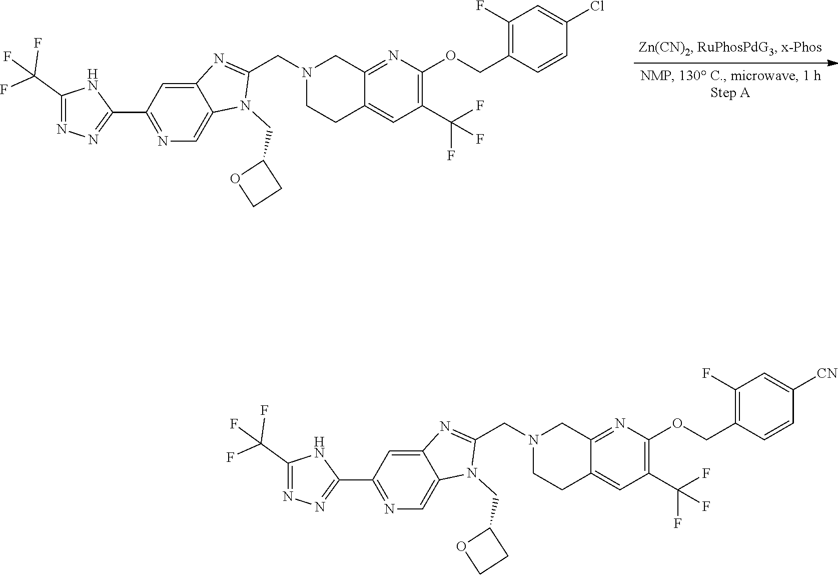 [SMILES: Fc1cc(Cl)ccc1COc1nc2c(cc1C(F)(F)F)CCN(Cc1nc3cc(-c4nnc(C(F)(F)F)[nH]4)ncc3n1C[C@@H]1CCO1)C2 ; N#Cc1ccc(COc2nc3c(cc2C(F)(F)F)CCN(Cc2nc4cc(-c5nnc(C(F)(F)F)[nH]5)ncc4n2C[C@@H]2CCO2)C3)c(F)c1]

A mixture of 2-[(4-chloro-2-fluorophenyl)methoxy]-7-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridine (100 mg, 0.144 mmol), Zn(CN)₂ (50 mg, 0.427 mmol), XPhos (7 mg, 0.0144 mmol), and RuPhos Pd G3 (12 mg, 0.0144 mmol) in NMP (1.5 mL) was stirred at 130° C. for 1 hr under microwave irradiation. The reaction mixture was filtered and the filtrate was purified directly by prep-HPLC (0.1% FA/H₂O/CH₃CN) to give 3-fluoro-4-[({7-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridin-2-yl}oxy)methyl]benzonitrile (21.21 mg, yield: 21.5%).

¹H NMR (400 MHz, DMSO-d₆) δ 9.17 (s, 1H), 8.32 (s, 1H), 7.95 (s, 1H), 7.89 (dd, J=10.0 Hz, 1.2 Hz, 1H), 7.74 (dd, J=8.0 Hz, 1.2 Hz, 1H), 7.66 (t, J=7.2 Hz, 1H), 5.53 (s, 2H), 5.07-5.13 (m, 1H), 4.85-4.95 (m, 1H), 4.72-4.77 (m, 1H), 4.34-4.52 (m, 2H), 4.21 (d, J=14.0 Hz, 1H), 4.10 (d, J=14.0 Hz, 1H), 3.68-3.78 (m, 2H), 2.77-2.93 (m, 4H), 2.62-2.71 (m, 1H), 2.35-2.44 (m, 1H). ¹⁹F NMR (376 MHz, DMSO-d₆) δ −61.64, −63.59, −115.21. LC-MS: m/z 688.3 (M+H)⁺.

### Procedure 20

### 2-[(4-chloro-2-fluorophenyl)methoxy]-7-[(3-{[(3R)-oxolan-3-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridine (compound 76)

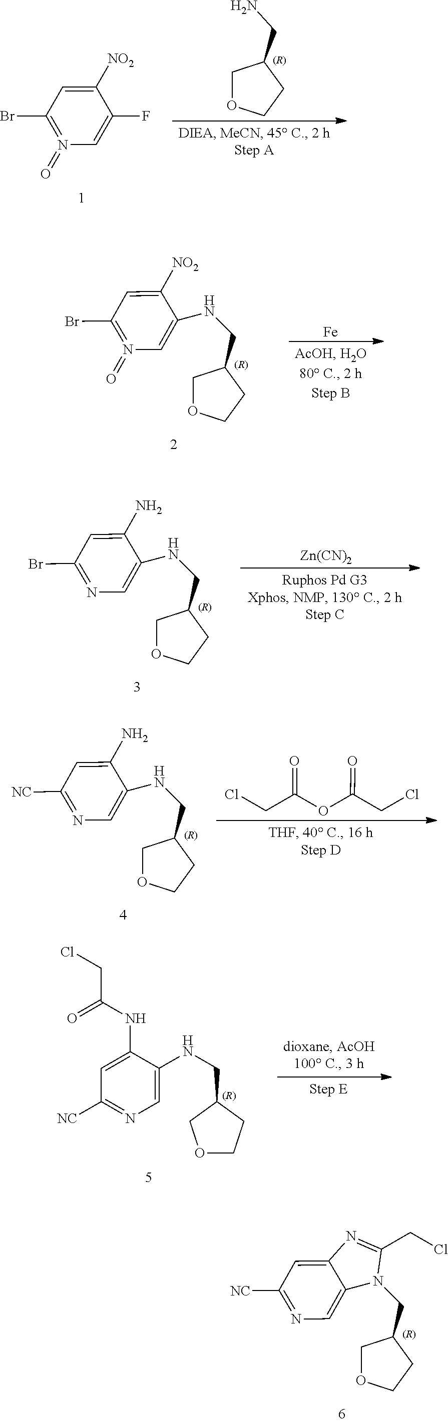

### Step A: (R)-2-bromo-4-nitro-5-(((tetrahydrofuran-3-yl)methyl)amino)pyridine 1-oxide

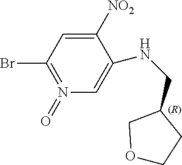

A mixture of 2-bromo-5-fluoro-4-nitropyridine 1-oxide (640 mg, 2.71 mmol), (R)-(tetrahydrofuran-3-yl)methanamine (274 mg, 2.71 mmol) and DIEA (1.05 g, 8.14 mmol) in MeCN (10 mL) was stirred at 45° C. for 2 hours. After the reaction was completed, the mixture was concentrated and purified by column chromatography on silica gel (PE/EA=4/1) to give (R)-2-bromo-4-nitro-5-(((tetrahydrofuran-3-yl)methyl)amino)pyridine 1-oxide (800 mg, yield: 93%). MS Calcd.: 317.00; MS Found: 318.1 [M+H]⁺.

### Step B: (R)-6-bromo-N3-((tetrahydrofuran-3-yl)methyl)pyridine-3,4-diamine

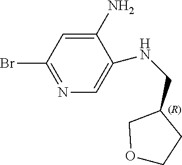

A mixture of (R)-2-bromo-4-nitro-5-(((tetrahydrofuran-3-yl)methyl)amino)pyridine 1-oxide (800 mg, 2.52 mmol) and Fe (1.41 g, 25.24 mmol) in AcOH (10 mL) and H₂O (10 mL) was stirred at 80° C. for 2 hours. The mixture was poured into sat. Na₂CO₃ and extracted with EtOAc (100 mL). The organic layer was filtered and purified by column chromatography on silica gel (DCM/MeOH=10/1) to give (R)-6-bromo-N3-((tetrahydrofuran-3-yl)methyl)pyridine-3,4-diamine (500 mg, yield: 73%). MS Calcd.: 271.03; MS Found: 272.3 [M+H]⁺.

### Step C: (R)-4-amino-5-(((tetrahydrofuran-3-yl)methyl)amino)picolinonitrile

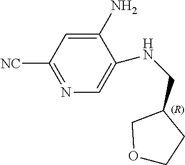

A mixture of (R)-6-bromo-N3-((tetrahydrofuran-3-yl)methyl)pyridine-3,4-diamine (420 mg, 1.55 mmol), Zn(CN)₂ (272 mg, 2.32 mmol), RuPhos Pd G3 (130 mg, 0.15 mmol), and XPhos (74 mg, 0.15 mmol) in NMP (8 mL) was stirred at 130° C. for 2 hours under Ar. The mixture was purified by reversed phase column chromatography (H₂O/MeCN=4/1) to give (R)-4-amino-5-(((tetrahydrofuran-3-yl)methyl)amino)picolinonitrile (100 mg, yield: 30%). MS Calcd.: 218.12; MS Found: 219.0 [M+H]⁺.

### Step D: (R)-2-chloro-N-(2-cyano-5-(((tetrahydrofuran-3-yl)methyl)amino)pyridin-4-yl)acetamide

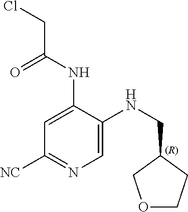

To a mixture of (R)-4-amino-5-(((tetrahydrofuran-3-yl)methyl)amino)picolinonitrile (100 mg, 0.46 mmol) in THF (6 mL) was added 2-chloroacetic anhydride (196 mg, 1.15 mmol) and stirred at 40° C. for 16 hours. The mixture was diluted with EtOAc (100 mL) and washed with sat. NaHCO₃ (100 mL), dried over Na₂SO₄, filtered, and concentrated to give (R)-2-chloro-N-(2-cyano-5-(((tetrahydrofuran-3-yl)methyl)amino)pyridin-4-yl)acetamide (100 mg, yield: 74%), which was used for the next step directly without further purification.

### Step E: (R)-2-(chloromethyl)-3-((tetrahydrofuran-3-yl)methyl)-3H-imidazo[4,5-c]pyridine-6-carbonitrile

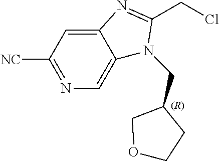

A mixture of (R)-2-chloro-N-(2-cyano-5-(((tetrahydrofuran-3-yl)methyl)amino)pyridin-4-yl)acetamide (100 mg, 0.34 mmol) in dioxane (8 mL) and AcOH (1 mL) was stirred at 100° C. for 3 hours. The mixture was poured into sat. Na₂CO₃, extracted with EtOAc (100 mL) and washed with brine (100 mL*3). The organic layer was separated and dried over Na₂SO₄, filtered, and purified by column chromatography on silica gel (PE/EA=1/1) to give (R)-2-(chloromethyl)-3-((tetrahydrofuran-3-yl)methyl)-3H-imidazo[4,5-c]pyridine-6-carbonitrile (70 mg, yield: 74%). MS Calcd.: 276.08; MS Found: 277.1 [M+H]⁺.

### 2-[(4-chloro-2-fluorophenyl)methoxy]-7-[(3-{[(3R)-oxolan-3-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridine (compound 76)

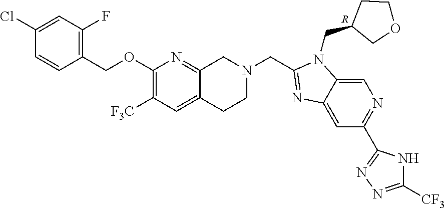

Compound 76 was then synthesized following the similar route of Procedure 3, using 2-((4-chloro-2-fluorobenzyl)oxy)-3-(trifluoromethyl)-5,6,7,8-tetrahydro-1,7-naphthyridine and (R)-2-(chloromethyl)-3-((tetrahydrofuran-3-yl)methyl)-3H-imidazo[4,5-c]pyridine-6-carbonitrile in step B.

¹H NMR (400 MHz, DMSO-d₆) δ 15.39 (s, 1H), 9.18 (s, 1H), 8.34 (s, 1H), 7.93 (s, 1H), 7.51 (t, J=8.0 Hz, 1H), 7.45 (dd, J=10.0 Hz, 1.6 Hz, 1H), 7.30 (dd, J=8.0 Hz, 1.2 Hz, 1H), 5.43 (s, 2H), 4.52 (d, J=7.6 Hz, 2H), 4.12-4.18 (m, 2H), 3.76-3.84 (m, 3H), 3.57-3.64 (m, 2H), 3.48-3.52 (m, 1H), 2.88-3.00 (m, 3H), 2.78-2.82 (m, 2H), 1.82-1.90 (m, 1H), 1.66-1.74 (m, 1H). ¹⁹F NMR (376 MHz, DMSO-d₆) δ −61.69, −63.68, −115.00. LC-MS: m/z 711.2 (M+H)⁺.

### 2-[(4-chloro-2-fluorophenyl)methoxy]-7-[(3-{1[(3S)-oxolan-3-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridine (compound 77)

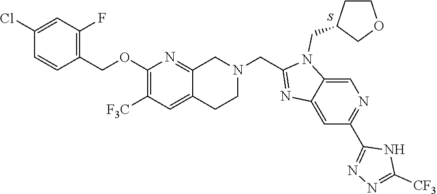

Compound 77 was synthesized following the similar route of Procedure 20, using (S)-(tetrahydrofuran-3-yl)methanamine in step A.

¹H NMR (400 MHz, DMSO-d₆) δ 15.27 (s, 1H), 9.18 (s, 1H), 8.34 (s, 1H), 7.93 (s, 1H), 7.51 (t, J=8.0 Hz, 1H), 7.45 (d, J=10.0 Hz, 1H), 7.30 (d, J=8.4 Hz, 1H), 5.43 (s, 2H), 4.52 (d, J=7.6 Hz, 2H), 4.14 (t, J=15.2 Hz, 2H), 3.76-3.84 (m, 3H), 3.57-3.64 (m, 2H), 3.48-3.52 (m, 1H), 2.93-3.00 (m, 1H), 2.88-2.91 (m, 2H), 2.81-2.82 (m, 2H), 1.82-1.90 (m, 1H), 1.65-1.74 (m, 1H). ¹⁹F NMR (376 MHz, DMSO-d₆) δ −61.68, −63.62, −114.99. LC-MS: m/z 711.2 (M+H)⁺.

### 2-[(4-chloro-2-fluorophenyl)methoxy]-7-[(3-{1[(2R)-oxolan-2-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridine (compound 78)

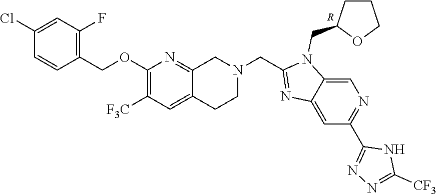

Compound 78 was synthesized following the similar route of Procedure 20, using (R)-(tetrahydrofuran-2-yl)methanamine in step A.

¹H NMR (400 MHz, DMSO-d₆) δ 15.43 (s, 1H), 9.12 (s, 1H), 8.32 (s, 1H), 7.93 (s, 1H), 7.51 (t, J=8.4 Hz, 1H), 7.46 (dd, J=10.0 Hz, 2.0 Hz, 1H), 7.31 (dd, J=8.4 Hz, 1.6 Hz, 1H), 5.42 (s, 2H), 4.66 (dd, J=14.8 Hz, 2.8 Hz, 1H), 4.54 (dd, J=14.8 Hz, 8.4 Hz, 1H), 4.22-4.28 (m, 2H), 4.08 (d, J=14.0 Hz, 1H), 3.68-3.83 (m, 3H), 3.60-3.65 (m, 1H), 2.82-2.87 (m, 4H), 1.97-2.05 (m, 1H), 1.74-1.91 (m, 2H), 1.57-1.66 (m, 1H). ¹⁹F NMR (376 MHz, DMSO-d₆) δ −61.69, −63.74, −115.00. LC-MS: m/z 711.2 (M+H)⁺.

### 2-[(4-chloro-2-fluorophenyl)methoxy]-7-[(3-{1[(2S)-oxolan-2-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridine (compound 79)

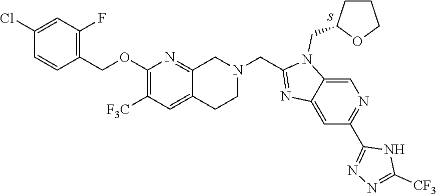

Compound 79 was synthesized following the similar route of Procedure 20, using (S)-(tetrahydrofuran-2-yl)methanamine in step A.

¹H NMR (400 MHz, DMSO-d₆) δ 15.44 (s, 1H), 9.12 (s, 1H), 8.32 (s, 1H), 7.93 (s, 1H), 7.51 (t, J=8.4 Hz, 1H), 7.46 (dd, J=10.0 Hz, 2.0 Hz, 1H), 7.31 (dd, J=8.4 Hz, 1.6 Hz, 1H), 5.42 (s, 2H), 4.66 (dd, J=14.8 Hz, 2.8 Hz, 1H), 4.54 (dd, J=14.8 Hz, 8.4 Hz, 1H), 4.22-4.25 (m, 2H), 4.08 (d, J=14.0 Hz, 1H), 3.72-3.83 (m, 3H), 3.60-3.65 (m, 1H), 2.82-2.87 (m, 4H), 1.99-2.03 (m, 1H), 1.77-1.86 (m, 2H), 1.61-1.66 (m, 1H). ¹⁹F NMR (376 MHz, DMSO-d₆) δ −61.69, −63.74, −115.00. LC-MS: m/z 711.2 (M+H)⁺.

### 2-[(4-chloro-2-fluorophenyl)methoxy]-7-{[3-(2-methoxyethyl)-6-[5-(trifluoromethyl)-4H-1,2,4-triazol-3-yl]-3H-imidazo[4,5-c]pyridin-2-yl]methyl}-3-(trifluoromethyl)-5,6,7,8-tetrahydro-1,7-naphthyridine (compound 80)

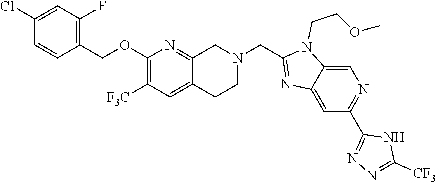 [SMILES: COCCn1c(CN2CCc3cc(C(F)(F)F)c(OCc4ccc(Cl)cc4F)nc3C2)nc2cc(-c3nnc(C(F)(F)F)[nH]3)ncc21]

Compound 80 was synthesized following the similar route of Procedure 20, using 2-methoxyethan-1-amine in step A.

¹H NMR (400 MHz, DMSO-d₆) δ 15.44 (s, 1H), 9.11 (s, 1H), 8.32 (s, 1H), 7.93 (s, 1H), 7.51 (t, J=8.0 Hz, 1H), 7.46 (dd, J=10.0 Hz, 2.0 Hz, 1H), 7.31 (dd, J=8.0 Hz, 1.6 Hz, 1H), 5.42 (s, 2H), 4.71 (t, J=4.8 Hz, 2H), 4.15 (s, 2H), 3.73-3.75 (m, 4H), 3.19 (s, 3H), 2.82-2.86 (m, 4H). ¹⁹F NMR (376 MHz, DMSO-d₆) δ −61.69, −63.72, −115.02. LC-MS: m/z 685.1 (M+H)⁺.

### 2-[(4-chloro-2-fluorophenyl)methoxy]-7-({3-[(2S)-2-methoxypropyl]-6-[5-(trifluoromethyl)-4H-1,2,4-triazol-3-yl]-3H-imidazo[4,5-c]pyridin-2-yl}methyl)-3-(trifluoromethyl)-5,6,7,8-tetrahydro-1,7-naphthyridine (compound 81)

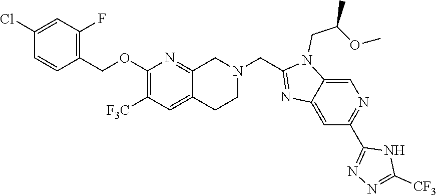 [SMILES: CO[C@H](C)Cn1c(CN2CCc3cc(C(F)(F)F)c(OCc4ccc(Cl)cc4F)nc3C2)nc2cc(-c3nnc(C(F)(F)F)[nH]3)ncc21]

Compound 81 was synthesized following the similar route of Procedure 20, using (S)-3-methoxybutan-1-amine hydrochloride in step A.

¹H NMR (400 MHz, DMSO-d₆) δ 15.38 (s, 1H), 9.10 (d, J=0.8 HZ 1H), 8.32 (d, J=0.8 HZ, 1H), 7.93 (s, 1H), 7.51 (t, J=8.0 Hz, 1H), 7.45 (dd, J=10.0 Hz, 2.0 Hz, 1H), 7.30 (dd, J=8.4 Hz, 2 Hz, 1H), 5.42 (s, 2H), 4.45-4.60 (m, 2H), 4.25 (d, J=13.6 HZ, 1H), 4.06 (d, J=13.6 HZ, 1H), 3.66-3.81 (m, 3H), 3.06 (s, 3H), 2.82-2.87 (m, 4H), 1.15 (d, J=6.4 HZ, 3H). ¹⁹F NMR (376 MHz, DMSO-d₆) δ −61.69, −63.69, −115.01. LC-MS: m/z 699.3 (M+H)⁺.

### Procedure 21

### 2-[(4-chloro-2-fluorophenyl)methoxy]-7-[1-(3-{[(2S)-oxetan-2-yl]methyl}-6-[5-(trifluoromethyl)-4H-1,2,4-triazol-3-yl]-3H-imidazo[4,5-c]pyridin-2-yl)ethyl]-3-(trifluoromethyl)-5,6,7,8-tetrahydro-1,7-naphthyridine (compound 82)

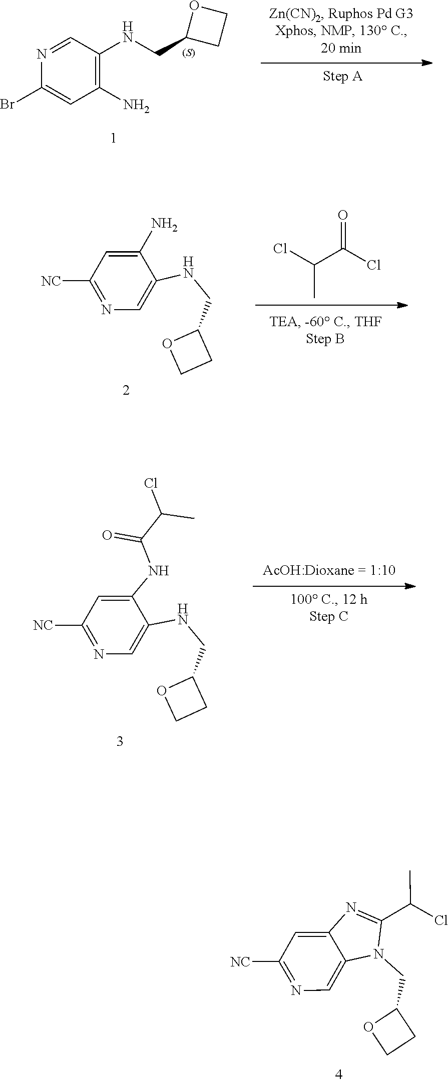

### Step A: (S)-4-amino-5-((oxetan-2-ylmethyl)amino)picolinonitrile

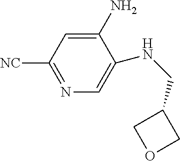

A mixture of (S)-6-bromo-N3-(oxetan-2-ylmethyl)pyridine-3,4-diamine (5 g, 19.46 mmol), Zn(CN)₂ (3.4 g, 29.18 mmol), RuPhos Pd G3 (1.6 g, 1.95 mmol), and XPhos (928 mg, 1.95 mmol) in NMP (30 mL) was stirred at 130° C. for 20 mins under Ar. The mixture was purified by reversed phase column chromatography (H₂O/MeCN=4/1) to give (S)-4-amino-5-((oxetan-2-ylmethyl)amino)picolinonitrile (1.2 g, yield: 30%). MS Calcd.: 204.1; MS Found: 205.1 [M+H]⁺.

### Step B: 2-chloro-N-(2-cyano-5-((((S)-oxetan-2-yl)methyl)amino)pyridin-4-yl)propanamide

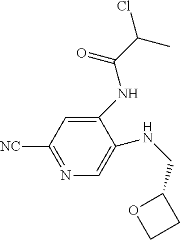 [SMILES: CC(Cl)C(=O)Nc1cc(C#N)ncc1NC[C@@H]1CCO1]

To a mixture of (S)-4-amino-5-((oxetan-2-ylmethyl)amino)picolinonitrile (1.2 g, 5.88 mmol) and TEA (1.78 g, 17.65 mmol) in THF (20 mL) was added 2-chloropropanoyl chloride (371 mg, 2.94 mmol) and the reaction mixture was stirred at −60° C. for 0.5 hours. The mixture was diluted with EtOAc (100 mL) and washed with sat. NaHCO₃ (100 mL), dried over Na₂SO₄, filtered, and concentrated to give 2-chloro-N-(2-cyano-5-((((S)-oxetan-2-yl)methyl)amino)pyridin-4-yl)propanamide (850 mg, yield: 49%) which was used in the next step directly. MS Calcd.: 294.1; MS Found: 295.1 [M+H]⁺.

### Step C: 2-(1-chloroethyl)-3-(((S)-oxetan-2-yl)methyl)-3H-imidazo[4,5-c]pyridine-6-carbonitrile

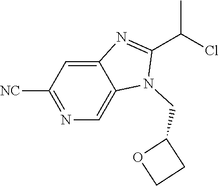 [SMILES: CC(Cl)c1nc2cc(C#N)ncc2n1C[C@@H]1CCO1]

A mixture of 2-chloro-N-(2-cyano-5-((((S)-oxetan-2-yl)methyl)amino)pyridin-4-yl)propanamide (850 mg, 2.89 mmol) in dioxane (8 mL) and AcOH (1 mL) was stirred at 100° C. for 12 hours. The mixture was poured into sat. Na₂CO₃, extracted with EtOAc (100 mL) and washed with brine (100 mL*3). The organic layer was separated and dried over Na₂SO₄, filtered, and purified by column chromatography on silica gel (PE/EA=1/1) to give 2-(1-chloroethyl)-3-(((S)-oxetan-2-yl)methyl)-3H-imidazo[4,5-c]pyridine-6-carbonitrile (600 mg, yield: 75%). MS Calcd.: 276.1; MS Found: 277.1 [M+H]⁺.

### 2-[(4-chloro-2-fluorophenyl)methoxy]-7-[l-(3-{[(2S)-oxetan-2-yl]methyl}-6-[5-(trifluoromethyl)-4H-1,2,4-triazol-3-yl]-3H-imidazo[4,5-c]pyridin-2-yl)ethyl]-3-(trifluoromethyl)-5,6,7,8-tetrahydro-1,7-naphthyridine (compound 82)

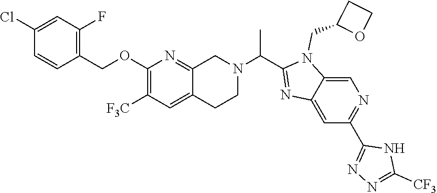 [SMILES: CC(c1nc2cc(-c3nnc(C(F)(F)F)[nH]3)ncc2n1C[C@@H]1CCO1)N1CCc2cc(C(F)(F)F)c(OCc3ccc(Cl)cc3F)nc2C1]

Compound 82 was then synthesized following the similar route of Procedure 3, using 2-((4-chloro-2-fluorobenzyl)oxy)-3-(trifluoromethyl)-5,6,7,8-tetrahydro-1,7-naphthyridine and 2-(1-chloroethyl)-3-(((S)-oxetan-2-yl)methyl)-3H-imidazo[4,5-c]pyridine-6-carbonitrile in step B.

¹H NMR (400 MHz, DMSO-d₆) δ 15.44 (s, 1H), 9.21 (s, 1H), 8.36 (s, 1H), 7.92 (s, 1H), 7.51 (t, J=8.0 Hz, 1H), 7.44 (dd, J=10.0 Hz, 2.0 Hz, 1H), 7.29 (dd, J=8.0 Hz, 1.6 Hz, 1H), 5.41 (s, 2H), 5.11-5.17 (m, 1H), 4.82-4.84 (m, 3H), 4.40-4.46 (m, 1H), 4.20-4.25 (m, 1H), 3.85-3.93 (m, 1H), 2.90-3.05 (m, 2H), 2.79-2.83 (m, 2H), 2.57-2.65 (m, 2H), 2.27-2.34 (m, 1H), 1.63 (d, J=6.0 Hz, 3H). ¹⁹F NMR (376 MHz, DMSO-d₆) δ −61.71, −63.70, −114.97. LC-MS: m/z 711.2 (M+H)⁺.

### 2-[(4-chloro-2,6-difluorophenyl)methoxy]-7-[1-(3-{1[(2S)-oxetan-2-yl]methyl}-6-[5-(trifluoromethyl)-4H-1,2,4-triazol-3-yl]-3H-imidazo[4,5-c]pyridin-2-yl)ethyl]-3-(trifluoromethyl)-5,6,7,8-tetrahydro-1,7-naphthyridine (compound 83)

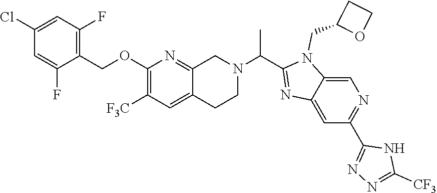 [SMILES: CC(c1nc2cc(-c3nnc(C(F)(F)F)[nH]3)ncc2n1C[C@@H]1CCO1)N1CCc2cc(C(F)(F)F)c(OCc3c(F)cc(Cl)cc3F)nc2C1]

Compound 83 was synthesized following the similar route of Procedure 3, using 2-((4-chloro-2,6-difluorobenzyl)oxy)-3-(trifluoromethyl)-5,6,7,8-tetrahydro-1,7-naphthyridine and 2-(1-chloroethyl)-3-(((S)-oxetan-2-yl)methyl)-3H-imidazo[4,5-c]pyridine-6-carbonitrile in step B.

¹H NMR (400 MHz, DMSO-d₆) δ 15.39 (s, 1H), 9.17 (d, J=18.4 Hz, 1H), 8.35 (d, J=6.4 Hz, 1H), 7.88 (d, J=11.2 Hz, 1H), 7.36 (t, J=6.8 Hz, 2H), 5.32-5.42 (m, 2H), 5.13-5.19 (m, 0.5H), 4.94-5.04 (m, 1H), 4.80-4.86 (m, 1H), 4.69-4.76 (m, 1H), 4.42-4.49 (m, 1H), 4.20-4.25 (m, 0.5H), 3.83 (t, J=16.4 Hz, 1H), 3.67 (t, J=15.2 Hz, 1H), 2.56-2.94 (m, 6H), 2.24-2.44 (m, 1H), 1.59 (t, J=4.8 Hz, 3H). ¹⁹F NMR (376 MHz, DMSO-d₆) δ −61.77, −63.70, −112.07. LC-MS: m/z 729.2 (M+H)⁺.

### 5-[2-(1-{2-[(4-chloro-2,6-difluorophenyl)methoxy]-3-(trifluoromethyl)-5,6,7,8-tetrahydro-1,7-naphthyridin-7-yl}ethyl)-3-{1[(2S)-oxetan-2-yl]methyl}-3H-imidazo[4,5-c]pyridin-6-yl]-4H-1,2,4-triazole-3-carbonitrile (compound 84)

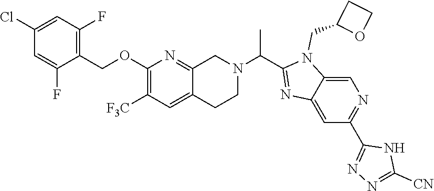 [SMILES: CC(c1nc2cc(-c3nnc(C#N)[nH]3)ncc2n1C[C@@H]1CCO1)N1CCc2cc(C(F)(F)F)c(OCc3c(F)cc(Cl)cc3F)nc2C1]

Compound 84 was synthesized following the similar route of Procedure 5, using 2-((4-chloro-2,6-difluorobenzyl)oxy)-3-(trifluoromethyl)-5,6,7,8-tetrahydro-1,7-naphthyridine and 2-(1-chloroethyl)-3-(((S)-oxetan-2-yl)methyl)-3H-imidazo[4,5-c]pyridine-6-carbonitrile in step B.

¹H NMR (400 MHz, DMSO-d₆) δ 15.74 (s, 1H), 9.17 (d, J=18.4 Hz, 1H), 8.37 (d, J=6.8 Hz, 1H), 7.88 (d, J=11.6 Hz, 1H), 7.35-7.38 (m, 2H), 5.15-5.39 (m, 3H), 4.95-4.99 (m, 1H), 4.81-4.82 (m, 1H), 4.71-4.76 (m, 1H), 4.20-4.49 (m, 2H), 3.63-3.82 (m, 3H), 2.54-2.92 (m, 4H), 2.40-2.44 (m, 1H), 1.57-1.60 (m, 3H). ¹⁹F NMR (376 MHz, DMSO-d₆) δ −61.77-112.08. LC-MS: m/z 686.2 (M+H)⁺.

### Procedure 22

### 2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-1H-1,3-benzodiazole-5-carboxylic acid (compound 28) and 2-[(4-chloro-2-fluorophenyl)methoxy]-7-[(3-{[(2S)-oxetan-2-yl]methyl}-6-(1H-1,2,3,4-tetrazol-5-yl)-3H-imidazo[4,5-c]pyridin-2-yl)methyl]-3-(trifluoromethyl)-5,6,7,8-tetrahydro-1,7-naphthyridine (compound 85)

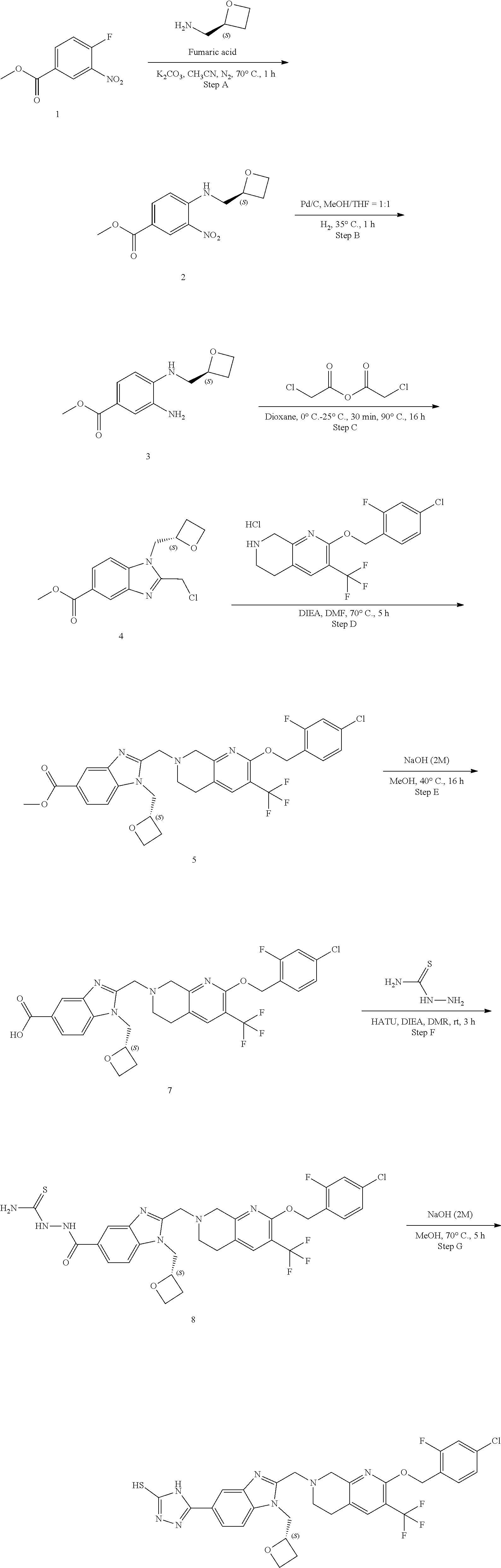

### Step A: methyl (S)-3-nitro-4-((oxetan-2-ylmethyl)amino)benzoate

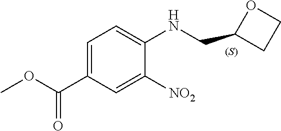

A mixture of methyl 4-fluoro-3-nitrobenzoate (6.00 g, 30.13 mmol), (S)-oxetan-2-ylmethanamine (5.68 g, 39.17 mmol, fumaric acid salt), and K₂CO₃ (10.39 g, 75.32 mmol) in ACN (25 mL) was stirred at 70° C. for 1 hour. Then the mixture was cooled down to room temperature. The mixture was filtered and the filtrate was concentrated under reduced pressure. The residue was purified by silica gel column chromatography, eluted with PE/EtOAc (1/1) to give methyl (S)-3-nitro-4-((oxetan-2-ylmethyl)amino)benzoate (7.93 g, yield: 98.9%). LC-MS: m/z 267.4 (M+H)⁺.

### Step B: methyl (S)-3-amino-4-((oxetan-2-ylmethyl)amino)benzoate

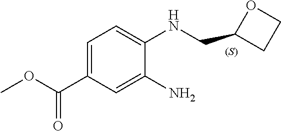

A mixture of methyl (S)-3-nitro-4-((oxetan-2-ylmethyl)amino)benzoate (7.93 g, 29.80 mmol) and Pd/C (2.64 g, 10%, 50% wet with water) in MeOH (40 mL) and THF (40 mL) was stirred at 35° C. for 1 hour under H₂ (1 atm). Then the mixture was cooled down to room temperature. The mixture was filtered and the filtrate was concentrated to give methyl (S)-3-amino-4-((oxetan-2-ylmethyl)amino)benzoate (6.81 g, yield: 96.8%). LC-MS: m/z 237.4 (M+H)⁺.

### Step C: methyl (S)-2-(chloromethyl)-1-(oxetan-2-ylmethyl)-1H-benzo[d]imidazole-5-carboxylate

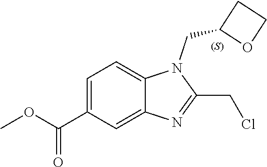

To a solution of methyl (S)-3-amino-4-((oxetan-2-ylmethyl)amino)benzoate (6.81 g, 28.84 mmol) in dioxane (100 mL) was added 2-chloroacetic anhydride (4.93 g, 28.84 mmol) at 0° C. The solution was stirred at 25° C. for 30 minutes, then the solution was stirred at 90° C. for 16 hours. The mixture was cooled down to room temperature and quenched with NaHCO₃ (150 mL, eq), and extracted with EtOAc (3×100 mL). The combined organic layers were washed with brine (80 mL), dried over anhydrous Na₂SO₄, filtered, and concentrated under reduced pressure. The residue was purified by silica gel column chromatography, eluted with DCM/MeOH (45/1) to give methyl (S)-2-(chloromethyl)-1-(oxetan-2-ylmethyl)-1H-benzo[d]imidazole-5-carboxylate (6.24 g, yield: 73.6%). LC-MS: m/z 295.2 (M+H)⁺.

### Step D: methyl (S)-2-((2-((4-chloro-2-fluorobenzyl)oxy)-3-(trifluoromethyl)-5,8-dihydro-1,7-naphthyridin-7(61H)-yl)methyl)-1-(oxetan-2-ylmethyl)-1H-benzo[d]imidazole-5-carboxylate

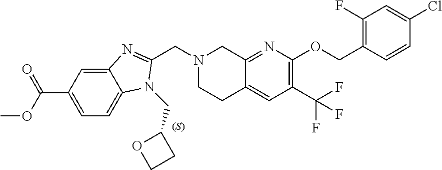

To a solution of 2-((4-chloro-2-fluorobenzyl)oxy)-3-(trifluoromethyl)-5,6,7,8-tetrahydro-1,7-naphthyridine, HCl salt (735 mg, 1.85 mmol) and DIEA (2.35 mL, 13.50 mmol) in DMF (10 mL) was added methyl (S)-2-(chloromethyl)-1-(oxetan-2-ylmethyl)-1H-benzo[d]imidazole-5-carboxylate (496 mg, 1.69 mmol). The solution was stirred at 70° C. for 5 hours. The solution was cooled down to room temperature, quenched with H₂O (50 mL), and extracted with EtOAc (3×50 mL). The combined organic layers were washed with brine (40 mL), dried over Na₂SO₄, filtered, and concentrated under reduced pressure. The residue was purified by silica gel column chromatography, eluted with DCM/MeOH (20/1) to give methyl (S)-2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-benzo[d]imidazole-5-carboxylate (866 mg, yield: 83.1%). LC-MS: m/z 619.2 (M+H)⁺.

### Step E: 2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-1H-1,3-benzodiazole-5-carboxylic acid (compound 28)

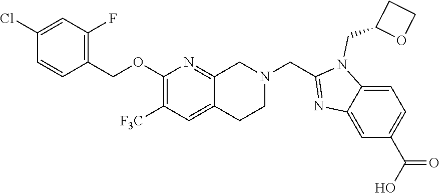 [SMILES: O=C(O)c1ccc2c(c1)nc(CN1CCc3cc(C(F)(F)F)c(OCc4ccc(Cl)cc4F)nc3C1)n2C[C@@H]1CCO1]

To a solution of methyl (S)-2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-benzo[d]imidazole-5-carboxylate (866 mg, 1.40 mmol) in MeOH (2.0 mL) was added NaOH (2.10 mL, 4.20 mmol, 2 M). The solution was stirred at 40° C. for 16 hours. The solution was cooled down to room temperature, quenched with H₂O (40 mL), and extracted with DCM/MeOH=15:1 (3×30 mL). The combined organic layers were washed with brine (30 mL), dried over Na₂SO₄, filtered, and concentrated under reduced pressure. The residue was purified by silica gel column chromatography, eluted with PE/EtOAc (3/1) to give 2-({2-[(4-chloro-2-fluorophenyl)methoxy]-3-(trifluoromethyl)-5,6,7,8-tetrahydro-1,7-naphthyridin-7-yl}methyl)-1-1{[(2S)-oxetan-2-yl]methyl}-1H-1,3-benzodiazole-5-carboxylic acid (820 mg, yield: 96.9%).

¹H NMR (400 MHz, DMSO-d₆) δ 8.21 (s, 1H), 7.93 (s, 1H), 7.87 (d, J=8.4 Hz, 1H), 7.69 (d, J=8.4 Hz, 1H), 7.42-7.56 (m, 2H), 7.31 (dd, J=8.0, 2.0 Hz, 1H), 5.41 (s, 2H), 5.00-5.11 (m, 1H), 4.74 (dd, J=15.2, 7.2 Hz, 1H), 4.60 (dd, J=15.6, 2.8 Hz, 1H), 4.40-4.49 (m, 1H), 4.30-4.39 (m, 1H), 4.14 (d, J=13.6 Hz, 1H), 4.02 (d, J=13.6 Hz, 1H), 3.61-3.75 (m, 2H), 2.79-2.90 (m, 4H), 2.57-2.65 (m, 1H), 2.29-2.41 (m, 1H). ¹⁹F NMR (376 MHz, DMSO-d₆) δ −61.70, −114.95. LC-MS: m/z 605.5 (M+H)⁺.

### Step F: (S)-2-(2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-benzo[d]imidazole-5-carbonyl)hydrazine-1-carbothioamide

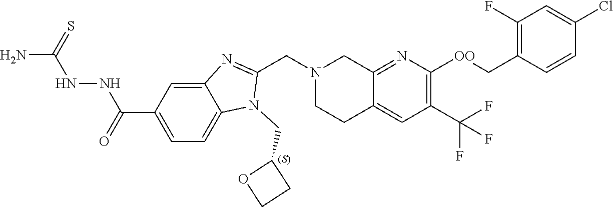

A solution of (S)-2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-benzo[d]imidazole-5-carboxylic acid (820 mg, 1.36 mmol), hydrazinecarbothioamide (185 mg, 2.04 mmol), HATU (775 mg, 2.04 mmol), and DIEA (0.71 mL, 4.07 mmol) in DMF (15 mL) was stirred at room temperature for 3 hours. The solution was quenched with H₂O (40 mL) and extracted with DCM/MeOH=15:1 (3×30 mL). The combined organic layers were washed with brine (30 mL), dried over Na₂SO₄, filtered, and concentrated under reduced pressure. The residue was purified by silica gel column chromatography, eluted with DCM/MeOH (10/1) to give (S)-2-(2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-benzo[d]imidazole-5-carbonyl)hydrazine-1-carbothioamide (398 mg, yield: 43.3%). LC-MS: m/z 678.3 (M+H)⁺.

### Step G: 5-[2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-1H-1,3-benzodiazol-5-yl]-4H-1,2,4-triazole-3-thiol (compound 85)

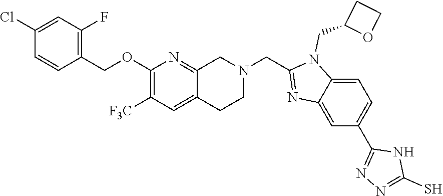 [SMILES: Fc1cc(Cl)ccc1COc1nc2c(cc1C(F)(F)F)CCN(Cc1nc3cc(-c4nnc(S)[nH]4)ccc3n1C[C@@H]1CCO1)C2]

To a solution of(S)-2-(2-((2-((4-chloro-2-fluorobenzyl)oxy)-3-(trifluoromethyl)-5,8-dihydro-1,7-naphthyridin-7(6H)-yl)methyl)-1-(oxetan-2-ylmethyl)-1H-benzo[d]imidazole-5-carbonyl)hydrazine-1-carbothioamide (30 mg, 0.04 mmol) in MeOH (2 mL) was added NaOH (0.05 mL, 0.09 mmol, 2 M). The solution was stirred at 70° C. for 5 hours. Then the solution was cooled down to room temperature, filtered, and the filtrate was purified by prep-HPLC (0.1% FA/H₂O/CH₃CN) to give 5-[2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-1H-1,3-benzodiazol-5-yl]-4H-1,2,4-triazole-3-thiol (1.12 mg, yield: 3.8%).

¹H NMR (400 MHz, DMSO-d₆) δ 13.58 (br s, 1H), 8.20 (s, 1H), 7.93 (s, 1H), 7.76-7.83 (m, 2H), 7.52 (t, J=8.0 Hz, 1H), 7.46 (dd, J=10.0, 1.6 Hz, 1H), 7.31 (d, J=8.4 Hz, 1H), 5.42 (s, 2H), 5.01-5.10 (m, 1H), 4.71-4.78 (m, 1H), 4.57-4.64 (m, 1H), 4.39-4.49 (m, 1H), 4.31-4.36 (m, 1H), 4.14 (d, J=13.2 Hz, 1H), 4.03 (d, J=13.2 Hz, 1H), 3.62-3.75 (m, 2H), 2.78-2.90 (m, 4H), 2.59-2.66 (m, 1H), 2.32-2.42 (m, 1H). ¹⁹F NMR (376 MHz, DMSO-d₆) δ −61.68, −114.96. LC-MS: m/z 660.1 (M+H)⁺.

### Procedure 23

### 2-[(4-chloro-2-fluorophenyl)methoxy]-7-({5-[5-(methylsulfanyl)-4H-1,2,4-triazol-3-yl]-1-{[(2S)-oxetan-2-yl]methyl}-1H-1,3-benzodiazol-2-yl}methyl)-3-(trifluoromethyl)-5,6,7,8-tetrahydro-1,7-naphthyridine (compound 86)

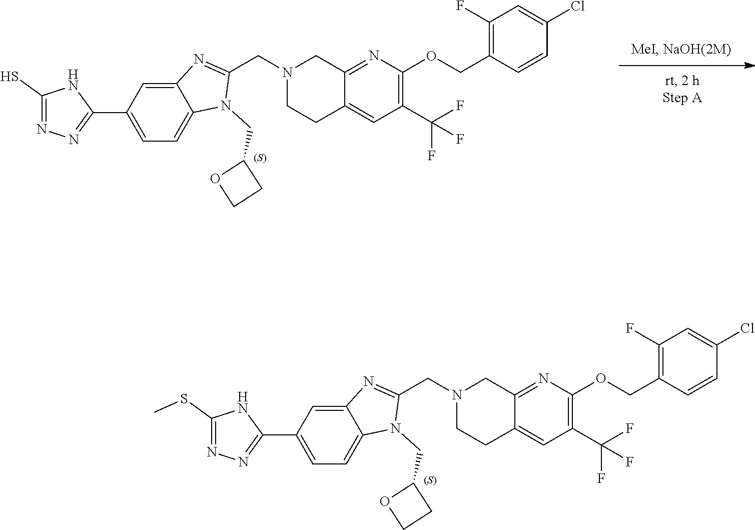

To a solution of 5-[2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-1H-1,3-benzodiazol-5-yl]-4H-1,2,4-triazole-3-thiol (20 mg, 0.03 mmol) and NaOH (0.03 mL, 0.06 mmol, 2 mol in H₂O) in MeOH (2 mL) was added Mel (8.6 mg, 0.03 mmol). The solution was stirred at room temperature for 2 hours. The solution was filtered and the filtrate was purified by prep-HPLC (0.1% FA/H₂O/CH₃CN) to give 2-[(4-chloro-2-fluorophenyl)methoxy]-7-({5-[5-(methylsulfanyl)-4H-1,2,4-triazol-3-yl]-1-{[(2S)-oxetan-2-yl]methyl}-1H-1,3-benzodiazol-2-yl}methyl)-3-(trifluoromethyl)-5,6,7,8-tetrahydro-1,7-naphthyridine (7.93 mg, yield: 38.8%).

¹H NMR (400 MHz, DMSO-d₆) δ 14.32 (br s, 1H), 8.22 (s, 1H), 7.93 (s, 1H), 7.77-7.88 (m, 2H), 7.51 (t, J=8.0 Hz, 1H), 7.46 (dd, J=10.0, 2.0 Hz, 1H), 7.31 (dd, J=8.4, 1.6 Hz, 1H), 5.42 (s, 2H), 5.02-5.10 (m, 1H), 4.70-4.79 (m, 1H), 4.56-4.65 (m, 1H), 4.42-4.50 (m, 1H), 4.32-4.39 (m, 1H), 4.01-4.22 (m, 2H), 3.62-3.75 (m, 2H), 2.83-2.96 (m, 4H), 2.59-2.71 (m, 1H), 2.58 (s, 3H), 2.30-2.45 (m, 1H). ¹⁹F NMR (376 MHz, DMSO-d₆) δ −61.69, −114.96. LC-MS: m/z 674.2 (M+H)⁺.

### Procedure 24

### 2-[(4-chloro-2-fluorophenyl)methoxy]-7-{[5-(5-methanesulfonyl-4H-1,2,4-triazol-3-yl)-1-{[(2S)-oxetan-2-yl]methyl}-1H-1,3-benzodiazol-2-yl]methyl}-3-(trifluoromethyl)-5,6,7,8-tetrahydro-1,7-naphthyridine (compound 87)

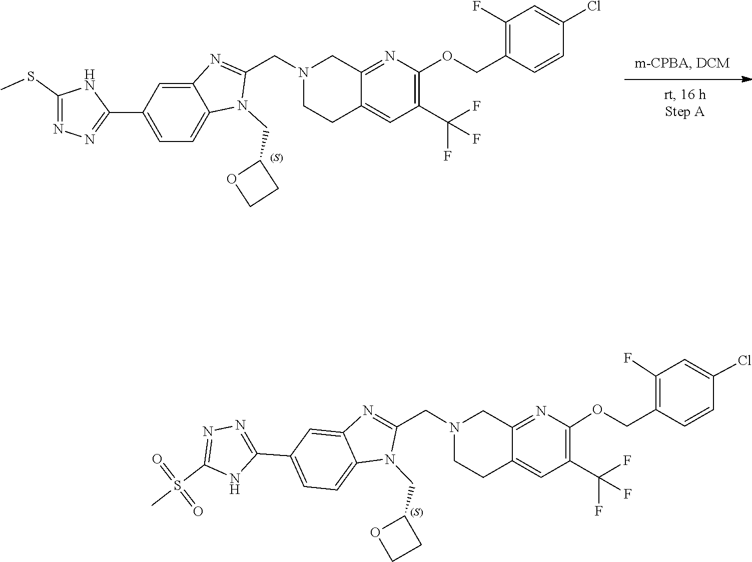

To a solution of 2-[(4-chloro-2-fluorophenyl)methoxy]-7-({5-[5-(methylsulfanyl)-4H-1,2,4-triazol-3-yl]-1-{[(2S)-oxetan-2-yl]methyl}-1H-1,3-benzodiazol-2-yl}methyl)-3-(trifluoromethyl)-5,6,7,8-tetrahydro-1,7-naphthyridine (73 mg, 0.11 mmol) in DCM (5 mL) was added m-CPBA (49 mg, 0.28 mmol). The solution was stirred at room temperature for 16 hours. The mixture was concentrated and purified by prep-HPLC (0.10% FA/H₂O/CH₃CN) to give 2-[(4-chloro-2-fluorophenyl)methoxy]-7-{[5-(5-methanesulfonyl-4H-1,2,4-triazol-3-yl)-1-{[(2S)-oxetan-2-yl]methyl}-1H-1,3-benzodiazol-2-yl]methyl}-3-(trifluoromethyl)-5,6,7,8-tetrahydro-1,7-naphthyridine (19.39 mg, yield: 25.4%).

¹H NMR (400 MHz, DMSO-d₆) δ 8.38 (s, 1H), 7.95-8.03 (m, 2H), 7.85 (d, J=8.0 Hz, 1H), 7.45-7.56 (m, 2H), 7.29-7.35 (m, 1H), 5.37-5.50 (m, 2H), 4.96-5.21 (m, 6H), 4.45-4.53 (m, 1H), 4.37-4.44 (m, 1H), 4.16-4.35 (m, 1H), 3.69-3.90 (m, 2H), 3.19-3.25 (m, 1H), 3.02 (s, 3H), 2.84-2.97 (m, 1H), 2.64-2.75 (m, 1H), 2.30-2.43 (m, 1H). ¹⁹F NMR (376 MHz, DMSO-d₆) δ −61.80, −114.94. LC-MS: m/z 706.2 (M+H)⁺.

### Procedure 25

### 2-[(4-chloro-2-fluorophenyl)methoxy]-7-[(4-fluoro-1-{[(2S)-oxetan-2-yl]methyl}-5-(1H-1,2,3,4-tetrazol-5-yl)-1H-1,3-benzodiazol-2-yl)methyl]-3-(trifluoromethyl)-5,6,7,8-tetrahydro-1,7-naphthyridine (compound 88)

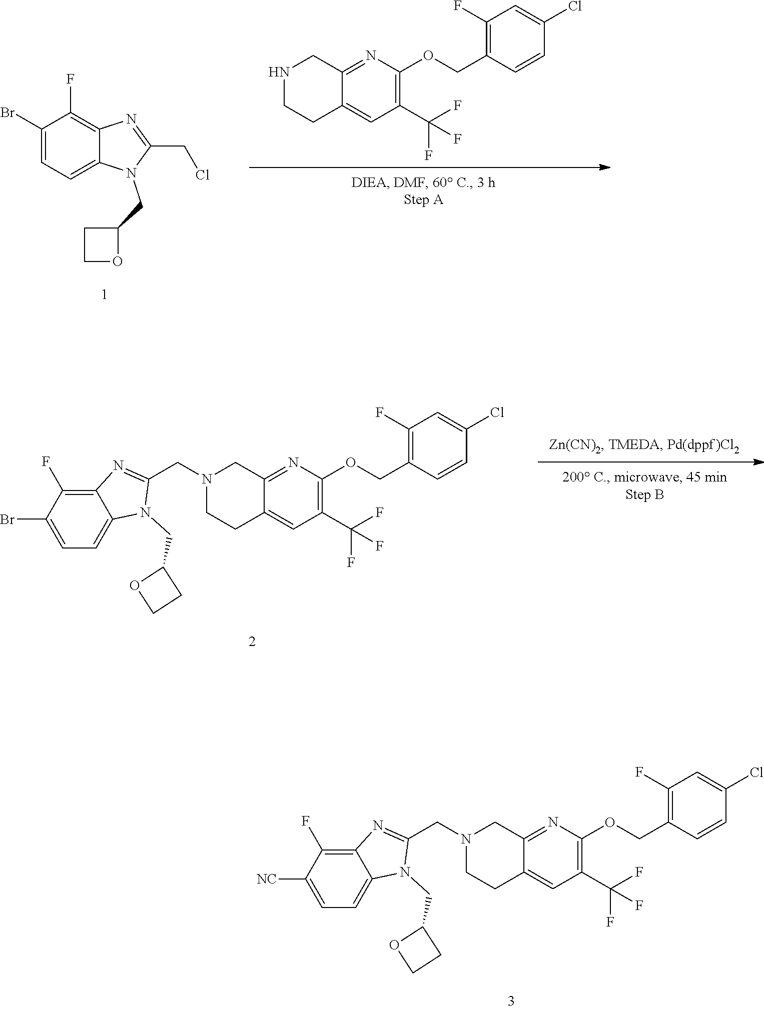 [SMILES: Fc1c(Br)ccc2c1nc(CCl)n2C[C@@H]1CCO1 ; Fc1cc(Cl)ccc1COc1nc2c(cc1C(F)(F)F)CCNC2 ; Fc1cc(Cl)ccc1COc1nc2c(cc1C(F)(F)F)CCN(Cc1nc3c(F)c(Br)ccc3n1C[C@@H]1CCO1)C2 ; N#Cc1ccc2c(nc(CN3CCc4cc(C(F)(F)F)c(OCc5ccc(Cl)cc5F)nc4C3)n2C[C@@H]2CCO2)c1F]

### Step A: (S)-7-((5-bromo-4-fluoro-1-(oxetan-2-ylmethyl)-1H-benzo[d]imidazol-2-yl)methyl)-2-((4-chloro-2-fluorobenzyl)oxy)-3-(trifluoromethyl)-5,6,7,8-tetrahydro-1,7-naphthyridine

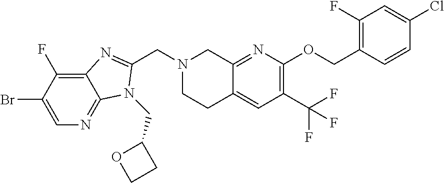 [SMILES: Fc1cc(Cl)ccc1COc1nc2c(cc1C(F)(F)F)CCN(Cc1nc3c(F)c(Br)cnc3n1C[C@@H]1CCO1)C2]

A mixture of (S)-5-bromo-2-(chloromethyl)-4-fluoro-1-(oxetan-2-ylmethyl)-1H-benzo[d]imidazole (100 mg, 0.3 mmol), 2-((4-chloro-2-fluorobenzyl)oxy)-3-(trifluoromethyl)-5,6,7,8-tetrahydro-1,7-naphthyridine (108.4 mg, 0.3 mmol) and DIEA (174.1 mg, 1.35 mmol) in DMF (4 mL) was stirred at 60° C. for 3 hrs. After the reaction was completed, water (20 mL) was added, and the mixture was extracted with EtOAc (10 mL*3). The combined organic layers were washed with brine (15 mL), dried over Na₂SO₄, filtered, and concentrated in vacuum. The residue was purified by TLC (eluting PE/EA=1/2) to give (S)-7-((5-bromo-4-fluoro-1-(oxetan-2-ylmethyl)-1H-benzo[d]imidazol-2-yl)methyl)-2-((4-chloro-2-fluorobenzyl)oxy)-3-(trifluoromethyl)-5,6,7,8-tetrahydro-1,7-naphthyridine (180 mg, 91.4% yield). MS Calcd.: 656.06; MS Found: 657.0 [M+H]⁺.

### Step B: (S)-2-((2-((4-chloro-2-fluorobenzyl)oxy)-3-(trifluoromethyl)-5,8-dihydro-1,7-naphthyridin-7(6H)-yl)methyl)-4-fluoro-1-(oxetan-2-ylmethyl)-1H-benzo[d]imidazole-5-carbonitrile

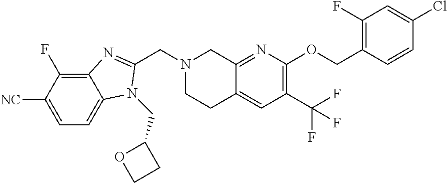 [SMILES: N#Cc1ccc2c(nc(CN3CCc4cc(C(F)(F)F)c(OCc5ccc(Cl)cc5F)nc4C3)n2C[C@@H]2CCO2)c1F]

To a solution of (S)-7-((5-bromo-4-fluoro-1-(oxetan-2-ylmethyl)-1H-benzo[d]imidazol-2-yl)methyl)-2-((4-chloro-2-fluorobenzyl)oxy)-3-(trifluoromethyl)-5,6,7,8-tetrahydro-1,7-naphthyridine (68 mg, 0.1 mmol) in DMA (6 mL) was added Zn(CN)₂ (35.2 mg, 0.3 mmol), TMEDA (5.81 mg, 0.05 mmol) and Pd(dppf)Cl₂ (7.26 mg, 0.01 mmol), and the reaction mixture was stirred at 200° C. for 45 min under microwave irradiation. After the reaction was completed, water (10 mL) was added and the mixture was extracted with EtOAc (10 mL*3). The combined organic layers were washed with brine (15 mL), dried over Na₂SO₄, filtered, and concentrated in vacuum. The residue was purified by TLC (eluting PE/EA=1/2) to give (S)-2-((2-((4-chloro-2-fluorobenzyl)oxy)-3-(trifluoromethyl)-5,8-dihydro-1,7-naphthyridin-7(6H)-yl)methyl)-4-fluoro-1-(oxetan-2-ylmethyl)-1H-benzo[d]imidazole-5-carbonitrile (50 mg, 83.0% yield). MS Calcd.: 603.15; MS Found: 604.2 [M+H]⁺.

### 2-[(4-chloro-2-fluorophenyl)methoxy]-7-[(4-fluoro-1-{[(2S)-oxetan-2-yl]methyl}-5-(1H-1,2,3,4-tetrazol-5-yl)-1H-1,3-benzodiazol-2-yl)methyl]-3-(trifluoromethyl)-5,6,7,8-tetrahydro-1,7-naphthyridine (compound 88)

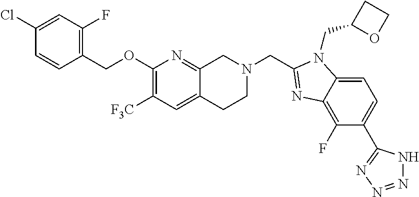 [SMILES: Fc1cc(Cl)ccc1COc1nc2c(cc1C(F)(F)F)CCN(Cc1nc3c(F)c(-c4nnn[nH]4)ccc3n1C[C@@H]1CCO1)C2]

Compound 88 was then synthesized following the similar route of Procedure 9, using (S)-2-((2-((4-chloro-2-fluorobenzyl)oxy)-3-(trifluoromethyl)-5,8-dihydro-1,7-naphthyridin-7(6H)-yl)methyl)-4-fluoro-1-(oxetan-2-ylmethyl)-1H-benzo[d]imidazole-5-carbonitrile in step A.

¹H NMR (400 MHz, DMSO-d₆) δ 7.97 (s, 1H), 7.90-7.87 (m, 1H), 7.32-7.30 (d, J=8 Hz, 1H), 7.54-7.47 (m, 1H), 7.5-7.44 (d, J=12 Hz, 1H), 7.31 (d, J=8 Hz, 1H), 5.43 (s, 2H), 5.07 (d, J=8 Hz, 1H), 4.79 (dd, J₁=4 Hz, J₂=8 Hz, 1H), 4.66 (d, J=16 Hz, 1H), 4.48-4.43 (m, 1H), 4.38-4.33 (m, 1H), 3.95-3.79 (m, 2H), 2.89-2.85 (m, 2H), 2.73-2.61 (m, 4H), 2.41-2.32 (m, 2H). ¹⁹F NMR (376 MHz, DMSO-d₆) δ −61.73, −114.97, −124.62. LC-MS: m/z 647.3 (M+H)⁺.

### 2-[(4-chloro-2,6-difluorophenyl)methoxy]-7-[(3-{[(2S)-oxetan-2-yl]methyl}-6-(11H-1,2,3,4-tetrazol-5-yl)-3H-imidazo[4,5-b]pyridin-2-yl)methyl]-3-(trifluoromethyl)-5,6,7,8-tetrahydro-1,7-naphthyridine (compound 89)

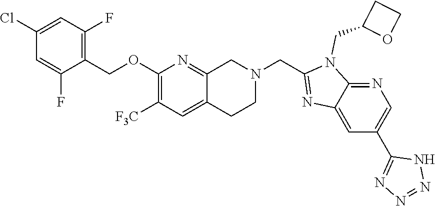 [SMILES: Fc1cc(Cl)cc(F)c1COc1nc2c(cc1C(F)(F)F)CCN(Cc1nc3cc(-c4nnn[nH]4)cnc3n1C[C@@H]1CCO1)C2]

Compound 89 was synthesized following the similar route of Procedure 9, using (S)-2-((2-((4-chloro-2,6-difluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-b]pyridine-6-carbonitrile in step A.

¹H NMR (400 MHz, DMSO-d₆) δ 9.03 (d, J=1.6 Hz, 1H), 8.63 (d, J=2.0 Hz, 1H), 7.91 (s, 1H), 7.36-7.41 (m, 2H), 5.40 (s, 2H), 5.13-5.19 (m, 1H), 4.82 (dd, J=14.8 Hz, J=6.4 Hz, 1H), 4.69 (dd, J=14.8 Hz, J=4.0 Hz, 1H), 4.45-4.50 (m, 1H), 4.32-4.4.37 (m, 1H), 4.16-4.23 (m, 2H), 3.74 (s, 2H), 2.86 (dd, J=15.2 Hz, J=4.4 Hz, 4H), 2.62-2.71 (m, 1H), 2.40-2.47 (m, 1H). ¹⁹F NMR (376 MHz, DMSO-d₆) δ −61.78, −112.07. LC-MS: m/z 648.2 (M+H)⁺.

### 2-[(4-chloro-2,6-difluorophenyl)methoxy]-7-[(3-{[(2S)-oxetan-2-yl]methyl}-6-(11H-1,2,3,4-tetrazol-5-yl)-3H-imidazo[4,5-c]pyridin-2-yl)methyl]-3-(trifluoromethyl)-5,6,7,8-tetrahydro-1,7-naphthyridine (compound 90)

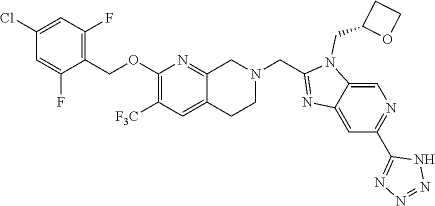 [SMILES: Fc1cc(Cl)cc(F)c1COc1nc2c(cc1C(F)(F)F)CCN(Cc1nc3cc(-c4nnn[nH]4)ncc3n1C[C@@H]1CCO1)C2]

Compound 90 was synthesized following the similar route of Procedure 9, using (S)-2-((2-((4-chloro-2,6-difluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbonitrile in step A.

¹H NMR (400 MHz, CD₃OD) δ 9.16 (s, 1H), 8.53 (s, 1H), 7.77 (s, 1H), 7.06 (d, J=7.2 Hz, 2H), 5.41 (s, 2H), 5.19 (m, 1H), 4.60-4.59 (m, 1H), 4.48-4.28 (m, 3H), 3.89-3.81 (m, 2H), 3.47-3.41 (m, 2H), 3.09-3.01 (m, 2H), 2.95-2.87 (m, 2H), 2.78-2.69 (m, 1H), 2.49-2.41 (m, 1H). ¹⁹F NMR (376 MHz, CD₃OD) δ −4.72, −113.91. LC-MS: m/z 648.3 (M+H)⁺.

### 2-[(4-chloro-2,6-difluorophenyl)methoxy]-3-methyl-7-[(3-{[(2S)-oxetan-2-yl]methyl}-6-(1H-1,2,3,4-tetrazol-5-yl)-3H-imidazo[4,5-c]pyridin-2-yl)methyl]-5,6,7,8-tetrahydro-1,7-naphthyridine (compound 91)

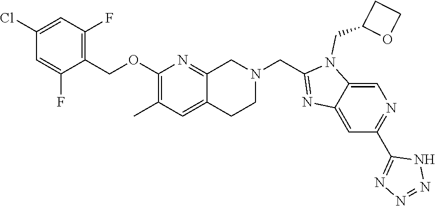 [SMILES: Cc1cc2c(nc1OCc1c(F)cc(Cl)cc1F)CN(Cc1nc3cc(-c4nnn[nH]4)ncc3n1C[C@@H]1CCO1)CC2]

Compound 91 was synthesized following the similar route of Procedure 9, using (S)-2-((2-((4-chloro-2,6-difluorobenzyl)oxy)-3-methyl-5,8-dihydro-1,7-naphthyridin-7(6H)-yl)methyl)-3-(oxetan-2-ylmethyl)-3H-imidazo[4,5-c]pyridine-6-carbonitrile in step A.

¹H NMR (400 MHz, DMSO-d₆) δ 9.27 (s, 1H), 8.52 (s, 1H), 7.42 (s, 1H), 7.31 (d, J=7.6 Hz, 2H), 5.28 (s, 2H), 5.04-5.13 (m, 1H), 4.84-4.94 (m, 1H), 4.69-4.76 (m, 1H), 4.43-4.51 (m, 1H), 4.30-4.38 (m, 1H), 4.15-4.22 (m, 2H), 3.35-3.47 (m, 2H), 2.88-2.97 (m, 2H), 2.65-2.76 (m, 2H), 2.27-2.42 (m, 2H), 2.05 (s, 3H). ¹⁹F NMR (376 MHz, DMSO-d₆) δ −112.09. LC-MS: m/z 594.3 (M+H)⁺.

### 2-({2-[(4-chloro-2-fluorophenyl)methoxy]-3-(trifluoromethyl)-5,6,7,8-tetrahydro-1,7-naphthyridin-7-yl}methyl)-4-fluoro-1-{[(2S)-oxetan-2-yl]methyl}-1H-1,3-benzodiazole-5-carboxylic acid (compound 27)

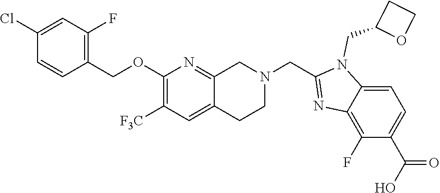 [SMILES: O=C(O)c1ccc2c(nc(CN3CCc4cc(C(F)(F)F)c(OCc5ccc(Cl)cc5F)nc4C3)n2C[C@@H]2CCO2)c1F]

Compound 27 was synthesized following the similar route of Procedure 22, using methyl (S)-2-(chloromethyl)-4-fluoro-1-(oxetan-2-ylmethyl)-1H-benzo[d]imidazole-5-carboxylate in step D.

¹H NMR (400 MHz, DMSO-d₆) δ 12.9 (s, 1H), 7.92 (s, 1H), 7.73 (dd, J=8.4, 6.4 Hz, 1H), 7.48-7.58 (m, 2H), 7.46 (dd, J=10.0, 2.0 Hz, 1H), 7.31 (dd, J=8.0, 2.0 Hz, 1H), 5.42 (s, 2H), 5.00-5.09 (m, 1H), 4.74 (dd, J=15.2, 7.2 Hz, 1H), 4.61 (dd, J=15.2, 3.2 Hz, 1H), 4.40-4.49 (m, 1H), 4.28-4.38 (m, 1H), 4.14 (d, J=13.6 Hz, 1H), 4.04 (d, J=13.6 Hz, 1H), 3.62-3.76 (m, 2H), 2.75-2.90 (m, 4H), 2.55-2.69 (m, 1H), 2.30-2.40 (m, 1H). ¹⁹F NMR (376 MHz, DMSO-d₆) δ −61.67, −114.96, −124.62. LC-MS: m/z 622.9 (M+H)⁺.

### 2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridine-6-carboxylic acid (compound 26)

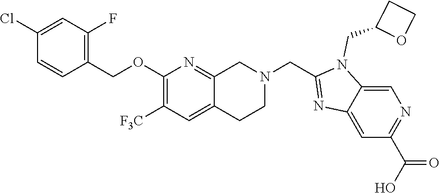 [SMILES: O=C(O)c1cc2nc(CN3CCc4cc(C(F)(F)F)c(OCc5ccc(Cl)cc5F)nc4C3)n(C[C@@H]3CCO3)c2cn1]

Compound 26 was synthesized following the similar route of Procedure 4, using (S)-6-bromo-2-(chloromethyl)-3-(oxetan-2-ylmethyl)-3H-imidazo[4,5-c]pyridine in step B.

¹H NMR (400 MHz, DMSO-d₆) δ 9.05 (s, 1H), 8.28 (s, 1H), 7.93 (s, 1H), 7.43-7.55 (m, 2H), 7.32 (dd, J=8.4, 2.0 Hz, 1H), 5.42 (s, 2H), 5.02-5.12 (m, 1H), 4.80-4.91 (m, 1H), 4.68-4.77 (m, 1H), 4.42-4.50 (m, 1H), 4.32-4.39 (m, 1H), 4.19 (d, J=13.6 Hz, 1H), 4.09 (d, J=13.6 Hz, 1H), 3.63-3.77 (m, 2H), 2.77-2.87 (m, 4H), 2.60-2.66 (m, 1H), 2.34-2.42 (m, 1H). ¹⁹F NMR (376 MHz, DMSO-d₆) δ −61.69, −114.99. LC-MS: m/z 606.2 (M+H)⁺.

### 2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-1H-imidazo[4,5-b]pyridine-5-carboxylic acid (compound

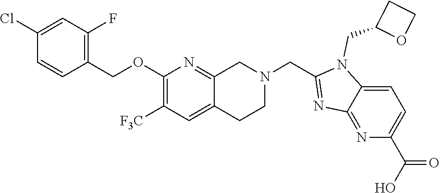 [SMILES: O=C(O)c1ccc2c(n1)nc(CN1CCc3cc(C(F)(F)F)c(OCc4ccc(Cl)cc4F)nc3C1)n2C[C@@H]1CCO1]

Compound 25 was synthesized following the similar route of Procedure 4, using (S)-5-bromo-2-(chloromethyl)-1-(oxetan-2-ylmethyl)-1H-imidazo[4,5-b]pyridine in step B.

¹H NMR (400 MHz, DMSO-d₆) δ 12.93 (s, 1H), 8.22 (d, J=8.4 Hz, 1H), 8.01 (d, J=8.4 Hz, 1H), 7.92 (s, 1H), 7.51 (t, J=8.0 Hz, 1H), 7.49-7.45 (m, 1H), 7.31 (dd, J=8.4, 2.0 Hz, 1H), 5.42 (s, 2H), 5.04-5.09 (m, 1H), 4.78 (dd, J=15.2, 7.2 Hz, 1H), 4.65 (dd, J=15.2, 2.8 Hz, 1H), 4.42-4.48 (m, 1H), 4.30-4.38 (m, 1H), 4.20 (d, J=14.0 Hz, 1H), 4.11 (d, J=14.0 Hz, 1H), 3.69-3.81 (m, 2H), 2.86-2.93 (m, 2H), 2.75-2.85 (m, 2H), 2.60-2.65 (m, 1H), 2.34-2.40 (m, 1H). ¹⁹F NMR (376 MHz, DMSO-d₆) δ −61.69, −114.96. LC-MS: m/z 606.2 (M+H)⁺.

### Procedure 26

### 2-({2-[(4-chloro-2-fluorophenyl)methoxy]-3-(trifluoromethyl)-5,6,7,8-tetrahydro-1,7-naphthyridin-7-yl}methyl)-4-fluoro-N-methyl-1-{[(2S)-oxetan-2-yl]methyl}-1H-1,3-benzodiazole-5-carboxamide (compound 22)

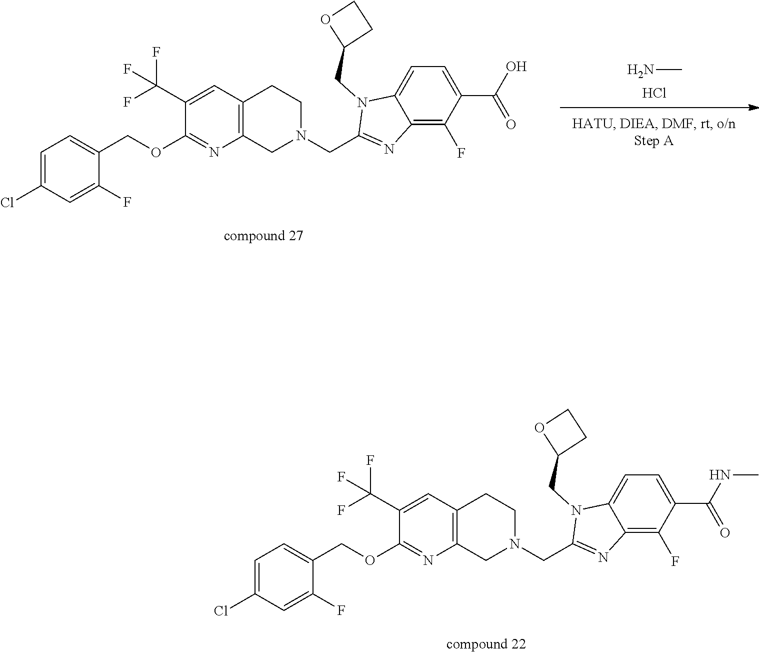

To a solution of 2-({2-[(4-chloro-2-fluorophenyl)methoxy]-3-(trifluoromethyl)-5,6,7,8-tetrahydro-1,7-naphthyridin-7-yl}methyl)-4-fluoro-1-{[(2S)-oxetan-2-yl]methyl}-1H-1,3-benzodiazole-5-carboxylic acid (100 mg, 0.16 mmol), CH₃NH₂·HCl (32.51 mg, 0.48 mmol), and DIEA (103.73 mg, 0.80 mmol) in DMF (5 mL) was added HATU (91.55 mg, 0.24 mmol) in portions at room temperature. The resulting mixture was stirred overnight at room temperature under nitrogen atmosphere. The resulting mixture was extracted with EtOAc (3×15 mL). The combined organic layers were washed with brine (2×10 mL) and dried over anhydrous Na₂SO₄. After filtration, the filtrate was concentrated under reduced pressure. The crude product was purified by Prep-HPLC with the following conditions (Column: Kinetex EVO prep C18, 30*150, 5 m; Mobile Phase A: Water (10 mmol/L NH₄HCO₃), Mobile Phase B: ACN; Flow rate: 60 mL/min; Gradient: 35% B to 68% B in 10 min, 68% B; Wave Length: 220 nm; RT1 (min): 9.53) to give 2-({2-[(4-chloro-2-fluorophenyl)methoxy]-3-(trifluoromethyl)-5,6,7,8-tetrahydro-1,7-naphthyridin-7-yl}methyl)-4-fluoro-N-methyl-1-{[(2S)-oxetan-2-yl]methyl}-1H-1,3-benzodiazole-5-carboxamide (67.09 mg, 64.73%).

¹H NMR (400 MHz, DMSO-d₆) δ 8.16 (t, J=3.6 Hz, 1H), 7.92 (s, 1H), 7.43-7.55 (m, 4H), 7.32 (dd, J=8.0, 1.6 Hz, 1H), 5.42 (s, 2H), 4.98-5.08 (m, 1H), 4.73 (dd, J=15.2, 7.2 Hz, 1H), 4.60 (dd, J=15.2, 3.2 Hz, 1H), 4.39-4.50 (m, 1H), 4.28-4.37 (m, 1H), 4.14 (d, J=13.6 Hz, 1H), 4.04 (d, J=13.6 Hz, 1H), 3.60-3.75 (m, 2H), 2.75-2.85 (m, 7H), 2.55-2.65 (m, 1H), 2.28-2.39 (m, 1H). ¹⁹F NMR (376 MHz, DMSO-d₆) δ −61.68, −115.00, −129.47. LC-MS: m/z 636.3 (M+H)⁺.

### 2-({2-[(4-chloro-2-fluorophenyl)methoxy]-3-(trifluoromethyl)-5,6,7,8-tetrahydro-1,7-naphthyridin-7-yl}methyl)-4-fluoro-1-{[(2S)-oxetan-2-yl]methyl}-N-(2,2,2-trifluoroethyl)-1H-1,3-benzodiazole-5-carboxamide (compound 21)

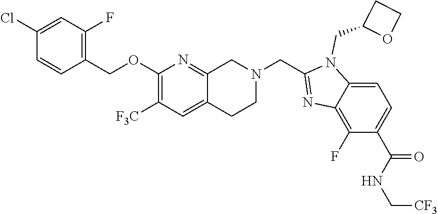 [SMILES: O=C(NCC(F)(F)F)c1ccc2c(nc(CN3CCc4cc(C(F)(F)F)c(OCc5ccc(Cl)cc5F)nc4C3)n2C[C@@H]2CCO2)c1F]

Compound 21 was synthesized following the similar route of Procedure 26, using 2,2,2-trifluoroethylamine in step A.

¹H NMR (400 MHz, DMSO-d₆) δ 8.83-8.91 (m, 1H), 7.92 (s, 1H), 7.57 (d, J=8.8 Hz, 1H), 7.43-7.55 (m, 3H), 7.32 (dd, J=8.4, 1.6 Hz, 1H), 5.42 (s, 2H), 4.98-5.10 (m, 1H), 4.75 (dd, J=15.2, 7.2 Hz, 1H), 4.62 (dd, J=15.2, 3.2 Hz, 1H), 4.39-4.49 (m, 1H), 4.28-4.38 (m, 1H), 4.01-4.18 (m, 4H), 3.61-3.76 (m, 2H), 2.76-2.88 (m, 4H), 2.56-2.68 (m, 1H), 2.28-2.41 (m, 1H). ¹⁹F NMR (376 MHz, DMSO-d₆) δ −61.69, −70.44, −115.00, −128.84. LC-MS: m/z 704.2 (M+H)⁺.

### 2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-1H-1,3-benzodiazole-5-carboxamide (compound 92)

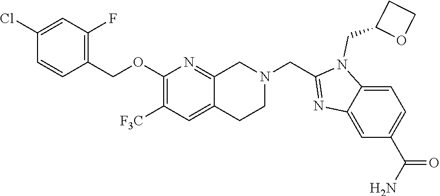 [SMILES: NC(=O)c1ccc2c(c1)nc(CN1CCc3cc(C(F)(F)F)c(OCc4ccc(Cl)cc4F)nc3C1)n2C[C@@H]1CCO1]

Compound 92 was synthesized following the similar route of Procedure 26, using 2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-1H-1,3-benzodiazole-5-carboxylic acid and NH₃(g) in MeOH aq. in step A.

¹H NMR (400 MHz, DMSO-d₆) δ 8.20 (s, 1H), 7.95 (s, 1H), 7.92 (s, 1H), 7.79-7.81 (m, 1H), 7.66 (d, J=8.4 Hz, 1H), 7.51 (t, J=8.0 Hz, 1H), 7.46 (dd, J=10.0 Hz, 2.0 Hz, 1H), 7.31 (dd, J=8.4 Hz, 2.0 Hz, 1H), 7.24 (s, 1H), 5.42 (s, 2H), 5.03-5.06 (m, 1H), 4.70-4.75 (m, 1H), 4.59 (dd, J=14.8 Hz, 2.8 Hz, 1H), 4.42-4.47 (m, 1H), 4.31-4.36 (m, 1H), 4.13 (d, J=13.6 Hz, 1H), 4.03 (d, J=13.6 Hz, 1H), 3.67-3.69 (m, 2H), 2.81-2.83 (m, 4H), 2.60-2.64 (m, 1H), 2.33-2.37 (m, 1H). ¹⁹F NMR (376 MHz, DMSO-d₆) δ −61.67, −114.97. LC-MS: m/z 604.3 (M+H)⁺.

### 2-({2-[(4-chloro-2-fluorophenyl)methoxy]-3-(trifluoromethyl)-5,6,7,8-tetrahydro-1,7-naphthyridin-7-yl}methyl)-N-methyl-3-{[(2S)-oxetan-2-yl]methyl}-3H-imidazo[4,5-c]pyridine-6-carboxamide (compound 24)

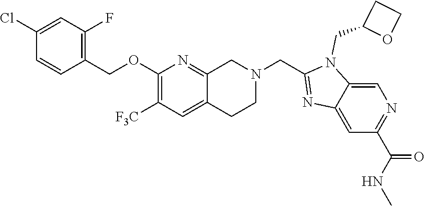 [SMILES: CNC(=O)c1cc2nc(CN3CCc4cc(C(F)(F)F)c(OCc5ccc(Cl)cc5F)nc4C3)n(C[C@@H]3CCO3)c2cn1]

Compound 24 was synthesized following the similar route of Procedure 26, using 2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridine-6-carboxylic acid in step A.

¹H NMR (400 MHz, DMSO-d₆) δ 9.02 (s, 1H), 8.71 (q, J=4.8 Hz, 1H), 8.24 (s, 1H), 7.93 (s, 1H), 7.43-7.56 (m, 2H), 7.28-7.34 (m, 1H), 5.42 (s, 2H), 5.03-5.12 (m, 1H), 4.87 (dd, J=15.2, 7.2 Hz, 1H), 4.72 (dd, J=15.2, 2.8 Hz, 1H), 4.41-4.49 (m, 1H), 4.31-4.39 (m, 1H), 4.19 (d, J=14.0 Hz, 1H), 4.09 (d, J=14.0 Hz, 1H), 3.72 (dd, J=24.0, 17.2 Hz, 2H), 2.89-2.78 (m, 6H), 2.60-2.70 (m, 1H), 2.32-2.42 (m, 1H). ¹⁹F NMR (376 MHz, DMSO-d₆) δ −61.68, −114.99. LC-MS: m/z 618.9 (M+H)⁺.

### 2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-N-(2,2,2-trifluoroethyl)-3H-imidazo[4,5-c]pyridine-6-carboxamide (compound 23)

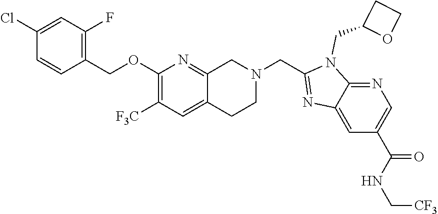 [SMILES: O=C(NCC(F)(F)F)c1cnc2c(c1)nc(CN1CCc3cc(C(F)(F)F)c(OCc4ccc(Cl)cc4F)nc3C1)n2C[C@@H]1CCO1]

Compound 23 was synthesized following the similar route of Procedure 26, using 2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridine-6-carboxylic acid and 2,2,2-trifluoroethylamine in step A.

¹H NMR (400 MHz, DMSO-d₆) δ 9.25 (t, J=6.8 Hz, 1H), 9.08 (s, 1H), 8.30 (s, 1H), 7.93 (s, 1H), 7.43-7.55 (m, 2H), 7.31 (dd, J=8.4, 2.4 Hz, 1H), 5.42 (s, 2H), 5.05-5.13 (m, 1H), 4.89 (dd, J=15.2, 7.2 Hz, 1H), 4.69-4.80 (m, 1H), 4.43-4.49 (m, 1H), 4.31-4.39 (m, 1H), 4.20 (d, J=14.0 Hz, 1H), 4.06-4.17 (m, 3H), 3.72 (dd, J=23.6, 16.8 Hz, 2H), 2.76-2.91 (m, 4H), 2.59-2.70 (m, 1H), 2.32-2.40 (m, 1H). ¹⁹F NMR (376 MHz, DMSO-d₆) δ −61.68, −70.40, −114.98. LC-MS: m/z 686.9 (M+H)⁺.

### Procedure 27

### 2-({2-[(4-chloro-2-fluorophenyl)methoxy]-3-(trifluoromethyl)-5,6,7,8-tetrahydro-1,7-naphthyridin-7-yl}methyl)-N-methanesulfonyl-1-{[(2S)-oxetan-2-yl]methyl}-1H-1,3-benzodiazole-5-carboxamide (compound 93)

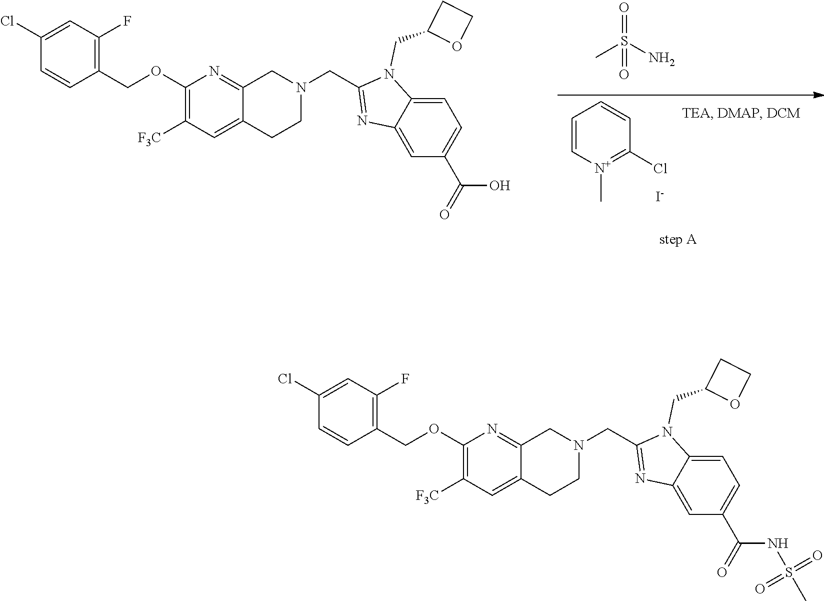

To a solution of 2-({2-[(4-chloro-2-fluorophenyl)methoxy]-3-(trifluoromethyl)-5,6,7,8-tetrahydro-1,7-naphthyridin-7-yl}methyl)-1-{[(2S)-oxetan-2-yl]methyl}-1H-1,3-benzodiazole-5-carboxylic acid (100 mg, 0.13 mmol), methanesulfonamide (47 mg, 0.5 mmol), TEA (51 mg, 0.5 mmol), and DMAP (10 mg, 0.083 mmol) in DCM (10 mL) was added 2-chloro-1-methylpyridinium iodide (52 mg, 0.2 mmol), and the mixture was stirred at room temperature for 1 hour. The reaction mixture was diluted with DCM (20 mL) and washed with brine (50 mL*3). The organic layer was separated and dried over Na₂SO₄, filtered, and purified by prep-HPLC to give 2-({2-[(4-chloro-2-fluorophenyl)methoxy]-3-(trifluoromethyl)-5,6,7,8-tetrahydro-1,7-naphthyridin-7-yl}methyl)-N-methanesulfonyl-1-{[(2S)-oxetan-2-yl]methyl}-1H-1,3-benzodiazole-5-carboxamide (46.22 mg, yield: 41%).

¹H NMR (400 MHz, DMSO-d₆) δ 12.04 (s, 1H), 8.28 (d, J=1.2 Hz, 1H), 7.92 (s, 1H), 7.86 (dd, J=8.4 Hz, 1.2 Hz, 1H), 7.74 (d, J=8.4 Hz, 1H), 7.51 (t, J=8.0 Hz, 1H), 7.46 (dd, J=10.0 Hz, 2.0 Hz, 1H), 7.31 (dd, J=8.0 Hz, 2.0 Hz, 1H), 5.42 (s, 2H), 5.02-5.08 (m, 1H), 4.72-4.78 (m, 1H), 4.61 (dd, J=15.2 Hz, 2.8 Hz, 1H), 4.42-4.47 (m, 1H), 4.32-4.37 (m, 1H), 4.03-4.17 (m, 2H), 3.64-3.74 (m, 2H), 3.27 (s, 3H), 2.78-2.85 (m, 4H), 2.56-2.67 (m, 1H), 2.33-2.45 (m, 1H). ¹⁹F NMR (376 MHz, DMSO-d₆) δ −61.68, −114.98. LC-MS: m/z 682.1 (M+H)⁺.

### Procedure 28

### 5-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-N-methyl-4H-1,2,4-triazole-3-carboxamide (compound 94)

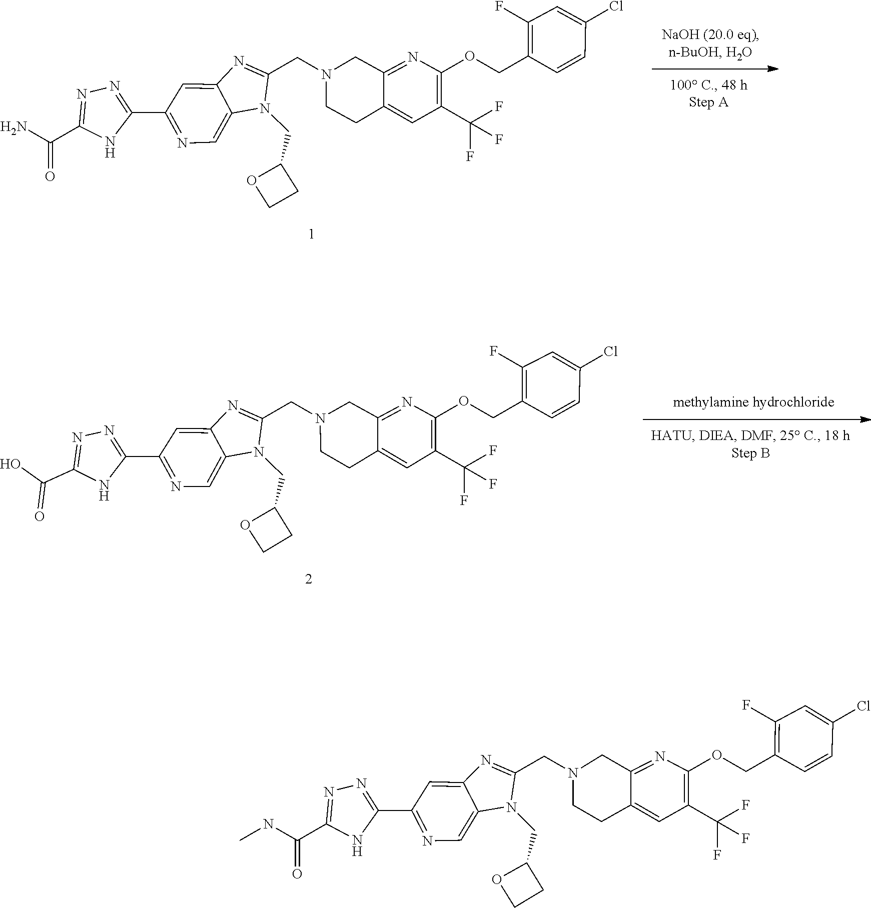

### Step A: (S)-5-(2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridin-6-yl)-4H-1,2,4-triazole-3-carboxylic acid

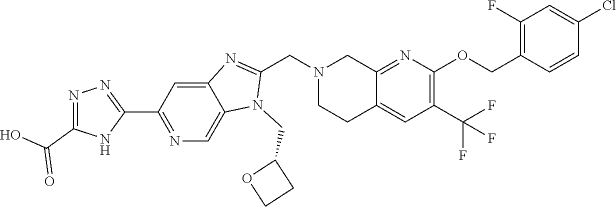 [SMILES: O=C(O)c1nnc(-c2cc3nc(CN4CCc5cc(C(F)(F)F)c(OCc6ccc(Cl)cc6F)nc5C4)n(C[C@@H]4CCO4)c3cn2)[nH]1]

To a solution of(S)-5-(2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridin-6-yl)-4H-1,2,4-triazole-3-carboxamide which was synthesized following the similar route of Procedure 5, using tert-butyl 2-((4-chloro-2-fluorobenzyl)oxy)-3-(trifluoromethyl)-5,8-dihydro-1,7-naphthyridine-7(6H)-carboxylate in step A (200 mg, 0.298 mmol), in n-BuOH (10 mL) and water (3 mL) was added NaOH (238 mg, 5.952 mmol). The reaction mixture was stirred at 100° C. for 48 hrs under N₂. The reaction mixture was concentrated in vacuum to give crude (S)-5-(2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridin-6-yl)-4H-1,2,4-triazole-3-carboxylic acid (120 mg, 59.91% yield). MS Calcd: 672.16; MS Found: 673.3 [M+H]⁺.

### Step B: 5-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-N-methyl-4H-1,2,4-triazole-3-carboxamide (compound 94)

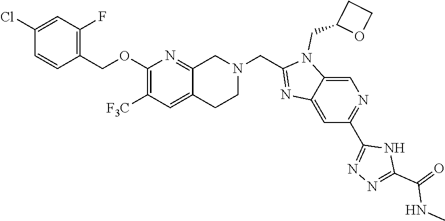 [SMILES: CNC(=O)c1nnc(-c2cc3nc(CN4CCc5cc(C(F)(F)F)c(OCc6ccc(Cl)cc6F)nc5C4)n(C[C@@H]4CCO4)c3cn2)[nH]1]

To a solution of(S)-5-(2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridin-6-yl)-4H-1,2,4-triazole-3-carboxylic acid (50 mg, 0.074 mmol, 1.0 eq) in DMF (5 mL) was added methylamine hydrochloride (7.52 mg, 0.111 mmol), HATU (56.5 mg, 0.149 mmol) and DIEA (48 mg, 0.371 mmol), and the reaction mixture was stirred at 25° C. for 18 hrs under N₂. The reaction mixture was concentrated in vacuum, the residue was purified by prep-HPLC to give 5-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-N-methyl-4H-1,2,4-triazole-3-carboxamide (1.04 mg, 1.87% yield).

¹H NMR (400 MHz, CD₃OD) δ 9.10 (s, 1H), 8.49 (s, 1H), 7.77 (s, 1H), 7.47-7.43 (t, J=15.6 Hz, 1H), 7.18-7.17 (m, 1H), 7.15 (s, 1H), 5.43 (s, 2H), 5.22-5.21 (m, 1H), 4.94-4.90 (m, 1H), 4.77-4.73 (m, 1H), 4.61-4.60 (m, 1H), 4.44-4.42 (m, 1H), 4.27 (d, J=13.2 Hz, 1H), 4.16 (d, J=13.2 Hz, 1H), 3.75-3.73 (m, 2H), 2.96 (s, 3H), 2.94-2.92 (m, 2H), 2.88-2.78 (m, 2H), 2.72-2.70 (m, 1H), 2.49-2.47 (m, 1H). ¹⁹F NMR (376 MHz, CD₃OD) δ −64.57, −117.37. LC-MS: m/z 686.1 (M+H)⁺.

### 5-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-N,N-dimethyl-4H-1,2,4-triazole-3-carboxamide (compound 95)

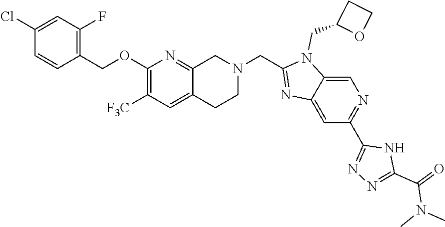 [SMILES: CN(C)C(=O)c1nnc(-c2cc3nc(CN4CCc5cc(C(F)(F)F)c(OCc6ccc(Cl)cc6F)nc5C4)n(C[C@@H]4CCO4)c3cn2)[nH]1]

Compound 95 was synthesized following the similar route of Procedure 26, using (S)-5-(2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridin-6-yl)-4H-1,2,4-triazole-3-carboxylic acid and dimethylamine hydrochloride in step A.

¹H NMR (400 MHz, CD₃OD) δ 9.11 (s, 1H), 8.45 (s, 1H), 7.78 (s, 1H), 7.47-7.43 (t, J=16.4 Hz, 1H), 7.17-7.15 (m, 2H), 5.43 (s, 2H), 5.23-5.21 (m, 1H), 4.95-4.91 (m, 1H), 4.78-4.74 (m, 1H), 4.63-4.58 (m, 1H), 4.46-4.41 (m, 1H), 4.28 (d, J=13.6 Hz, 1H), 4.17 (d, J=13 Hz, 1H), 3.80-3.70 (m, 2H), 3.44 (s, 3H), 3.17 (s, 3H), 2.94-2.92 (m, 2H), 2.89-2.85 (m, 2H), 2.75-2.70 (m, 1H), 2.50-2.45 (m, 1H). ¹⁹F NMR (376 MHz, CD₃OD) δ −64.57, −117.37. LC-MS: m/z 700.2 (M+H)⁺.

### Procedure 29

### 5-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-2,3-dihydro-1,3,4-oxadiazol-2-one (compound 96)

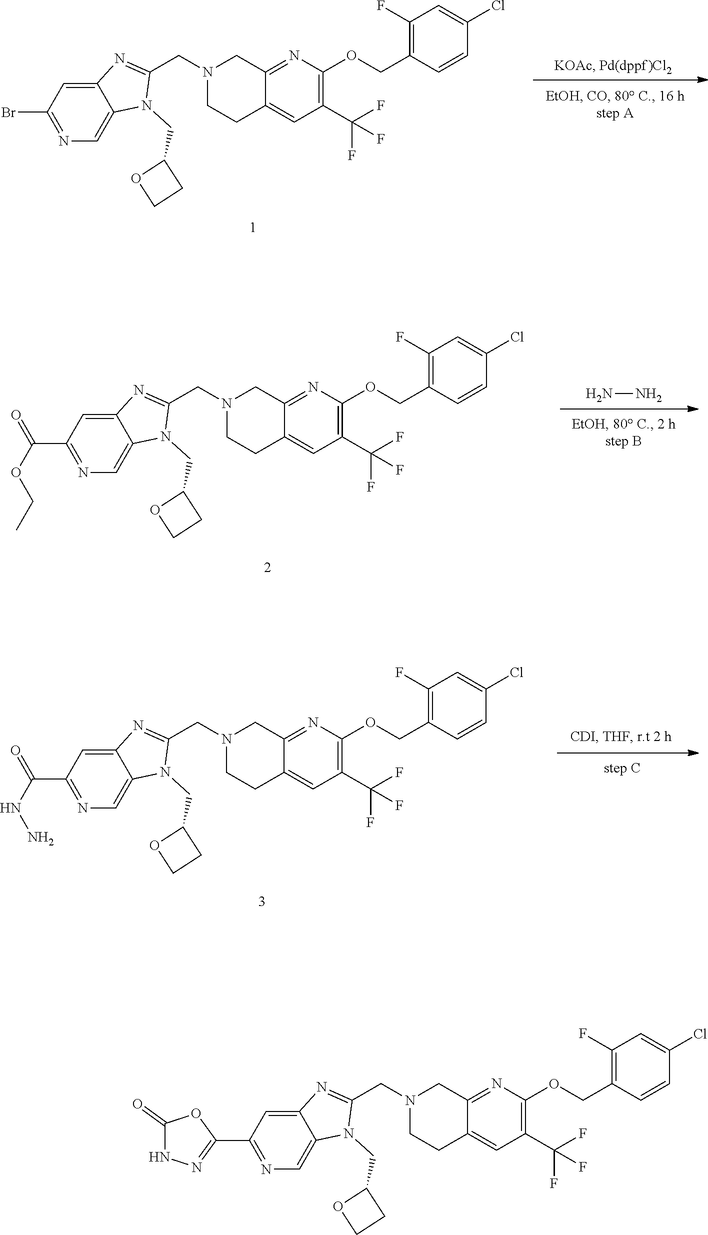

### Step A: ethyl (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carboxylate

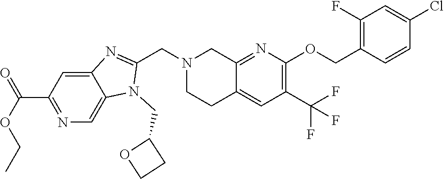 [SMILES: CCOC(=O)c1cc2nc(CN3CCc4cc(C(F)(F)F)c(OCc5ccc(Cl)cc5F)nc4C3)n(C[C@@H]3CCO3)c2cn1]

To a solution of (S)-7-((6-bromo-3-(oxetan-2-ylmethyl)-3H-imidazo[4,5-c]pyridin-2-yl)methyl)-2-((4-chloro-2-fluorobenzyl)oxy)-3-(trifluoromethyl)-5,6,7,8-tetrahydro-1,7-naphthyridine (200 mg, 0.327 mmol) in EtOH (30 mL) was added PdCl₂(dppf) (24 mg, 0.033 mmol) and KOAc (96 mg, 0.98 mmol). The mixture was stirred at 80° C. for 16 hours under CO. After the reaction was completed, the reaction mixture was filtered, and the filtrate evaporated to dryness. The residue was purified by column chromatography on silica gel (PE/EA=1/2) to give ethyl (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carboxylate (170 mg, yield: 82%). MS Calcd.: 633.18; MS Found: 634.3 [M+H]⁺.

### Step B: (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbohydrazide

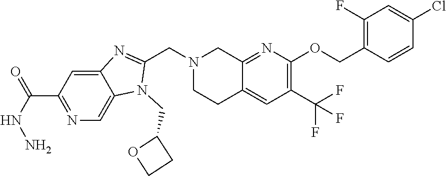 [SMILES: NNC(=O)c1cc2nc(CN3CCc4cc(C(F)(F)F)c(OCc5ccc(Cl)cc5F)nc4C3)n(C[C@@H]3CCO3)c2cn1]

To a solution of ethyl (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carboxylate (170 mg, 0.268 mmol) in EtOH (20 mL) was added N₂H₄H₂O (134 mg, 2.68 mmol). The mixture was stirred at 80° C. for 2 hours. The mixture was diluted with EtOAc (80 mL) and was washed with brine (50 mL*3). The organic layer was separated and dried over Na₂SO₄, filtered, and purified by prep-TLC (DCM/MeOH=10/1) to give (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbohydrazide (80 mg, yield: 48%). MS Calcd.: 619.17; MS Found: 620.3 [M+H]⁺.

### Step C: 5-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-2,3-dihydro-1,3,4-oxadiazol-2-one (compound 96)

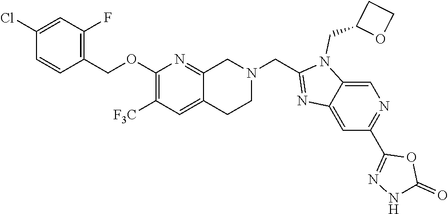 [SMILES: O=c1[nH]nc(-c2cc3nc(CN4CCc5cc(C(F)(F)F)c(OCc6ccc(Cl)cc6F)nc5C4)n(C[C@@H]4CCO4)c3cn2)o1]

To a mixture of (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbohydrazide (80 mg, 0.129 mmol) in THF (1.5 mL) was added CDI (31 mg, 0.193 mmol). The reaction mixture was stirred at room temperature for 2 hours. The reaction mixture was purified by prep-HPLC to give 5-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-2,3-dihydro-1,3,4-oxadiazol-2-one (15.38 mg, yield: 18.5%).

¹H NMR (400 MHz, DMSO-d₆) δ 12.59 (s, 1H), 9.12 (s, 1H), 8.12 (s, 1H), 7.93 (s, 1H), 7.51 (t, J=8.2 Hz, 1H), 7.46 (dd, J=10.0 Hz, 2.0 Hz, 1H), 7.32 (dd, J=8.0 Hz, 1.6 Hz, 1H), 5.42 (s, 2H), 5.07-5.09 (m, 1H), 4.85-4.90 (m, 1H), 4.72 (dd, J=15.2 Hz, 2.4 Hz, 1H), 4.46-4.48 (m, 1H), 4.36-4.39 (m, 1H), 4.20 (d, J=13.6 Hz, 1H), 4.10 (d, J=13.6 Hz, 1H), 3.71-3.73 (m, 2H), 2.82-2.86 (m, 4H), 2.64-2.68 (m, 1H), 2.33-2.40 (m, 1H). ¹⁹F NMR (376 MHz, DMSO-d₆) δ −61.67, −115.00. LC-MS: m/z 646.2 (M+H)⁺.

### 3-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-4,5-dihydro-1H-1,2,4-triazol-5-one (compound 97)

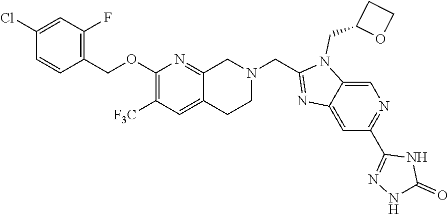 [SMILES: O=c1[nH]nc(-c2cc3nc(CN4CCc5cc(C(F)(F)F)c(OCc6ccc(Cl)cc6F)nc5C4)n(C[C@@H]4CCO4)c3cn2)[nH]1]

Compound 97 was synthesized following the similar route of Procedure 29, using methyl (S)-2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbaminate in step B.

¹H NMR (400 MHz, DMSO-d₆) δ 11.89 (br s, 1H), 11.62 (br s, 1H), 9.04 (s, 1H), 8.10 (s, 1H), 7.93 (s, 1H), 7.43-7.54 (m, 2H), 7.31 (d, J=7.2 Hz, 1H), 5.42 (s, 2H), 5.04-5.11 (m, 1H), 4.81-4.91 (m, 1H), 4.66-4.74 (m, 1H), 4.42-4.50 (m, 1H), 4.32-4.40 (m, 1H), 4.19 (d, J=14.0 Hz, 1H), 4.08 (d, J=14.0 Hz, 1H), 3.65-3.76 (m, 2H), 2.78-2.89 (m, 4H), 2.62-2.71 (m, 1H), 2.35-2.41 (m, 1H). ¹⁹F NMR (376 MHz, DMSO-d₆) δ −61.68, 114.99. LC-MS: m/z 645.2 (M+H)⁺.

### Procedure 30

### 2-[(4-chloro-2-fluorophenyl)methoxy]-7-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-3-(trifluoromethyl)quinoline (compound 98)

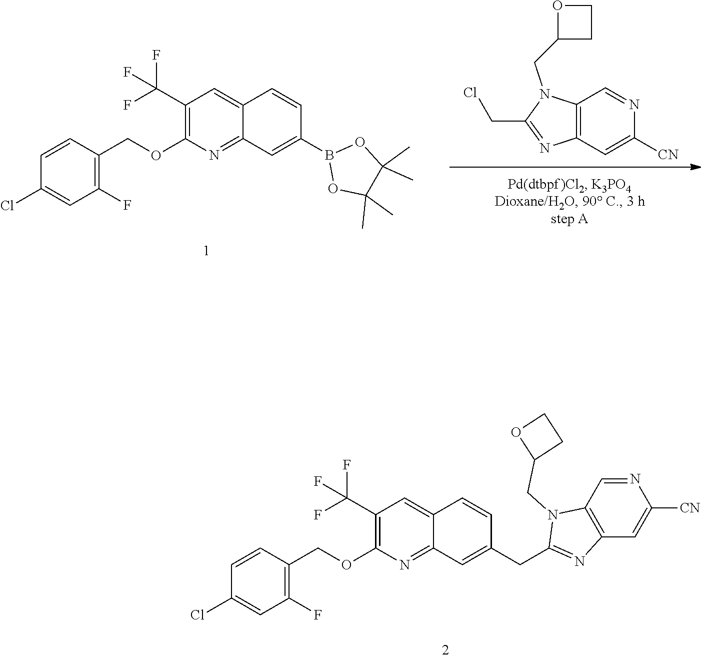 [SMILES: CC1(C)OB(c2ccc3cc(C(F)(F)F)c(OCc4ccc(Cl)cc4F)nc3c2)OC1(C)C ; N#Cc1cc2nc(CCl)n(CC3CCO3)c2cn1 ; N#Cc1cc2nc(Cc3ccc4cc(C(F)(F)F)c(OCc5ccc(Cl)cc5F)nc4c3)n(CC3CCO3)c2cn1]

### Step A: 2-((2-((4-chloro-2-fluorobenzyl)oxy)-3-(trifluoromethyl)quinolin-7-yl)methyl)-3-(oxetan-2-ylmethyl)-3H-imidazo[4,5-c]pyridine-6-carbonitrile

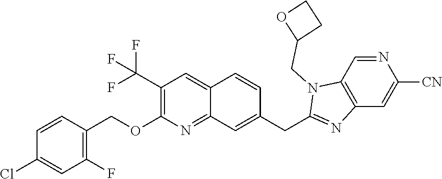 [SMILES: N#Cc1cc2nc(Cc3ccc4cc(C(F)(F)F)c(OCc5ccc(Cl)cc5F)nc4c3)n(CC3CCO3)c2cn1]

To a flame-dried tube was charged 2-(chloromethyl)-3-(oxetan-2-ylmethyl)-3H-imidazo[4,5-c]pyridine-6-carbonitrile (131 mg, 0.5 mmol), Pd(dtbpf)Cl₂ (33 mg, 0.05 mmol), K₃PO₄ (318 mg, 1.5 mmol), and 2-((4-chloro-2-fluorobenzyl)oxy)-7-(4,4,5,5-tetramethyl-1,3,2-dioxaborolan-2-yl)-3-(trifluoromethyl)quinoline (240 mg, 0.5 mmol). The tube was evacuated three times under vacuum and backfilled with N₂. Degassed 1,4-dioxane (2 mL) and H₂O (0.4 mL) were injected via syringe. The mixture was stirred at 90° C. for 3 hrs. After cooling to room temperature, the mixture was extracted with diethyl ether (3×10 mL). The combined organic layers were evaporated under reduced pressure. The residue was purified by column chromatography, eluted with CH₂Cl₂/MeOH (10:1) to give 2-((2-((4-chloro-2-fluorobenzyl)oxy)-3-(trifluoromethyl)quinolin-7-yl)methyl)-3-(oxetan-2-ylmethyl)-3H-imidazo[4,5-c]pyridine-6-carbonitrile (290 mg, 50.0% yield). LC-MS: m/z 581.9 (M+H)⁺.

### 2-[(4-chloro-2-fluorophenyl)methoxy]-7-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]-3-(trifluoromethyl)quinoline (compound 98)

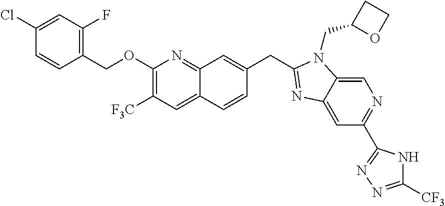 [SMILES: Fc1cc(Cl)ccc1COc1nc2cc(Cc3nc4cc(-c5nnc(C(F)(F)F)[nH]5)ncc4n3C[C@@H]3CCO3)ccc2cc1C(F)(F)F]

Compound 98 was then synthesized following the similar route of Procedure 3, using 2-((2-((4-chloro-2-fluorobenzyl)oxy)-3-(trifluoromethyl)quinolin-7-yl)methyl)-3-(oxetan-2-ylmethyl)-3H-imidazo[4,5-c]pyridine-6-carbonitrile in step C.

¹H NMR (400 MHz, DMSO-d₆) δ 9.14 (d, J=0.8 Hz, 1H), 8.85 (s, 1H), 8.26 (d, J=0.8 Hz, 1H), 8.08 (d, J=8.4 Hz, 1H), 7.86 (d, J=1.6 Hz, 1H), 7.66-7.56 (m, 2H), 7.51 (dd, J=10.0, 2.0 Hz, 1H), 7.35 (dd, J=8.4, 2.0 Hz, 1H), 5.63 (s, 2H), 5.02 (qd, J=7.2, 2.8 Hz, 1H), 4.86 (dd, J=15.2, 7.2 Hz, 1H), 4.75-4.65 (m, 3H), 4.51-4.44 (m, 1H), 4.38 (dt, J=9.2, 6.0 Hz, 1H), 2.70-2.64 (m, 1H), 2.40-2.33 (m, 1H), 1.23 (s, 1H). ¹⁹F NMR (376 MHz, DMSO-d₆) δ −62.07, −63.63, −114.93. LC-MS: m/z 692.1 (M+H)⁺.

### Procedure 31

### 3-{4-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-b]pyridin-6-yl]-1H-1,2,3-triazol-1-yl}propanoic acid (compound 99)

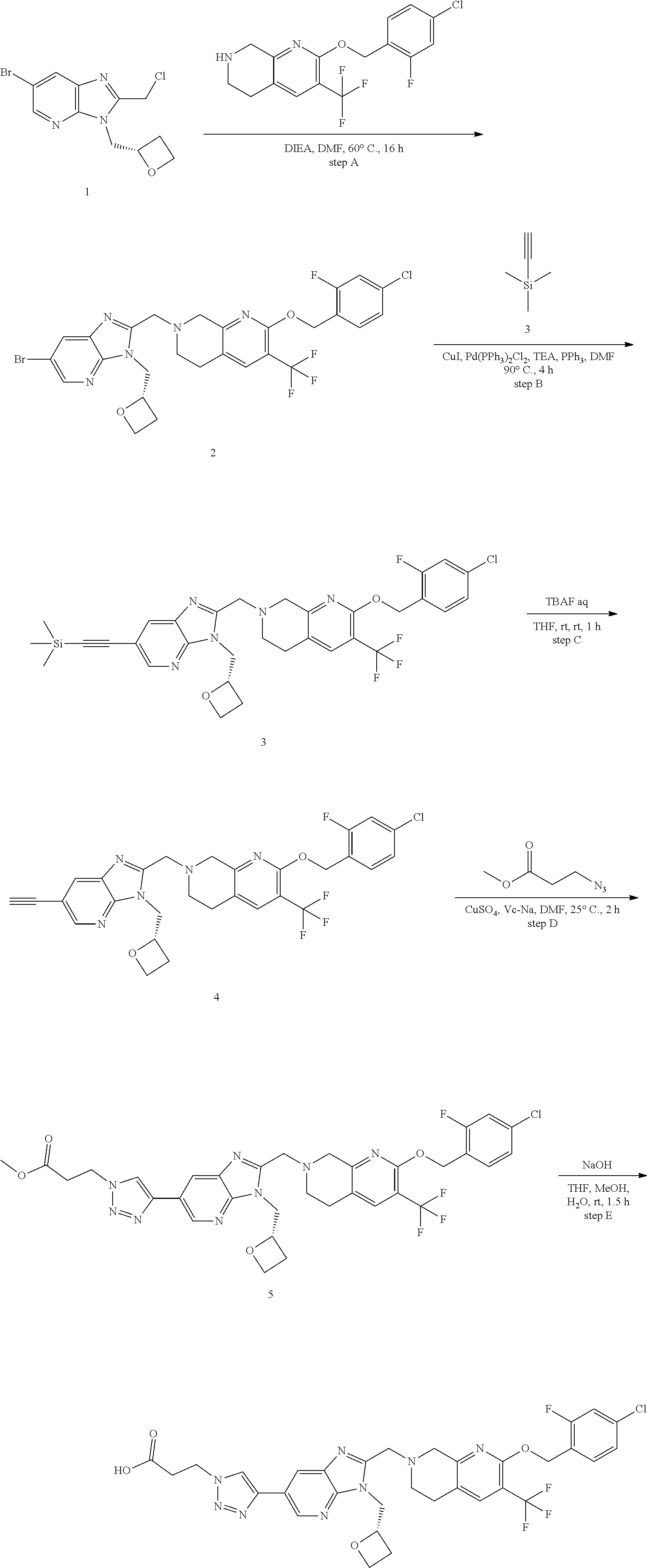

### Step A: (S)-7-((6-bromo-3-(oxetan-2-ylmethyl)-3H-imidazo[4,5-b]pyridin-2-yl)methyl)-2-((4-chloro-2-fluorobenzyl)oxy)-3-(trifluoromethyl)-5,6,7,8-tetrahydro-1,7-naphthyridine

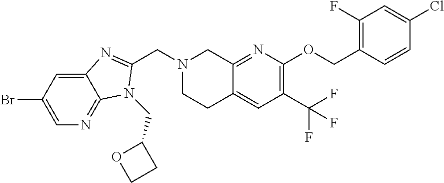 [SMILES: Fc1cc(Cl)ccc1COc1nc2c(cc1C(F)(F)F)CCN(Cc1nc3cc(Br)cnc3n1C[C@@H]1CCO1)C2]

A mixture of (S)-6-bromo-2-(chloromethyl)-3-(oxetan-2-ylmethyl)-3H-imidazo[4,5-b]pyridine (160 mg, 0.505 mmol), 2-((4-chloro-2-fluorobenzyl)oxy)-3-(trifluoromethyl)-5,6,7,8-tetrahydro-1,7-naphthyridine (127 mg, 0.353 mmol), and DIEA (0.74 mL, 1.575 mmol) in DMF (4 mL) was stirred at 60° C. for 4 hrs. After the reaction was completed, the mixture was concentrated under reduced pressure, and the residue was purified by flash column chromatography (eluting PE/EA=30/1-10/1) to give (S)-7-((6-bromo-3-(oxetan-2-ylmethyl)-3H-imidazo[4,5-b]pyridin-2-yl)methyl)-2-((4-chloro-2-fluorobenzyl)oxy)-3-(trifluoromethyl)-5,6,7,8-tetrahydro-1,7-naphthyridine (123 mg, 38% yield). MS Calcd.: 639.07; MS Found: 640.2 [M+H]⁺.

### Step B: (S)-2-((4-chloro-2-fluorobenzyl) oxy)-7-((3-(oxetan-2-ylmethyl)-6-((trimethylsilyl)ethynyl)-3H-imidazo[4,5-b]pyridin-2-yl)methyl)-3-(trifluoromethyl)-5,6,7,8-tetrahydro-1,7-naphthyridine

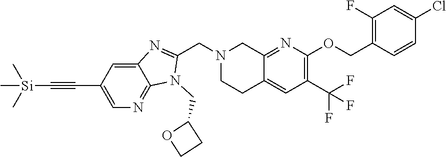 [SMILES: C[Si](C)(C)C#Cc1cnc2c(c1)nc(CN1CCc3cc(C(F)(F)F)c(OCc4ccc(Cl)cc4F)nc3C1)n2C[C@@H]1CCO1]

A mixture of (S)-7-((6-bromo-3-(oxetan-2-ylmethyl)-3H-imidazo[4,5-b]pyridin-2-yl) methyl)-2-((4-chloro-2-fluorobenzyl)oxy)-3-(trifluoromethyl)-5,6,7,8-tetrahydro-1,7-naphthyridine (100 mg, 0.156 mmol), ethynyltrimethylsilane (0.07 mL, 0.468 mmol), TEA (0.06 mL, 0.468 mmol), PPh₃ (0.8 mg, 0.003 mmol), bis(triphenylphosphine)palladium(II) chloride (2 mg, 0.003 mmol), and CuI (0.38 mg, 0.002 mmol) in DMF (4 mL) was stirred at 90° C. for 4 hrs under Ar. After the reaction was completed, the mixture was added to water (20 mL) and extracted with EtOAc (20 mL*2). The combined organic layers were washed with brine (20 mL*2), dried over Na₂SO₄, filtered, and concentrated in vacuum. The residue was purified by flash column chromatography (eluting PE/EA=10/1 to 1/1) to give (S)-2-((4-chloro-2-fluorobenzyl)oxy)-7-((3-(oxetan-2-ylmethyl)-6-((trimethylsilyl)ethynyl)-3H-imidazo[4,5-b]pyridin-2-yl)methyl)-3-(trifluoromethyl)-5,6,7,8-tetrahydro-1,7-naphthyridine (86 mg, 84% yield). MS Calcd.: 657.19; MS Found: 658.3 [M+H]⁺.

### Step C: (S)-2-((4-chloro-2-fluorobenzyl)oxy)-7-((6-ethynyl-3-(oxetan-2-ylmethyl)-3H-imidazo[4,5-b]pyridin-2-yl)methyl)-3-(trifluoromethyl)-5,6,7,8-tetrahydro-1,7-naphthyridine

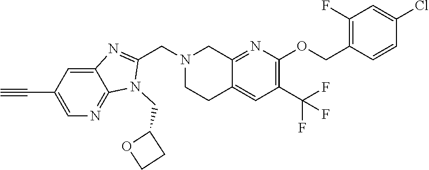 [SMILES: C#Cc1cnc2c(c1)nc(CN1CCc3cc(C(F)(F)F)c(OCc4ccc(Cl)cc4F)nc3C1)n2C[C@@H]1CCO1]

To a solution of (S)-2-((4-chloro-2-fluorobenzyl) oxy)-7-((3-(oxetan-2-ylmethyl)-6-((trimethylsilyl)ethynyl)-3H-imidazo[4,5-b]pyridin-2-yl)methyl)-3-(trifluoromethyl)-5,6,7,8-tetrahydro-1,7-naphthyridine (86 mg, 0.131 mmol) in THF (4 mL) was added TBAF (1M in THF, 0.2 ml, 0.196 mmol), and the reaction mixture was stirred at rt for 1 hr under Ar. After the reaction was completed, the reaction was quenched with a saturated aqueous ammonium chloride solution (4 mL) and extracted with EtOAc (20 mL*2). The combined organic layers were dried over Na₂SO₄, concentrated in vacuum, and the residue was purified by flash column chromatography (eluting PE/EA=10/1-1/1) to give (S)-2-((4-chloro-2-fluorobenzyl)oxy)-7-((6-ethynyl-3-(oxetan-2-ylmethyl)-3H-imidazo[4,5-b]pyridin-2-yl)methyl)-3-(trifluoromethyl)-5,6,7,8-tetrahydro-1,7-naphthyridine (50 mg, 65.3% yield). MS Calcd.: 585.16; MS Found: 586.3 [M+H]⁺.

### Step D: methyl (S)-3-(4-(2-((2-((4-chloro-2-fluorobenzyl)oxy)-3-(trifluoromethyl)-5,8-dihydro-1,7-naphthyridin-7(6H)-ylmethyl)-3-(oxetan-2-ylmethyl)-3H-imidazo[4,5-b]pyridin-6-yl)-1H-1,2,3-triazol-1-yl)propanoate

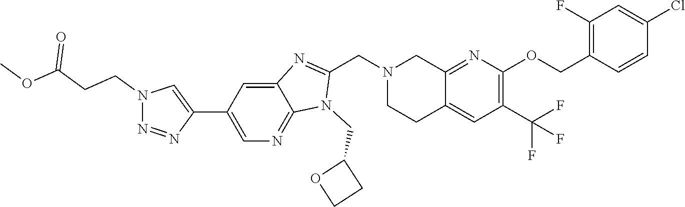 [SMILES: COC(=O)CCn1cc(-c2cnc3c(c2)nc(CN2CCc4cc(C(F)(F)F)c(OCc5ccc(Cl)cc5F)nc4C2)n3C[C@@H]2CCO2)nn1]

A solution of (S)-2-((4-chloro-2-fluorobenzyl)oxy)-7-((6-ethynyl-3-(oxetan-2-ylmethyl)-3H-imidazo[4,5-b]pyridin-2-yl)methyl)-3-(trifluoromethyl)-5,6,7,8-tetrahydro-1,7-naphthyridine (50 mg, 0.085 mmol), methyl 3-azidopropanoate (7 mg, 0.103 mmol), copper sulfate pentahydrate (2 mg, 0.009 mmol), and sodium ascorbate (2 mg, 0.009 mmol) in DMF (3 mL) and H₂O (3 mL) was stirred at rt for 2 hrs under Ar. After the reaction was completed, the mixture was added to water (20 mL) and extracted with EtOAc (20 mL*3). The combined organic layers were dried over Na₂SO₄, filtered, and concentrated in vacuum. The residue was purified by flash column chromatography (eluting DCM/MeOH=30/1) to give methyl (S)-3-(4-(2-((2-((4-chloro-2-fluorobenzyl)oxy)-3-(trifluoromethyl)-5,8-dihydro-1,7-naphthyridin-7(6H)-yl) methyl)-3-(oxetan-2-ylmethyl)-3H-imidazo[4,5-b]pyridin-6-yl)-1H-1,2,3-triazol-1-yl) propanoate (50 mg, 81.9% yield). MS Calcd.: 714.21; MS Found: 715.2 [M+H]⁺.

### Step E: 3-{4-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl-3H-imidazo[4,5-b]pyridin-6-yl]-1H-1,2,3-triazol-1-yl}propanoic acid (compound 99)

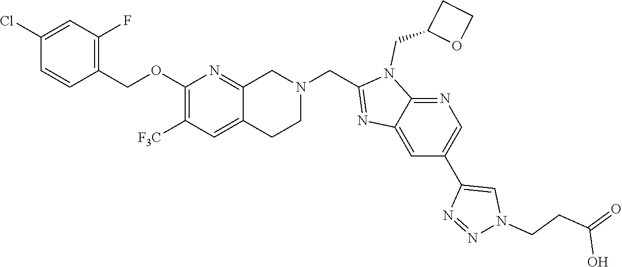 [SMILES: O=C(O)CCn1cc(-c2cnc3c(c2)nc(CN2CCc4cc(C(F)(F)F)c(OCc5ccc(Cl)cc5F)nc4C2)n3C[C@@H]2CCO2)nn1]

To a solution of methyl (S)-3-(4-(2-((2-((4-chloro-2-fluorobenzyl)oxy)-3-(trifluoromethyl)-5,8-dihydro-1,7-naphthyridin-7(6H)-yl)methyl)-3-(oxetan-2-ylmethyl)-3H-imidazo[4,5-b]pyridin-6-yl)-1H-1,2,3-triazol-1-yl)propanoate (25 mg, 0.035 mmol) in MeOH (3 mL) and THF (3 mL) was added a solution of NaOH (14 mg, 0.350 mmol) in H₂O (3 mL). The reaction mixture was stirred at 25° C. for 1.5 hrs. After the reaction was completed, 1N HCl was added to the mixture until pH<7, then the solvent was removed under reduced pressure. The residue was extracted with EtOAc (20 mL*2), and the combined organic layers were dried over Na₂SO₄, filtered, and concentrated in vacuum. The residue was purified by prep-HPLC to give 3-{4-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-b]pyridin-6-yl]-1H-1,2,3-triazol-1-yl}propanoic acid (1.77 mg, 7.22% yield).

¹H NMR (400 MHz, CD₃OD) δ 8.66 (s, 1H), 8.46 (d, J=7.6 Hz, 2H), 7.77 (s, 1H), 7.46 (t, J=8.0 Hz, 1H), 7.17 (d, J=9.2 Hz, 2H), 5.44 (s, 2H), 5.27-5.24 (m, 1H), 4.93-4.89 (m, 2H), 4.74-4.73 (m, 2H), 4.63-4.57 (m, 1H), 4.42-4.36 (m, 1H), 4.25 (s, 2H), 3.76 (s, 2H), 3.06-3.03 (m, 2H), 2.93-2.89 (m, 4H), 2.75-2.71 (m, 1H), 2.53-2.49 (m, 1H). ¹⁹F NMR (376 MHz, CD₃OD) δ −64.54, 117.35. LC-MS: m/z 701.4 (M+H)⁺.

### Procedure 32

### 4-[2-({2-[(4-chloro-2-fluorophenyl)methoxy]-3-(trifluoromethyl)-5,6,7,8-tetrahydro-1,7-naphthyridin-7-yl}methyl)-3-{1[(2S)-oxetan-2-yl]methyl}-3H-imidazo[4,5-c]pyridin-6-yl]-1,2-dihydropyridin-2-one (compound 100)

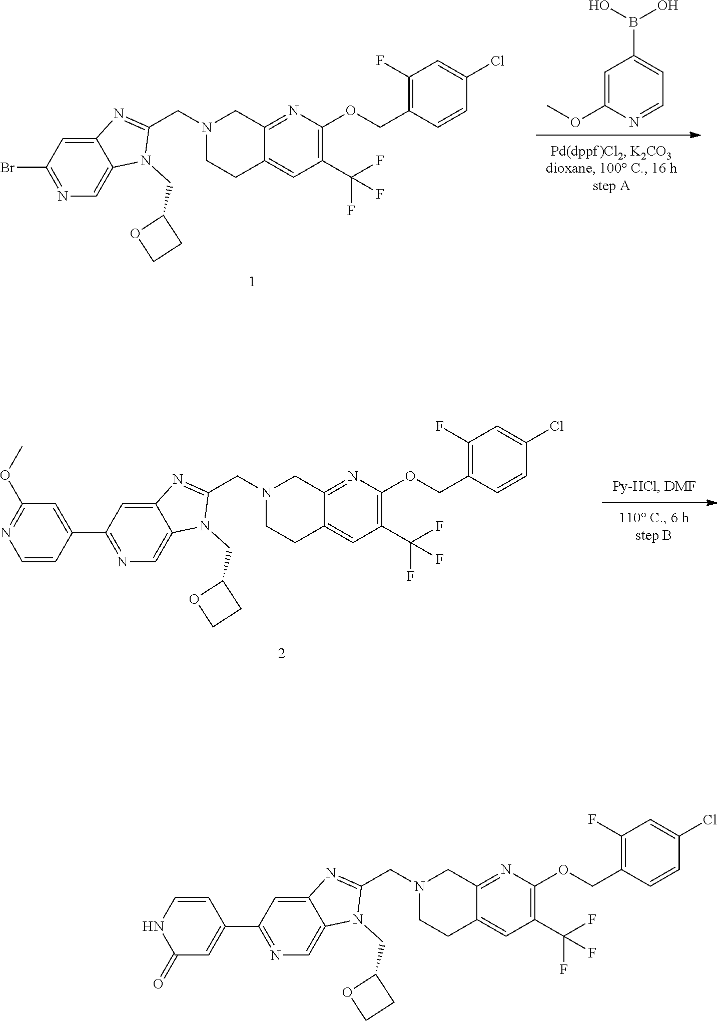 [SMILES: Fc1cc(Cl)ccc1COc1nc2c(cc1C(F)(F)F)CCN(Cc1nc3cc(Br)ncc3n1C[C@@H]1CCO1)C2 ; COc1cc(B(O)O)ccn1 ; COc1cc(-c2cc3nc(CN4CCc5cc(C(F)(F)F)c(OCc6ccc(Cl)cc6F)nc5C4)n(C[C@@H]4CCO4)c3cn2)ccn1 ; O=c1cc(-c2cc3nc(CN4CCc5cc(C(F)(F)F)c(OCc6ccc(Cl)cc6F)nc5C4)n(C[C@@H]4CCO4)c3cn2)cc[nH]1]

### Step A: (S)-2-((4-chloro-2-fluorobenzyl)oxy)-7-((6-(2-methoxypyridin-4-yl)-3-(oxetan-2-ylmethyl)-3H-imidazo[4,5-c]pyridin-2-yl)methyl)-3-(trifluoromethyl)-5,6,7,8-tetrahydro-1,7-naphthyridine

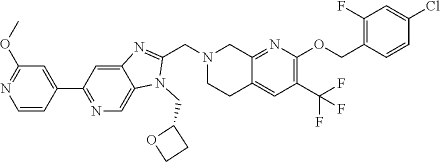 [SMILES: COc1cc(-c2cc3nc(CN4CCc5cc(C(F)(F)F)c(OCc6ccc(Cl)cc6F)nc5C4)n(C[C@@H]4CCO4)c3cn2)ccn1]

To a solution of (S)-7-((6-bromo-3-(oxetan-2-ylmethyl)-3H-imidazo[4,5-c]pyridin-2-yl)methyl)-2-((4-chloro-2-fluorobenzyl)oxy)-3-(trifluoromethyl)-5,6,7,8-tetrahydro-1,7-naphthyridine (50 mg, 0.078 mmol) in dioxane (20 mL) and H₂O (4 mL) was added (2-methoxypyridin-4-yl)boronic acid (13 mg, 0.086 mmol), K₂CO₃ (33 mg, 0.23 mmol), and Pd(dppf)Cl₂ (6 mg, 0.0078 mmol). The reaction mixture was stirred at 100° C. for 16 hours under Ar. After the reaction was completed, the mixture was concentrated in vacuum, and the residue was purified by prep-TLC (eluting DCM/MeOH=20/1) to give (S)-2-((4-chloro-2-fluorobenzyl)oxy)-7-((6-(2-methoxypyridin-4-yl)-3-(oxetan-2-ylmethyl)-3H-imidazo[4,5-c]pyridin-2-yl)methyl)-3-(trifluoromethyl)-5,6,7,8-tetrahydro-1,7-naphthyridine (30 mg, yield: 57.69%). MS Calcd.: 668.19; MS Found: 669.3 [M+H]⁺.

### Step B: 4-[2-({2-[(4-chloro-2-fluorophenyl)methoxy)-5,6,7,8-tetrahydro-1,7-naphthyridin-7-yl}methyl)-3-{[(2S)-oxetan-2-yl]methyl)-3H-imidazo[4,5-c]pyridin-6-yl]-1,2-dihydropyridin-2-one (compound 100)

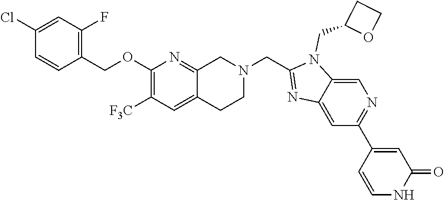 [SMILES: O=c1cc(-c2cc3nc(CN4CCc5cc(C(F)(F)F)c(OCc6ccc(Cl)cc6F)nc5C4)n(C[C@@H]4CCO4)c3cn2)cc[nH]1]

To a solution of (S)-2-((4-chloro-2-fluorobenzyl)oxy)-7-((6-(2-methoxypyridin-4-yl)-3-(oxetan-2-ylmethyl)-3H-imidazo[4,5-c]pyridin-2-yl)methyl)-3-(trifluoromethyl)-5,6,7,8-tetrahydro-1,7-naphthyridine (30 mg, 0.045 mmol) in DMF (5 mL) was added pyridine hydrochloride (26 mg, 0.225 mmol), and the reaction mixture was stirred at 110° C. for 6 hours. After the reaction was completed, the mixture was concentrated in vacuum, and the residue was purified by prep-HPLC to give 4-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-1,2-dihydropyridin-2-one (1.75 mg, yield: 5.96%).

¹H NMR (400 MHz, CD₃OD) δ 9.15 (s, 1H), 8.32 (s, 1H), 8.12 (s, 1H), 7.57-7.50 (m, 2H), 7.29-7.23 (m, 3H), 7.16-7.14 (m, 1H), 5.58 (s, 2H), 5.16-5.13 (m, 1H), 5.06-5.01 (m, 2H), 4.97-4.95 (m, 2H), 4.92-4.90 (m, 2H), 4.64-4.59 (m, 1H), 4.49-4.41 (m, 1H), 4.00-3.94 (m, 2H), 3.71-3.67 (m, 1H), 3.55-3.47 (m, 1H), 2.77-2.65 (m, 1H), 2.41-2.25 (m, 1H). ¹⁹F NMR (376 MHz, CD₃OD) δ −65.05, −117.37. LC-MS: m/z 655.2 (M+H)⁺.

### 3-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-1,2-dihydropyridin-2-one (compound 101)

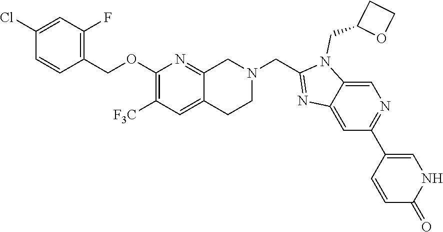 [SMILES: O=c1ccc(-c2cc3nc(CN4CCc5cc(C(F)(F)F)c(OCc6ccc(Cl)cc6F)nc5C4)n(C[C@@H]4CCO4)c3cn2)c[nH]1]

Compound 101 was synthesized following the similar route of Procedure 32, using (2-hydroxypyridin-3-yl) boronic acid in step A.

¹H NMR (400 MHz, DMSO-d₆) δ 9.01 (s, 1H), 8.91 (s, 1H), 8.47 (d, J=7.2 Hz, 1H), 7.91 (s, 1H), 7.51 (t, J=8.0 Hz, 1H), 7.45 (d, J=8.4 Hz, 2H), 7.30 (d, J=8.8 Hz, 1H), 6.39-6.37 (m, 1H), 5.42 (s, 2H), 5.09-5.07 (m, 1H), 4.83-4.77 (m, 1H), 4.68-4.65 (m, 1H), 4.47-4.43 (m, 1H), 4.38-4.35 (m, 1H), 4.16 (d, J=13.6 Hz, 1H), 4.06 (d, J=13.6 Hz, 1H), 3.72-3.70 (m, 2H), 2.85-2.81 (m, 4H), 2.67-2.62 (m, 1H), 2.32-2.29 (m, 1H). ¹⁹F NMR (376 MHz, DMSO-d₆) δ −61.67, 114.96. LC-MS: m/z 655.5 (M+H)⁺.

### 5-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]-1,2-dihydropyridin-2-one (compound 102)

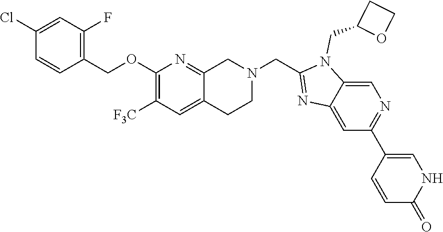 [SMILES: O=c1ccc(-c2cc3nc(CN4CCc5cc(C(F)(F)F)c(OCc6ccc(Cl)cc6F)nc5C4)n(C[C@@H]4CCO4)c3cn2)c[nH]1]

Compound 102 was synthesized following the similar route of Procedure 32, using (6-methoxypyridin-3-yl)boronic acid in step A.

¹H NMR (400 MHz, CD₃OD) δ 9.05 (s, 1H), 8.55 (brs, 1H), 8.32 (dd, J1=2.8 Hz, J2=2.4 Hz, 1H), 8.18-8.17 (m, 1H), 8.11-8.07 (m, 2H), 7.57-7.53 (m, 1H), 7.28-7.24 (m, 2H), 6.68 (d, J=9.6 Hz, 1H), 5.62-5.52 (m, 2H), 5.18-5.10 (m, 2H), 4.93-4.90 (m, 2H), 4.60-4.56 (m, 2H), 4.51-4.43 (m, 1H), 4.31-4.23 (m, 1H), 3403-3.97 (m, 1H), 3.47-3.24 (m, 4H), 2.65-2.58 (m, 1H), 2.38 (m, 1H). ¹⁹F NMR (376 MHz, CD₃OD) δ −65.07, 117.13. LC-MS: m/z 655.2 (M+H)⁺.

### Procedure 33

### N-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]pyridine-3-carboxamide (compound 103)

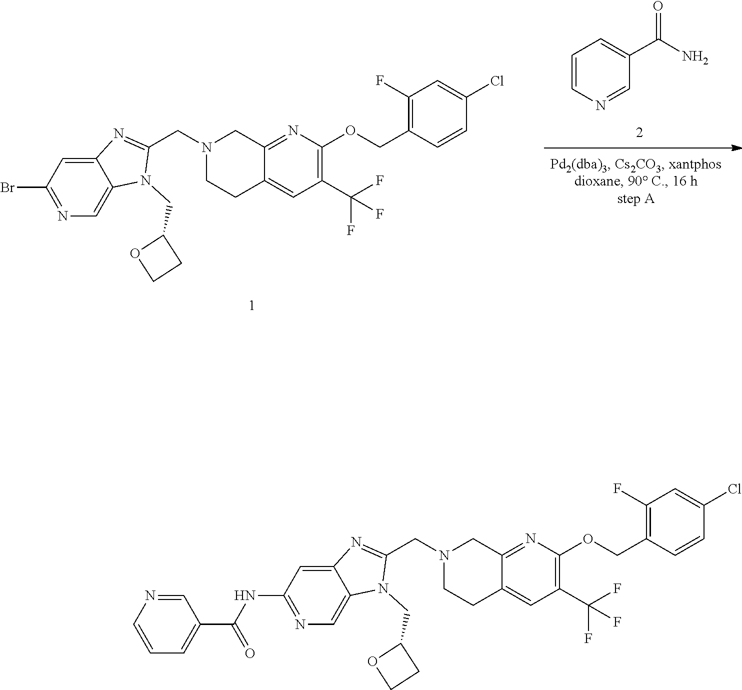 [SMILES: Fc1cc(Cl)ccc1COc1nc2c(cc1C(F)(F)F)CCN(Cc1nc3cc(Br)ncc3n1C[C@@H]1CCO1)C2 ; NC(=O)c1cccnc1 ; O=C(Nc1cc2nc(CN3CCc4cc(C(F)(F)F)c(OCc5ccc(Cl)cc5F)nc4C3)n(C[C@@H]3CCO3)c2cn1)c1cccnc1]

A mixture of (S)-7-((6-bromo-3-(oxetan-2-ylmethyl)-3H-imidazo[4,5-c]pyridin-2-yl)methyl)-2-((4-chloro-2-fluorobenzyl)oxy)-3-(trifluoromethyl)-5,6,7,8-tetrahydro-1,7-naphthyridine (20 mg, 0.031 mmol), nicotinamide (4 mg, 0.031 mmol), tris(dibenzylideneacetone) dipalladium (6 mg, 0.006 mmol), Cs₂CO₃ (21 mg, 0.063 mmol) and xantphos (3 mg, 0.006 mmol) in dioxane (10 mL) was stirred at 90° C. for 18 hrs under Ar. After the reaction was completed, the mixture was concentrated and the residue was purified by prep-HPLC to give N-[2-({2-[(4-chloro-2-fluorophenyl)methoxy]-3-(trifluoromethyl)-5,6,7,8-tetrahydro-1,7-naphthyridin-7-yl}methyl)-3-{[(2S)-oxetan-2-yl]methyl}-3H-imidazo[4,5-c]pyridin-6-yl]pyridine-3-carboxamide (2.39 mg, 11.22% yield).

¹H NMR (400 MHz, CD₃OD) δ 9.17 (s, 1H), 8.89 (s, 1H), 8.79 (d, J=4.8 Hz, 1H), 8.49-8.47 (m, 2H), 7.92 (s, 1H), 7.69-7.65 (m, 1H), 7.50-7.46 (t, J=16 Hz, 1H), 7.21-7.20 (m, 2H), 5.48 (s, 2H), 5.21-5.20 (m, 1H), 4.80-4.75 (m, 2H), 4.72-4.69 (m, 2H), 4.65-4.60 (m, 1H), 4.45-4.40 (m, 1H), 4.31-4.26 (m, 2H), 3.49-3.45 (m, 2H), 3.15-3.08 (m, 2H), 2.81-2.76 (m, 1H), 2.52-2.47 (m, 1H). ¹⁹F NMR (376 MHz, CD₃OD) δ −64.78, 117.28. LC-MS: m/z 682.4 (M+H)⁺.

### Procedure 34

### 3-{2-[(2-{[(4-chloro-2-fluorophenyl)methyl]amino}-3-(trifluoromethyl)-5,6,7,8-tetrahydro-1,7-naphthyridin-7-yl)methyl]-3-{[(2S)-oxetan-2-yl]methyl}-3H-imidazo[4,5-c]pyridin-6-yl}-4,5-dihydro-1,2,4-oxadiazol-5-one (compound 104)

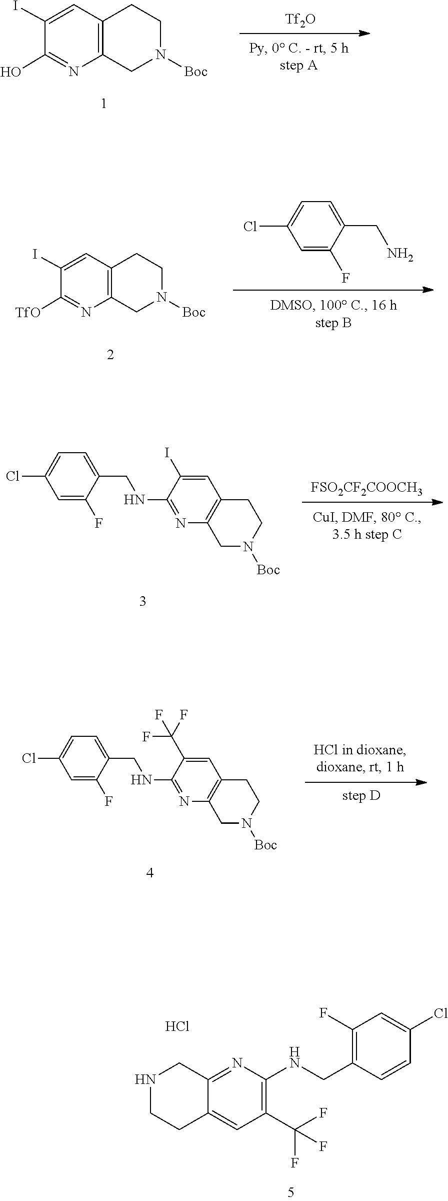

### Step A: tert-butyl 3-iodo-2-(((trifluoromethyl)sulfonyl)oxy)-5,8-dihydro-1,7-naphthyridine-7(6H)-carboxylate

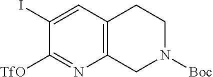 [SMILES: CC(C)(C)OC(=O)N1CCc2cc(I)c(OS(=O)(=O)C(F)(F)F)nc2C1]

To a mixture of tert-butyl 2-hydroxy-3-iodo-5,8-dihydro-1,7-naphthyridine-7(6H)-carboxylate (3.30 g, 8.77 mmol) in pyridine (20.0 mL) was added Tf₂O (4.95 g, 17.55 mmol) at 0° C. The mixture was stirred at room temperature for 5 hrs. After the reaction was completed, the mixture was diluted with H₂O (250 mL) and extracted with EA (100 mL×3). The combined organic layer was dried over anhydrous sodium sulfate, filtered, and evaporated to dryness. The residue was purified by silica gel column chromatography, eluted with (PE/EA=5/1) to give tert-butyl 3-iodo-2-(((trifluoromethyl)sulfonyl)oxy)-5,8-dihydro-1,7-naphthyridine-7(6H)-carboxylate (3.4 g, yield: 76.2%). MS Calcd.: 507.98; MS Found: 453.0 [M−56+H]⁺.

### Step B: tert-butyl 2-((4-chloro-2-fluorobenzyl)amino)-3-iodo-5,8-dihydro-1,7-naphthyridine-7(6H)-carboxylate

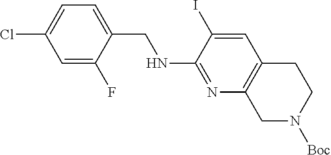 [SMILES: CC(C)(C)OC(=O)N1CCc2cc(I)c(NCc3ccc(Cl)cc3F)nc2C1]

To a mixture of tert-butyl 3-iodo-2-(((trifluoromethyl)sulfonyl)oxy)-5,8-dihydro-1,7-naphthyridine-7(6H)-carboxylate (1.00 g, 1.97 mmol) in DMSO (15.0 mL) was added (4-chloro-2-fluorophenyl)methanamine (629.54 mg, 3.94 mmol) at room temperature. The mixture was stirred at 100° C. for 16 hrs. After the reaction was completed, the mixture was diluted with H₂O (50 mL) and extracted with EA (40 mL×2). The combined organic layer was dried over anhydrous sodium sulfate, filtered, and evaporated to dryness. The residue was purified by silica gel column chromatography, eluted with (PE/EA=20/1) to give tert-butyl 2-((4-chloro-2-fluorobenzyl)amino)-3-iodo-5,8-dihydro-1,7-naphthyridine-7(6H)-carboxylate (523 mg, yield: 51.3%). MS Calcd.: 517.04; MS Found: 518.0 [M+H]⁺.

### Step C: tert-butyl 2-((4-chloro-2-fluorobenzyl)amino)-3-(trifluoromethyl)-5,8-dihydro-1,7-naphthyridine-7(6H)-carboxylate

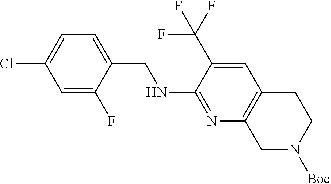 [SMILES: CC(C)(C)OC(=O)N1CCc2cc(C(F)(F)F)c(NCc3ccc(Cl)cc3F)nc2C1]

To a mixture of tert-butyl 2-((4-chloro-2-fluorobenzyl)amino)-3-iodo-5,8-dihydro-1,7-naphthyridine-7(6H)-carboxylate (523.00 mg, 1.01 mmol) and CuI (19.22 mg, 0.10 mmol) in DMF (4.0 mL) was added methyl 2,2-difluoro-2-(fluorosulfonyl)acetate (582.68 mg, 3.03 mmol) at room temperature. The mixture was stirred at 80° C. for 3.5 hrs. After the reaction was completed, the mixture was diluted with H₂O (40 mL) and extracted with EA (30 mL×2). The combined organic layer was dried over anhydrous sodium sulfate, filtered, and evaporated to dryness. The residue was purified by column chromatography on C₁₈, eluted with (CH₃CN/H₂O=9/1) to give tert-butyl 2-((4-chloro-2-fluorobenzyl)amino)-3-(trifluoromethyl)-5,8-dihydro-1,7-naphthyridine-7(6H)-carboxylate (150 mg, yield: 32.3%). MS Calcd.: 459.13; MS Found: 460.2 [M+H]⁺.

### Step D: N-(4-chloro-2-fluorobenzyl)-3-(trifluoromethyl)-5,6,7,8-tetrahydro-1,7-naphthyridin-2-amine, HCl Salt

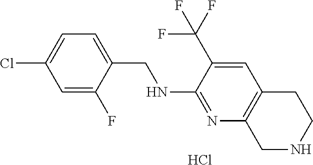 [SMILES: Fc1cc(Cl)ccc1CNc1nc2c(cc1C(F)(F)F)CCNC2 ; Cl]

To a solution of tert-butyl 2-((4-chloro-2-fluorobenzyl)amino)-3-(trifluoromethyl)-5,8-dihydro-1,7-naphthyridine-7(6H)-carboxylate (150 mg, 0.33 mmol) in dioxane (2 mL) was added 4 M HCl in dioxane (2 mL). The solution was stirred at room temperature for 1 hr. The reaction was dried in vacuo to give N-(4-chloro-2-fluorobenzyl)-3-(trifluoromethyl)-5,6,7,8-tetrahydro-1,7-naphthyridin-2-amine, HCl salt (170 mg, crude) which was used in the next step without further purification. MS Calcd.: 359.08; MS Found: 360.1 [M+H]⁺.

### 3-{2-[(2-{[(4-chloro-2-fluorophenyl)methyl]amino}-3-(trifluoromethyl)-5,6,7,8-tetrahydro-1,7-naphthyridin-7-yl)methyl]-3-{[(2S)-oxetan-2-yl]methyl-3H-imidazo[4,5-c]pyridin-6-yl-4,5-dihydro-1,2,4-oxadiazol-5-one (compound 104)

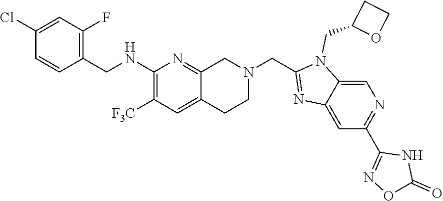 [SMILES: O=c1[nH]c(-c2cc3nc(CN4CCc5cc(C(F)(F)F)c(NCc6ccc(Cl)cc6F)nc5C4)n(C[C@@H]4CCO4)c3cn2)no1]

Compound 104 was then synthesized following the route of Procedure 1, using (4-chloro-2-fluorophenyl)methanamine in step B.

¹H NMR (400 MHz, DMSO-d₆) δ 13.00 (br s, 1H), 9.22 (s, 1H), 8.25 (s, 1H), 7.69 (s, 1H), 7.23-7.34 (m, 2H), 7.16 (dd, J=8.4 Hz, 2.0 Hz, 1H), 7.01-7.12 (m, 1H), 5.02-5.13 (m, 1H), 4.82-4.92 (m, 1H), 4.66-4.76 (m, 1H), 4.52-4.60 (m, 2H), 4.41-4.50 (m, 1H), 4.28-4.38 (m, 1H), 3.82-4.06 (m, 4H), 2.76-2.89 (m, 2H), 2.57-2.74 (m, 3H), 2.28-2.40 (m, 1H). ¹⁹F NMR (376 MHz, DMSO-d₆) δ −62.09, −116.00. LC-MS: m/z 645.2 (M+H)⁺.

### 3-{2-[(2-{[(4-chloro-2,6-difluorophenyl)methyl]amino}-3-(trifluoromethyl)-5,6,7,8-tetrahydro-1,7-naphthyridin-7-yl)methyl]-3-{[(2S)-oxetan-2-yl]methyl}-3H-imidazo[4,5-c]pyridin-6-yl}-4,5-dihydro-1,2,4-oxadiazol-5-one (compound 105)

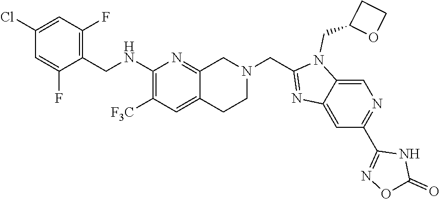 [SMILES: O=c1[nH]c(-c2cc3nc(CN4CCc5cc(C(F)(F)F)c(NCc6c(F)cc(Cl)cc6F)nc5C4)n(C[C@@H]4CCO4)c3cn2)no1]

Compound 105 was synthesized following the similar route of Procedure 34, using (4-chloro-2,6-difluorophenyl)methanamine in step B.

¹H NMR (400 MHz, DMSO-d₆) δ 13.01 (br s, 1H), 9.23 (s, 1H), 8.28 (s, 1H), 7.64 (s, 1H), 7.10-7.23 (m, 2H), 6.86 (s, 1H), 5.04-5.17 (m, 1H), 4.84-4.95 (m, 1H), 4.69-4.80 (m, 1H), 4.41-4.63 (m, 4H), 4.29-4.39 (m, 1H), 3.82-4.03 (m, 3H), 3.03-3.33 (m, 2H), 2.76-2.91 (m, 2H), 2.64-2.74 (m, 1H), 2.28-2.42 (m, 1H). ¹⁹F NMR (376 MHz, DMSO-d₆) δ −62.01, −111.80. LC-MS: m/z 663.3 (M+H)⁺.

### 3-{2-[(2-{[(4-chloro-2,6-difluorophenyl)methyl]amino}-3-methyl-5,6,7,8-tetrahydro-1,7-naphthyridin-7-yl)methyl]-3-{[(2S)-oxetan-2-yl]methyl}-3H-imidazo[4,5-c]pyridin-6-yl}-4,5-dihydro-1,2,4-oxadiazol-5-one (compound 35)

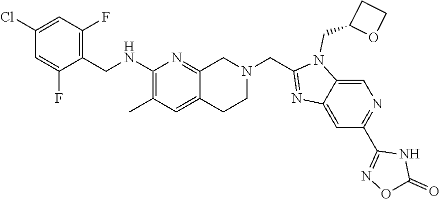 [SMILES: Cc1cc2c(nc1NCc1c(F)cc(Cl)cc1F)CN(Cc1nc3cc(-c4noc(=O)[nH]4)ncc3n1C[C@@H]1CCO1)CC2]

Compound 35 was synthesized following the similar route of Procedure 34, using (4-chloro-2,6-difluorophenyl)methanamine in step B and 2,4,6-trimethyl-1,3,5,2,4,6-trioxatriborinane, PdCl₂(dppf). DCM, Cs₂CO₃ in step C.

¹H NMR (400 MHz, DMSO-d₆) δ 9.09 (s, 1H), 8.15 (s, 1H), 7.19 (d, J=7.2 Hz, 2H), 6.98 (s, 1H), 6.11 (t, J=4.8 Hz, 1H), 5.04-5.12 (m, 1H), 4.83-4.90 (m, 1H), 4.68-4.77 (m, 1H), 4.45-4.49 (m, 1H), 4.39-4.44 (m, 2H), 4.32-4.38 (m, 1H), 4.12 (d, J=13.6 Hz, 1H), 4.01 (d, J=13.6 Hz, 1H), 3.41-3.49 (m, 2H), 2.68-2.79 (m, 2H), 2.56-2.61 (m, 3H), 2.32-2.39 (m, 1H), 1.98 (s, 3H). ¹⁹F NMR (376 MHz, DMSO-d₆) δ −111.92. LC-MS: m/z 609.2 (M+H)⁺.

### 3-{2-[(2-{[(4-chloro-2-fluorophenyl)methyl]amino}-3-(trifluoromethyl)-5,6,7,8-tetrahydro-1,7-naphthyridin-7-yl)methyl]-4-fluoro-1-{[(2S)-oxetan-2-yl]methyl}-1H-1,3-benzodiazol-5-yl}-4,5-dihydro-1,2,4-oxadiazol-5-one (compound 106)

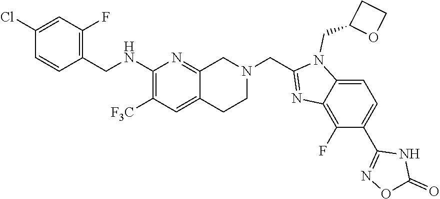 [SMILES: O=c1[nH]c(-c2ccc3c(nc(CN4CCc5cc(C(F)(F)F)c(NCc6ccc(Cl)cc6F)nc5C4)n3C[C@@H]3CCO3)c2F)no1]

Compound 106 was synthesized following the similar route of Procedure 2, using N-(4-chloro-2,6-difluorobenzyl)-3-(trifluoromethyl)-5,6,7,8-tetrahydro-1,7-naphthyridin-2-amine HCl Salt and (S)-2-(chloromethyl)-4-fluoro-1-(oxetan-2-ylmethyl)-1H-benzo[d]imidazole-5-carbonitrile in step A.

¹H NMR (400 MHz, DMSO-d₆) δ 7.64 (d, J=8.8 Hz, 1H), 7.54-7.61 (m, 2H), 7.22-7.33 (m, 2H), 7.16 (dd, J=8.4 Hz, 1.6 Hz, 1H), 6.91 (t, J=6.0 Hz, 1H), 4.99-5.08 (m, 1H), 4.70-4.79 (m, 1H), 4.51-4.65 (m, 3H), 4.40-4.48 (m, 1H), 4.28-4.36 (m, 1H), 4.07 (d, J=13.6 Hz, 1H), 3.98 (d, J=13.6 Hz, 1H), 3.42-3.55 (m, 2H), 2.70-2.78 (m, 2H), 2.56-2.69 (m, 3H), 2.26-2.38 (m, 1H). ¹⁹F NMR (376 MHz, DMSO-d₆) δ −61.92, −116.03, −126.07. LC-MS: m/z 662.2 (M+H)⁺.

### 5-{2-[(2-{[(4-chloro-2-fluorophenyl)methyl]amino}-3-(trifluoromethyl)-5,6,7,8-tetrahydro-1,7-naphthyridin-7-yl)methyl]-3-{[(2S)-oxetan-2-yl]methyl}-3H-imidazo[4,5-c]pyridin-6-yl}-4H-1,2,4-triazole-3-carboxamide (compound 107) and 5-{2-[(2-{[(4-chloro-2-fluorophenyl)methyl]amino}-3-(trifluoromethyl)-5,6,7,8-tetrahydro-1,7-naphthyridin-7-yl)methyl]-3-{[(2S)-oxetan-2-yl]methyl}-3H-imidazo[4,5-c]pyridin-6-yl}-4H-1,2,4-triazole-3-carbonitrile (compound 108)

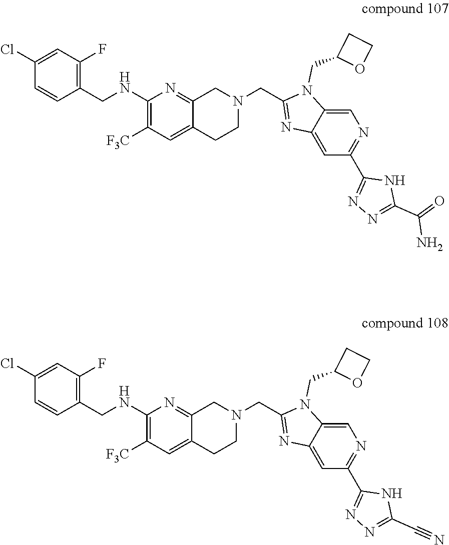 [SMILES: NC(=O)c1nnc(-c2cc3nc(CN4CCc5cc(C(F)(F)F)c(NCc6ccc(Cl)cc6F)nc5C4)n(C[C@@H]4CCO4)c3cn2)[nH]1 ; N#Cc1nnc(-c2cc3nc(CN4CCc5cc(C(F)(F)F)c(NCc6ccc(Cl)cc6F)nc5C4)n(C[C@@H]4CCO4)c3cn2)[nH]1]

Compound 107 and 108 were synthesized following the similar route of Procedure 5, using tert-butyl 2-((4-chloro-2-fluorobenzyl)amino)-3-(trifluoromethyl)-5,8-dihydro-1,7-naphthyridine-7(6H)-carboxylate in step A.

Compound 107: ¹H NMR (400 MHz, DMSO-d₆) δ 9.11 (s, 1H), 8.30 (s, 1H), 7.88 (s, 1H), 7.59 (s, 2H), 7.21-7.32 (m, 2H), 7.15-7.17 (m, 1H), 6.92 (t, J=5.6 Hz, 1H), 5.03-5.13 (m, 1H), 4.81-4.91 (m, 1H), 4.66-4.75 (m, 1H), 4.51-4.57 (m, 2H), 4.42-4.50 (m, 1H), 4.31-4.39 (m, 1H), 4.13 (d, J=13.6 Hz, 1H), 4.02 (d, J=13.6 Hz, 1H), 3.44-3.58 (m, 2H), 2.72-2.79 (m, 2H), 2.58-2.70 (m, 3H), 2.30-2.42 (m, 1H). ¹⁹F NMR (376 MHz, DMSO-d₆) δ −61.92, −116.05. LC-MS: m/z 671.3 (M+H)⁺.

Compound 108: ¹H NMR (400 MHz, DMSO-d₆) δ 9.11 (s, 1H), 8.30 (s, 1H), 7.88 (s, 1H), 7.59 (s, 2H), 7.21-7.32 (m, 2H), 7.15-7.17 (m, 1H), 6.92 (t, J=5.6 Hz, 1H), 5.03-5.13 (m, 1H), 4.81-4.91 (m, 1H), 4.66-4.75 (m, 1H), 4.51-4.57 (m, 2H), 4.42-4.50 (m, 1H), 4.31-4.39 (m, 1H), 4.13 (d, J=13.6 Hz, 1H), 4.02 (d, J=13.6 Hz, 1H), 3.44-3.58 (m, 2H), 2.72-2.79 (m, 2H), 2.58-2.70 (m, 3H), 2.30-2.42 (m, 1H). ¹⁹F NMR (376 MHz, DMSO-d₆) δ −62.24, −115.98. LC-MS: m/z 653.3 (M+H)⁺.

### N-[(4-chloro-2-fluorophenyl)methyl]-7-[(3-{1[(2S)-oxetan-2-yl]methyl}-6-[5-(trifluoromethyl)-4H-1,2,4-triazol-3-yl]-3H-imidazo[4,5-c]pyridin-2-yl)methyl]-3-(trifluoromethyl)-5,6,7,8-tetrahydro-1,7-naphthyridin-2-amine (compound 109)

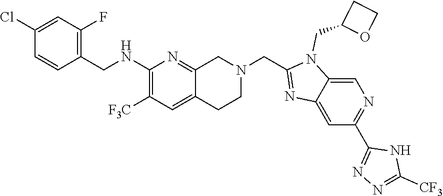 [SMILES: Fc1cc(Cl)ccc1CNc1nc2c(cc1C(F)(F)F)CCN(Cc1nc3cc(-c4nnc(C(F)(F)F)[nH]4)ncc3n1C[C@@H]1CCO1)C2]

Compound 109 was synthesized following the similar route of Procedure 3, using tert-butyl 2-((4-chloro-2-fluorobenzyl)amino)-3-(trifluoromethyl)-5,8-dihydro-1,7-naphthyridine-7(6H)-carboxylate in step A and (S)-2-(chloromethyl)-3-(oxetan-2-ylmethyl)-3H-imidazo[4,5-c]pyridine-6-carbonitrile in step B.

¹H NMR (400 MHz, DMSO-d₆) δ 9.14 (s, 1H), 8.32 (s, 1H), 7.59 (s, 1H), 7.23-7.29 (m, 2H), 7.15-7.17 (m, 1H), 6.92 (t, J=6.0 Hz, 1H), 5.08-5.09 (m, 1H), 4.84-4.89 (m, 1H), 4.70-4.73 (m, 1H), 4.54-4.55 (m, 2H), 4.44-4.49 (m, 1H), 4.34-4.38 (m, 1H), 4.01-4.15 (m, 2H), 3.46-3.56 (m, 2H), 2.75-2.76 (m, 2H), 2.63-2.68 (m, 3H), 2.34-2.39 (m, 1H). ¹⁹F NMR (376 MHz, DMSO-d₆) δ −61.92, −63.47, −116.04. LC-MS: m/z 696.3 (M+H)⁺.

### N-[(4-chloro-2,6-difluorophenyl)methyl]-7-{[6-(5-cyclopropyl-4H-1,2,4-triazol-3-yl)-3-{[(2S)-oxetan-2-yl]methyl}-3H-imidazo[4,5-c]pyridin-2-yl]methyl}-3-(trifluoromethyl)-5,6,7,8-tetrahydro-1,7-naphthyridin-2-amine (compound 110)

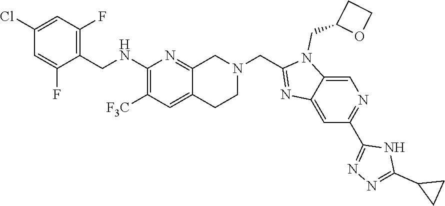 [SMILES: Fc1cc(Cl)cc(F)c1CNc1nc2c(cc1C(F)(F)F)CCN(Cc1nc3cc(-c4nnc(C5CC5)[nH]4)ncc3n1C[C@@H]1CCO1)C2]

Compound 110 was synthesized following the similar route of Procedure 13, using (S)-2-((2-((4-chloro-2-fluorobenzyl)amino)-3-(trifluoromethyl)-5,8-dihydro-1,7-naphthyridin-7(6H)-yl)methyl)-3-(oxetan-2-ylmethyl)-3H-imidazo[4,5-c]pyridine-6-carbonitrile in step A.

¹H NMR (400 MHz, DMSO-d₆) δ 14.02 (s, 0.7H), 9.08 (s, 1H), 8.18 (s, 1H), 7.90 (s, 1H), 7.38 (d, J=7.2 Hz, 2H), 5.39 (s, 2H), 5.09-5.08 (m, 1H), 4.89-4.85 (m, 1H), 4.73-4.68 (m, 1H), 4.48-4.46 (m, 1H), 4.39-4.35 (m, 1H), 4.21-4.17 (m, 1H), 4.10-4.07 (m, 1H), 3.72-3.71 (m, 2H), 2.85-2.81 (m, 4H), 2.76-2.66 (m, 1H), 2.40-2.32 (m, 1H), 2.02 (m, 1H), 0.92-0.88 (m, 4H). ¹⁹F NMR (376 MHz, DMSO-d₆) δ −61.76, −112.07. LC-MS: m/z 686.4 (M+H)⁺.

### BIOLOGICAL EXAMPLES

### Biological Example 1: cAMP Assays

Activation of GLP-1 receptor is known to stimulate cyclic AMP (cAMP) production in cells which indicates primary coupling to the Gas subunit of the G protein heterotrimeric complex. Evidence suggests signaling through Gas induced cAMP stimulation elicits the desired pharmacological response regarding insulin release from pancreatic β-cells.

To optimize functional activity directed toward Gas coupling, a HEK293/CRE-Luc cell line developed by HDB stably expressing the GLP-1 Receptor was used. 200× concentration of compound working solutions were prepared (Agilent Technologies Bravo) with 1/2 log serial dilution in 384-well Echo LDV plate (Labcyte, Cat #LP-0200). 50 nL/well 200× concentration of compound working solutions were moved to 384-well white low volume plate (Greiner, Cat #784075) using Labcyte ECHO550. 1×105 cells/mL HEK293/GLP1R/CRE-LUC (HD Biosciences) cell suspensions prepared with assay buffer [DPBS containing 0.5 mM IBMX (Sigma, Cat #15879) and 0.10% BSA (GENVIEW, Cat #FA016-100g)], 10 μL cell suspensions were added to each well of previous generated assay plate which already contains 50 nL compound at 200× concentration using ThermoFisher Multidrop Combi (1000 cells/well). Seal the plate and incubate at 37° C. with 5% CO₂ for 30 min.

After incubation the cAMP assay signal was generated using cAMP dynamic 2 Kit (Cisbio). 5 μL cAMP-d2 working solution was added to each well, followed with 5 μL Anti-cAMP antibody-cryptate working solution added to each well using ThermoFisher Multidrop Combi. Incubate at room temperature for 1 hour protected from light. Read the fluorescence at 665 and 615 nm with Reader PerkinElmer EnVision.

$$\% Activity = 100 \% \times ( mean RLU of test sample - mean RLU of vehicle control ) / ( mean RLU of MAX control - mean RLU of vehicle control ) )$$

Table 2 shows the biological activity of compounds in GLP-1R agonist cAMP stimulation assay (EC₅₀). Activity of the tested compounds is provided in Table 2 below as follows: +++=EC₅₀<1 nM; ++=EC₅₀ 1-300 nM; +=EC₅₀>300 nM.

TABLE 2

| No. | Activity |
|---|---|
| 1 | ++ |
| 2 | ++ |
| 3 | ++ |
| 4 | +++ |
| 5 | +++ |
| 6 | +++ |
| 7 | +++ |
| 8 | +++ |
| 9 | +++ |
| 10 | ++ |
| 11 | + |
| 12 | + |
| 13 | + |
| 14 | +++ |
| 15 | +++ |
| 16 | +++ |
| 17 | +++ |
| 18 | +++ |
| 19 | +++ |
| 20 | +++ |
| 21 | ++ |
| 22 | ++ |
| 23 | ++ |
| 24 | ++ |
| 25 | ++ |
| 26 | +++ |
| 27 | ++ |
| 28 | +++ |
| 29 | +++ |
| 30 | +++ |
| 31 | +++ |
| 32 | +++ |
| 33 | +++ |
| 34 | +++ |
| 35 | +++ |
| 36 | +++ |
| 37 | +++ |
| 38 | ++ |
| 39 | +++ |
| 40 | +++ |
| 41 | +++ |
| 42 | +++ |
| 43 | +++ |
| 44 | +++ |
| 45 | +++ |
| 46 | +++ |
| 47 | +++ |
| 48 | ++ |
| 49 | +++ |
| 50 | +++ |
| 51 | +++ |
| 52 | +++ |
| 53 | +++ |
| 54 | +++ |
| 55 | +++ |
| 56 | +++ |
| 57 | +++ |
| 58 | +++ |
| 59 | +++ |
| 60 | ++ |
| 61 | +++ |
| 62 | +++ |
| 63 | +++ |
| 64 | ++ |
| 65 | +++ |
| 66 | +++ |
| 67 | ++ |
| 68 | +++ |
| 69 | +++ |
| 70 | +++ |
| 71 | +++ |
| 72 | +++ |
| 73 | +++ |
| 74 | +++ |
| 75 | +++ |
| 76 | ++ |
| 77 | ++ |
| 78 | ++ |
| 79 | ++ |
| 80 | ++ |
| 81 | ++ |
| 82 | +++ |
| 83 | +++ |
| 84 | +++ |
| 85 | +++ |
| 86 | +++ |
| 87 | ++ |
| 88 | +++ |
| 89 | +++ |
| 90 | +++ |
| 91 | +++ |
| 92 | ++ |
| 93 | +++ |
| 94 | +++ |
| 95 | ++ |
| 96 | +++ |
| 97 | +++ |
| 98 | +++ |
| 99 | ++ |
| 100 | + |
| 101 | ++ |
| 102 | + |
| 103 | +++ |
| 104 | +++ |
| 105 | +++ |
| 106 | +++ |
| 107 | +++ |
| 108 | +++ |
| 109 | +++ |
| 110 | +++ |

What is claimed is:

1. A compound of Formula I: 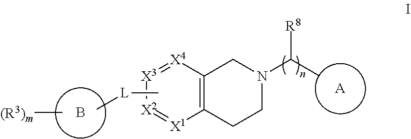
or a pharmaceutically acceptable salt or solvate thereof, wherein:
ring A is 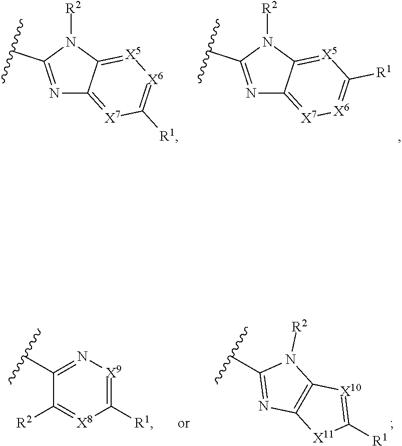
ring B is C₃₋₁₀ cycloalkyl, heterocyclyl, aryl, or heteroaryl;
one of X¹, X², X³, and X⁴ is C covalently bonded to ring B via L; and the remaining of X¹, X², X³, and X⁴ are each independently N or CR⁴; provided that no more than two of X¹, X², X³, and X⁴ are N;
X⁵, X⁶ and X⁷ are each independently N or CR⁵;
X⁸ and X⁹ are each independently N or CR⁶;
X¹⁰ is N or CR⁶ and X¹ is S, O, or NR⁹;
n is 1, 2, or 3;
m is 0, 1, 2, 3, 4, or 5;
R¹ is —C(O)OR⁹, —C(O)N(R⁹)₂, —C(O)N(R⁹)S(O)₂R⁹, —NR⁹C(O)R⁹, 5- to 10-membered heterocyclyl or 5- to 10-membered heteroaryl, wherein the 5- to 10-membered heteroaryl or 5- to 10-membered heterocyclyl is optionally substituted with 1-4 R¹¹;
R² is C₁₋₉ alkyl optionally substituted with —O—(C₁₋₉ alkyl), —S—(C₁₋₉ alkyl), —S(O)₂—(C₁₋₉ alkyl), C₃₋₆ cycloalkyl, 3- to 6-membered heterocyclyl, phenyl, or 5- to 6-membered heteroaryl; wherein each C₁₋₉ alkyl, —O—(C₁₋₉ alkyl), —S—(C₁₋₉alkyl), —S(O)₂—(C₁₋₉ alkyl), C₃₋₆ cycloalkyl, 3- to 6-membered heterocyclyl, phenyl, or 5- to 6-membered heteroaryl of R² is further optionally substituted with one to five Z¹;
L is a bond, C₉ alkylene, C₂₋₉ alkenylene, C₂₋₉ alkynylene, —O—C₁₋₉ alkylene, —NR¹⁰—C₁₋₉ alkylene, —C(O)NR¹⁰—C₁₋₉alkylene, —NR¹⁰C(O)—C₁₋₉ alkylene, 3- to 6-membered heterocyclylene, —O—, —S—, —S(O)—, —S(O)₂—, —NR¹⁰—, —C(O)NR¹⁰—, —NR¹⁰C(O)—, —C(O)—, —OC(O)—, —C(O)O—, —NR¹⁰S(O)—, —S(O)NR¹⁰—, —NR¹⁰S(O)NR¹⁰—, —NR¹⁰S(O)₂NR¹⁰—, —NR¹⁰C(O)NR¹⁴—, —OC(O)NR¹⁰—, or —NR¹⁰C(O)O—; wherein each C₁₋₉ alkylene, C₂₋₉ alkenylene, C₂₋₉ alkynylene, —O—C₁₋₉ alkylene, —NR¹⁰—C₁₋₉ alkylene, —C(O)NR¹⁴—C₁₋₉ alkylene, —NR¹⁰C(O)—C₁₋₉ alkylene, or 3- to 6-membered heterocyclylene of L is independently optionally substituted with one to five Z¹;
each R³ is independently halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹⁰C(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R³ is independently optionally substituted with one to five Z¹;
each R⁴ is independently hydrogen, halo, cyano, nitro, oxo, —OR¹⁰, —SR¹⁰, —N(R¹⁰)₂, —C(O)R¹⁰, —C(O)OR¹⁰, —OC(O)R¹⁰, —OC(O)OR¹⁰, —C(O)N(R¹⁰)₂, —NR¹⁰C(O)R¹⁰, —OC(O)N(R¹⁰)₂, —NR¹⁰C(O)OR¹⁰, —NR¹⁰C(O)N(R¹⁰)₂, —S(O)R¹⁰, —S(O)₂R¹⁰, —S(O)N(R¹⁰)₂, —S(O)₂N(R¹⁰)₂, —NR¹⁰S(O)R¹⁰, —NR¹⁰S(O)₂R¹⁰, —NR¹⁰S(O)N(R¹⁰)₂, —NR¹⁰S(O)₂N(R¹⁰)₂, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R⁴ is independently optionally substituted with one to five Z¹;
each R⁵ is independently hydrogen, halo, cyano, nitro, oxo, —OH, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl; wherein each —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, or C₂₋₆ alkynyl of R⁵ is independently optionally substituted with one to five substituents independently selected from halo, hydroxy, and cyano;
each R⁶ is independently hydrogen, halo, cyano, nitro, oxo, —OH, —SH, —NH₂, —NH—C₁₋₆ alkyl, —N(C₁₋₆ alkyl)₂, —S—C₁₋₆ alkyl, C₁₋₆ alkoxy, C₁₋₆, alkyl, C₂₋₆ alkenyl, or C₂₋₆ alkynyl; wherein each —NH—C₁₋₆ alkyl, —N(C₁₋₆ alkyl)₂, —S—C₁₋₆ alkyl, C₁₋₆ alkoxy, C₁₋₆, alkyl, C₂₋₆ alkenyl, or C₂₋₆ alkynyl of R⁶ is independently optionally substituted with one to five substituents independently selected from halo, hydroxy, and cyano;
each R⁸ is independently hydrogen or C₁₋₉ alkyl;
each R⁹ is independently hydrogen, C₁₋₉ alkyl, C₂₋₉ alkenyl, C₂₋₉ alkynyl, C₃₋₁₀ cycloalkyl, heterocyclyl, aryl, heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R⁹ is independently optionally substituted with one to five R¹¹;
each R¹⁰ is independently hydrogen, C₁₋₉ alkyl, C₂₋₉ alkenyl, C₂₋₉ alkynyl, C₃₋₁₀ cycloalkyl, heterocyclyl, aryl, heteroaryl, —C(O)R²⁰, —C(O)OR²⁰, —C(O)N(R²⁰)₂, —S(O)R²⁰, —S(O)₂R²⁰, —S(O)N(R²⁰)₂, or —S(O)₂N(R²⁰)₂; wherein each C₁₋₉ alkyl, C₂₋₉ alkenyl, C₂₋₉ alkynyl, C₃₋₁₀ cycloalkyl, heterocyclyl, aryl, or heteroaryl of R¹⁰ is independently optionally substituted with one to five Z^{1a};
each R¹¹ is independently oxo, cyano, halo, hydroxy, C₁₋₉alkyl, C₁₋₉ alkoxy, C₁₋₉haloalkyl, C₁₋₉ haloalkoxy, C₃₋₉ cycloalkyl, —C₁₋₉alkyl-C(O)OR¹², —C(O)OR², —C(O)N(R¹²)₂, —SR², or —S(O)₂R¹²;
each R¹² is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R¹² is independently optionally substituted with one to five Z^{1a};
each Z¹ is independently halo, cyano, nitro, oxo, C₁₋₉ alkyl, C₂₋₉ alkenyl, C₂₋₉ alkynyl, C₃₋₁₀ cycloalkyl, heterocyclyl, aryl, heteroaryl, -L¹-C₁₋₉ alkyl, -L¹-C₂₋₉ alkenyl, -L¹-C₂₋₉ alkynyl, -L¹-C₃₋₁₀ cycloalkyl, -L¹-heterocyclyl, -L¹-aryl, or -L¹-heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of Z¹ is independently optionally substituted with one to five Z^{1a};
each L¹ is independently —O—, —S—, —NR²⁰—, —C(O)—, —C(O)O—, —OC(O)—, —OC(O)O—, —C(O)NR²⁰—, —NR²⁰C(O)—, —OC(O)NR²⁰—, —NR²⁰C(O)O—, —NR²⁰C(O)NR²⁰—, —S(O)—, —S(O)₂—, —S(O)NR²⁰—, —S(O)₂NR²⁰—, —NR²⁰S(O)—, —NR²⁰S(O)₂—, —NR²⁰S(O)NR²⁰—, or —NR²⁰S(O)₂NR²⁰—;
each R²⁰ is independently hydrogen, C₁₋₉ alkyl, C₂₋₉ alkenyl, C₂₋₉ alkynyl, C₃₋₁₀ cycloalkyl, heterocyclyl, aryl, or heteroaryl; wherein each C₁₋₉ alkyl, C₂₋₉ alkenyl, C₂₋₉ alkynyl, C₃₋₁₀ cycloalkyl, heterocyclyl, aryl, or heteroaryl of R²⁰ is independently optionally substituted with one to five Z^{1a};
each Z^{1a} is independently halo, hydroxy, cyano, nitro, oxo, —SH, —NH₂, —NH—C₁₋₆ alkyl, —N(C₁₋₆ alkyl)₂, —S—C₁₋₆ alkyl, C₁₋₆ alkoxy, C₁₋₆ alkyl, C₂₋₆ alkenyl, C₂₋₆ alkynyl, C₃₋₆ cycloalkyl, heterocyclyl, aryl, or heteroaryl; wherein each C₁₋₆ alkyl, C₂₋₆ alkenyl, C₂₋₆ alkynyl, C₃₋₆ cycloalkyl, heterocyclyl, aryl, or heteroaryl of Z^{1a} is independently optionally substituted with one to five substituents selected from C₁₋₉ alkyl, oxo, halo, hydroxy, and cyano;
provided that when ring A is 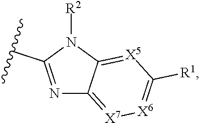
then R¹ is other than —C(O)OH.

2. The compound of claim 1, represented by Formula II: 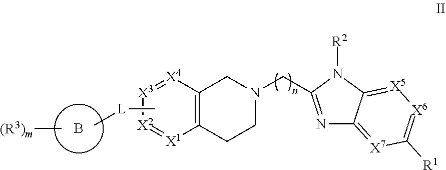
or a pharmaceutically acceptable salt or solvate thereof.

3. The compound of claim 1 or 2, wherein X⁶ is N.

4. The compound of any one of claims 1-3, wherein X⁵ is N.

5. The compound of any one of claims 1-3, wherein X⁵ is N or CR⁵, X⁶ is N, and X⁷ is CR⁵.

6. The compound of claim 1, represented by Formula III: 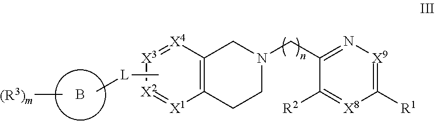
or a pharmaceutically acceptable salt or solvate thereof.

7. The compound of claim 1 or 6, wherein X⁹ is CR⁶.

8. The compound of any one of claims 1, 6, or 7, wherein X⁹ is N.

9. The compound of any one of claims 1, 6, or 7, wherein X⁸ is CR⁶, and X⁹ is N or CR⁶.

10. The compound of claim 1, represented by Formula IV: 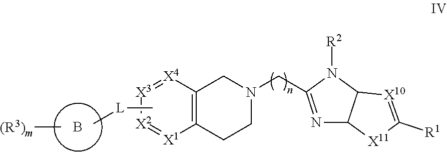
or a pharmaceutically acceptable salt or solvate thereof.

11. The compound of claim 1 or 10, wherein X¹⁰ is CR⁷.

12. The compound of any one of claims 1, 10, or 11, wherein X¹¹ is S.

13. The compound of claim 1, represented by Formula V: 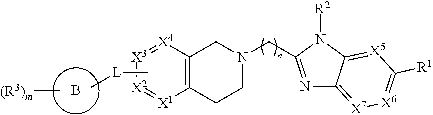
or a pharmaceutically acceptable salt or solvate thereof.

14. The compound of claim 1 or 13, wherein X⁵, X⁶, and X⁷ are CR⁷.

15. The compound of any one of claims 1-14, wherein n is 1.

16. The compound of any one of claims 1-15, wherein R¹ is 5-membered heteroaryl, optionally substituted with one to five R¹¹.

17. The compound of any one of claims 1-16, wherein R¹ is

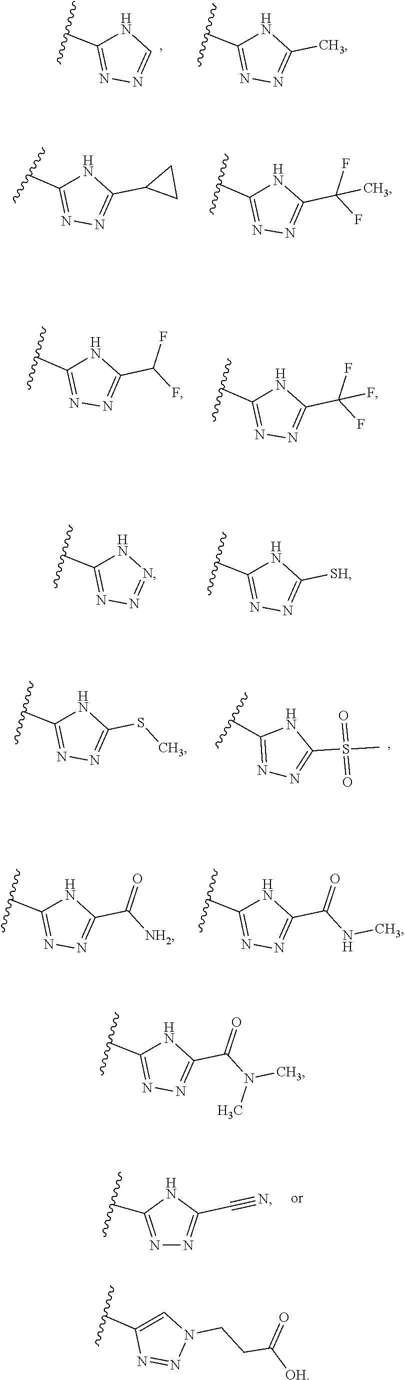

18. The compound of any one of claims 1-15, wherein R¹ is 5- to 10-membered heterocyclyl optionally substituted with one to five R¹¹, wherein the heterocyclyl comprises an endocyclic

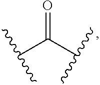 [SMILES: *C(*)=O]

19. The compound of any one of claims 1-15 or 18, wherein R¹ is

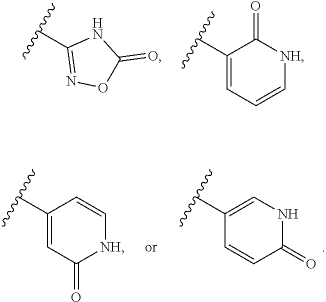 [SMILES: *c1noc(=O)[nH]1 ; *c1ccc[nH]c1=O ; *c1cc[nH]c(=O)c1 ; *c1ccc(=O)[nH]c1]

20. The compound of any one of claims 1-15, wherein R¹ is —C(O)OR⁹, —C(O)N(R⁹)₂,
—C(O)N(R⁹)S(O)₂R⁹, or —NR⁹C(O)R⁹.

21. The compound of any one of claims 1-15 or 20, wherein R¹ is —C(O)OH.

22. The compound of any one of claims 1-15 or 20, wherein R¹ is —C(O)NH₂.

23. The compound of any one of claims 1-15, wherein R¹ is —C(O)NHR⁹, or —NHC(O)R⁹, and R⁹ is C₁₋₉ alkyl, C₃₋₁₀ cycloalkyl, or heteroaryl; wherein the C₁₋₉ alkyl, C₃₋₁₀ cycloalkyl, or heteroaryl is independently optionally substituted with one to five R¹¹.

24. The compound of any one of claims 1-15 or 23, wherein R¹ is —C(O)NHR⁹, or —NHC(O)R⁹, and R⁹ is methyl, 2,2,2-trifluoroethyl, cyclopropyl substituted with cyano, or pyridyl.

25. The compound of any one of claims 1-15 or 20, wherein R¹ is —C(O)NHS(O)₂R⁹, and R⁹ is methyl.

26. The compound of any one of claims 1-19, wherein one of X¹, X², and X³ is C covalently bonded to ring B via L; X⁴ is N; and the remaining of X¹, X², and X³, are each independently CR⁴.

27. The compound of any one of claims 1-19, wherein one of X¹, X², X³, and X⁴ is C covalently bonded to ring B via L; and the remaining of X¹, X², X³, and X⁴ are each independently CR⁴.

28. The compound of any one of claims 1-27, wherein X³ is C covalently bonded to ring B via L.

29. The compound of any one of claims 1-28, wherein each R⁴ is independently hydrogen, halo, C₁₋₉ haloalkyl, or C₃₋₁₀ cycloalkyl.

30. The compound of any one of claims 1-29, wherein X² is CR⁴, and R⁴ is hydrogen, halo, C₁₋₉ haloalkyl, or C₃₋₁₀ cycloalkyl.

31. The compound of any one of claims 1-30, wherein each R⁴ is independently hydrogen, chloro, —CF₃, or cyclopropyl.

32. The compound of any one of claims 1-31, wherein X¹ is CH.

33. The compound of any one of claims 1-32, wherein each R⁵ is independently hydrogen or halo.

34. The compound of any one of claims 1-33, wherein each R⁶ is independently hydrogen or halo.

35. The compound of any one of claims 1-24, wherein L is —O—C₁₋₉ alkylene, —NR¹⁰—C₁₋₉ alkylene, —C(O)NR¹⁰—C₁₋₉ alkylene, or —NR¹⁰C(O)—C₁₋₉ alkylene.

36. The compound of any one of claims 1-35, wherein L is —O—CH₂—.

37. The compound of any one of claims 1-36, wherein ring B is C₃₋₆ cycloalkyl, phenyl, a 5- or 9-membered heterocyclyl, or a 5- or 9-membered heteroaryl.

38. The compound of any one of claims 1-37, wherein ring B is phenyl.

39. The compound of claim 1, represented by Formula IIG: 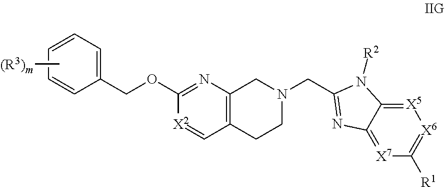
or a pharmaceutically acceptable salt or solvate thereof.

40. The compound of claim 1, represented by Formula HID: 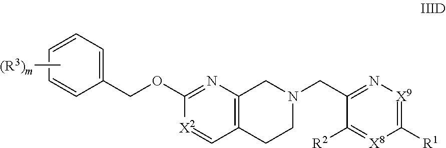
or a pharmaceutically acceptable salt or solvate thereof.

41. The compound of claim 1, represented by Formula IVD: 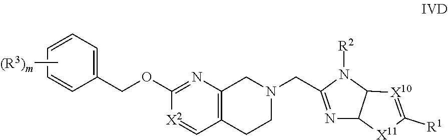
or a pharmaceutically acceptable salt or solvate thereof.

42. The compound of claim 1, represented by Formula VD: 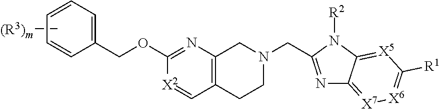
or a pharmaceutically acceptable salt or solvate thereof.

43. The compound of any one of claims 1-42, wherein R² is C₁₋₉ alkyl, C₁₋₉ alkyl substituted with a 3- to 6-membered heterocyclyl, or C₁₋₉ alkyl substituted with a C₃₋₆ cycloalkyl which is substituted with cyano.

44. The compound of any one of claims 1-43, wherein R² is C₁₋₉ alkyl.

45. The compound of any one of claims 1-44, wherein R² is methyl.

46. The compound of any one of claims 1-43, wherein R² is C₁₋₉ alkyl substituted with 3- to 6-membered heterocyclyl or C₁₋₉ alkyl substituted with a C₃₋₆ cycloalkyl which is substituted with cyano.

47. The compound of any one of claims 1-43 or 46, wherein R² is

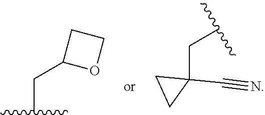 [SMILES: *CC1CCO1 ; *CC1(C#N)CC1]

48. The compound of any one of claims 1-47, wherein m is 1, 2, or 3.

49. The compound of any one of claims 1-48, wherein each R³ is independently halo, cyano, —OR¹⁰, —C(O)N(R¹⁰)₂, —S(O)₂R¹⁰, C₁₋₉ alkyl, C₃₋₁₀ cycloalkyl, or heteroaryl; wherein each C₁₋₉ alkyl of R³ is independently optionally substituted with one to five halo.

50. The compound of any one of claims 1-49, wherein each R³ is independently halo.

51. A compound selected from Table 1, or a pharmaceutically acceptable salt, stereoisomer, mixture of stereoisomers, or prodrug thereof.

52. A pharmaceutical composition comprising a compound of any preceding claim, or a pharmaceutically acceptable salt or solvate thereof, and a pharmaceutically acceptable excipient.

53. A method for treating a GLP-1 associated disease, disorder, or condition, the method comprising administering to a patient in need thereof an effective amount of a compound of any one of claims 1-51, or a pharmaceutically acceptable salt or solvate thereof, or the pharmaceutical composition according to claim 52.

54. The method of claim 53, wherein the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof.

55. A method of treating type 2 diabetes mellitus in a patient in need thereof, the method comprising administering to a patient in need thereof an effective amount of a compound of any one of claims 1-51, or a pharmaceutically acceptable salt or solvate thereof, or the pharmaceutical composition according to claim 52.

56. A method for modulating insulin levels in a patient in need of such modulating, the method comprising administering to a patient in need thereof an effective amount of a compound of any one of claims 1-51, or a pharmaceutically acceptable salt or solvate thereof, or the pharmaceutical composition according to claim 52.

57. A method for modulating glucose levels in a patient in need of such modulating, the method comprising administering to a patient in need thereof an effective amount of a compound of any one of claims 1-51, or a pharmaceutically acceptable salt or solvate thereof, or the pharmaceutical composition according to claim 52.

58. The method of any one of claims 53-57, further comprising administering an additional therapy or therapeutic agent to the patient.

59. The method of claim 58, wherein the additional therapy or therapeutic agent is selected from the group consisting of an antidiabetic agent, an anti-obesity agent, a GLP-1 receptor agonist, an anti-emetic agent, an agent to treat non-alcoholic steatohepatitis (NASH), gastric electrical stimulation, dietary monitoring, physical activity, or a combination thereof.

60. A process for preparing the compound of Formula I as in claim 1, or a pharmaceutically acceptable salt or solvate thereof, comprising contacting a compound of Formula II-1, III-1, IV-1, or V-1: 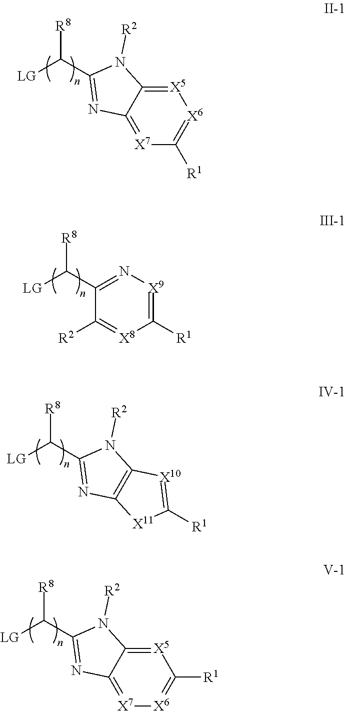
with a compound of Formula I-1: 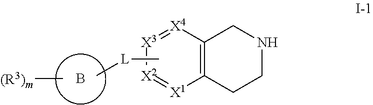
under conditions sufficient to provide the compound of Formula I, or a pharmaceutically acceptable salt or solvate thereof.
